(19) United States
(12) Patent Application Publication
(10) Pub. No.: US 2023/0405002 A1
(43) Pub. Date: Dec. 21, 2023

(54) COMBINATIONS OF DIACYLGLYCEROL ACYLTRANSFERASE 2 INHIBITORS AND ACETYL-COA CARBOXYLASE INHIBITOR

(71) Applicant: Pfizer Inc., New York, NY (US)

(72) Inventors: Neeta Balkrishan Amin, Cambridge, MA (US); Arthur James Bergman, Salem, CT (US); Roberto Arnaldo Calle, East Lyme, CT (US); Robert Gregory Dullea, Groton, MA (US); David James Edmonds, Riehen (CH); William Paul Esler, Clinton, CT (US); Kevin James Filipski, Reading, MA (US); James Richard Gosset, Concord, MA (US); Albert Myung Kim, Newton, MA (US); Jeffrey Allen Pfefferkorn, Acton, MA (US); Patrick Robert Verhoest, Newton, MA (US)

(73) Assignee: Pfizer Inc., New York, NY (US)

(21) Appl. No.: 17/904,554

(22) PCT Filed: Feb. 22, 2021
(86) PCT No.: PCT/IB2021/051478

§ 371 Date: Aug. 18, 2022

Related U.S. Application Data
(60) Provisional application No. 62/980,845, filed on Feb. 24, 2020.

(51) Int. Cl. A61K 31/506; A61K 31/4545; A61P 1/16; A61P 3/04

(52) U.S. Cl. CPC A61K 31/506; A61K 31/4545; A61P 1/16; A61P 3/04

(57) ABSTRACT

Described herein are pharmaceutical compositions comprising 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, for treatment of liver disease and diseases related thereto. Also described are compositions comprising 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof and 4-(4-(1-Isopropyl-7-oxo-1,4,6,7-tetrahydrospiro[indazole-5,4′-piperidine]-1′-carbonyl)-6-methoxypyridin-2-yl)benzoic acid, or pharmaceutically acceptable salt thereof, for treatment of liver disease and diseases related thereto.

### FIELD OF THE INVENTION

The invention relates to new pharmaceutical compositions comprising 2-{5-[(3-ethoxypyridin-2-yl)oxy]pyridin-3-yl}-N-[(3S,5S)-5-fluoropiperidin-3-yl]pyrimidine-5-carboxamide, or a pharmaceutically acceptable salt thereof, for treatment of liver disease and diseases related thereto. The invention also relates to a new pharmaceutical compositions comprising 2-{5-[(3-ethoxypyridin-2-yl)oxy]pyridin-3-yl}-N-[(3S,5S)-5-fluoropiperidin-3-yl]pyrimidine-5-carboxamide, or a pharmaceutically acceptable salt thereof and 4-(4-(1-Isopropyl-7-oxo-1,4,6,7-tetrahydrospiro[indazole-5,4′-piperidine]-1′-carbonyl)-6-methoxypyridin-2-yl)benzoic acid, or pharmaceutically acceptable salt thereof, for treatment of liver disease and diseases related thereto.

### BACKGROUND OF THE INVENTION

Nonalcoholic steatohepatitis (NASH) is a clinical and histological subset of non-alcoholic fatty liver disease (NAFLD, defined as presence of ≥5% hepatic steatosis) that is associated with increased all cause mortality, cirrhosis and end stage liver disease, increased cardiovascular mortality, and increased incidence of both liver related and non-liver related cancers (Sanyal et al, Hepatology 2015; 61(4):1392-1405). NAFLD is the hepatic manifestation of metabolic syndrome, and is a spectrum of hepatic conditions encompassing steatosis, NASH, fibrosis, cirrhosis and ultimately hepatocellular carcinoma. NAFLD and NASH are considered the primary fatty liver diseases as they account for the greatest proportion of individuals with elevated hepatic lipids. The severity of NAFLD/NASH is based on the presence of lipid, inflammatory cell infiltrate, hepatocyte ballooning, and the degree of fibrosis. At the present time, treatment options are limited to management of associated conditions (EASL-EASD-EASO Clinical Practice Guidelines, J. Hepatol. 2016; 64(6):1388-1402).

Alterations in lipid metabolism have been hypothesized to contribute to the molecular pathogenesis of NAFLD and NASH. Steatosis is a necessary but not sufficient component of the pathogenesis of NASH (Day C, and James O., Hepatology. 1998; 27(6):1463-6). Consistent with this, multiple studies have demonstrated that the severity of steatosis predicts the risk of concomitant steatohepatitis as well as the risk of progression to cirrhosis (Sorensen et al, Lancet. 1984; 2(8397): 241-4; Wanless I and Lentz J, Hepatology 1990; 12(5):1106-10; Reeves H, et al, J. Hepatol. 1996; 25(5): 677-83). Hepatic steatosis is a consequence of an imbalance in triglyceride production/uptake into the liver and clearance/removal (Cohen J C, et al, Science. 2011; 332(6037):1519-1523). It is hypothesized that reducing steatosis, the metabolic driver underpinning the development of NAFLD/NASH, will result in subsequent improvements in hepatic inflammation and fibrosis.

Acetyl-CoA Carboxylase (ACC) and diacylglycerol acyltransferase 2 (DGAT2) are two key enzymes regulating lipid metabolism. ACC catalyzes an essential and rate limiting step in the process of de novo lipogenesis (DNL) (Saggerson D, Annu. Rev. Nutr. 2008; 28:253-72). Further, ACC also regulates mitochondrial beta-oxidation of fatty acids through allosteric regulation of the enzyme carnitine palmitoyltransferase 1 (CPT1) (Saggerson, 2008; Waite M, and Wakil S J. J. Biol. Chem. 1962; 237:2750-2757). Emerging data also suggest that suppression of DNL through ACC inhibition may directly reduce inflammation by restraining the formation of the inflammatory interleukin-17 (IL-17) secreting T-cells of the T helper 17 lineage (Th17 cells) and favoring the development of anti-inflammatory FoxP3(+) regulatory T (Treg) cells (Berod L, et al. Nat. Med. 2014; 20(11): 1327-33).

Inhibition of ACC activity is hypothesized to be beneficial to patients with NASH by at least two independent mechanisms. As summarized above, humans with NAFLD show marked elevations in hepatic DNL and normalization of this increased flux through pharmacologic hepatic ACC inhibition is hypothesized to reduce steatosis. In addition, the effect of ACC inhibitors to increase fatty acid oxidation may also contribute to reduce liver fat content. Consistent with this, ACC inhibitors have been shown to inhibit DNL. See Griffith D A, et al. J. Med. Chem. 2014; 57(24):10512-10526; Kim C W, et al. Cell Metab. 2017; 26, 394-406; Stiede K, et al. Hepatology. 2017; 66(2):324-334; Lawitz E J, et al. Clin Gastroenterol Hepatol. 2018 (https://doi.org/10.1016/j.cgh.2018.04.042). In addition, inhibition of DNL in IL-17 secreting T-cells is expected to suppress hepatic inflammation by restraining the formation of the inflammatory Th17 cells (Berod et al., 2014), a pathway that may be important in NASH pathogenesis (Rau M, et al. J. Immunol. 2016; 196(1):97-105), and favoring the development of anti-inflammatory Treg cells. Further, ACC inhibition may reduce stellate cell activation and fibrosis (Ross et al., 2019).

Triglycerides or triacylglycerols (TG) represent a major form of energy storage in mammals. TG's are formed by the sequential esterification of glycerol with three fatty acids of varying chain lengths and degrees of saturation (Coleman, R. A., and Mashek, D. G. 2011. Chem. Rev. 111: 6359-6386). TG synthesized in the intestine or liver are packaged into chylomicrons or very low-density lipoprotein (VLDL), respectively, and exported to peripheral tissues where they are hydrolyzed to their constituent fatty acids and glycerol by lipoprotein lipase (LPL). The resultant non-esterified fatty acids (NEFA) can either be metabolized further to produce energy or reesterified and stored.

Under normal physiological conditions, the energy-dense TG remains sequestered in various adipose depots until there is a demand for its release, whereupon, it is hydrolyzed to glycerol and free fatty acids which are then released into the blood stream. This process is tightly regulated by the opposing actions of insulin and hormones such as catecholamines which promote the deposition and mobilization of TG stores under various physiological conditions. In the post-prandial setting, insulin acts to inhibit lipolysis, thereby, restraining the release of energy in the form of NEFA and ensuring the appropriate storage of dietary lipids in adipose depots. However, in patients with type 2 diabetes, the ability of insulin to suppress lipolysis is ameliorated and NEFA flux from adipocytes is inappropriately elevated. This, in turn, results in increased delivery of lipid to tissues such as muscle and liver. In the absence of energetic demand the TG and other lipid metabolites, such as diacylglycerol (DAG) can accumulate and cause a loss of insulin sensitivity (Erion, D. M., and Shulman, G. I. 2010. Nat Med 16: 400-402). Insulin resistance in muscle is characterized by reduced glucose uptake and glycogen storage, whilst in the liver, loss of insulin signaling gives rise to dysregulated glucose output and over-production of TG-rich VLDL, a hallmark of type 2 diabetes (Choi, S. H., and Ginsberg, H. N. 2011. Trends Endocrinol. Metab. 22: 353-363). Elevated secretion of TG-enriched VLDL, so called VLDL1 particles, is thought to stimulate the production of small, dense low-density lipoprotein (sdLDL), a proatherogenic subfraction of LDL that is associated with elevated risk of coronary heart disease (St-Pierre, A. C. et. al. 2005. Arterioscler. Thromb. Vasc. Biol. 25: 553-559).

Diacylglycerol acyltransferases (DGAT) catalyze the terminal step in triacylglyceride (TAG) synthesis, specifically, the esterification of a fatty acid with diacylglycerol resulting in the formation of TAG. In mammals, two DGAT enzymes (DGAT1 and DGAT2) have been characterized. Although these enzymes catalyze the same enzymatic reaction their respective amino acid sequences are unrelated and they occupy distinct gene families. DGAT2 is highly expressed in liver and adipose, and unlike DGAT1, exhibits exquisite substrate specificity for diacylglyceride (DAG). Deletion of the DGAT2 gene in rodents results in defective intrauterine growth, severe lipemia, impaired skin barrier function, and early post-natal death. It is clear that suppression of DGAT2 results in a down-regulation of the expression of multiple genes encoding proteins involved in lipogensis, including sterol regulatory element-binding proteins 1c (SREBP1c) and stearoyl CoA-desaturase 1 (SCD1). In parallel, oxidative pathways are induced as evidenced by increased expression of genes such as carnitine palmitoyl transfersase 1 (CPT1). The net result of these changes is to decrease the levels of hepatic DAG and TAG lipid which, in turn, leads to improved insulin responsiveness in the liver. Furthermore, DGAT2 inhibition suppresses hepatic VLDL TAG secretion and leads to reduction in circulating cholesterol levels. Finally, plasma apolipoprotein B (APOB) levels are suppressed, possibly due to decreased supply of TAG for lipidation of the newly synthesized APOB protein. The beneficial effects of DGAT2 inhibition on both glycemic control and plasma cholesterol profile suggest that this target is valuable in the treatment of metabolic disease (Choi, C. S. et. al. 2007. J Biol Chem 282: 22678-22688).

In addition, the observation that suppression of DGAT2 activity results in reduced hepatic lipid accumulation suggests that inhibitors of this enzyme might have utility in the treatment of non-alcoholic steatohepatitis (NASH), a highly prevalent liver disease characterized by the deposition of excess fat in the liver.

In recent years, several small molecule inhibitors of DGAT2 have been reported in the literature and patent applications (WO2013150416, WO2013137628, US20150259323, WO2015077299, WO2016036633, WO2016036638, WO2016036636). Recently, commonly assigned PCT application PCT/IB2017/054862 was published as WO2018/033832 on Feb. 22, 2018 disclosing small molecule inhibitors of DGAT2.

Nevertheless, there remains a need for pharmaceutical agents that have DGAT2 inhibiting activity and are useful in the treatment, prevention or diminution of the manifestations of the maladies described herein. Moreover, there remains a need for medicaments, for example, oral medicaments, containing a combination of the DGAT2 compounds described herein (DGAT2 inhibitors), and in the same or a separate composition comprising the ACC inhibitor 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof. The specific combinations described herein satisfy the existing need.

### SUMMARY OF THE INVENTION

The present invention is directed to a pharmaceutical composition comprising a therapeutically effective amount of from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and a pharmaceutically acceptable excipient.

The present invention is also directed to a pharmaceutical composition comprising 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

The present invention is also directed to a pharmaceutical composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and a therapeutically effective amount of from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

The present invention is also directed to a method for treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma, the method comprising administering to a human in need of such treatment a first composition comprising a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof in combination with a second composition comprising a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

It is to be understood that both the foregoing general description and the following detailed description are exemplary and explanatory only and are not restrictive of the invention, as claimed.

### BRIEF DESCRIPTION OF THE DRAWINGS

FIG. 1 shows an illustrative PXRD pattern of Form 1 of Example 26 (Compound A) carried out on a Bruker AXS D4 Endeavor diffractometer equipped with a Cu radiation source.

FIG. 2 shows an illustrative Raman spectra of Form 1 of Example 26 (Compound A) collected using a Nicolet NXR FT-Raman accessory attached to the FT-IR bench.

FIG. 3 shows an illustrative ¹³C ssNMR pattern of Form 1 of Example 26 (Compound A) conducted on a Bruker-BioSpin CPMAS probe positioned into a Bruker-BioSpin Avance III 500 MHz (¹H frequency) NMR spectrometer.

FIG. 4 shows an illustrative PXRD pattern of Form 2 of Example 26 (Compound A) carried out on a Bruker AXS D4 Endeavor diffractometer equipped with a Cu radiation source.

FIG. 5 shows an illustrative Raman spectra of Form 2 of Example 26 (Compound A) collected using a Nicolet NXR FT-Raman accessory attached to the FT-IR bench.

FIG. 6 shows an illustrative ¹³C ssNMR pattern of Form 2 of Example 26 (Compound A) conducted on a Bruker-BioSpin CPMAS probe positioned into a Bruker-BioSpin Avance III 500 MHz (¹H frequency) NMR spectrometer.

FIG. 7 shows an illustrative single crystal structure of Form 2 of Example 26 (Compound A).

FIG. 8 summarizes the effects of oral administration as monotherapy and in combination of Compound A and Compound D on plasma triglyceride levels in Western diet fed Sprague Dawley rats, measured at the fed state.

FIG. 9 summarizes the effects of oral administration as monotherapy and in combination Compound A and Compound D on plasma triglyceride levels in Western diet fed Sprague Dawley rats measured at the fasted state.

FIG. 10 summarizes the effect of administration of Compound A and Compound D as monotherapy and in combination on SREBP-1 nuclear localization in Western diet fed rats.

FIG. 11 summarizes the effect of administration of Compound A and Compound D as monotherapy and in combination on hepatic lipogenic gene expression in Western diet fed rats, specifically acetyl-CoA carboxylase (ACC1).

FIG. 12 summarizes the effect of administration of Compound A and Compound D as monotherapy and in combination on hepatic lipogenic gene expression in Western diet fed rats, specifically fatty acid synthase (FASN).

FIG. 13 summarizes the effect of administration of Compound A and Compound D as monotherapy and in combination on hepatic lipogenic gene expression in Western diet fed rats, specifically sterol-CoA desaturase (SCD1).

FIG. 14 summarizes the effect of administration of Compound A and Compound D as monotherapy and in combination on hepatic lipogenic gene expression in Western diet fed rats, specifically sterol regulatory element-binding protein 1c (SREBP-1c).

FIG. 15 summarizes the effect of administration of Compound A and Compound D as monotherapy and in combination on hepatic lipogenic gene expression in Western diet fed rats, specifically proprotein convertase subtilisin/kexin type 9 (PCSK9).

FIG. 16 summarizes the effects of oral administration as monotherapy and in combination of Compound A and Compound D on hepatic triglyceride levels in Western diet fed Sprague Dawley rats.

FIG. 17 summarizes the effects of oral administration as monotherapy and in combination of Compound A and Compound D on elasticity of the liver, a marker of hepatic inflammation and fibrosis, in choline deficient and high fat diet (CDAHFD) fed Male Wistar Hann rats.

FIG. 18 summarizes the effects of oral administration as monotherapy and in combination of Compound A and Compound D on hepatic alpha smooth actin (αSMA) immunohistochemistry, a marker of myofibroblast activation and fibrogenesis, in CDAHFD fed Male Wistar Hann rats.

FIG. 19 summarizes the effects of oral administration as monotherapy and in combination of Compound A and Compound D on hepatic Picosirius red staining in CDAHFD fed Male Wistar Hann rats.

FIG. 20 summarizes the effects of oral administration as monotherapy and in combination of Compound A and Compound D on hepatic alpha smooth actin (αSMA) gene expression in CDAHFD fed Male Wistar Hann rats.

FIG. 21 summarizes the effects of oral administration as monotherapy and in combination of Compound A and Compound D on hepatic collagen 1A1 gene expression in CDAHFD fed Male Wistar Hann rats.

FIG. 22 summarizes the effects of oral administration as monotherapy and in combination of Compound A and Compound D on Ionized Calcium-Binding Adapter Molecule 1 Staining in CDAHFD fed Male Wistar Hann rats.

FIG. 23a shows a Box-and-Whisker plot of the WLF data by treatment arm for the Phase 2A study described herein.

FIG. 23b shows % of subjects with greater than or equal to 30% liver fat reduction.

FIG. 23c shows % of subjects with greater than or equal to 50% liver fat reduction.

FIG. 24 shows a plot of least square means and 90% Cls for percent change from baseline in serum triglycerides for the Phase 2A study described herein.

FIG. 25a shows a plot of least square means and 90% Cls for percent change from baseline in alaninine aminotransferase (ALT) for the Phase 2A study described herein.

FIG. 25b shows a plot of least square means and 90% Cls for percent change from baseline in aspartate aminotransferase (AST) for the Phase 2A study described herein.

FIG. 25c shows a plot of least square means and 90% Cls for percent change from baseline in alkaline phosphotase for the Phase 2A study described herein.

FIG. 25d shows a plot of least square means and 90% Cls for percent change from baseline in gamma glutamyl transferase (GGT) for the Phase 2A study described herein.

FIG. 26 is a characteristic x-ray powder diffraction pattern showing Example 4, Form 1 (Vertical Axis: Intensity (CPS); Horizontal Axis: Two theta (degrees)).

FIG. 27 is a characteristic x-ray powder diffraction pattern showing Example 4, hydrochloride salt Form 1 (Vertical Axis: Intensity (CPS); Horizontal Axis: Two theta (degrees)).

FIG. 28 is a characteristic x-ray powder diffraction pattern showing Example 4, p-toluenesulfonate salt, Anhydrous, Form 1 (Vertical Axis: Intensity (CPS); Horizontal Axis: Two theta (degrees)).

FIG. 29 plots the multiple dose effects of Example 4 on plasma triglyceride in Western diet fed Sprague-Dawley rats (Vertical Axis: plasma triglyceride (mg/dL), Horizontal Axis: Western Diet BID dosing (mg/kg)).

FIG. 30 plots the multiple dose effects of Example 4 on hepatic triglyceride in Western diet fed Sprague-Dawley rats (Vertical Axis: hepatic triglyceride (μg/mg), Horizontal Axis: Western Diet BID dosing (mg/kg)).

FIG. 31 is a characteristic x-ray powder diffraction pattern showing Preparation P1, Form 1 (Vertical Axis: Intensity (CPS); Horizontal Axis: Two theta (degrees)).

### DETAILED DESCRIPTION OF THE INVENTION

The present invention may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers. For the field of this disclosure, this level of approximation is appropriate unless the value is specifically stated to require a tighter range.

“Compounds” when used herein includes the compounds described herein or any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

The term “alkyl”, alone or in combination, means an acyclic, saturated hydrocarbon group of the formula CnH2n+1 which may be linear or branched. Examples of such groups include, but are not limited to, methyl, ethyl, n-propyl, isopropyl, butyl, sec-butyl, isobutyl and t-butyl. The carbon atom content of alkyl and various other hydrocarbon-containing moieties is indicated by a prefix designating a lower and upper number of carbon atoms in the moiety, that is, the prefix Ci-Cj indicates a moiety of the integer “i” to the integer “j” carbon atoms, inclusive. Thus, for example, C₁-C₃ alkyl refers to alkyl of one to three carbon atoms, inclusive.

“Fluoroalkyl” means an alkyl as defined herein substituted with one, two or three fluoro atoms. Exemplary (C₁)fluoroalkyl compounds include fluoromethyl, difluoromethyl and trifluoromethyl; exemplary (C₂)fluoroalkyl compounds include 1-fluoroethyl, 2-fluoroethyl, 1,1-difluoroethyl, 1,2-difluoroethyl, 1,1,1-trifluoroethyl, 1,1,2-trifluoroethyl, and the like.

“Hydroxyalkyl” means an alkyl as defined herein substituted with one atoms. Exemplary hydroxyalkyl compounds include hydroxymethyl, 1-hydroxyethyl, 2-hydroxyethyl, and the like.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

By “fluoroalkoxy” means an alkoxy as defined herein substituted with one, two or three fluoro atoms. Exemplary (C₁)fluoroalkoxy compounds include fluoromethoxy, difluoromethoxy and trifluoromethoxy; exemplary (C₂)fluoroalkyl compounds include 1-fluoroethoxy, 2-fluoroethoxy, 1,1-difluoroethoxy, 1,2-difluoroethoxy, 1,1,1-trifluoroethoxy, 1,1,2-trifluoroethoxy, and the like.

“Patient” refers to warm blooded animals such as, for example, guinea pigs, mice, rats, gerbils, cats, rabbits, dogs, cattle, goats, sheep, horses, monkeys, chimpanzees, and humans.

The term “pharmaceutically acceptable” means the substance (e.g., the compounds of the invention) and any salt thereof, or composition containing the substance or salt of the invention that is suitable for administration to a patient.

As used herein, the expressions “reaction-inert solvent” and “inert solvent” refer to a solvent or a mixture thereof which does not interact with starting materials, reagents, intermediates or products in a manner which adversely affects the yield of the desired product.

As used herein, the following terms have the general meaning for administration of pharmaceutical agents: QD means once daily and BID means twice daily.

As used herein, the term “selectivity” or “selective” refers to a greater effect of a compound in a first assay, compared to the effect of the same compound in a second assay. For example, in “gut selective” compounds, the first assay is for the half life of the compound in the intestine and the second assay is for the half life of the compound in the liver.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “treating”, “treat” or “treatment” as used herein embraces preventative, i.e., prophylactic; palliative treatment, i.e., relieve, alleviate, or slow the progression of the patient's disease (or condition) or any tissue damage associated with the disease (or condition); and reversal where the patient's disease (or condition) is not only alleviated but any tissue damage associated with the disease (or condition) is placed in a better state then when treatment was initiated. This latter could occur, for example and not limitation, from any one or more of the following: demonstration of NASH resolution and/or from an improvement in the fibrosis score based on liver biopsy; lower incidence of progression to cirrhosis, hepatocellular carcinoma, and/or other liver related outcomes; a reduction or improvement of the level of serum or imaging based markers of nonalcoholic steatohepatitis activity; reduction or improvement of nonalcoholic steatohepatitis disease activity; or reduction in the medical consequences of nonalcoholic steatohepatitis.

### Compositions

In a first aspect, the present invention is directed to compositions comprising compounds of Formula (I)

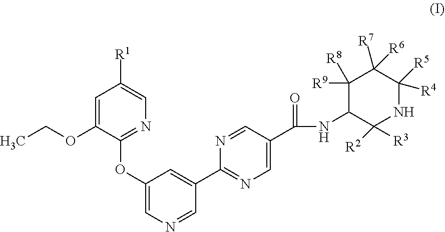

wherein
R¹ is H or fluoro;
R², R³, R⁴ and R⁵ are each independently selected from H, (C₁-C₃)alkyl, (C₁-C₃)fluoroalkyl, (C₁-C₃)hydroxyalkyl, and —(C₁-C₃)alkyl-(C₁-C₂)alkoxy; and
R⁶, R⁷, R⁸, and R⁹ are each independently selected from H, fluoro, hydroxyl, (C₁-C₃)alkyl, (C₁-C₃)fluoroalkyl, (C₁-C₃)hydroxyalkyl, (C₁-C₂)alkoxy, (C₁-C₂)fluoroalkoxy, and —(C₁-C₃)alkyl-(C₁-C₂)alkoxy; and
wherein 0, 1 or 2 of R², R³, R⁴, R⁵, R⁶, R⁷, R⁸, and R⁹ are other than H;
or a pharmaceutically acceptable salt thereof.

In a second aspect, the present invention is further directed to compositions comprising compounds of Formula (II)

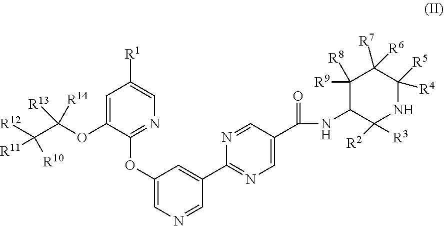

wherein:
R¹ is H or fluoro;
R², R³, R⁴ and R⁵ are each independently selected from H, (C₁-C₃)alkyl, (C₁-C₃)fluoroalkyl, (C₁-C₃)hydroxyalkyl, and —(C₁-C₃)alkyl-(C₁-C₂)alkoxy;
R⁶, R⁷, R⁶, and R⁹ are each independently selected from H, fluoro, hydroxyl, (C₁-C₃)alkyl, (C₁-C₃)fluoroalkyl, (C₁-C₃)hydroxyalkyl, (C₁-C₂)alkoxy, (C₁-C₂)fluoroalkoxy, and —(C₁-C₃)alkyl-(C₁-C₂)alkoxy; and
R¹⁰, R¹¹, R¹², R¹³ and R¹⁴ are each independently selected from H and deuterium; and
wherein 0, 1 or 2 of R², R³, R⁴, R⁵, R⁶, R⁷, R⁸, and R⁹ are other than H;
or a pharmaceutically acceptable salt thereof.

One embodiment of the first and second aspects of the present invention includes a composition comprising compounds of Formula (I) or (II) wherein R², R³, R⁴ and R⁵ are each independently selected from H and (C₁)fluoroalkyl and R⁶, R⁷, R⁸, and R⁹ are each independently selected from H, (C₁)fluoroalkyl, and fluoro; wherein 0, 1 or 2 of R², R³, R⁴, R⁵, R⁶, R⁷, R⁸, and R⁹ are other than H; or a pharmaceutically acceptable salt thereof.

Another embodiment of the present invention includes a composition comprising compounds of Formula (I) or (II) wherein R², R³, R⁴, R⁵, R⁶, and R⁷ are H; and R⁸ and R⁹ are independently selected from H, (C₁)fluoroalkyl and fluoro; wherein at least one of R⁸, and R⁹ are (C₁)fluoroalkyl or fluoro; or a pharmaceutically acceptable salt thereof.

Another embodiment of the first and second aspects of the present invention includes a composition comprising compounds of Formula (I) or (II) wherein R², R³, R⁴, R⁵, R⁸, and R⁹ are H; and R⁶ and R⁷ are each independently selected from H, (C₁)fluoroalkyl and fluoro wherein at least one of R⁶ and R⁷ are (C₁)fluoroalkyl or fluoro; or a pharmaceutically acceptable salt thereof.

Another embodiment of the first and second aspects of the present invention includes a composition comprising compounds of Formula (I) or (II) wherein R², R³, R⁴, R⁵, R⁶, and R⁹ are H; and R⁷ and R⁸ are each independently selected from H, (C₁)fluoroalkyl and fluoro wherein at least one of R⁷ and R⁸ are (C₁)fluoroalkyl or fluoro; or a pharmaceutically acceptable salt thereof.

Another embodiment of the first and second aspects of the present invention includes a composition comprising compounds of Formula (I) or (II) wherein R², R³, R⁴, R⁵, R⁶, R⁸ and R⁹ are H; and R⁷ is (C₁)fluoroalkyl or fluoro; or a pharmaceutically acceptable salt thereof.

Another embodiment of the first and second aspects of the present invention includes a composition comprising compounds of Formula (I) or (II) wherein R², R³, R⁴, R⁵, R⁶, R⁸ and R⁹ are H; and R⁷ is fluoro; or a pharmaceutically acceptable salt thereof.

Another embodiment of the first and second aspects of the present invention includes a composition comprising compound selected from
2-(5-((3-ethoxy-5-fluoropyridin-2-yl)oxy)pyridin-3-yl)-N-((3R,4S)-4-fluoropiperidin-3-yl)pyrimidine-5-carboxamide;
2-(5-((3-ethoxy-5-fluoropyridin-2-yl)oxy)pyridin-3-yl)-N-((3S,5S)-5-fluoropiperidin-3-yl)pyrimidine-5-carboxamide;
2-(5-((3-ethoxypyridin-2-yl)oxy)pyridin-3-yl)-N-((3R,4S)-4-fluoropiperidin-3-yl)pyrimidine-5-carboxamide;
2-(5-((3-ethoxypyridin-2-yl)oxy)pyridin-3-yl)-N-((3R,4R)-4-fluoropiperidin-3-yl)pyrimidine-5-carboxamide;
2-(5-((3-ethoxy-5-fluoropyridin-2-yl)oxy)pyridin-3-yl)-N-((3R,4R)-4-fluoropiperidin-3-yl)pyrimidine-5-carboxamide; and
2-(5-((3-ethoxypyridin-2-yl)oxy)pyridin-3-yl)-N-((3S,5S)-5-fluoropiperidin-3-yl)pyrimidine-5-carboxamide;
   or a pharmaceutically acceptable salt thereof.

Another embodiment of the first and second aspects of the present invention includes a composition comprising a compound having the structure:

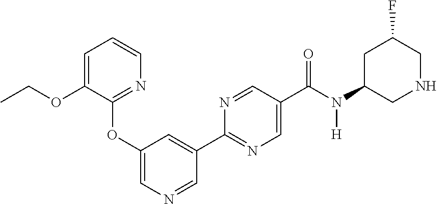 [SMILES: CCOc1cccnc1Oc1cncc(-c2ncc(C(=O)N[C@@H]3CNC[C@@H](F)C3)cn2)c1]

or a pharmaceutically acceptable salt thereof and crystals including said compound or pharmaceutically acceptable salt thereof.

In another embodiment, the composition comprises a compound is 2-(5-((3-ethoxypyridin-2-yl)oxy)pyridin-3-yl)-N-(5-fluoropiperidin-3-yl)pyrimidine-5-carboxamide.

Every Example or pharmaceutically acceptable salt thereof may be claimed individually or grouped together in any combination with any number of each and every embodiment described herein.

Another embodiment of the first and second aspects of the present invention includes the use of the compositions comprising a compound of Formula (I) or (II) or a pharmaceutically acceptable salt of said compound for use as a medicament, particularly wherein said medicament is for use in the treatment of fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma including administering to a mammal, such as a human, in need of such treatment a therapeutically effective amount.

Another embodiment of the first and second aspects of the present invention includes the use of a composition comprising a compound of Formula (I) or (II) or a pharmaceutically acceptable salt of said compound for the manufacture of a medicament in treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma including administering to a mammal, such as a human, in need of such treatment a therapeutically effective amount.

Another embodiment of the first and second aspects of the present invention includes the use of composition comprising a compound of Formula (I) or (II) or a pharmaceutically acceptable salt of said compound for use as a medicament, particularly wherein said medicament is for use in the treatment of heart failure, congestive heart failure, coronary heart disease, peripheral vascular disease, renovascular disease, pulmonary hypertension, vasculitis, acute coronary syndromes and modification of cardiovascular risk including administering to a mammal, such as a human, in need of such treatment a therapeutically effective amount of a compound of Formula (I) or (II) or a pharmaceutically acceptable salt of said compound.

Another embodiment of the first and second aspects of the present invention includes the use of a composition comprising a compound of Formula (I) or (II) or a pharmaceutically acceptable salt of said compound for the manufacture of a medicament in treating heart failure, congestive heart failure, coronary heart disease, peripheral vascular disease, renovascular disease, pulmonary hypertension, vasculitis, acute coronary syndromes and modification of cardiovascular risk including administering to a mammal, such as a human, in need of such treatment a therapeutically effective amount of a compound of Formula (I) or (II) or a pharmaceutically acceptable salt of said compound.

Another embodiment of the first and second aspects of the present invention includes the use of a composition comprising a compound of Formula (I) or (II) or a pharmaceutically acceptable salt of said compound for use as a medicament, particularly wherein said medicament is for use in the treatment of Type 1 diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, diabetic retinopathy, macular degeneration, cataract, diabetic nephropathy, glomerulosclerosis, chronic renal failure, diabetic neuropathy, metabolic syndrome, syndrome X, hyperglycemia, hyperinsulinemia, hypertriglyceridemia, insulin resistance, impaired glucose metabolism, skin and connective tissue disorders, foot ulcerations and ulcerative colitis, endothelial dysfunction and impaired vascular compliance, hyper apo B lipoproteinemia, and maple syrup urine disease including administering to a mammal, such as a human, in need of such treatment a therapeutically effective amount of a compound of Formula (I) or (II) or a pharmaceutically acceptable salt of said compound.

Another embodiment of the first and second aspects of the present invention includes the use of a composition comprising a compound of Formula (I) or (II) or a pharmaceutically acceptable salt of said compound for the manufacture of a medicament in treating Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, diabetic retinopathy, macular degeneration, cataract, diabetic nephropathy, glomerulosclerosis, chronic renal failure, diabetic neuropathy, metabolic syndrome, syndrome X, hyperglycemia, hyperinsulinemia, hypertriglyceridemia, insulin resistance, impaired glucose metabolism, skin and connective tissue disorders, foot ulcerations and ulcerative colitis, endothelial dysfunction and impaired vascular compliance, hyper apo B lipoproteinemia, and maple syrup urine disease including administering to a mammal, such as a human, in need of such treatment a therapeutically effective amount of a compound of Formula (I) or (II) or a pharmaceutically acceptable salt of said compound.

Another embodiment of the first and second aspects of the present invention includes the use of a composition comprising a compound of Formula (I) or (II) or a pharmaceutically acceptable salt of said compound for use as a medicament, particularly wherein said medicament is for use in the treatment of hepatocellular carcinoma, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, or lung adenocarcinoma comprising administering to a mammal, such as a human, in need of such treatment a therapeutically effective amount of a compound of Formula (I) or (II) or a pharmaceutically acceptable salt of said compound.

Another embodiment of the first and second aspects of the present invention includes the use of a composition comprising a compound of Formula (I) or (II) or a pharmaceutically acceptable salt of said compound for the manufacture of a medicament in treating hepatocellular carcinoma, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, or lung adenocarcinoma comprising administering to a mammal, such as a human, in need of such treatment a therapeutically effective amount of a compound of Formula (I) or (II) or a pharmaceutically acceptable salt of said compound.

The compositions of the present invention may contain the compounds described herein in asymmetric or chiral centers, and, therefore, exist in different stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans-forms, as well as mixtures, are embraced within the scope of the invention.

Chiral compounds (and chiral precursors thereof) may be obtained in enantiomerically-enriched form using chromatography, typically high pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture. In the case where SFC is used, the mobile phase may consist of a supercritical fluid, typically carbon dioxide, containing 2-50% of an alcohol, such as methanol, ethanol or isopropanol.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g. chiral auxiliary such as a chiral alcohol or Mosher's acid chloride), separating the diastereoisomers and converting (e.g. hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

Where the compositions of the present invention contain compounds that possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds contained in the compositions of the invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

It is also possible that the intermediates and compounds described herein may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations.

Valence tautomers include interconversions by reorganization of some of the bonding electrons.

Included in the scope of all compounds described herein, and contained in the compositions of the present invention, include all stereoisomers, geometric isomers and tautomeric forms of the compounds of Formula (I) or (II), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The compositions of the present invention include all pharmaceutically acceptable isotopically-labelled compounds of Formula (I) or (II) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I) or (II), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labelled compounds of Formula (I) or (II) can generally be prepared by conventional techniques known to those skilled in the art or by processes analogous to those described in the accompanying Examples and Preparations using an appropriate isotopically-labelled reagents in place of the non-labelled reagent previously employed.

The compounds contained in the compositions of the present invention may be isolated and used per se, or when possible, in the form of its pharmaceutically acceptable salt. The term “salts” refers to inorganic and organic salts of a compound of the present invention. These salts can be prepared in situ during the final isolation and purification of a compound, or by separately treating the compound with a suitable organic or inorganic acid and isolating the salt thus formed.

Salts encompassed within the term “pharmaceutically acceptable salts” refer to the compounds of this invention which are generally prepared by reacting the free base with a suitable organic or inorganic acid to provide a salt of the compound of the invention that is suitable for administration to a patient. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. See e.g. Berge, et al. J. Pharm. Sci. 66, 1-19 (1977); Handbook of Pharmaceutical Salts: Properties, Selection, and Use by Stahl and Wermuth (Wiley-VCH, 2002).

The compounds of Formula (I) or (II), and pharmaceutically acceptable salts thereof, contained in the compositions of the present invention, may exist in unsolvated and solvated forms. The term ‘solvate’ is used herein to describe a molecular complex comprising the compound of Formula (I) or (II), or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are the use of multi-component complexes (other than salts and solvates) in the compositions, wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compositions of the invention include compounds of Formula (I) or (II) as hereinbefore defined, polymorphs, and isomers thereof (including optical, geometric and tautomeric isomers) as hereinafter defined and isotopically labelled compounds of Formula (I) or (II).

The compounds contained in the compositions of the present invention may be administered as prodrugs. Thus certain derivatives of compounds of Formula (I) or (II) which may have little or no pharmacological activity themselves can, when administered into or onto the body, be converted into compounds of Formula (I) or (II) having the desired activity, for example, by hydrolytic cleavage. Such derivatives are referred to as ‘prodrugs’. [Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E. B. Roche, American Pharmaceutical Association).]

Prodrugs can, for example, be produced by replacing appropriate functionalities present in the compounds of Formula (I) or (II) with certain moieties known to those skilled in the art as ‘pro-moieties’ as described, for example, in “Design of Prodrugs” by H. Bundgaard (Elsevier, 1985).

Some examples of such prodrugs include:
(i) where the compound of Formula (I) or (II) contains an alcohol functionality (—OH), an ether thereof, for example, replacement of the hydrogen with (C₁-C₆)alkanoyloxymethyl; or a phosphate ester (PO₃H₂) or pharmaceutically acceptable salts thereof; and
(ii) an amide or carbamate of the amino functionality present in Formula (I) or (II), wherein the hydrogen of the amino NH group is replaced with (C₁-C₁₀)alkanoyl or (C₁-C₁₀)alkoxycarbonyl, respectively.

Also included within the scope of the invention are compositions containing active metabolites of compounds of Formula (I) or (II) (including prodrugs), that is, compounds formed in vivo upon administration of the drug, often by oxidation or dealkylation. Some examples of metabolites in accordance with the invention include:
(i) where the compound of Formula (I) or (II) contains a methyl group, a hydroxymethyl derivative thereof (—CH₃->—CH₂OH) and
(ii) where the compound of Formula (I) or (II) contains an alkoxy group, a hydroxy derivative thereof (—OR->—OH).

The compositions of the present invention may contain compounds in one or more crystal forms (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

In general the compounds contained in the compositions of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Specific synthetic schemes for preparation of the compounds of Formula (I) or (II) are outlined below. Note that tetrazoles are generally a high energy functional group and care should be taken in the synthesis and handling of tetrazole containing molecules.

As an initial note, in the preparation of the Formula (I) or (II) compounds it is noted that some of the preparation methods useful for the preparation of the compounds described herein may require protection of remote functionality (e.g., primary amine, secondary amine, carboxyl in Formula (I) or (II) precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-tert-butoxycarbonyl, benzyloxycarbonyl, and 9-fluorenylmethylenoxycarbonyl for amines and lower alkyl or benzyl esters for carboxylic acids), which are generally not chemically reactive under the reaction conditions described and can typically be removed without chemically altering other functionality in the Formula (I) or (II) compound.

The compounds contained in the compositions of the present invention may be synthesized by synthetic routes that include processes analogous to those well-known in the chemical arts, particularly in light of the description contained herein. The starting materials are generally available from commercial sources such as MilliporeSigma (Milwaukee, WI) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)). Many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

A detailed description of the individual reaction steps is provided in the Example section below. Those skilled in the art will appreciate that other synthetic routes may be used to synthesize the compounds. Although specific starting materials and reagents are discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

A third aspect of the present invention is directed to a pharmaceutical composition comprising a therapeutically effective amount of from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and a pharmaceutically acceptable excipient. In certain embodiments, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 10 mg. In certain embodiments, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 20 mg. In certain other embodiments, the therapeutically effective amount of the 2-{5-[(3-ethoxypyridin-2-yl)oxy]pyridin-3-yl}-N-[(3S,5S)-fluoropiperidin-3-yl]pyrimidine-5-carboxamide, or pharmaceutically acceptable salt thereof is about 40 mg. In certain other embodiments, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 80 mg.

A fourth aspect of the present invention is directed to a pharmaceutical composition comprising 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

A fifth aspect of the present invention is directed to a pharmaceutical composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and a therapeutically effective amount of from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof. In certain embodiments, the 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof is administered in an amount from about 10 mg, from about 20 mg, from about 40 mg, or from about 80 mg. In certain other embodiments, the 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof is administered in an amount from about 5 mg, from about 10 mg, from about 15 mg, or from about 20 mg. In certain other embodiments, the pharmaceutical composition is administered once a day. In certain other embodiments, the pharmaceutical composition is administered twice a day.

In any one of the above-mentioned aspects of the invention, the pharmaceutical composition may contain 2-{5-[(3-ethoxypyridin-2-yl)oxy]pyridin-3-yl}-N-[(3S,5S)-5-fluoropiperidin-3-yl]pyrimidine-5-carboxamide as a crystal having the structure:

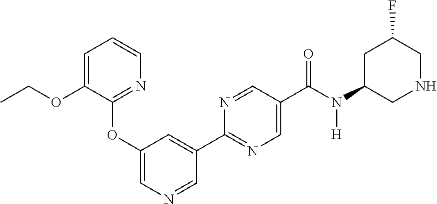 [SMILES: CCOc1cccnc1Oc1cncc(-c2ncc(C(=O)N[C@@H]3CNC[C@@H](F)C3)cn2)c1]

or a pharmaceutically acceptable salt thereof. In certain embodiments, the crystal structure of above has a powder x-ray diffraction pattern comprising 2-theta values of (CuKα radiation, wavelength of 1.54056 Å) 7.2±0.2, 14.5 0.2, 15.8 0.2, and 27.7±0.2. In certain embodiments, the crystal comprises a p-toluenesulfonate salt of the compound. In certain other embodiments, crystal has a powder x-ray diffraction pattern comprising 2-theta values of (CuKα radiation, wavelength of 1.54056 Å) 3.8±0.2, 7.7±0.2, 8.8±0.2, 22.4±0.2, and 24.6±0.2.

In any one of the above-mentioned aspects of the invention, the pharmaceutical composition further comprises 4-(4-(1-Isopropyl-7-oxo-1,4,6,7-tetrahydrospiro[indazole-5,4′-piperidine]-1′-carbonyl)-6-methoxypyridin-2-yl)benzoic acid is as a crystalline solid of structure:

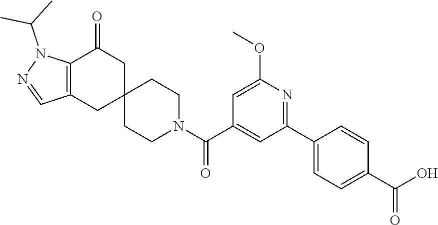 [SMILES: COc1cc(C(=O)N2CCC3(CC2)CC(=O)c2c(cnn2C(C)C)C3)cc(-c2ccc(C(=O)O)cc2)n1]

or a pharmaceutically acceptable salt thereof. In certain embodiments, the crystalline solid is 2-amino-2-(hydroxymethyl) propane-1,3-diol salt of 4-(4-(1-isopropyl-7-oxo-1,4,6,7-tetrahydrospiro[indazole-5,4′-piperidine]-1′-carbonyl)-6-methoxypyridin-2-yl)benzoic acid.

4-(4-(1-Isopropyl-7-oxo-1,4,6,7-tetrahydrospiro[indazole-5,4′-piperidine]-1′-carbonyl)-6-methoxypyridin-2-yl)benzoic acid (also referred to as “Compound A) is a selective ACC inhibitor and was prepared as the free acid in Example 9 of U.S. Pat. No. 8,859,577, which is the U.S. national phase of International Application No. PCT/IB2011/054119, all of which are hereby incorporated herein by reference in their entireties for all purposes. Crystalline forms of the compound are described in International patent application no. PCT/IB2018/058966, published as WO 2019/102311 on 31 May 2019.

It appears that the administration of an ACC inhibitor may have positive effects to lower hepatic triglycerides and potentially other beneficial effects on treatment of NASH. Increases in circulating triglycerides levels has been reported to be a mechanistic consequence of hepatic ACC inhibition (Kim et al, 2017), though doses of ACC inhibitors that only partially inhibit DNL may not produce elevations in circulating triglycerides (Bergman et al., (2018) J. of Hepatology, Volume 68, S582). WO2016/112305 provides methods of treating, stabilizing or lessening the severity or progression of a non-alcoholic fatty liver disease using an ACC inhibitor alone or with one or more additional therapeutic agents. It has been discovered that administration of 4-(4-(1-isopropyl-7-oxo-1,4,6,7-tetrahydrospiro[indazole-5,4′-piperidine]-1′-carbonyl)-6-methoxypyridin-2-yl)benzoic acid, optionally administered as a pharmaceutically acceptable salt, has a potential to result in elevations in circulating triglycerides (generally measured from plasma) in Western diet fed Sprague Dawley rats as was observed in human subjects. In addition, decreases in platelets has been reported with administration of 4-(4-(1-Isopropyl-7-oxo-1,4,6,7-tetrahydrospiro[indazole-5,4′-piperidine]-1′-carbonyl)-6-methoxypyridin-2-yl)benzoic acid (Bergman, A., et al., “Safety, tolerability, pharmacokinetics and pharmacodynamics of a liver-targeting ACC inhibitor following single and multiple oral doses”, J. Hepatology, April 2018, Vol. 68, Supp 1, Pg. S582.

### Methods of Treatment

While liver biopsy remains the standard for identification of NASH patients, non-invasive methods for identifying patients with inflammatory liver disease have been described by Drescher, H., et al., (“Current status in testing for nonalcoholic fatty liver disease (NAFLD) and non-alcoholic steatohepatitis (NASH), Cells 2019, 8, 845). These non-invasive surrogate markers include, blood tests, liver function tests, and imaging which have been successfully relied upon as a means to identify inflammatory liver disease (hepatic steatosis, steatohepatitis, and fibrosis) and a measure for efficacy of a specific therapy.

Hepatic steatosis (steatosis) is a key factor in NAFLD. While there is no specific serum marker existing today, there are several blood biomarkers panels that can be utilized to assess steatosis. These blood biomarkers may include, but are not limited to: i) NAFLD ridge score (parameters include ALT, HDL, cholesterol, triglycerides, HbA1c, leukocyte count hypertension); ii) NAFLD Liver Fat Score (NLFS) (parameters include liver fat content, metabolic syndrome, type-2 diabetes, AST, AST:ALT, fasting insulin); iii) Hepatic Steatosis Index (HIS) (parameters include AST, ALT, BMI, diabetes, sex); iv) Fatty Liver Index (FLI) (parameters include BMI, waist circumference, triglycerides, γ-glutamyl transferase); v) lipid accumulation product index (LAP) (parameters include sex, triglycerides, weight circumference); vi) Fatty Liver Inhibition of Progression (FLIP) algorithm (parameters include histological steatosis, disease activity, fibrosis score); vii) CHek score (parameters include age, HbA1c, γ-glutamyl transferase, adiponectin, M30); viii) NAFLD Fibrosis Score (NFS) (parameters include AST:ALT, albumin, platelet count, age, BMI, hyperglycemia); ix) Fibrosis-4-Score (FIB-4) (parameters include AST, ALT, platelet count, age); x) AST to Platelet Ratio Index (APRI) (parameters include AST, platelet count); xi) BARD Score (parameters include BMI, AST:ALT, diabetes); xii) Enhanced Liver Fibrosis panel (ELF) (parameters include age, TIMP-1, PIIINP, hyaluronic acid); xiii) Hepascore (parameters include bilirubin, γ-glutamyl transferase, hyaluronic acid, α₂ macroglobilin, age, gender); xiv) Fibro-Test-FibroSURE/Acti-Test (parameters include α2 macroglobulin, haptoglobin, γ-glutamyl transferase, total bilirubin, apolipoprotein A1, ALT, age, gender); and xv) FibroMeter NAFLD index (parameters include platelet count, prothrombin index, ferritin, AST, ALT, body weight, age, liver stiffness determined by vibration controlled transient elastography). The parameters identified for each biomarker assist in the assessment of liver damage/dysfunction (e.g., AST, ALT, γ-GT, platelet count, haptoglobin), lipid metabolism disorders (e.g., cholesterol, triglycerides), diabetes (e.g., HbA1c, fasting insulin level), inflammation (e.g., α₂ macroglobilin, ferritin).

Imaging techniques can also be used in conjunction with biopsy and blood biomarkers to identify NAFLD/NASH patients. Imaging techniques include, but are not limited to ultrasound (e.g., contrast-enhanced ultrasound (CEUS)); ultrasound-based elastography (e.g., vibration-controlled transient elastography (VCTE; FibroScan), real-time shear wave elastography (SWE), acoustic radiation force impulse elastography (ARFI), supersonic shear imaging (SSI)); controlled attenuation parameters; magnetic resonance imaging (MRI) such as MRI proton density fat fraction (MRI-PDFF); and magnetic resonance elastography (MRE).

In addition to the above-mentioned methods and means for identifying inflammatory liver disease in a patient, regulatory authority recognized conditional approval for Phase Ill studies in NASH is based on histological surrogate markers obtained by liver biopsy. These generally accepted surrogates are i) resolution of NASH without worsening of fibrosis (i.e. a numerical increase in fibrosis stage); ii) a one or more stage reduction in fibrosis without worsening of NASH. Details may be found in: Ratziu, A critical review of endpoints for non-cirrhotic NASH therapeutic trials, Journal of Hepatology, 2018, 68. 353-361, and references therein.

Additionally, regulatory authorities look to a change in the Nonalcoholic Fatty Liver Disease (NAFLD) Activity Score (NAS) from baseline. The NAFLD Activity Score (NAS) is a composite score equal to the sum of the steatosis grade (0-3), lobular inflammation grade (0-3), and hepatocellular ballooning grade (0-2), from centralized pathologist scoring of liver biopsies. The overall scale of the NAS is 0-8, with higher scores indicating more severe disease. The outcome measure, change from baseline in NAFLD Activity Score (NAS), has a possible range from −8 to +8, with negative values indicating a better outcome (improvement) and positive values indicating a worse outcome. Components of the NAS are scored as follows: Steatosis grade 0=<5% steatosis, 1=5-33% steatosis, 2=34-66% steatosis, 3=>66% steatosis. Lobular inflammation grade=amount of lobular inflammation (combines mononuclear, fat granulomas, and polymorphonuclear (pmn) foci): 0=0, 1=<2 under 20× magnification, 2=2-4 under 20× magnification, 3=>4 under 20× magnification. Hepatocellular ballooning 0=none, 1=mild, 2=more than mild.

In certain embodiments, the present invention is directed to a method of treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma, the method comprising administering to a human in need of such treatment a therapeutically effective amount of any of the compositions described in aspects 1-5 above.

In certain embodiments, the present invention is directed to a method of treating fatty liver, alcoholic fatty liver disease, alcoholic steatohepatitis, alcoholic steatohepatitis with liver fibrosis, alcoholic steatohepatitis with cirrhosis or alcoholic steatohepatitis with cirrhosis and hepatocellular carcinoma, the method comprising administering to a human in need of such treatment a therapeutically effective amount of any of the compositions described in aspects 1-5 above.

In certain embodiments, the present invention is directed to a method for the reduction of at least one point in severity of nonalcoholic fatty liver disease (NAFLD) Activity Score (NAS) from baseline comprising the step of measuring the baseline NAS in a human, administering to said human a therapeutically effective amount of any of the compositions described in aspects 1-5 above, and measuring the NAS of said human.

In certain embodiments, the present invention is directed to a method for the reduction of at least two points in severity of nonalcoholic fatty liver disease (NAFLD) Activity Score (NAS) from baseline comprising the step of measuring the baseline NAS in a human, administering to said human a therapeutically effective amount of any of the compositions described in aspects 1-5 above, and measuring the NAS of said human.

In certain embodiments, the present invention is directed to a method of treating a cardiovascular disease or condition selected from atherosclerosis, stroke, myocardial infarction, aortic vascular disease, cerebral vascular disease, renal vascular disease, heart failure, atrial fibrillation, or coronary heart disease comprising administering to a human in need of such treatment a therapeutically effective amount of the compositions described in aspects 1-5 above, and measuring the NAS of said human.

In certain embodiments, the present invention is directed to a method of treating a metabolic disease or condition selected from obesity, dyslipidemia, type 2 diabetes mellitus, glycemic control in patients with type 2 diabetes mellitus, conditions of impaired glucose tolerance (IGT), conditions of impaired fasting plasma glucose, metabolic syndrome, syndrome X, hyperglycemia, hyperinsulinemia, insulin resistance, or impaired glucose metabolism, comprising administering to a human in need of such treatment a therapeutically effective amount of the compositions described in aspects 1-5 above, and measuring the NAS of said human.

In certain embodiments, the present invention is directed to a method of treating hypertriglyceridemia, atherosclerosis, myocardial infarction, dyslipidemia, coronary heart disease, hyper apo B lipoproteinemia, ischemic stroke, type 2 diabetes mellitus, glycemic control in patients with type 2 diabetes mellitus, conditions of impaired glucose tolerance (IGT), conditions of impaired fasting plasma glucose, metabolic syndrome, syndrome X, hyperglycemia, hyperinsulinemia, insulin resistance, impaired glucose metabolism, comprising administering to a human in need of such treatment a therapeutically effective amount of the compositions described in aspects 1-5 above, and measuring the NAS of said human.

Treatments with Fixed-Dose Combinations

In a sixth aspect, the present invention is directed to a method for treating a disease or condition selected from fatty liver, nonalcoholic fatty liver disease, nonalcoholic steatohepatitis, nonalcoholic steatohepatitis with liver fibrosis, nonalcoholic steatohepatitis with cirrhosis, and nonalcoholic steatohepatitis with cirrhosis and with hepatocellular carcinoma or with a metabolic-related disease, the method comprising administering to a human in need thereof a therapeutically effective amount of a composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof and from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In a seventh aspect, the present invention is directed to a method for treating a disease or condition selected from fatty liver; alcoholic fatty liver disease; alcoholic steatohepatitis; alcoholic steatohepatitis with liver fibrosis; alcoholic steatohepatitis with cirrhosis; and alcoholic steatohepatitis with cirrhosis and with hepatocellular carcinoma or with a metabolic-related disease, the method comprising administering to a human in need thereof a therapeutically effective amount of a composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof and from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In an eighth aspect, the present invention is directed to a method for treating a cardiovascular disease or condition selected from atherosclerosis, stroke, myocardial infarction, aortic vascular disease, cerebral vascular disease, renal vascular disease, heart failure, atrial fibrillation, or coronary heart disease, the method comprising the steps of administering to a human in need thereof a therapeutically effective amount of a composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof and from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In a ninth aspect, the present invention is directed to a method for treating a metabolic disease or condition selected from obesity, dyslipidemia, type 2 diabetes mellitus, glycemic control in patients with type 2 diabetes mellitus, conditions of impaired glucose tolerance (IGT), conditions of impaired fasting plasma glucose, metabolic syndrome, syndrome X, hyperglycemia, hyperinsulinemia, insulin resistance, or impaired glucose metabolism, the method comprising administering to a human in need thereof a therapeutically effective amount of a composition comprising a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof and from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In certain embodiments, in any one of aspects six through nine, the composition is administered once a day. In certain other embodiments, the composition is administered twice a day.

In certain embodiments, in any one of aspects six through nine, the pharmaceutical composition contains 2-{5-[(3-ethoxypyridin-2-yl)oxy]pyridin-3-yl}-N-[(3S,5S)-5-fluoropiperidin-3-yl]pyrimidine-5-carboxamide as a crystal having the structure:

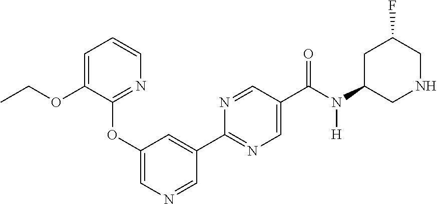 [SMILES: CCOc1cccnc1Oc1cncc(-c2ncc(C(=O)N[C@@H]3CNC[C@@H](F)C3)cn2)c1]

or a pharmaceutically acceptable salt thereof. In certain embodiments, the crystal structure of above has a powder x-ray diffraction pattern comprising 2-theta values of (CuKα radiation, wavelength of 1.54056 Å) 7.2±0.2, 14.5±0.2, 15.8±0.2, and 27.7±0.2.

In certain embodiments, the crystal comprises a p-toluenesulfonate salt of the compound. In certain other embodiments, crystal has a powder x-ray diffraction pattern comprising 2-theta values of (CuKα radiation, wavelength of 1.54056 Å) 3.8±0.2, 7.7±0.2, 8.8±0.2, 22.4±0.2, and 24.6±0.2.

In certain embodiments, in any one of aspects six through nine, the pharmaceutical composition contains 4-(4-(1-Isopropyl-7-oxo-1,4,6,7-tetrahydrospiro[indazole-5,4′-piperidine]-1′-carbonyl)-6-methoxypyridin-2-yl)benzoic acid as a crystalline solid of structure:

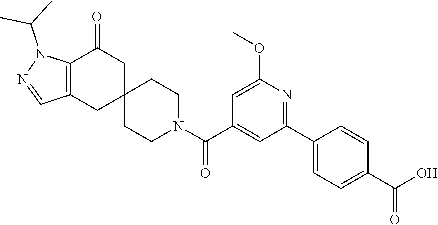 [SMILES: COc1cc(C(=O)N2CCC3(CC2)CC(=O)c2c(cnn2C(C)C)C3)cc(-c2ccc(C(=O)O)cc2)n1]

or a pharmaceutically acceptable salt thereof. In certain embodiments, the crystalline solid is 2-amino-2-(hydroxymethyl) propane-1,3-diol salt of 4-(4-(1-isopropyl-7-oxo-1,4,6,7-tetrahydrospiro[indazole-5,4′-piperidine]-1′-carbonyl)-6-methoxypyridin-2-yl)benzoic acid.

In certain embodiments, in any one of aspects six through nine, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 10 mg. In certain embodiments, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 20 mg. In certain other embodiments, the therapeutically effective amount of the 2-{5-[(3-ethoxypyridin-2-yl)oxy]pyridin-3-yl}-N-[(3S,5S)-fluoropiperidin-3-yl]pyrimidine-5-carboxamide, or pharmaceutically acceptable salt thereof is about 40 mg. In certain other embodiments, the therapeutically effective amount of the 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof is about 80 mg.

Treatment with Combinations

In a tenth aspect, the present invention is also directed to a method for treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma, the method comprising administering to a human in need of such treatment a first composition comprising a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof in combination with a second composition comprising a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof. In certain embodiments, the first and second compositions are administered simultaneously. In certain other embodiments, the first and second compositions are administered sequentially.

In an eleventh aspect, the present invention is also directed at a method for the reduction of at least one point in severity of nonalcoholic fatty liver disease or nonalcoholic steatohepatitis grading scoring systems, reduction of the level of serum markers of nonalcoholic steatohepatitis activity, reduction of nonalcoholic steatohepatitis disease activity or reduction in the medical consequences of nonalcoholic steatohepatitis in humans comprising the step of administering to a patient in need of such reduction a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, in combination with at least a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In a twelfth aspect, the present invention is also directed at a method for treating fatty liver, nonalcoholic fatty liver disease, nonalcoholic steatohepatitis, nonalcoholic steatohepatitis with liver fibrosis, nonalcoholic steatohepatitis with cirrhosis, or nonalcoholic steatohepatitis with cirrhosis and with hepatocellular carcinoma in humans comprising the step of administering to a human in need of such treatment a therapeutically effective amount of a first and second compositions and optionally a third composition wherein
i. the first composition comprises 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, and a pharmaceutically acceptable excipient;
ii. the second composition comprises 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof, and a pharmaceutically acceptable excipient; and
iii. the third composition comprises a pharmaceutical agent selected from the group consisting of an anti-inflammatory agent, an anti-diabetic agent, an anti-fibrotic agent, an anti-steatiotic agent, a cholesterol/lipid modulating agent, and an anti-diabetic agent, and a pharmaceutically acceptable excipient.

In a thirteenth aspect, the present invention is also directed to a method of treating heart failure, congestive heart failure, coronary heart disease, peripheral vascular disease, renovascular disease, pulmonary hypertension, vasculitis, acute coronary syndromes and modification of cardiovascular risk comprising administering to a human in need of such treatment a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, in combination with at least a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In a fourteenth aspect, the present invention is also directed to a method of treating obesity, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, diabetic retinopathy, macular degeneration, cataract, diabetic nephropathy, glomerulosclerosis, chronic renal failure, diabetic neuropathy, metabolic syndrome, syndrome X, hyperglycemia, hyperinsulinemia, hypertriglyceridemia, insulin resistance, impaired glucose metabolism, skin and connective tissue disorders, foot ulcerations and ulcerative colitis, endothelial dysfunction and impaired vascular compliance, hyper apo B lipoproteinemia, and maple syrup urine disease comprising administering to a human in need of such treatment a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, in combination with at least a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In a fifteenth aspect, the present invention is also directed to a method of treating hepatocellular carcinoma, kidney renal clear cell carcinoma, head and neck squamous cell carcinoma, colorectal adenocarcinoma, mesothelioma, stomach adenocarcinoma, adrenocortical carcinoma, kidney papillary cell carcinoma, cervical and endocervical carcinoma, bladder urothelial carcinoma, lung adenocarcinoma comprising administering to a human in need of such treatment a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, in combination with at least a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

In certain embodiments, in any one of aspects ten through fifteen, the 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, and the 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof are administered once a day.

In another embodiment, in any one of aspects ten through fifteen, the 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, and the 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof are administered twice a day.

In another embodiment, in any one of aspects ten through fifteen, the 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, and the 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof are administered simulataneously. In certain embodiments, the 2-{5-[(3-ethoxypyridin-2-yl)oxy]pyridin-3-yl}-N-[(3S,5S)-5-fluoropiperidin-3-yl]pyrimidine-5-carboxamide or a pharmaceutically acceptable salt thereof, and the 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof are administered sequentially, and in any order.

In any of the above-mentioned aspect six through fifteen, the administration of the combination achieves a change in whole liver fat from baseline equal to or greater than about 30%. In other instances, the administration of the combination achieves a change in whole liver fat from baseline equal to or greater than about 50%.

In any of the above-mentioned aspects six through fifteen, identification of a patient may be through use of one or more blood marker panels. Suitable blood marker panels include, but are not limited to the group consisting of NAFLD ridge score, NAFLD Liver Fat Score (NLFS), Hepatic Steatosis Index (HIS), Fatty Liver Index (FLI), Lipid accumulation product index (LAP), Fatty Liver Inhibition of Progress (FLIP) algorithm, CHeK score, NALFD Fibrosis Score (NFS), Fibrosis-4 Score (Fib-4), AST to Platelet Ratio Index (APRI), BARD score, Enhanced Liver Fibrosis panel (ELF), Hepascore, FibroTest-FibroSURE/ActiTest, ibroMeter NAFLD index, and any combinations of the foregoing.

In certain embodiments, when a patient is identified as having hepatic steatosis, the blood marker panel utilized is the NAFLD ridge score. In another embodiment, the blood marker panel is NAFLD Liver Fat Score (NLFS). In another embodiment, the blood marker panel is Fatty Liver Index (FLI).

In certain embodiments, when the patient is identified as having steatohepatitis, the blood marker panel utilized is the Fatty Liver Inhibition of Progress (FLIP) algorithm. In another embodiment, the blood marker panel is the CHeK score.

In certain embodiments, when a patient is identified as having fibrosis, the blood marker panel utilized is the NAFLD Fibrosis Score (NFS). In another embodiment, the blood marker panel is the Fibrosis-4 score (Fib-4). In another embodiment, the blood marker panel is the AST to Platelet Ratio Index (APRI). In another embodiment, the blood marker panel is the BARD score.

In certain other embodiments, in the methods described above, the step of identifying a patient with hepatic steatosis, steatohepatitis or both further includes the use of imaging. The imaging may include, but is not limited to, ultrasound, ultrasound-based elastography, controlled attenuation parameter (CAP), magnetic resonance imaging (MRI), magnetic resonance elastography, or a combination of the foregoing. In one embodiment, the imaging is contrast-enhanced ultrasound (CEUS). In another embodiment, the imaging is ultrasound-based elastography is selected from vibration-controlled transient elastography (VCTE), acoustic radiation force impulse elastography (ARFI), supersonic shear imaging (SSI), or a combination of the foregoing. In another embodiment, the imaging is magnetic resonance imaging (MRI) is MRI proton density fat fraction (MRI-PDFF). In another embodiment, the imaging is magnetic resonance elastography.

In any of the above-mentioned aspects, the methods further comprise administration of at least one additional pharmaceutical agent selected from the group consisting of an anti-inflammatory agent, an anti-diabetic agent, and a cholesterol/lipid modulating agent.

### Combination Agents

The compositions of the present invention can be administered alone or in combination with one or more additional therapeutic agents. By “administered in combination” or “combination therapy” it is meant that a composition of the present invention and one or more additional therapeutic agents are administered concurrently to the mammal being treated. When administered in combination, each component may be administered at the same time or sequentially in any order at different points in time. Thus, each component may be administered separately but sufficiently closely in time so as to provide the desired therapeutic effect. The phrases “concurrent administration,” “co-administration,” “simultaneous administration,” and “administered simultaneously” mean that the compounds are administered in combination. Thus, the methods of prevention and treatment described herein include use of combination agents.

The combination agents are administered to a mammal in a therapeutically effective amount. By “therapeutically effective amount” it is meant an amount of a compound of the present invention that, when administered alone or in combination with an additional therapeutic agent to a mammal, is effective to treat the desired disease/condition (e.g., NASH, heart failure or diabetes).

Given the NASH/NAFLD activity of the compositions of this invention, they may be co-administered with other agents for the treatment of non-alcoholic steatohepatitis (NASH) and/or non-alcoholic fatty liver disease (NAFLD) and associated disease/conditions, such as Orlistat, TZDs and other insulin-sensitizing agents, FGF21 analogs, Metformin, Omega-3-acid ethyl esters (e.g. Lovaza), Fibrates, HMG CoA-reductase Inhibitors, Ezetimibe, Probucol, Ursodeoxycholic acid, TGR5 agonists, FXR agonists, Vitamin E, Betaine, Pentoxifylline, CB1 antagonists, Carnitine, N-acetylcysteine, Reduced glutathione, lorcaserin, the combination of naltrexone with buproprion, SGLT2 inhibitors (including dapagliflozin, canagliflozin, empagliflozin, tofogliflozin, ertugliflozin, ASP-1941, THR1474, TS-071, ISIS388626 and LX4211 as well as those in WO2010023594), Phentermine, Topiramate, GLP-1 receptor agonists, GIP receptor agonists, dual GLP-1 receptor/glucagon receptor agonists (e.g., OPK88003, MED10382, JNJ-64565111, NN9277, BI 456906), dual GLP-1 receptor/GIP receptor agonists (e.g., Tirzepatide (LY3298176), NN9423), Angiotensin-receptor blockers an acetyl-CoA carboxylase (ACC) inhibitor, a BCKDK inhibitor, a ketohexokinase (KHK) inhibitor, ASK1 inhibitors, branched-chain alpha keto acid dehydrogenase kinase inhibitors (BCKDK inhibitors), inhibitors of CCR2 and/or CCR5, PNPLA3 inhibitors, DGAT1 inhibitors, an FGF21 analog, FGF19 analogs, PPAR agonists, FXR agonists, AMPK activators (e.g. ETC-1002 (bempedoic acid)), SCD1 inhibitors or MPO inhibitors.

Exemplary GLP-1 receptor agonists include liraglutide, albiglutide, exenatide, albiglutide, lixisenatide, dulaglutide, semaglutide, HM15211, LY3298176, Medi-0382, NN-9924, TTP-054, TTP-273, efpeglenatide, those described in WO2018109607, those described in PCT/IB2019/054867 filed Jun. 11, 2019, and those described in PCT/IB2019/054961 filed Jun. 13, 2019, including the following:
2-({4-[2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-1,3-benzodioxol-4-yl]piperidin-1-yl}methyl)-7-fluoro-1-[(2S)-oxetan-2-ylmethyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-Chloro-2-fluorophenyl)-2-methyl-1,3-benzodioxol-4-yl]piperidin-1-yl}methyl)-3-(1,3-oxazol-2-ylmethyl)-3H-imidazo[4,5-b]pyridine-5-carboxylic acid;
2-({4-[2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-4-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(pyridin-3-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-5-ylmethyl)-1H-benzimidazole-6-carboxylic acid;
2-({4-[2-(4-chloro-2-fluorophenyl)-2-methyl-1,3-benzodioxol-4-yl]piperidin-1-yl}methyl)-1-[(1-ethyl-1H-1,2,3-triazol-5-yl)methyl]-1H-benzimidazole-6-carboxylic acid;
2-({4-[2-(4-chloro-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[2-(4-chloro-2-fluorophenyl)-7-fluoro-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(4-cyano-2-fluorophenyl)-2-methyl-1,3-benzodioxol-4-yl]piperidin-1-yl}methyl)-1-(1,3-oxazol-2-ylmethyl)-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-7-fluoro-1-[(2S)-oxetan-2-ylmethyl]-1H-benzimidazole-6-carboxylic acid;
2-({4-[(2S)-2-(4-Cyano-2-fluorophenyl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[(2R)-2-(4-chloro-2-fluorophenyl)-2-methyl-1,3-benzodioxol-4-yl]piperidin-1-yl}methyl)-1-[(1-ethyl-1H-imidazol-5-yl)methyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2S)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[(2R)-2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid;
2-({4-[2-(5-Chloropyridin-2-yl)-2-methyl-1,3-benzodioxol-4-yl]piperidin-1-yl}methyl)-1-[(2S)-oxetan-2-ylmethyl]-1H-benzimidazole-6-carboxylic acid, DIAST-X2;
2-[(4-{2-[(4-chloro-2-fluorobenzyl)oxy]pyridin-3-yl}piperidin-1-yl)methyl]-1-[(2S)-oxetan-2-ylmethyl]-1H-benzimidazole-6-carboxylic acid;
2-[(4-{2-[(4-chloro-2-fluorobenzyl)oxy]pyridin-3-yl}piperidin-1-yl)methyl]-1-(1,3-oxazol-2-ylmethyl)-1H-benzimidazole-6-carboxylic acid;
2-[(4-{2-[(4-cyano-2-fluorobenzyl)oxy]pyridin-3-yl}piperidin-1-yl)methyl]-1-(1,3-oxazol-2-ylmethyl)-1H-benzimidazole-6-carboxylic acid;
2-[(4-{2-[(4-cyano-2-fluorobenzyl)oxy]pyridin-3-yl}piperidin-1-yl)methyl]-1-[(2S)-oxetan-2-ylmethyl]-1H-benzimidazole-6-carboxylic acid;
2-[(4-{3-[(4-chloro-2-fluorobenzyl)oxy]pyrazin-2-yl}piperidin-1-yl)methyl]-1-[(2S)-oxetan-2-ylmethyl]-1H-benzimidazole-6-carboxylic acid;
2-(6-{6-[(4-cyano-2-fluorobenzyl)oxy]pyridin-2-yl}-6-azaspiro[2.5]oct-1-yl)-1-[(2S)-oxetan-2-ylmethyl]-1H-benzimidazole-6-carboxylic acid;
2-(6-{2-[(4-chloro-2-fluorobenzyl)oxy]-5-fluoropyrimidin-4-yl}-6-azaspiro[2.5]oct-1-yl)-1-[(2S)-oxetan-2-ylmethyl]-1H-benzimidazole-6-carboxylic acid;
2-(6-{2-[(4-chloro-2-fluorobenzyl)oxy]-5-fluoropyrimidin-4-yl}-6-azaspiro[2.5]oct-1-yl)-1-(1,3-oxazol-2-ylmethyl)-1H-benzimidazole-6-carboxylic acid;
2-(6-{6-[(4-cyano-2-fluorobenzyl)oxy]-5-fluoropyridin-2-yl}-6-azaspiro[2.5]oct-1-yl)-1-[(2S)-oxetan-2-ylmethyl]-1H-benzimidazole-6-carboxylic acid;
2-(6-{6-[(4-cyano-2-fluorobenzyl)oxy]-3-fluoropyridin-2-yl}-6-azaspiro[2.5]oct-1-yl)-1-[(2S)-oxetan-2-ylmethyl]-1H-benzimidazole-6-carboxylic acid;
2-[(4-{2-[(4-chloro-2-fluorobenzyl)oxy]pyrimidin-4-yl}piperidin-1-yl)methyl]-1-[(2S)-oxetan-2-ylmethyl]-1H-benzimidazole-6-carboxylic acid;
2-{[(2S)-4-{2-[(4-chloro-2-fluorobenzyl)oxy]-5-fluoropyrimidin-4-yl}-2-methylpiperazin-1-yl]methyl}-1-[(2S)-oxetan-2-ylmethyl]-1H-benzimidazole-6-carboxylic acid;
2-{[(2S)-4-{2-[(4-chloro-2-fluorobenzyl)oxy]pyrimidin-4-yl}-2-methylpiperazin-1-yl]methyl}-1-[(2S)-oxetan-2-ylmethyl]-1H-benzimidazole-6-carboxylic acid; and
2-[(4-{6-[(4-Cyano-2-fluorobenzyl)oxy]pyridin-2-yl}piperidin-1-yl)methyl]-1-[(2S)-oxetan-2-ylmethyl]-1H-benzimidazole-6-carboxylic acid, and pharmaceutically acceptable salts thereof.

Exemplary ACC inhibitors include 4-(4-[(1-isopropyl-7-oxo-1,4,6,7-tetrahydro-1′H-spiro[indazole-5,4′-piperidin]-1′-yl)carbonyl]-6-methoxypyridin-2-yl)benzoic acid, gemcabene, and firsocostat (GS-0976) and phamaceutally acceptable salts thereof.

Exemplary FXR Agonists include tropifexor (2-[(1R,3R,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), cilofexor (GS-9674), obeticholic acid, LY2562175, Met409, TERN-101 and EDP-305 and pharmaceutically acceptable salts thereof.

Exemplary KHK inhibitors include [(1R,5S,6R)-3-{2-[(2S)-2-methylazetidin-1-yl]-6-(trifluoromethyl)pyrimidin-4-yl}-3-azabicyclo[3.1.0]hex-6-yl]acetic acid and pharmaceutically acceptable salts thereof.

Exemplary BCKDK inhibitors include those described in U.S. Ser. No. 62/868,057 filed Jun. 28, 2019 and U.S. Ser. No. 62/868,542 filed Jun. 28, 2019 including the following:
5-(5-chloro-4-fluoro 3-methylthiophen-2-yl)-1H-tetrazole;
5-(5-chloro-3-difluoromethylthiophen-2-yl)-1H-tetrazole;
5-(5-fluoro-3-methylthiophen-2-yl)-1H-tetrazole;
5-(5-chloro-3-methylthiophen-2-yl)-1H-tetrazole;
5-(3,5-dichlorothiophen-2-yl)-1H-tetrazole;
5-(4-bromo-3-methylthiophen-2-yl)-1H-tetrazole;
5-(4-bromo-3-ethylthiophen-2-yl)-1H-tetrazole;
5-(4-chloro-3-ethylthiophen-2-yl)-1H-tetrazole;
3-chloro-5-fluorothieno[3,2-b]thiophene-2-carboxylic acid;
3-bromo-5-fluorothieno[3,2-b]thiophene-2-carboxylic acid;
3-(difluoromethyl)-5-fluorothieno[3,2-b]thiophene-2-carboxylic acid;
5,6-difluorothieno[3,2-b]thiophene-2-carboxylic acid; and
3,5-difluorothieno[3,2-b]thiophene-2-carboxylic acid;

or a pharmaceutically acceptable salt thereof.

Given the anti-diabetic activity of the compositions of this invention they may be co-administered with other anti-diabetic agents. Suitable anti-diabetic agents include insulin, metformin, GLP-1 receptor agonists (described herein above), an acetyl-CoA carboxylase (ACC) inhibitor (described herein above), SGLT2 inhibitors (described herein above), monoacylglycerol O-acyltransferase inhibitors, phosphodiesterase (PDE)-10 inhibitors, AMPK activators (e.g. ETC-1002 (bempedoic acid)), sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, α-amylase inhibitors (e.g., tendamistat, trestatin and AL-3688), an α-glucoside hydrolase inhibitor (e.g., acarbose), α-glucosidase inhibitors (e.g., adiposine, camiglibose, emiglitate, miglitol, voglibose, pradimicin-Q, and salbostatin), PPARγ agonists (e.g., balaglitazone, ciglitazone, darglitazone, englitazone, isaglitazone, pioglitazone and rosiglitazone), PPAR α/γ agonists (e.g., CLX-0940, GW-1536, GW-1929, GW-2433, KRP-297, L-796449, LR-90, MK-0767 and SB-219994), protein tyrosine phosphatase-1 B (PTP-1 B) inhibitors (e.g., trodusquemine, hyrtiosal extract, and compounds disclosed by Zhang, S., et al., Drug Discovery Today, 12(9/10), 373-381 (2007)), SIRT-1 activators (e.g., resveratrol, GSK2245840 or GSK184072), dipeptidyl peptidease IV (DPP-IV) inhibitors (e.g., those in WO2005116014, sitagliptin, vildagliptin, alogliptin, dutogliptin, linagliptin and saxagliptin), insulin secreatagogues, a fatty acid oxidation inhibitors, A2 antagonists, c-jun amino-terminal kinase (JNK) inhibitosr, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 orGKM-001, insulin, insulin mimetics, glycogen phosphorylase inhibitors (e.g. GSK1362885), VPAC2 receptor agonists, glucagon receptor modulators such as those described in Demong, D. E. et al. Annual Reports in Medicinal Chemistry 2008, 43, 119-137, GPR119 modulators, particularly agonists, such as those described in WO2010140092, WO2010128425, WO2010128414, WO2010106457, Jones, R. M. et al. in Medicinal Chemistry 2009, 44, 149-170 (e.g. MBX-2982, GSK1292263, APD597 and PSN821), FGF21 derivatives or analogs such as those described in Kharitonenkov, A. et al. et al., Current Opinion in Investigational Drugs 2009, 10(4) 359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high affinity nicotinic acid receptor (HM74A) activators, and SGLT1 inhibitors, such as GSK1614235. A further representative listing of anti-diabetic agents that can be combined with the compounds of the present invention can be found, for example, at page 28, line 35 through page 30, line 19 of WO2011005611.

Other antidiabetic agents could include inhibitors or modulators of carnitine palmitoyl transferase enzymes, inhibitors of fructose 1,6-diphosphatase, inhibitors of aldose reductase, mineralocorticoid receptor inhibitors, inhibitors of TORC2, inhibitors of CCR2 and/or CCR5, inhibitors of PKC isoforms (e.g. PKC□□ PKC□, PKC□), inhibitors of fatty acid synthetase, inhibitors of serine palmitoyl transferase, modulators of GPR81, GPR39, GPR43, GPR41, GPR105, Kv1.3, retinol binding protein 4, glucocorticoid receptor, somatostain receptors (e.g. SSTR1, SSTR2, SSTR3 and SSTR5), inhibitors or modulators of PDHK2 or PDHK4, inhibitors of MAP4K4, modulators of IL1 family including IL1 beta, modulators of RXRalpha. In addition suitable anti-diabetic agents include mechanisms listed by Carpino, P. A., Goodwin, B. Expert Opin. Ther. Pat, 2010, 20(12), 1627-51.

The compositions of the present invention may be co-administered with anti-heart failure agents such as ACE inhibitors (e.g. captopril, enalapril, fosinopril, lisinopril, perindopril, quinapril, ramipril, trandolapril), Angiotensin II receptor blockers (e.g., candesartan, losartan, valsartan), Angiotensin-receptor neprilysin inhibitors (sacubitril/valsartan), I_{f}channel blocker Ivabradine, Beta-Adrenergic blocking agents (e.g., bisoprolol, metoprolol succinate, carvedilol), Aldosterone antagonists (e.g., spironolactone, eplerenone), hydralazine and isosorbide dinitrate, diuretics (e.g., furosemide, bumetanide, torsemide, chlorothiazide, amiloride, hydrochlorothiazide, Indapamide, Metolazone, Triamterene), or digoxin.

The compositions of the present invention may also be co-administered with cholesterol or lipid lowering agents including the following exemplary agents: HMG CoA reductase inhibitors (e.g., pravastatin, pitavastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates (e.g., gemfibrozil, pemafibrate, fenofibrate, clofibrate); bile acid sequestrants (such as questran, colestipol, colesevelam); ACAT inhibitors; MTP inhibitors; lipooxygenase inhibitors; cholesterol absorption inhibitors (e.g., ezetimibe); nicotinic acid agents (e.g., niacin, niacor, slo-niacin); omega-3 fatty acids (e.g., epanova, fish oil, eicosapentaenoic acid); cholesteryl ester transfer protein inhibitors (e.g., obicetrapib) and PCSK9 modulators (e.g., alirocumab, evolocumab, bococizumab, ALN-PCS (inclisiran)).

The compositions of the present invention may also be used in combination with antihypertensive agents and such antihypertensive activity is readily determined by those skilled in the art according to standard assays (e.g., blood pressure measurements). Examples of suitable anti-hypertensive agents include: alpha adrenergic blockers; beta adrenergic blockers; calcium channel blockers (e.g., diltiazem, verapamil, nifedipine and amlodipine); vasodilators (e.g., hydralazine), diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, torsemide, furosemide, musolimine, bumetanide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/All antagonist (e.g., compounds disclosed in WO 00/01389); neutral endopeptidase (NEP) inhibitors; vasopepsidase inhibitors (dual NEP-ACE inhibitors) (e.g., gemopatrilat and nitrates). An exemplary antianginal agent is ivabradine.

Examples of suitable calcium channel blockers (L-type or T-type) include diltiazem, verapamil, nifedipine and amlodipine and mybefradil.

Examples of suitable cardiac glycosides include digitalis and ouabain.

In one embodiment, the composition containing a Formula (I) or (II) compound may be co-administered with one or more diuretics. Examples of suitable diuretics include (a) loop diuretics such as furosemide (such as LASIX™), torsemide (such as DEMADEX™), bemetanide (such as BUMEX™), and ethacrynic acid (such as EDECRIN™); (b) thiazide-type diuretics such as chlorothiazide (such as DIURIL™ ESIDRIX™ or HYDRODIURIL™), hydrochlorothiazide (such as MICROZIDE™ or ORETIC™), benzthiazide, hydroflumethiazide (such as SALURON™) bendroflumethiazide, methychlorthiazide, polythiazide, trichlormethiazide, and indapamide (such as LOZOL™); (c) phthalimidine-type diuretics such as chlorthalidone (such as HYGROTON™), and metolazone (such as ZAROXOLYN™)-(d) quinazoline-type diuretics such as quinethazone; and (e) potassium-sparing diuretics such as triamterene (such as DYRENIUM™), and amiloride (such as MIDAMOR™ or MODURETIC™).

In another embodiment, the composition containing a compound of Formula (I) or (II) may be co-administered with a loop diuretic. In still another embodiment, the loop diuretic is selected from furosemide and torsemide. In still another embodiment, one or more compounds of Formula (I) or (II) may be co-administered with furosemide. In still another embodiment, one or more compounds of Formula (I) or (II) may be co-administered with torsemide which may optionally be a controlled or modified release form of torsemide.

In another embodiment, the composition containing a compound of Formula (I) or (II) may be co-administered with a thiazide-type diuretic. In still another embodiment, the thiazide-type diuretic is selected from the group consisting of chlorothiazide and hydrochlorothiazide. In still another embodiment, one or more compounds of Formula (I) or (II) may be co-administered with chlorothiazide. In still another embodiment, one or more compounds of Formula (I) or (II) may be co-administered with hydrochlorothiazide.

In another embodiment, the composition containing one or more compounds of Formula (I) or (II) may be co-administered with a phthalimidine-type diuretic. In still another embodiment, the phthalimidine-type diuretic is chlorthalidone.

Examples of suitable mineralocorticoid receptor antagonists include sprionolactone and eplerenone.

Examples of suitable phosphodiesterase inhibitors include: PDE Ill inhibitors (such as cilostazol); and PDE V inhibitors (such as sildenafil).

Those skilled in the art will recognize that the the composition containing compounds of this invention may also be used in conjunction with other cardiovascular or cerebrovascular treatments including PCI, stenting, drug-eluting stents, stem cell therapy and medical devices such as implanted pacemakers, defibrillators, or cardiac resynchronization therapy.

Particularly when provided as a single dosage unit, the potential exists for a chemical interaction between the combined active ingredients. For this reason, when a the composition containing a Formula (I) or (II) compound and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that effects a sustained release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

In combination therapy treatment, both the compounds of this invention and the other drug therapies are administered to mammals (e.g., humans, male or female) by conventional methods. A Formula (I) or (II) compound and the salts thereof are all adapted to therapeutic use as agents that inhibit diacylglycerol acyltransferases 2 in mammals, particularly humans, and thus are useful for the treatment of the various conditions (e.g., those described herein) in which such action is implicated.

The disease/conditions that can be treated in accordance with the present invention include, but are not limited to, cardiovascular conditions, diabetes (e.g., type II) and diabetic complications, vascular conditions, NASH (non-alcoholic steatatohepatitis), NAFLD (non-alcoholic fatty liver disease) and renal diseases.

It is believed that DGAT2 inhibition exhibits beneficial effects on both glycemic control and plasma cholesterol profile such that this target is valuable in the treatment of metabolic disease (Choi, C. S. et. al. 2007. J Biol Chem 282: 22678-22688). Given the positive correlation between inhibition of DGAT2 with metabolic and associated disease/conditions, composition containing a Formula (I) or (II) compound, by virtue of the pharmacologic action, are useful for the prevention, arrestment and/or regression of metabolic and associated disease states (e.g., type II diabetes; NASH; NAFLD).

Hepatic triglycerides (TGs) are derived from 3 principal sources: de novo lipogenesis (DNL), re esterification from fatty acids (FAs) supplied by the adipose, and dietary intake (Cohen J. C. et al. 2011, Science, 332, 1519-1523). While the largest contribution to the hepatic TG pool appears to be derived from lipolytic products originating in adipocytes, the lipogenic pathway plays an important role in the development of NAFLD and progression to NASH. The contribution of DNL to disease progression in NAFLD is supported by analyzing the FA composition of TGs in subjects with and without NAFLD. Data demonstrate an increased level of saturated FAs in those with NAFLD, implicating the DNL pathway as an important contributor to hepatic steatosis as saturated FAs represent the primary product of DNL. These findings are consistent with the increased inclusion of DNL derived TGs in VLDL particles in NAFLD where 15% of TG produced originated from DNL compared to only 2 to 5% in normal subjects consuming a typical Western diet (Sanders, F. W. B. and, Griffin J. L., 2016, Biol. Rev., 91, 452-468). Additionally, elevated rates of hepatic DNL have been reported to be a distinctive characteristic of NAFLD. Human subjects with elevated liver fat showed a more than 3-fold increase in the rate of hepatic DNL relative to subjects with normal liver fat, but no differences between the groups were detected in adipose free fatty acid (FFA) flux or in production of VLDL from FFAs. Consequently, when comparing the absolute sources of FAs incorporated into VLDL TG, elevated hepatic DNL was the only source significantly elevated in subjects with high liver fat (Lambert J. E. and Ramos-Roman, M. A., 2014, Gastroenterology, 146, 726-735).

Diacylglycerol acyltransferases (DGATs) catalyze the terminal step in TG synthesis, specifically, the esterification of a fatty acid (FA) with diacylglycerol (DAG) resulting in the formation of TG (Yen, C. L. et al. 2008, J. Lipid Res., 49, 2283-2301). In mammals, two structurally unrelated DGAT enzymes (DGAT1 and DGAT2) have been characterized. DGAT1 is highly expressed in the intestine and plays a central role in fat absorption (Buhman, K. K., et al., 2002, J. Biol. Chem., 277, 25474-25479). DGAT2 is highly expressed in liver and adipose (Cases, S., et al., 2001, J. Biol. Chem., 276, 38870-38876). In preclinical models, blockade of hepatic DGAT2 using antisense oligonucleotides results in both down regulation of the expression of multiple genes encoding proteins involved in lipogenesis and parallel induction in oxidative pathways. The net result of these changes is a decrease in the levels of hepatic DAG and TG lipid which, in turn, reduces hepatocyte lipid burden and decreases hepatic very low density lipoprotein (VLDL) TG secretion (Choi, C. S. et. al. 2007. J Biol Chem 282: 22678-22688 and Yu, X. X. et al. 2005, Hepatology, 42, 362-371). Thus it is believed that pharmacological inhibition of DGAT2 will exhibit beneficial effects for the treatment of NAFLD/NASH and other metabolic diseases including glycemic control, and plasma cholesterol.

In particular, given the positive correlation between inhibition of DGAT2 with NASH/NAFLD and associated disease/conditions, compositions containing a Formula (I) or (II) compound, by virtue of the pharmacologic action, are useful for the prevention, arrestment and/or regression of NASH/NAFLD and associated disease states

Further, regulatory authority recognized conditional approval for Phase Ill studies in NASH is based on histological surrogate markers obtained by liver biopsy. These generally accepted surrogates are i) resolution of NASH without worsening of fibrosis (i.e. a numerical increase in fibrosis stage); ii) a one or more stage reduction in fibrosis without worsening of NASH. Details may be found in: Ratziu, A critical review of endpoints for non-cirrhotic NASH therapeutic trials, Journal of Hepatology, 2018, 68. 353-361, and references therein.

Additionally, regulatory authorities look to a change in the Nonalcoholic Fatty Liver Disease (NAFLD) Activity Score (NAS) from baseline. The NAFLD Activity Score (NAS) is a composite score equal to the sum of the steatosis grade (0-3), lobular inflammation grade (0-3), and hepatocellular ballooning grade (0-2), from centralized pathologist scoring of liver biopsies. The overall scale of the NAS is 0-8, with higher scores indicating more severe disease. The outcome measure, change from baseline in NAFLD Activity Score (NAS), has a possible range from −8 to +8, with negative values indicating a better outcome (improvement) and positive values indicating a worse outcome. Components of the NAS are scored as follows: Steatosis grade 0=<5% steatosis, 1=5-33% steatosis, 2=34-66% steatosis, 3=>66% steatosis. Lobular inflammation grade=amount of lobular inflammation (combines mononuclear, fat granulomas, and polymorphonuclear (pmn) foci): 0=0, 1=<2 under 20× magnification, 2=2-4 under 20× magnification, 3=>4 under 20× magnification. Hepatocellular ballooning 0=none, 1=mild, 2=more than mild.

Due to its pharmacologic action the composition containing Formula (I) or (II) compounds are useful for treating hyperlipidemia, Type I diabetes, Type II diabetes mellitus, idiopathic Type I diabetes (Type Ib), latent autoimmune diabetes in adults (LADA), early-onset Type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, angina pectoris, thrombosis, atherosclerosis, transient ischemic attacks, stroke, vascular restenosis, hyperglycemia, hyperinsulinemia, hypertrygliceridemia, insulin resistance, impaired glucose metabolism, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, non-alcoholic steatohepatitis (NASH), or non-alcoholic fatty liver disease (NAFLD).

Administration of the compositions of this invention can be via any method which delivers a compound of this invention systemically and/or locally. These methods include oral routes, parenteral, intraduodenal routes, buccal, intranasal etc. Generally, the compositions of this invention are administered orally, but parenteral administration (e.g., intravenous, intramuscular, subcutaneous or intramedullary) may be utilized, for example, where oral administration is inappropriate for the target or where the patient is unable to ingest the drug.

For administration to human patients, an oral daily dose of the compositions herein may be in the range 1 mg to 5000 mg depending, of course, on the mode of and frequency of administration, the disease state, and the age and condition of the patient, etc. An oral daily dose is in the range of 3 mg to 2000 mg may be used. A further oral daily dose is in the range of 5 mg to 1000 mg. For convenience, the compositions of the present invention can be administered in a unit dosage form. If desired, multiple doses per day of the compositions can be used to increase the total daily dose. The compositions, for example, may be a tablet or capsule containing about 0.1, 0.5, 1, 5, 10, 15, 20, 25, 30, 35, 40, 45, 50, 55, 60, 65, 70, 75, 80, 85, 90, 95, 100, 125, 150, 175, 200, 250, 300, 500, or 1000 mg of the compositions of the present invention. The total daily dose may be administered in single or divided doses and may, at the physician's discretion, fall outside of the typical ranges given herein.

For administration to human patients, an infusion daily dose of the compositions herein may be in the range 1 mg to 2000 mg depending, of course, on the mode of and frequency of administration, the disease state, and the age and condition of the patient, etc. A further infusion daily dose is in the range of 5 mg to 1000 mg. The total daily dose may be administered in single or divided doses and may, at the physician's discretion, fall outside of the typical ranges given herein.

The dosage of each therapeutic agent, e.g., Compound A, Compound D, and the compound of Example 4, and any additional therapeutic agent, is generally dependent upon a number of factors including the health of the subject being treated, the extent of treatment desired, the nature and kind of concurrent therapy, if any, and the frequency of treatment and the nature of the effect desired. In general, the dosage range of each therapeutic agent is in the range of from about 0.001 mg to about 100 mg per kilogram body weight of the individual per day, preferably from about 0.1 mg to about 10 mg per kilogram body weight of the individual per day. However, some variability in the general dosage range may also be required depending upon the age and weight of the subject being treated, the intended route of administration, the particular anti-obesity agent being administered and the like. The determination of dosage ranges and optimal dosages for a particular patient is also well within the ability of one of ordinary skill in the art having the benefit of the instant disclosure.

According to the methods of treatment of the invention, a compositions of the present invention or a combination of a compositions of the present invention and at least one additional pharmaceutical agent (referred to herein as a “combination”) is administered to a subject in need of such treatment, preferably in the form of a pharmaceutical composition. In the combination aspect of the invention, the compositions of the present invention and at least one other pharmaceutical agent (e.g., another anti-obesity agent,) may be administered either separately or in a pharmaceutical composition comprising both. It is generally preferred that such administration be oral.

When a combination of a compositions of the present invention and at least one other pharmaceutical agent are administered together, such administration may be sequential in time or simultaneous. Simultaneous administration of drug combinations is generally preferred. For sequential administration, a compositions of the present invention and the additional pharmaceutical agent may be administered in any order. It is generally preferred that such administration be oral. It is especially preferred that such administration be oral and simultaneous. When a compositions of the present invention and the additional pharmaceutical agent are administered sequentially, the administration of each may be by the same or by different methods.

According to the methods of the invention, a compositions of the present invention is preferably administered in the form of a pharmaceutical composition.

Accordingly, a compound of the present invention or a combination can be administered to a patient separately or together in any conventional oral, rectal, transdermal, parenteral (e.g., intravenous, intramuscular or subcutaneous), intracisternal, intravaginal, intraperitoneal, topical (e.g., powder, ointment, cream, spray or lotion), buccal or nasal dosage form (e.g., spray, drops or inhalant).

The compounds contained in the compositions of the invention or combinations can be administered alone but will generally be administered in an admixture with one or more suitable pharmaceutical excipients, adjuvants, diluents or carriers known in the art and selected with regard to the intended route of administration and standard pharmaceutical practice. The compositions of the invention or combination may be formulated to provide immediate-, delayed-, modified-, sustained-, pulsed- or controlled-release dosage forms depending on the desired route of administration and the specificity of release profile, commensurate with therapeutic needs.

The pharmaceutical composition comprises a compound of the invention or a combination in an amount generally in the range of from about 1% to about 75%, 80%, 85%, 90% or even 95% (by weight) of the composition, usually in the range of about 1%, 2% or 3% to about 50%, 60% or 70%, more frequently in the range of about 1%, 2% or 3% to less than 50% such as about 25%, 30% or 35%.

Methods of preparing various pharmaceutical compositions with a specific amount of active compound are known to those skilled in this art. For examples, see Remington: The Practice of Pharmacy, Lippincott Williams and Wilkins, Baltimore Md. 20.sup.th ed. 2000.

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers or diluents (including solvents and vehicles) include water, ethanol, polyols (propylene glycol, polyethylene glycol, glycerol, and the like), suitable mixtures thereof, triglycerides including vegetable oils such as olive oil, and injectable organic esters such as ethyl oleate. A preferred carrier is Miglyol.® brand caprylic/capric acid ester with glycerine or propylene glycol (e.g., Miglyol.® 812, Miglyol.® 829, Miglyol.® 840) available from Condea Vista Co., Cranford, N.J. Proper fluidity can be maintained, for example, by the use of a coating such as lecithin, by the maintenance of the required particle size in the case of dispersions, and by the use of surfactants.

These compositions for parenteral injection may also contain excipients such as preserving, wetting, emulsifying, and dispersing agents. Prevention of microorganism contamination of the compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and gelatin.

Solid dosage forms for oral administration include capsules, tablets, chews, lozenges, pills, powders, and multi-particulate preparations (granules). In such solid dosage forms, a compound of the present invention or a combination is admixed with at least one inert excipient, diluent or carrier. Suitable excipients, diluents or carriers include materials such as sodium citrate or dicalcium phosphate and/or (a) one or more fillers or extenders (e.g., microcrystalline cellulose (available as Avicel.™. from FMC Corp.) starches, lactose, sucrose, mannitol, silicic acid, xylitol, sorbitol, dextrose, calcium hydrogen phosphate, dextrin, alpha-cyclodextrin, beta-cyclodextrin, polyethylene glycol, medium chain fatty acids, titanium oxide, magnesium oxide, aluminum oxide and the like); (b) one or more binders (e.g., carboxymethylcellulose, methylcellulose, hydroxypropylcellulose, hydroxypropylmethylcellulose, gelatin, gum arabic, ethyl cellulose, polyvinyl alcohol, pullulan, pregelatinized starch, agar, tragacanth, alginates, gelatin, polyvinylpyrrolidone, sucrose, acacia and the like); (c) one or more humectants (e.g., glycerol and the like); (d) one or more disintegrating agents (e.g., agar-agar, calcium carbonate, potato or tapioca starch, alginic acid, certain complex silicates, sodium carbonate, sodium lauryl sulphate, sodium starch glycolate (available as Explotab.™. from Edward Mendell Co.), cross-linked polyvinyl pyrrolidone, croscarmellose sodium A-type (available as Ac-di-sol.™), polyacrilin potassium (an ion exchange resin) and the like); (e) one or more solution retarders (e.g., paraffin and the like); (f) one or more absorption accelerators (e.g., quaternary ammonium compounds and the like); (g) one or more wetting agents (e.g., cetyl alcohol, glycerol monostearate and the like); (h) one or more adsorbents (e.g., kaolin, bentonite and the like); and/or lone or more lubricants (e.g., talc, calcium stearate, magnesium stearate, stearic acid, polyoxyl stearate, cetanol, talc, hydrogenated caster oil, sucrose esters of fatty acid, dimethylpolysiloxane, microcrystalline wax, yellow beeswax, white beeswax, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules may be prepared with coatings and shells, such as enteric coatings and others well known in the art. They may also contain opacifying agents, and can also be of such composition that they release the compound of the present invention and/or the additional pharmaceutical agent in a delayed manner. Examples of embedding compositions that can be used are polymeric substances and waxes. The drug may also be in micro-encapsulated form, if appropriate, with one or more of the above-mentioned excipients.

For tablets, the active agent will typically comprise less than 50% (by weight) of the formulation, for example less than about 10% such as 5% or 2.5% by weight. The predominant portion of the formulation comprises fillers, diluents, disintegrants, lubricants and optionally, flavors. The composition of these excipients is well known in the art. Frequently, the fillers/diluents will comprise mixtures of two or more of the following components: microcrystalline cellulose, mannitol, lactose (all types), starch, and di-calcium phosphate. The filler/diluent mixtures typically comprise less than 98% of the formulation and preferably less than 95%, for example 93.5%. Preferred disintegrants include Ac-di-sol.™., Explotab.™., starch and sodium lauryl sulphate. When present a disintegrant will usually comprise less than 10% of the formulation or less than 5%, for example about 3%. A preferred lubricant is magnesium stearate. When present a lubricant will usually comprise less than 5% of the formulation or less than 3%, for example about 1%.

Tablets may be manufactured by standard tabletting processes, for example, direct compression or a wet, dry or melt granulation, melt congealing process and extrusion. The tablet cores may be mono or multi-layer(s) and can be coated with appropriate overcoats known in the art.

Liquid dosage forms for oral administration include pharmaceutically acceptable emulsions, solutions, suspensions, syrups, and elixirs. In addition to the compound of the present invention or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), Miglyole.® (available from CONDEA Vista Co., Cranford, N.J.), glycerol, tetrahydrofurfuryl alcohol, polyethylene glycols and fatty acid esters of sorbitan, or mixtures of these substances, and the like.

Besides such inert diluents, the composition may also include excipients, such as wetting agents, emulsifying and suspending agents, sweetening, flavoring, and perfuming agents.

Oral liquid forms of the compositions of the invention or combinations include solutions, wherein the active compound is fully dissolved. Examples of solvents include all pharmaceutically precedented solvents suitable for oral administration, particularly those in which the compounds of the invention show good solubility, e.g., polyethylene glycol, polypropylene glycol, edible oils and glyceryl- and glyceride-based systems. Glyceryl- and glyceride-based systems may include, for example, the following branded products (and corresponding generic products): Captex.™. 355 EP (glyceryl tricaprylate/caprate, from Abitec, Columbus Ohio), Crodamol.™. GTC/C (medium chain triglyceride, from Croda, Cowick Hall, UK) or Labrafac.™. CC (medium chain triglyides, from Gattefosse), Captex.™. 500P (glyceryl triacetate i.e. triacetin, from Abitec), Capmul.™. MCM (medium chain mono- and diglycerides, fromAbitec), Migyol.™. 812 (caprylic/capric triglyceride, from Condea, Cranford N.J.), Migyol.™. 829 (caprylic/capric/succinic triglyceride, from Condea), Migyol.™. 840 (propylene glycol dicaprylate/dicaprate, from Condea), Labrafil™ M1944CS (oleoyl macrogol-6 glycerides, from Gattefosse), Peceol.™. (glyceryl monooleate, from Gattefosse) and Maisine.™. 35-1 (glyceryl monooleate, from Gattefosse). Of particular interest are the medium chain (about C.sub.8 to C.sub.10) triglyceride oils. These solvents frequently make up the predominant portion of the composition, i.e., greater than about 50%, usually greater than about 80%, for example about 95% or 99%. Adjuvants and additives may also be included with the solvents principally as taste-mask agents, palatability and flavoring agents, antioxidants, stabilizers, texture and viscosity modifiers and solubilizers.

Suspensions, in addition to the compound of the present invention or the combination, may further comprise carriers such as suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by mixing a compound of the present invention or a combination with suitable non-irritating excipients or carriers, such as cocoa butter, polyethylene glycol or a suppository wax which are solid at ordinary room temperature, but liquid at body temperature, and therefore, melt in the rectum or vaginal cavity thereby releasing the active component(s).

Dosage forms for topical administration of the compositions of the present invention or combinations include ointments, creams, lotions, powders and sprays. The drugs are admixed with a pharmaceutically acceptable excipient, diluent or carrier, and any preservatives, buffers, or propellants that may be required.

Some of the present compounds contained in the compositions of the invention may be poorly soluble in water, e.g., less than about 1 μg/mL. Therefore, liquid compositions in solubilizing, non-aqueous solvents such as the medium chain triglyceride oils discussed above are a preferred dosage form for these compounds.

Solid amorphous dispersions, including dispersions formed by a spray-drying process, are also a preferred dosage form for the poorly soluble compounds contained in the compositions of the invention. By “solid amorphous dispersion” is meant a solid material in which at least a portion of the poorly soluble compound is in the amorphous form and dispersed in a water-soluble polymer. By “amorphous” is meant that the poorly soluble compound is not crystalline. By “crystalline” is meant that the compound exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC).

Preferably, at least a major portion (i.e., at least about 60 wt %) of the poorly soluble compound in the solid amorphous dispersion is amorphous. The compound can exist within the solid amorphous dispersion in relatively pure amorphous domains or regions, as a solid solution of the compound homogeneously distributed throughout the polymer or any combination of these states or those states that lie intermediate between them. Preferably, the solid amorphous dispersion is substantially homogeneous so that the amorphous compound is dispersed as homogeneously as possible throughout the polymer. As used herein, “substantially homogeneous” means that the fraction of the compound that is present in relatively pure amorphous domains or regions within the solid amorphous dispersion is relatively small, on the order of less than 20 wt %, and preferably less than 10 wt % of the total amount of drug.

Water-soluble polymers suitable for use in the solid amorphous dispersions should be inert, in the sense that they do not chemically react with the poorly soluble compound in an adverse manner, are pharmaceutically acceptable, and have at least some solubility in aqueous solution at physiologically relevant pHs (e.g. 1-8). The polymer can be neutral or ionizable, and should have an aqueous-solubility of at least 0.1 mg/mL over at least a portion of the pH range of 1-8.

Water-soluble polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

Exemplary water-soluble polymers include hydroxypropyl methyl cellulose acetate succinate (HPMCAS), hydroxypropyl methyl cellulose (HPMC), hydroxypropyl methyl cellulose phthalate (HPMCP), carboxy methyl ethyl cellulose (CMEC), cellulose acetate phthalate (CAP), cellulose acetate trimellitate (CAT), polyvinylpyrrolidone (PVP), hydroxypropyl cellulose (HPC), methyl cellulose (MC), block copolymers of ethylene oxide and propylene oxide (PEO/PPO, also known as poloxamers), and mixtures thereof. Especially preferred polymers include HPMCAS, HPMC, HPMCP, CMEC, CAP, CAT, PVP, poloxamers, and mixtures thereof. Most preferred is HPMCAS. See European Patent Application Publication No. 0 901 786 A2, the disclosure of which is incorporated herein by reference.

The solid amorphous dispersions may be prepared according to any process for forming solid amorphous dispersions that results in at least a major portion (at least 60%) of the poorly soluble compound being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray coating and spray drying. See, for example, the following U.S. Patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564, which describe forming dispersions by milling processes; and U.S. Pat. Nos. 5,707,646 and 4,894,235, which describe forming dispersions by melt congeal processes. In a preferred process, the solid amorphous dispersion is formed by spray drying, as disclosed in European Patent Application Publication No. 0 901 786 A2. In this process, the compound and polymer are dissolved in a solvent, such as acetone or methanol, and the solvent is then rapidly removed from the solution by spray drying to form the solid amorphous dispersion. The solid amorphous dispersions may be prepared to contain up to about 99 wt % of the compound, e.g., 1 wt %, 5 wt %, 10 wt %, 25 wt %, 50 wt %, 75 wt %, 95 wt %, or 98 wt % as desired.

The solid dispersion may be used as the dosage form itself or it may serve as a manufacturing-use-product (MUP) in the preparation of other dosage forms such as capsules, tablets, solutions or suspensions. An example of an aqueous suspension is an aqueous suspension of a 1:1 (w/w) compound/HPMCAS-HF spray-dried dispersion containing 2.5 mg/mL of compound in 2% polysorbate-80. Solid dispersions for use in a tablet or capsule will generally be mixed with other excipients or adjuvants typically found in such dosage forms. For example, an exemplary filler for capsules contains a 2:1 (w/w) compound/HPMCAS-MF spray-dried dispersion (60%), lactose (fast flow) (15%), microcrystalline cellulose (e.g., Avicel.sup.(R0-102) (15.8%), sodium starch (7%), sodium lauryl sulfate (2%) and magnesium stearate (1%).

The HPMCAS polymers are available in low, medium and high grades as Aqoa.sup.(R)-LF, Aqoat.sup.(R)-MF and Aqoat.sup.(R)-HF respectively from Shin-Etsu Chemical Co., LTD, Tokyo, Japan. The higher MF and HF grades are generally preferred.

The following paragraphs describe exemplary formulations, dosages, etc. useful for non-human animals. The administration of Compound A, or a pharmaceutically acceptable salt thereof, in combination with Compound D, or a pharmaceutically acceptable salt thereof, as the two agents or in combinations with another agent can be effected orally or non-orally.

An amount of 4-(4-(1-Isopropyl-7-oxo-1,4,6,7-tetrahydrospiro[indazole-5,4′-piperidine]-1′-carbonyl)-6-methoxypyridin-2-yl)benzoic acid, or a pharmaceutically acceptable salt thereof, with 2-{5-[(3-ethoxypyridin-2-yl)oxy]pyridin-3-yl}-N-[(3S,5S)-5-fluoropiperidin-3-yl]pyrimidine-5-carboxamide, or a pharmaceutically acceptable salt thereof, together or in combination with an another agent is administered such that an effective dose is received. Generally, a daily dose that is administered orally to an animal is between about 0.01 and about 1,000 mg/kg of body weight, e.g., between about 0.01 and about 300 mg/kg or between about 0.01 and about 100 mg/kg or between about 0.01 and about 50 mg/kg of body weight, or between about 0.01 and about 25 mg/kg, or about 0.01 and about 10 mg/kg or about 0.01 and about 5 mg/kg. A daily dose of 4-(4-(1-Isopropyl-7-oxo-1,4,6,7-tetrahydrospiro[indazole-5,4′-piperidine]-1′-carbonyl)-6-methoxypyridin-2-yl)benzoic acid, or a pharmaceutically acceptable salt thereof (Compound A) that is administered may be 2 mg, 3 mg, 5 mg, 10 mg, 15 mg, 20 mg, 25 mg, 30 mg, or 50 mg. The daily dose may be divided into multiple doses, such as twice a day (e.g., “BID” or “q12” hour dosing interval). For example, in certain instances the daily dose of Compound A may be administered as 15 mg twice a day (e.g., q12 hours). A daily dose of 2-{5-[(3-ethoxypyridin-2-yl)oxy]pyridin-3-yl}-N-[(3S,5S)-5-fluoropiperidin-3-yl]pyrimidine-5-carboxamide, or a pharmaceutically acceptable salt thereof (Compound of Example 4) that is administered may be 10 mg, 20 mg, 40 mg, or 80 mg The daily dose may be divided into multiple doses, such as twice a day (e.g., “BID” or “q12 hour dosing interval”. For example, in certain instances the daily dose of Compound D may be administered as 300 mg twice a day (e.g., q12 hours).

Conveniently, a compositions of the present invention (or combination) can be carried in the drinking water so that a therapeutic dosage of the compound is ingested with the daily water supply. The compositions can be directly metered into drinking water, preferably in the form of a liquid, water-soluble concentrate (such as an aqueous solution of a water-soluble salt).

These compositions may also be administered to animals other than humans, for example, for the indications detailed above. The precise dosage administered of each active ingredient will vary depending upon any number of factors, including but not limited to, the type of animal and type of disease state being treated, the age of the animal, and the route(s) of administration.

A dosage of the composition containing the Formula (I) or (II) compounds is used that is effective for the indication being treated. Such dosages can be determined by standard assays such as those referenced above and provided herein.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

The present invention further comprises use of a compound of Formula (I) or (II) for use as a medicament (such as a unit dosage tablet or unit dosage capsule). In another embodiment, the present invention comprises the use of a compound of Formula (I) or (II) for the manufacture of a medicament (such as a unit dosage tablet or unit dosage capsule) to treat one or more of the conditions previously identified in the above sections discussing methods of treatment.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

These agents and compounds of the invention can be combined with pharmaceutically acceptable vehicles such as saline, Ringer's solution, dextrose solution, and the like. The particular dosage regimen, i.e., dose, timing and repetition, will depend on the particular individual and that individual's medical history.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or Igs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Liposomes containing these agents and/or compounds of the invention are prepared by methods known in the art, such as described in U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

These agents and/or the compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing the compound of the invention, which matrices are in the form of shaped articles, e.g., films, or microcapsules. Examples of sustained-release matrices include polyesters, hydrogels (for example, poly(2-hydroxyethyl-methacrylate), or ′poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as those used in LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a compound of the invention with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

The compositions herein may be formulated for oral, buccal, intranasal, parenteral (e.g., intravenous, intramuscular or subcutaneous) or rectal administration or in a form suitable for administration by inhalation. The compositions of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition to be administered will contain a quantity of a compound(s) according to the invention in an amount effective to treat the disease/condition of the subject being treated.

Since the present invention has an aspect that relates to the treatment of the disease/conditions described herein with a composition of active ingredients which may be administered separately, the invention also relates to combining separate pharmaceutical compositions in kit form. The kit comprises two separate pharmaceutical compositions: a compound of Formula (I) or (II) a prodrug thereof or a salt of such compound or prodrug and a second compound as described above. The kit comprises a means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula (I) or (II) compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Also, as the present invention has an aspect that relates to the treatment of the disease/conditions described herein with a composition of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

The active ingredient may be delivered as a solution in an aqueous or non-aqueous vehicle, with or without additional solvents, co-solvents, excipients, or complexation agents selected from pharmaceutically acceptable diluents, excipients, vehicles, or carriers.

The active ingredient may be formulated as a solid dispersion or as a self emulsified drug delivery system (SEDDS) with pharmaceutically acceptable excipients.

The active ingredient may be formulated as an immediate release or suspended release tablet or capsule. Alternatively, the active ingredient may be delivered as the active ingredient alone within a capsule shell, without additional excipients.

### EXAMPLES

### Experimental Procedures

Unless specified otherwise, starting materials are generally available from commercial sources such as Aldrich Chemicals Co. (Milwaukee, WI), Lancaster Synthesis, Inc. (Windham, NH), Acros Organics (Fairlawn, NJ), Maybridge Chemical Company, Ltd. (Cornwall, England) and Tyger Scientific (Princeton, NJ). Certain common abbreviations and acronyms have been employed which may include: AcOH (acetic acid), DBU (1,8-diazabicyclo[5.4.0]undec-7-ene), CDI (1,1′-carbonyldiimidazole), DCM (dichloromethane), DEA (diethylamine), DIPEA (N,N-diisopropylethylamine), DMAP (4-dimethylaminopyridine), DMF (N,N-dimethylformamide), DMSO (dimethyl sulfoxide), EDCl (1-[3-(dimethylamino)propyl]-3-ethylcarbodiimide hydrochloride), Et₂O (diethyl ether), EtOAc (ethyl acetate), EtOH (ethanol), G or g (gram), HATU (0-(7-azabenzotriazol-1-yl)-N,N,N′,N′-tetramethyluronium hexafluorophosphate), HBTU (0-benzotriazol-1-yl-N,N,N′,N′-tetramethyluronium hexafluorophosphate), HOBT (1-hydroxybenzotriazole), H or h (hour), IPA (isopropyl alcohol), iPrOH (2-propanol), KHMDS (potassium bis(trimethylsilyl)amide), MeOH (methanol), MTBE (tert-butyl methyl ether), NaBH(OAc)₃ (sodium triacetoxyborohydride), NaHMDS (sodium bis(trimethylsilyl)amide), NMP (N-methylpyrrolidone), RH (relative humidity), RT or rt (room temperature which is the same as ambient temperature (about 20 to 25° C.)), SEM ([2-(trimethylsilyl)ethoxy]methyl), TEA (triethylamine), TFA (trifluoroacetic acid), THE (tetrahydrofuran), and T₃P (propane phosphonic acid anhydride, 2,4,6-tripropyl-1,3,5,2,4,6-trioxatriphosphinane 2,4,6-trioxide).

¹H Nuclear magnetic resonance (NMR) spectra were in all cases consistent with the proposed structures. Characteristic chemical shifts (5) are given in parts-per-million (ppm) relative to the residual proton signal in the deuterated solvent (CHCl₃ at 7.27 ppm; CD₂HOD at 3.31 ppm) and are reported using conventional abbreviations for designation of major peaks: e.g. s, singlet; d, doublet; t, triplet; q, quartet; m, multiplet; br, broad.
ssNMR means solid-state NMR.
PXRD means Powder X-ray Diffraction.

The term “substantially the same” when used to describe X-ray powder diffraction patterns is mean to include patterns in which peaks are within a standard deviation of +/−0.2⁰ 2⊖.

As used herein, the term “substantially pure” with reference to a particular crystalline form means that the crystalline form includes less than 10%, preferably less than 5%, preferably less than 3%, preferably less than 1% by weight of any other physical form of Compound A or Compound D.

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wisconsin or DriSolv™ products from EMD Chemicals, Gibbstown, NJ) were employed. In some cases, commercial solvents were passed through columns packed with 4 Å molecular sieves, until the following QC standards for water were attained: a)<100 ppm for dichloromethane, toluene, N,N-dimethylformamide, and tetrahydrofuran; b)<180 ppm for methanol, ethanol, 1,4-dioxane, and diisopropylamine. For very sensitive reactions, solvents were further treated with metallic sodium, calcium hydride, or molecular sieves, and distilled just prior to use. Other commercial solvents and reagents were used without further purification. For syntheses referencing procedures in other Examples or Methods, reaction conditions (reaction time and temperature) may vary. Products were generally dried under vacuum before being carried on to further reactions or submitted for biological testing.

When indicated, reactions were heated by microwave irradiation using Biotage Initiator or Personal Chemistry Emrys Optimizer microwaves. Reaction progress was monitored using thin-layer chromatography (TLC), liquid chromatography-mass spectrometry (LCMS), and/or high-performance liquid chromatography (HPLC). TLC was performed on pre-coated silica gel plates with a fluorescence indicator (254 nm excitation wavelength) and visualized under UV light and/or with 12, KMnO₄, CoCl₂, phosphomolybdic acid, and/or ceric ammonium molybdate stains. LCMS data were acquired on an Agilent 1100 Series instrument with a Leap Technologies autosampler, Gemini C18 columns, MeCN/water gradients, and either TFA, formic acid, or ammonium hydroxide modifiers. The column eluent was analyzed using a Waters ZQ mass spectrometer scanning in both positive and negative ion modes from 100 to 1200 Da. Other similar instruments were also used. HPLC data were acquired on an Agilent 1100 Series instrument using Gemini or XBridge C18 columns, MeCN/water gradients, and either TFA or ammonium hydroxide modifiers. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Chiral purifications were performed by chiral supercritical fluid chromatography (SFC) using Berger or Thar instruments; ChiralPAK-AD, -AS, —IC, Chiralcel-OD, or-OJ columns; and CO₂ mixtures with MeOH, EtOH, iPrOH, or MeCN, alone or modified using TFA or iPrNH₂. UV detection was used to trigger fraction collection. For syntheses referencing procedures in other Examples or Methods, purifications may vary: in general, solvents and the solvent ratios used for eluents/gradients were chosen to provide appropriate R_{fS} or retention times.

Mass spectrometry data are reported from LCMS analyses. Mass spectrometry (MS) was performed via atmospheric pressure chemical ionization (APCI), electrospray Ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on on 300, 400, 500, or 600 MHz Varian, Bruker, or Jeol spectrometers. Chemical shifts are expressed in parts per million (ppm, δ) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The peak shapes are described as follows: s, singlet; d, doublet; t, triplet; q, quartet; quin, quintet; m, multiplet; br s, broad singlet; app, apparent. Analytical SFC data were acquired on a Berger analytical instrument as described above. Optical rotation data were acquired on a PerkinElmer model 343 polarimeter using a 1 dm cell. Silica gel chromatography was performed primarily using medium-pressure Biotage or ISCO systems using columns pre-packaged by various commercial vendors including Biotage and ISCO. Microanalyses were performed by Quantitative Technologies Inc. and were within 0.4% of the calculated values.

Unless otherwise noted, chemical reactions were performed at room temperature (about 23 degrees Celsius).

Unless noted otherwise, all reactants were obtained commercially without further purifications or were prepared using methods known in the literature.

The terms “concentrated”, “evaporated”, and “concentrated in vacuo” refer to the removal of solvent at reduced pressure on a rotary evaporator with a bath temperature less than 60° C. The abbreviation “min” and “h” stand for “minutes” and “hours” respectively. The term “TLC” refers to thin-layer chromatography, “room temperature or ambient temperature” means a temperature between 18 to 25° C., “LCMS” refers to liquid chromatography-mass spectrometry, “UPLC” refers to ultra-performance liquid chromatography and “HPLC” refers to high-performance liquid chromatography, “SFC” refers to supercritical fluid chromatography.

Hydrogenation may be performed in a Parr Shaker under pressurized hydrogen gas, or in Thales-nano H-Cube flow hydrogenation apparatus at full hydrogen and a flow rate between 1-2 mL/min at specified temperature.

HPLC, UPLC, LCMS, and SFC retention times were measured using the methods noted in the procedures.

In some examples, chiral separations were carried out to separate enantiomers of certain compounds of the invention (in some examples, the separated enantiomers are designated as ENT-1 and ENT-2, according to their order of elution; similarly, separated diastereomers are designated as DIAST-1 and DIAST-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds are indicated either by the absence of drawn or described stereochemistry, or by the presence of (+/−) adjacent to the structure; in this latter case, the indicated stereochemistry represents just one of the two enantiomers that make up the racemic mixture.

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

The compounds and intermediates described below were named using the naming convention provided with ACD/ChemSketch 2017.2.1, File Version C40H41, Build 99535 (Advanced Chemistry Development, Inc., Toronto, Ontario, Canada). The naming convention provided with ACD/ChemSketch 2017.2.1 is well known by those skilled in the art and it is believed that the naming convention provided with ACD/ChemSketch 2017.2.1 generally comports with the IUPAC (International Union for Pure and Applied Chemistry) recommendations on Nomenclature of Organic Chemistry and the CAS Index rules.

### Preparation P1

### 2-{5-[(3-Ethoxypyridin-2-yl)oxy]pyridin-3-yl}pyrimidine-5-carboxylic acid (P1)

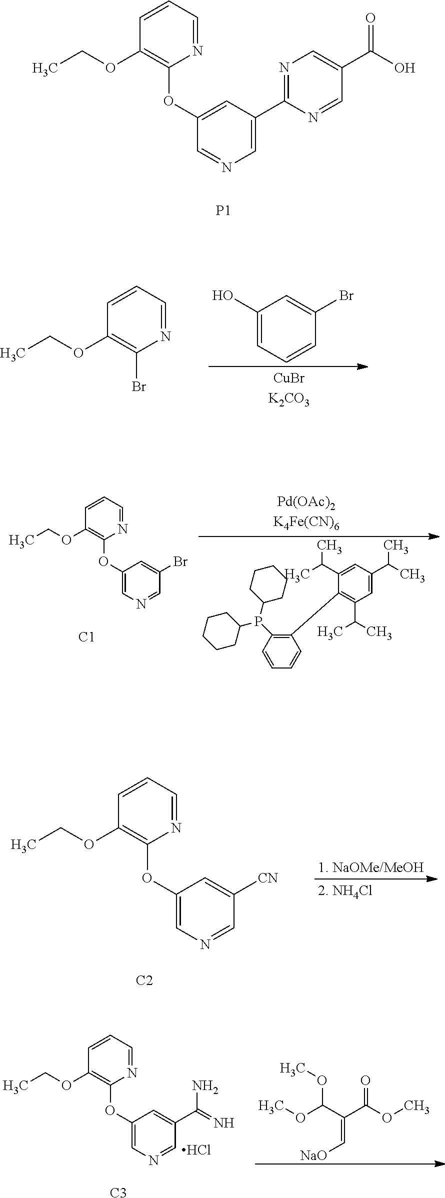

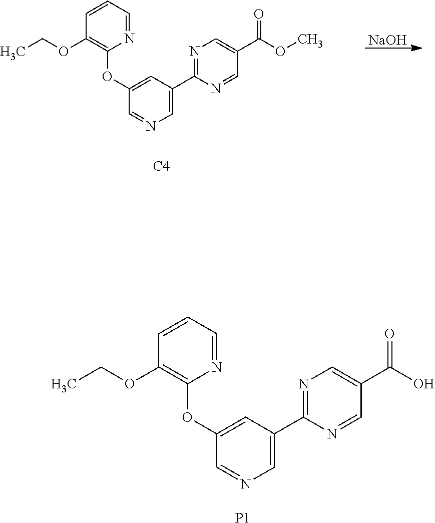

### Step 1. Synthesis of 2-[(5-bromopyridin-3-yl)oxy]-3-ethoxypyridine (C1)

To a solution of 2-bromo-3-ethoxypyridine (841 g, 4.16 mol) in N,N-dimethylformamide (5.4 L) was added copper(I) bromide (538 g, 3.75 mol), followed by potassium carbonate (1.04 kg, 7.52 mol). The resulting mixture was stirred at 25° C., and 5-bromopyridin-3-ol (652 g, 3.75 mol) was added in one portion, whereupon the reaction mixture was heated at 120° C. for 16 hours. It was then cooled to 30° C., and slowly poured into a mixture of crushed ice (9.0 kg) and a 10% solution of ammonia in water (7.0 L). After the resulting suspension had been stirred for 1 hour, the precipitate was collected by filtration, and the filter cake was washed with water (3×5 L). It was then stirred for 1 hour in ethyl acetate (8 L) and filtered; the filtrate was washed with saturated aqueous sodium chloride solution (5 L), dried over sodium sulfate, filtered, and concentrated in vacuo. The residue was stirred in petroleum ether (2 L), and the solid was collected by filtration to provide C1. The filtrate was concentrated under reduced pressure, and the residue was triturated with petroleum ether (200 mL) to afford additional C1. The two batches were combined to provide C1 as a yellow solid. Yield: 835 g, 2.83 mol, 75%. ¹H NMR (400 MHz, chloroform-d) δ 8.48 (d, J=1.9 Hz, 1H), 8.44 (d, J=2.4 Hz, 1H), 7.71 (dd, J=4.9, 1.5 Hz, 1H), 7.69 (dd, J=2.2, 2.1 Hz, 1H), 7.24 (dd, J=7.9, 1.5 Hz, 1H), 7.03 (dd, J=7.9, 4.9 Hz, 1H), 4.14 (q, J=7.0 Hz, 2H), 1.47 (t, J=7.0 Hz, 3H).

### Step 2. Synthesis of 5-[(3-ethoxypyridin-2-yl)oxy]pyridine-3-carbonitrile (C2)

To a solution of C1 (324 g, 1.10 mol) in N,N-dimethylformamide (3.0 L) was added potassium ferrocyanide (II) trihydrate (697 g, 1.65 mol) in one portion, followed by water (300 mL). The resulting suspension was degassed under vacuum and then purged with nitrogen; this evacuation-purge cycle was carried out a total of four times. Palladium(II) acetate (4.94 g, 22.0 mmol) and 2-dicyclohexylphosphino-2′,4′,6′-triisopropylbiphenyl (XPhos; 18.7 g, 39.2 mmol) were then added, the evacuation-purge cycle was carried out five times, and the reaction mixture was heated at 105° C. for 18 hours. After the reaction mixture had been cooled to 50° C., it was filtered, and the filtrate was poured into ice water (3 L) and then stirred for approximately 1 hour. The resulting solid was collected by filtration, dissolved in ethyl acetate (1.5 L), washed with saturated aqueous sodium chloride solution (1 L), dried, filtered, and concentrated in vacuo to provide C2 (209 g) as a yellow solid. The aqueous filtrate from above was extracted with tert-butyl methyl ether (2×3 L), and the combined organic layers were washed with saturated aqueous sodium chloride solution (0.5 L), dried, filtered, and concentrated under reduced pressure to provide additional C2 (20 g). Combined yield: 229 g, 0.949 mol, 86%. ¹H NMR (400 MHz, chloroform-d) δ 8.71 (d, J=2.7 Hz, 1H), 8.68 (d, J=1.8 Hz, 1H), 7.80 (dd, J=2.7, 1.7 Hz, 1H), 7.70 (dd, J=4.9, 1.5 Hz, 1H), 7.29-7.25 (m, 1H, assumed; partially obscured by solvent peak), 7.08 (dd, J=8.0, 4.9 Hz, 1H), 4.16 (q, J=7.0 Hz, 2H), 1.49 (t, J=7.0 Hz, 3H).

### Step 3. Synthesis of 5-[(3-ethoxypyridin-2-yl)oxy]pyridine-3-carboximidamide, hydrochloride Salt (C3)

To a 0° C. to 5° C. suspension of C2 (180 g, 0.746 mol) in methanol (1.3 L) was added a solution of sodium methoxide in methanol (5 M; 30 mL, 150 mmol). Methanol (500 mL) was added to facilitate stirring, whereupon the reaction mixture was allowed to warm to room temperature (18° C.) and stirred for 20 hours. It was then cooled to −40° C. and treated with ammonium chloride (48.0 g, 897 mmol) in one portion, whereupon the mixture was warmed to room temperature (18° C.) and stirred for 40 hours. After concentration in vacuo to remove approximately half of the methanol, the resulting white precipitate (inorganic salt) was removed via filtration. The filtrate was diluted with ethyl acetate (400 mL) and concentrated under reduced pressure to a volume of approximately 400 mL. This ethyl acetate dilution-concentration procedure was repeated twice to effect exchange of methanol with ethyl acetate. Filtration of the resulting suspension afforded C3 as a white solid. Yield: 175 g, 0.594 mol, 80%. ¹H NMR (400 MHz, DMSO-d₆) δ 8.82 (d, J=2 Hz, 1H), 8.77 (d, J=2.6 Hz, 1H), 8.05 (dd, J=2, 2 Hz, 1H), 7.66 (dd, J=4.9, 1.5 Hz, 1H), 7.57 (dd, J=7.9, 1.5 Hz, 1H), 7.18 (dd, J=7.9, 4.9 Hz, 1H), 4.17 (q, J=7.0 Hz, 2H), 1.37 (t, J=7.0 Hz, 3H).

### Step 4. Synthesis of methyl 2-{5-[(3-ethoxypyridin-2-yl)oxy]pyridin-3-yl}pyrimidine-5-carboxylate (C4)

Sodium 2-(dimethoxymethyl)-3-methoxy-3-oxoprop-1-en-1-olate (201 g, 1.01 mol) was added in one portion to a solution of C3 (249 g, 0.845 mol) in methanol (1.75 L). The reaction mixture, a thick suspension, was stirred at 18° C. for 20 hours, whereupon the precipitate was collected by filtration to afford C4 as a white solid. Yield: 259 g, 0.735 mol, 87%. ¹H NMR (400 MHz, DMSO-d₆) δ 9.40 (d, J=1.8 Hz, 1H), 9.35 (s, 2H), 8.67 (d, J=2.7 Hz, 1H), 8.37 (dd, J=2.8, 1.8 Hz, 1H), 7.69 (dd, J=4.8, 1.5 Hz, 1H), 7.58 (dd, J=8.0, 1.5 Hz, 1H), 7.19 (dd, J=8.0, 4.8 Hz, 1H), 4.18 (q, J=7.0 Hz, 2H), 3.93 (s, 3H), 1.36 (t, J=7.0 Hz, 3H).

### Step 5. Synthesis of 2-{5-[(3-ethoxypyridin-2-yl)oxy]pyridin-3-yl}pyrimidine-5-carboxylic acid (P1)

This reaction was carried out in two parallel batches: C4 (321 g, 0.911 mol) was suspended in methanol (1.6 L), cooled in a bath of ice water, and treated in a drop-wise manner with sodium hydroxide solution (2 M; 684 mL, 1.37 mol). After completion of the addition, the reaction mixture was stirred at 14° C. for 16 hours, whereupon the two batches were combined. The resulting suspension was diluted with water (3.2 L), cooled with an ice water bath (approximately 5° C. to 10° C.), and adjusted to pH 3 to 4 by drop-wise addition of 1 M hydrochloric acid. The mixture was stirred at 14° C. for 2 hours, then filtered; the filter cake was washed sequentially with water (2×1 L) and ethyl acetate (1 L), affording P1 as a white solid. Combined yield: 557 g, 1.65 mol, 91%. LCMS m/z 338.9 [M+H]⁺. ¹H NMR (400 MHz, DMSO-d₆) δ 9.39 (d, J=1.8 Hz, 1H), 9.31 (s, 2H), 8.65 (d, J=2.7 Hz, 1H), 8.36 (dd, J=2.7, 1.8 Hz, 1H), 7.68 (dd, J=4.9, 1.5 Hz, 1H), 7.56 (dd, J=8.0, 1.5 Hz, 1H), 7.17 (dd, J=8.0, 4.8 Hz, 1H), 4.16 (q, J=7.0 Hz, 2H), 1.36 (t, J=7.0 Hz, 3H).

Preparation P1 material that was made using an analogous procedure as described above was further analyzed using powder X-ray diffraction analysis conducted on a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 3.00 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.01 degrees and a step time of 1.0 second. The antiscatter screen was set to a fixed distance of 1.5 mm. Samples were rotated at 15/min during collection. Samples were prepared by placing them in a silicon low background sample holder and rotated during collection.

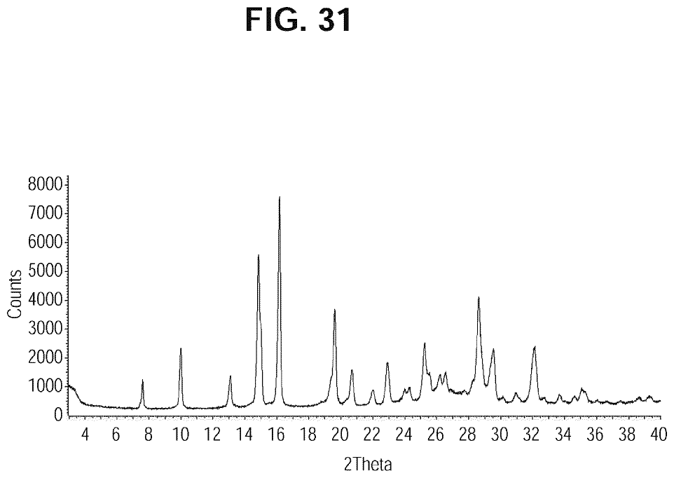

Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity ≥3% were generally chosen. The peaks which were not resolved or were consistent with noise were not selected. A typical error associated with the peak position from PXRD stated in USP up to ±0.2° 2θ (USP-941). Some variation in relative peak heights is expected based on changes with crystal sizes and morphologies. Characteristic x-ray powder diffraction patterns is provided in FIG. 31. The PXRD data from this figure is further described below.

TABLE AA
PXRD peaks for crystalline material of Preparation P1, Form 1

| Angle | Relative | Angle | Relative | Angle | Relative |
|---|---|---|---|---|---|
| 2Θ (°) | intensity (%) | 2Θ (°) | intensity (%) | 2Θ (°) | intensity (%) |
| 7.6 | 13.9 | 22.0 | 7.0 | 26.6 | 13.1 |
| 10.0 | 28.9 | 22.9 | 19.3 | 28.7 | 48.8 |
| 13.1 | 15.0 | 24.0 | 4.1 | 29.6 | 24.8 |
| 14.9 | 72.4 | 24.3 | 4.2 | 32.1 | 25.8 |
| 16.2 | 100.0 | 25.3 | 27.1 | 35.1 | 3.7 |
| 19.6 | 46.0 | 25.6 | 12.8 | 35.3 | 3.9 |
| 20.7 | 16.9 | 26.2 | 11.8 | | |

TABLE AB
Key PXRD peaks to characterize crystalline material of Preparation
P1, Form 1
Preparation P1, Form 1

Angle 2Θ (°) ± 0.2°
7.6, 10.0, 14.9, 16.2, 19.6

### Alternate Synthesis of C2

### 5-[(3-Ethoxypyridin-2-yl)oxy]pyridine-3-carbonitrile (C2)

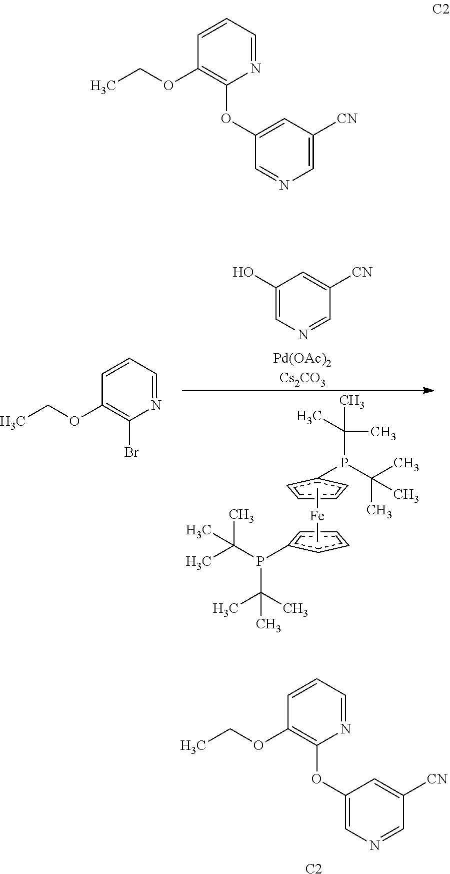

A solution of 2-bromo-3-ethoxypyridine (10.0 g, 49.5 mmol) in 1,4-dioxane (250 mL) was flushed with nitrogen for 2 minutes. 5-Hydroxypyridine-3-carbonitrile (6.54 g, 54.4 mmol), palladium(II) acetate (556 mg, 2.48 mmol), 1,1′-bis(di-tert-butylphosphino)ferrocene (1.41 g, 2.97 mmol), and cesium carbonate (32.3 g, 99.1 mmol) were then added, and the reaction mixture was stirred at 105° C. for 16 hours, whereupon it was combined with a similar reaction carried out using 2-bromo-3-ethoxypyridine (7.00 g, 34.6 mmol) and cooled to room temperature. After dilution with ethyl acetate (200 mL), the combined reaction mixtures were filtered through diatomaceous earth and concentrated to dryness in vacuo. The residue was diluted with ethyl acetate (200 mL), washed with saturated aqueous sodium chloride solution (2×200 mL), dried over sodium sulfate, filtered, and concentrated under reduced pressure. Silica gel chromatography (Gradient: 0% to 35% ethyl acetate in petroleum ether) afforded C2 as a yellow solid. Combined yield: 18.0 g, 74.6 mmol, 89%. LCMS m/z 242.1 [M+H]⁺. ¹H NMR (400 MHz, chloroform-d) δ 8.70 (d, J=2.7 Hz, 1H), 8.66 (d, J=1.8 Hz, 1H), 7.79 (dd, J=2.7, 1.8 Hz, 1H), 7.69 (dd, J=4.9, 1.5 Hz, 1H), 7.26 (dd, J=8.0, 1.5 Hz, 1H), 7.07 (dd, J=8.0, 4.9 Hz, 1H), 4.15 (q, J=7.0 Hz, 2H), 1.47 (t, J=7.0 Hz, 3H).

### Preparation P2

### 2-{5-[(3-Ethoxy-5-fluoropyridin-2-yl)oxy]pyridin-3-yl}pyrimidine-5-carboxylic acid (P2)

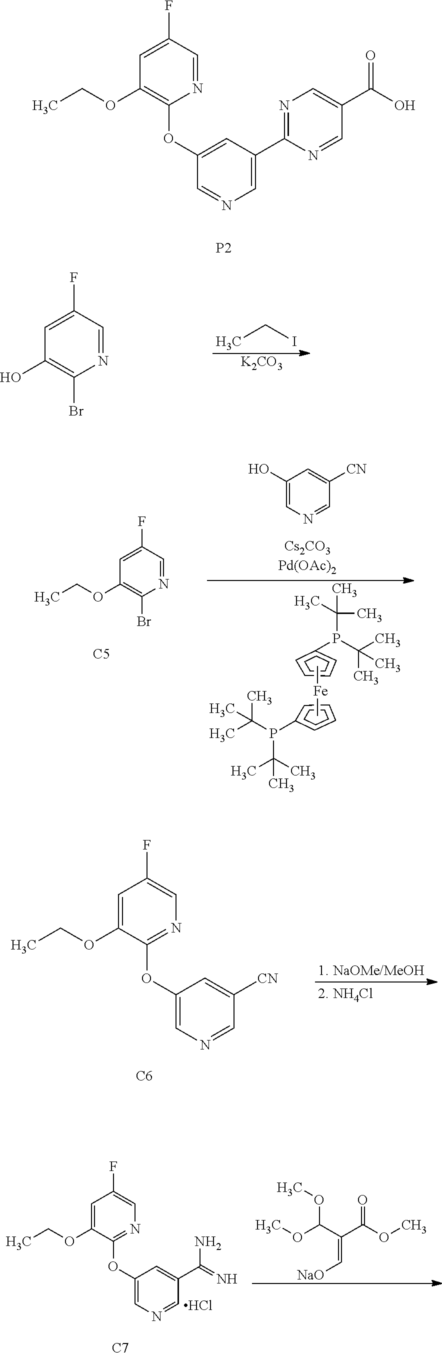

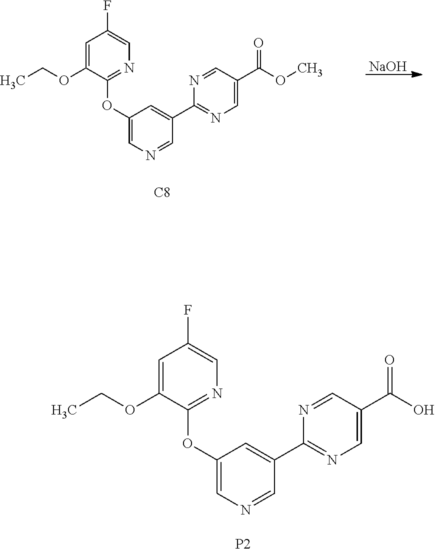

### Step 1. Synthesis of 2-bromo-3-ethoxy-5-fluoropyridine (C5)

Potassium carbonate (697 g, 5.04 mol) was added to a solution of 2-bromo-5-fluoropyridin-3-ol (745 g, 3.88 mol) in N,N-dimethylformamide (4 L), whereupon the reaction vessel was evacuated and charged with nitrogen. This evacuation cycle was repeated twice, and the reaction mixture was then heated to 35° C. Iodoethane (372 mL, 4.65 mol) was added drop-wise over 30 minutes, and after the reaction mixture had been stirred at 35° C. for 16 hours, it was cooled to 25° C. and diluted with water (6 L). Stirring was continued for 15 minutes at 25° C.; the resulting solids were collected via filtration, and the filtrate was extracted with tert-butyl methyl ether (3×1.5 L). The combined organic layers were dried over sodium sulfate, filtered, and concentrated in vacuo. The residue was combined with the solid obtained from the first filtration, affording C5 as a brown solid. Yield: 756 g, 3.44 mol, 89%. ¹H NMR (400 MHz, chloroform-d) δ 7.88 (d, J=2.5 Hz, 1H), 6.90 (dd, J=9.5, 2.5 Hz, 1H), 4.10 (q, J=7.0 Hz, 2H), 1.51 (t, J=7.0 Hz, 3H).

### Step 2. Synthesis of 5-[(3-ethoxy-5-fluoropyridin-2-yl)oxy]pyridine-3-carbonitrile (C6)

This reaction was carried out in four parallel batches. 5-Hydroxypyridine-3-carbonitrile (112.6 g, 937.5 mmol) and cesium carbonate (389 g, 1.19 mol) were added in one portion to a solution of C5 (187.5 g, 852.1 mmol) in 1,4-dioxane (2.5 L). The reaction vessel was evacuated and charged with nitrogen. This evacuation cycle was repeated twice, whereupon palladium(II) acetate (9.57 g, 42.6 mmol) and 1,1′-bis(di-tert-butylphosphino)ferrocene (20.2 g, 42.6 mmol) were added into the reaction mixture. The evacuation-nitrogen cycles were repeated as above, and the reaction mixture was then stirred at 85° C. for 16 hours. Any reactions that were not complete after 16 hours were treated with additional palladium(II) acetate (9.57 g, 42.6 mmol) and 1,1′-bis(di-tert-butylphosphino)ferrocene (20.2 g, 42.6 mmol) and stirred at 85° C. for an additional 16 hours. At this point, the four reaction mixtures were combined and filtered; the filtrate was concentrated in vacuo, and the residue was dissolved in ethyl acetate (3 L), and then diluted with water (6 L). The resulting mixture was stirred at 25° C. for 15 minutes, whereupon the layers were separated, and the aqueous layer was extracted with ethyl acetate (3×1 L). After the combined organic layers had been washed with saturated aqueous sodium chloride solution (2×3 L), they were dried over sodium sulfate, filtered, and concentrated in vacuo. The residue was stirred in a mixture of tert-butyl methyl ether (1 L) and petroleum ether (2 L) at 25° C. for 20 minutes, and the resulting solid was collected via filtration to afford C6 (813 g) as a solid. The filtrate was concentrated in vacuo and subjected to chromatography on silica gel (Gradient: 0% to 30% ethyl acetate in petroleum ether) to provide additional C6 (52 g) as a brown solid. Combined yield: 865 g, 3.34 mol, 98%. LCMS m/z 259.8 [M+H]⁺. ¹H NMR (400 MHz, chloroform-d) δ 8.73-8.64 (m, 2H), 7.77 (br s, 1H), 7.56 (d, J=2.5 Hz, 1H), 7.06 (dd, J=9.0, 2.5 Hz, 1H), 4.14 (q, J=7.0 Hz, 2H), 1.50 (t, J=7.0 Hz, 3H).

### Step 3. Synthesis of 5-[(3-ethoxy-5-fluoropyridin-2-yl)oxy]pyridine-3-carboximidamide, hydrochloride Salt (C7)

A solution of C6 (919 g, 3.54 mol) in ethyl acetate (3 L) was treated with activated carbon (approximately 200 g), and then stirred at 77° C. for 3 hours. After the resulting mixture had been filtered, the filtrate was concentrated in vacuo to provide C6 (894 g, 3.45 mol). The following reaction was then carried out in three parallel batches. Sodium methoxide (12.4 g, 230 mmol) was added to a 0° C. solution of C6 (298 g, 1.15 mol) in methanol (2 L). The reaction mixture was allowed to warm to 25° C. and was then stirred at 25° C. for 20 hours before being cooled to −40° C. and treated with ammonium chloride (67.6 g, 1.26 mol). At this point, the reaction mixture was allowed to warm to 25° C. and was stirred at 25° C. for 40 hours, whereupon the three batches were combined and concentrated in vacuo, affording C7 as a brown gum (1.15 kg). This material was used directly in the following step. LCMS m/z 276.8 [M+H]⁺.

### Step 4. Synthesis of methyl 2-{5-[(3-ethoxy-5-fluoropyridin-2-yl)oxy]pyridin-3-yl}pyrimidine-5-carboxylate (C8)

This reaction was carried out in three parallel batches. Sodium 2-(dimethoxymethyl)-3-methoxy-3-oxoprop-1-en-1-olate (357 g, 1.80 mol) was added in one portion to a 25° C. solution of C7 (from the previous step, 383 g, ≤1.15 mol) in methanol (2.5 L). After stirring at 25° C. for 16 hours, two of the three reactions were complete, as assessed by LCMS analysis. The third (incomplete) reaction was treated with additional sodium 2-(dimethoxymethyl)-3-methoxy-3-oxoprop-1-en-1-olate (54.9 g, 277 mmol), and stirring was continued at 25° C. for 6 hours. At this point, the three batches were combined, diluted with water (8 L), and filtered. After the filter cake had been washed with water (2 ×2 L), it was stirred in a mixture of methanol (1 L) and tert-butyl methyl ether (1 L) for 3 hours at 25° C., whereupon the suspension was filtered to provide C8 as a brown solid. Yield: 1.0 kg, 2.7 mol, 78% over 2 steps. LCMS m/z 370.8 [M+H]⁺. ¹H NMR (400 MHz, chloroform-d) δ 9.54 (d, J=1.8 Hz, 1H), 9.32 (s, 2H), 8.63 (d, J=2.7 Hz, 1H), 8.55 (dd, J=2.7, 1.8 Hz, 1H), 7.57 (d, J=2.5 Hz, 1H), 7.05 (dd, J=9.2, 2.5 Hz, 1H), 4.16 (q, J=7.0 Hz, 2H), 4.00 (s, 3H), 1.51 (t, J=7.0 Hz, 3H).

### Step 5. Synthesis of 2-{5-[(3-ethoxy-5-fluoropyridin-2-yl)oxy]pyridin-3-yl}pyrimidine-5-carboxylic acid (P2)

A suspension of C8 (500 g, 1.35 mol) in methanol (2 L) was treated in a drop-wise manner with sodium hydroxide solution (2 M; 1.01 L, 2.02 mol). After completion of the addition, the reaction mixture was stirred at 25° C. for 16 hours, whereupon it was diluted with water (2 L). The pH was then adjusted to 3 to 4 by drop-wise addition of hydrochloric acid (1 M; approximately 1.5 L); the resulting mixture was stirred at 25° C. for 1 hour, and the solid was collected by filtration. The filter cake was washed with water (2×1 L) to provide P2 as an off-white solid. Yield: 405 g, 1.14 mol, 84%. LCMS m/z 356.8 [M+H]⁺. ¹H NMR (400 MHz, DMSO-d₆) δ 9.39 (d, J=1.7 Hz, 1H), 9.31 (s, 2H), 8.65 (d, J=2.7 Hz, 1H), 8.37-8.34 (m, 1H), 7.71 (d, component of AB quartet, J=2.6 Hz, 1H), 7.68 (dd, component of ABX system, J=9.8, 2.6 Hz, 1H), 4.19 (q, J=7.0 Hz, 2H), 1.36 (t, J=7.0 Hz, 3H).

### Preparation P3

### tert-Butyl (3S,5S)-3-amino-5-fluoropiperidine-1-carboxylate (P3)

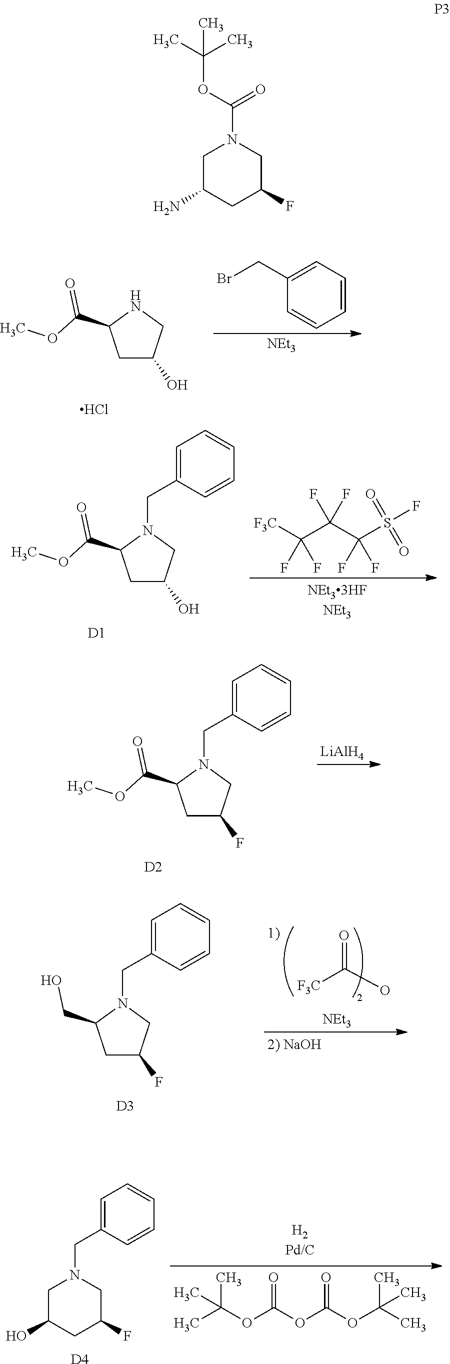

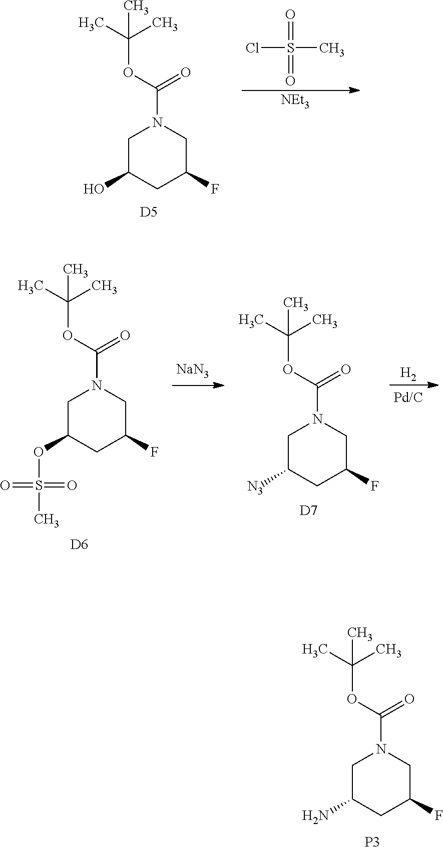

This entire sequence was carried out on large scale. In general, before reactions, as well as after addition of reagents, reactors were evacuated to ≤−0.07 MPa and then filled with nitrogen to normal pressure. This process was generally repeated 3 times, and then oxygen content was assessed to ensure that it was ≤1.0%. For the processes of quenching, extraction, and washing of organic layers, mixtures were generally stirred for 15 to 60 minutes and then allowed to settle for 15 to 60 minutes before separation of layers.

### Step 1. Synthesis of methyl (2S,4R)-1-benzyl-4-hydroxypyrrolidine-2-carboxylate (D1)

Triethylamine (93.55 kg, 924.5 mol) was charged into a 15° C. to 25° C. mixture of dichloromethane (968 kg) and methyl (2S,4R)-4-hydroxypyrrolidine-2-carboxylate, hydrochloride salt (56.05 kg, 308.6 mol) at a reference rate of 80 to 120 kg/hour. After the mixture had been stirred for 10 to 20 minutes, benzyl bromide (79.00 kg, 461.9 mol) was added at a reference rate of 20 to 30 kg/hour. The reaction mixture was stirred at 15° C. to 25° C.; after 12 hours, it was sampled every 2 to 6 hours for analysis via HPLC, until the area percent of the starting material was less than 2% (HPLC conditions. Column: Waters XBridge BEH C18, 4.6×150 mm, 3.5 μm; Mobile phase A: water containing 0.1% heptafluorobutyric acid; Mobile phase B: acetonitrile; Gradient: 10% B for 3 minutes, then 10% to 40% B over 10 minutes, then 40% to 90% B over 5 minutes; Flow rate: 1.0 mL/minute). Typical retention time of methyl (2S,4R)-4-hydroxypyrrolidine-2-carboxylate: 3.8 minutes.

Water (280 kg) was then added to the reactor at a reference rate of 100 to 150 kg/hour and 15° C. to 25° C. The aqueous layer was extracted once with dichloromethane (372 kg, 281 L, 5 volumes); the combined organic layers were washed with a solution of sodium chloride (93.0 kg) in water (280 kg) and then concentrated at −0.08 MPa to a volume of 100 to 150 L while the temperature was maintained below 35° C. Tetrahydrofuran (497 kg) was charged into the resulting mixture in two portions. The mixture was again concentrated at −0.08 MPa to a volume of 100 to 150 L while the temperature was maintained below 35° C. Karl Fischer analysis and sampling for residual dichloromethane were carried out to ensure that water content was 50.30% and residual dichloromethane was ≤4%. The resulting solution was adjusted to 15° C. to 30° C., affording a yellow solution containing D1. Yield: 136.8 kg of solution, containing D1 (69.14 kg, 293.9 mol, 95%). HPLC purity: 90% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Retention time of D1: 4.72 minutes. ¹H NMR (401 MHz, chloroform-d) δ 7.34-7.21 (m, 5H), 4.48-4.39 (m, 1H), 3.90 (d, J=12.9 Hz, 1H), 3.65 (d, J=12.9 Hz, 1H), 3.65 (s, 3H), 3.59 (dd, J=7.9, 7.8 Hz, 1H), 3.32 (dd, J=10.1, 5.7 Hz, 1H), 2.57 (br s, 1H), 2.45 (dd, J=10.2, 4.1 Hz, 1H), 2.24 (ddd, J=13.2, 7.7, 6.9 Hz, 1H), 2.07 (ddd, J=13.3, 8.0, 3.2 Hz, 1H).

### Step 2. Synthesis of methyl (2S,4S)-1-benzyl-4-fluoropyrrolidine-2-carboxylate (D2)

A solution of D1 in tetrahydrofuran (128.4 kg, containing 64.90 kg, 275.8 mol of D1) was added to a reactor containing tetrahydrofuran (575 kg) at 15° C. to 25° C. Triethylamine (114.2 kg, 1129 mol) was then added at a reference addition rate of 35 to 45 kg/hour. Triethylamine trihydrofluoride (66.70 kg, 413.7 mol) was charged into the mixture, followed by nonafluorobutane-1-sulfonyl fluoride (128.0 kg, 423.7 mol) at a reference rate of 60 to 80 kg/hour, and the reaction was allowed to proceed at 15° C. to 25° C.

After 4 hours, the reaction mixture was sampled every 2 to 6 hours for analysis via HPLC, until the area percent of D1 was less than 1% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Typical retention time of D1: 5.0 minutes.

The mixture was then cooled to 10° C. to 15° C., and water (325 kg) was added at a reference rate of 100 to 120 kg/hour, followed by tert-butyl methyl ether (240.5 kg) at 15° C. to 25° C. The organic phase was washed twice with a solution of sodium bicarbonate (18.2 kg, 217 mol) in water (198 kg), and then washed twice with a solution of sodium chloride (47.2 kg) in water (130 kg). It was then concentrated at 5-0.08 MPa to a volume of 150 to 200 L while the temperature was maintained below 45° C. After the resulting mixture had been adjusted to 20° C. to 30° C., tert-butyl methyl ether (123 kg, 166 L, 2.5 volumes) and n-heptane (112 kg, 163 L, 2.5 volumes) were added. The resulting mixture was filtered through a silica gel column (112 kg of silica gel), until nearly all of the material had been filtered. The reactor was then rinsed with tert-butyl methyl ether (481 kg, 10 volumes) and n-heptane (444 kg, 10 volumes) at 20° C. to 30° C., and this rinsing liquor was also filtered through the silica gel column. The combined filtrates were eluted through a fresh column containing silica gel (40 kg), and the eluent was concentrated at ≤−0.08 MPa to a volume of 80 to 100 L while the temperature was maintained below 45° C. The resulting solution was adjusted to 20° C. to 30° C., affording a yellow solution containing D2. Yield: 104.3 kg of solution, containing D2 (52.64 kg, 221.8 mol, 80%). HPLC purity: 83% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Retention time for D2: 7.34 minutes. ¹H NMR (401 MHz, chloroform-d) δ 7.37-7.22 (m, 5H), 5.10 (br ddd, J=54.8, 5, 5 Hz, 1H), 4.03 (d, J=13.0 Hz, 1H), 3.70 (s, 3H), 3.59 (d, J=13.0 Hz, 1H), 3.34-3.21 (m, 2H), 2.65-2.42 (m, 2H), 2.29 (br ddd, J=29.8, 14.8, 6.3 Hz, 1H).

### Step 3. Synthesis of [(2S,4S)-1-benzyl-4-fluoropyrrolidin-2-yl]Methanol (D3)

Tetrahydrofuran (352 kg) was added to lithium aluminum hydride (8.20 kg, 216 mol) at 15° C. to 25° C., under nitrogen. After completion of the addition, the mixture was stirred for 10 to 15 minutes, and nitrogen was bubbled in from the lower port of the reactor for 3 to 5 minutes. The mixture was adjusted to 8° C. to 15° C., and then a solution of D2 in tetrahydrofuran (99.10 kg, containing 50.02 kg, 210.8 mol of D2) was added portion-wise at a reference rate of 35 to 45 kg/hour at 8° C. to 15° C. After one-third of the substrate had been added, the reaction mixture was stirred for 0.5 to 1 hour, and then sampled for analysis. Additional material was not added until 510% of the D2 charge remained. Upon completion of the entire D2 addition, the reaction was allowed to proceed at 8° C. to 15° C.; after 1 hour, it was sampled for HPLC analysis every 1 to 3 hours until ≤2% of D2 was observed (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Typical retention time for D2: 7.5 minutes.

The reaction was then quenched via addition of a mixture of water (8.00 kg) and tetrahydrofuran (44.5 kg); this was added at 0° C. to 20° C. at a reference rate of 6 to 8 kg/hour. A solution of sodium hydroxide (1.40 kg, 35 mol) in water (30.0 kg) was then charged into the mixture at 10° C. to 25° C., at a reference rate of 10 to 20 kg/hour. After this addition, the mixture was stirred for 0.5 to 1 hour. Nitrogen was then bubbled into the mixture from the lower port of the reactor for 4 to 6 hours at 15° C. to 25° C. The mixture was filtered, and the collected solids were stirred with tetrahydrofuran (317 kg) at 15° C. to 25° C. for 1 to 2 hours; this mixture was then filtered. The combined filtrates were concentrated to 1 to 1.2 volumes, at 5-0.08 MPa, while the temperature was maintained below 45° C. 2-Methyltetrahydrofuran (388 kg) was charged into the mixture in portions, while the temperature was maintained below 45° C. After each addition, the mixture was stirred for 5 to 10 minutes, and then concentrated as above to 1 to 1.2 volumes. Sampling was carried out to ensure that residual tetrahydrofuran was ≤2%, and water content (by Karl Fischer analysis) was 50.1%. The resulting mixture was adjusted to 15° C. to 25° C. and treated with 2-methyltetrahydrofuran (43.0 kg); stirring provided a yellow solution containing D3. Yield: 102.7 kg of solution, containing D3 (41.05 kg, 196.2 mmol, 93%). HPLC purity: 90% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Retention time for D3: 5.38 minutes. ¹H NMR (401 MHz, chloroform-d), characteristic peaks: δ 7.37-7.23 (m, 5H), 5.05 (br ddd, J=54.5, 4.7, 4.5 Hz, 1H), 4.04 (d, J=13.3 Hz, 1H), 3.54-3.45 (m, 1H), 3.33 (d, J=13.3 Hz, 1H), 3.23 (br dd, J=18.5, 11.7 Hz, 1H), 2.83-2.75 (m, 1H), 2.63 (br d, J=9.2 Hz, 1H), 2.49-2.10 (m, 3H).

### Step 4. Synthesis of (3R,5S)-1-benzyl-5-fluoropiperidin-3-ol (D4)

A solution of D3 in 2-methyltetrahydrofuran (102.6 kg, containing 41.03 kg, 196.1 mol of D3) was added to 2-methyltetrahydrofuran (160 kg) at 15° C. to 25° C. Trifluoroacetic anhydride (42.20 kg, 200.9 mol) was then added at a reference rate of 35 to 45 kg/hour, followed by triethylamine (61.1 kg, 604 mol) at a reference rate of 35 to 45 kg/hour. The reaction mixture was maintained at 15° C. to 25° C. for 1 hour, then heated to 77° C. to 82° C. After 12 hours, the reaction was sampled every 1 to 12 hours for HPLC analysis, until the area percent of D3 was ≤3% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Typical retention time for D3: 5.8 minutes.

At that time, the reaction mixture was cooled to 10° C. to 20° C. and treated with a solution of sodium hydroxide (3.30 kg, 82 mol) in water (41.1 kg) at a reference rate of 34 to 45 kg/hour, while the temperature was maintained between 10° C. to 30° C. After completion of the addition, the mixture was stirred for 1 hour, whereupon it was washed with a solution of sodium chloride (23.1 kg) in water (82.2 kg) at 15° C. to 30° C. The aqueous layer was extracted once with tert-butyl methyl ether (150 kg, 203 L, 5 volumes) at 15° C. to 30° C., and the combined organic layers were concentrated at ≤−0.08 MPa to 1 to 1.2 volumes, while the temperature was maintained below 45° C. The mixture was washed twice with water (162 kg) at 15° C. to 30° C., and the organic phase was sampled for triethylamine to ensure that the triethylamine level was ≤3%. It was then concentrated at ≤−0.08 MPa to a volume of 1 to 1.2 volumes, while the temperature was maintained below 45° C. Tetrahydrofuran (110 kg) was added, and the resulting mixture was again concentrated at ≤−0.08 MPa to a volume of 1 to 1.2 volumes, while the temperature was maintained below 45° C. The resulting material was cooled to 20° C. to 30° C., affording a dark brown solution containing D4. Yield: 153.5 kg of solution, containing D4 (36.50 kg, 174.4 mmol, 89%). HPLC purity: 85% (HPLC conditions. Column: Waters XBridge BEH C18, 4.6×150 mm, 3.5 μm; Mobile phase A: 10 mM ammonium acetate in water; Mobile phase B: acetonitrile; Gradient: 20% to 95% B over 10 minutes; Flow rate: 1.0 mL/minute). Retention time for D4: 5.88 minutes. ¹H NMR (401 MHz, chloroform-d) δ 7.36-7.23 (m, 5H), 4.84-4.66 (m, 1H), 3.90-3.81 (m, 1H), 3.62-3.59 (m, 2H), 2.92-2.76 (m, 2H), 2.69 (dd, J=11.5, 5.0 Hz, 1H), 2.54-2.39 (m, 2H), 2.10-1.98 (m, 1H), 1.95-1.78 (m, 1H).

### Step 5. Synthesis of tert-butyl (3S,5R)-3-fluoro-5-hydroxypiperidine-1-carboxylate (D5)

A mixture of D4 in tetrahydrofuran (containing 30.03 kg, 143.5 mol of D4), tetrahydrofuran (218 kg), and di-tert-butyl dicarbonate (47.10 kg, 215.8 mol), at 15° C. to 30° C., was purged with nitrogen via a subsurface pipe to 0.2 to 0.3 MPa, then vented to 0.02 to 0.05 MPa. This purge and vent procedure was repeated between 5 and 8 times. Palladium on charcoal (10%, 3.00 kg) was charged into the reactor at 15° C. to 30° C., and the solid addition funnel was then rinsed with water (0.26 kg). The reaction mixture was purged with nitrogen via subsurface pipe to 0.1 to 0.2 MPa, then vented to 0.02 to 0.05 MPa at 15° C. to 30° C.; this purge and vent procedure was repeated between 5 and 8 times. An identical purge-vent protocol was then carried out using hydrogen. After the final hydrogen exchange, the pressure was increased to 0.1 to 0.2 MPa with hydrogen. The reaction mixture was then exchanged with nitrogen twice every 1 to 3 hours, and purged with hydrogen via subsurface pipe to 0.1 to 0.2 MPa, followed by venting to 0.02 to 0.05 MPa. After the exchange, the hydrogen pressure was increased to 0.1 to 0.2 MPa, and the reaction mixture was maintained at 20° C. to 30° C. After 8 hours, the reaction was sampled for HPLC analysis every 1 to 12 hours, until the level of D4 was ≤1.0% (HPLC conditions. Column: Waters XSelect Phenyl-Hexyl, 4.6×150 mm, 3.5 μm; Mobile phase A: water containing 0.1% trifluoroacetic acid; Mobile phase B: acetonitrile containing 0.1% trifluoroacetic acid; Gradient: 5% to 35% B over 15 minutes; Flow rate: 1.0 mL/minute).

The mixture was then purged with nitrogen via subsurface pipe to 0.2 to 0.3 MPa, and vented to 0.02 to 0.05 MPa at 15° C. to 30° C.; this cycle was repeated not less than 9 times. The reaction mixture was passed through a stainless steel nutsche filter at 20° C. to 30° C., and the filter cake was rinsed with tetrahydrofuran (26.6 kg, 29.9 L, 1 volume); the combined filtrates were passed through a filter loaded with silica gel (15.1 kg), and the silica filter cake was rinsed with tetrahydrofuran (52.7 kg, 59.3 L, 2 volumes). These combined filtrates were passed through an in-line filter at 15° C. to 30° C. and concentrated at ≤−0.08 MPa to a volume of 1.1 to 1.4 volumes, while the temperature was maintained below 45° C. The resulting mixture was treated with n-heptane (102 kg) at 15° C. to 45° C. and stirred for 10 minutes, whereupon the mixture was sampled to ensure that residual tetrahydrofuran was <8%. Tetrahydrofuran (6.90 kg) and n-heptane (101 kg) were added at 15° C. to 45° C., and the mixture was heated to 50° C. to 55° C., then cooled to 18° C. to 25° C. and stirred for 1 hour. Seed crystals of D5 (0.06 kg; see origin below) were charged into the mixture, which was then was stirred for 1 to 2 hours while the temperature was maintained at 18° C. to 25° C. Stirring was continued at 15° C. to 20° C. for 8 to 12 hours for crystallization. Nitrogen was bubbled in from the lower port of the reactor every 2 to 3 hours to effect concentration. The mixture was then filtered, using a stainless steel nutsche filter; the filter cake was rinsed with a mixture of n-heptane (20.4 kg) and tetrahydrofuran (0.81 kg) and then dried at 20° C. to 30° C. until sampling indicated residual tetrahydrofuran 5720 ppm and residual n-heptane ≤5000 ppm. Product D5 was obtained as an off-white solid. Yield: 12.15 kg, 97.5% by assay; corrected weight: 11.84 kg, 54.00 mol. Additional material from mother liquor recovery: 5.17 kg, 23.6 mmol. Combined yield: 54%. HPLC purity: 94% (HPLC conditions. Column: Waters XSelect Phenyl-Hexyl, 4.6×150 mm, 3.5 μm; Mobile phase A: water containing 0.1% trifluoroacetic acid; Mobile phase B: acetonitrile containing 0.1% trifluoroacetic acid; Gradient: 5% to 35% B over 15 minutes; Flow rate: 1.0 mL/minute). Retention time for D5: 12.58 minutes. ¹H NMR (401 MHz, chloroform-d) δ 4.69 (br d, J_{HF}=47.2 Hz, 1H), 3.86-3.76 (m, 1H), 3.62-3.35 (m, 3H), 2.64-2.44 (m, 1H), 2.18-1.92 (m, 2H), 1.46 (s, 9H).

Preparation of seed crystal of D5: A smaller-scale hydrogenation reaction of D4 was carried out as above; after removal of the palladium on carbon, the resulting solution of D5 in tetrahydrofuran was concentrated to approximately 1 to 1.2 volumes (based on the quantity of D4 used). The resulting mixture was treated with tetrahydrofuran (0.2 volumes) and n-heptane (15 volumes), heated to 50° C. to 55° C., and then allowed to cool to 15° C. to 20° C. After the suspension had been stirred for 6 to 8 hours, it was filtered to afford D5 as a solid; this material was used as the seed crystals.

### Step 6. Synthesis of tert-butyl (3S,5R)-3-fluoro-5-[(methylsulfonyl)oxy]piperidine-1-carboxylate (D6)

Dichloromethane (229 kg) was charged into a reactor at 15° C. to 25° C., followed by D5 (17.40 kg; corrected from assay results: 16.96 kg, 77.35 mol); nitrogen was then bubbled in through the lower port. Triethylamine (9.80 kg, 96.8 mol) was added at 15° C. to 25° C., whereupon the reaction mixture was cooled to 0° C. to 5° C. While the mixture was maintained at 0° C. to 10° C., methanesulfonyl chloride (10.18 kg, 88.88 mol) was added, and the reaction was allowed to proceed at 0° C. to 10° C. After 1 hour, it was sampled every 1 to 3 hours until the area percent of D5 was less than 1% (HPLC conditions. Column: Waters XBridge BEH C18, 4.6×150 mm, 3.5 μm; Mobile phase A: 10 mM ammonium acetate in water; Mobile phase B: acetonitrile; Gradient: 30% to 95% B over 10 minutes; Flow rate: 1.0 mL/minute). Typical retention time for D5: 4.31 minutes.

At that point, water (52.0 kg) was charged into the mixture at 0° C. to 5° C., at a reference rate of 20 to 38 kg/hour. The aqueous phase of the resulting mixture was extracted with dichloromethane (70.4 kg, 53.1 L, 3 volumes) at 0° C. to 10° C., and the combined organic layers were washed with a solution of sodium chloride (4.30 kg) in water (17.4 kg) at 0° C. to 15° C., and subsequently concentrated at ≤−0.09 MPa to 2 to 3 volumes while the temperature was maintained below 30° C. The residue was diluted with tert-butyl methyl ether (128 kg) in portions at 0° C. to 30° C., and then the mixture was concentrated at ≤−0.09 MPa to 2 to 3 volumes while the temperature was maintained below 30° C. tert-Butyl methyl ether (65.0 kg) was again charged into the mixture portion-wise at 0° C. to 30° C., and the mixture was again concentrated at ≤−0.09 MPa to 2 to 3 volumes while the temperature was maintained below 30° C. This protocol was repeated until analysis for dichloromethane provided a residual dichloromethane level of 52%. The mixture was then heated to 35° C. to 40° C., and n-heptane (116 kg) was added at a reference rate of 40 to 60 kg/hour, whereupon the mixture was slowly cooled to 0° C. to 10° C. at a reference rate of 10° C. to 15° C./hour. It was then stirred at 0° C. to 10° C. for 2 hours. Filtration provided a filter cake, which was rinsed with n-heptane (22.5 kg), and then dried at 35° C. to 40° C. under a flow of nitrogen. The resulting solid was mixed with tert-butyl methyl ether (59.4 kg) at 15° C. to 30° C., and then heated to 35° C. to 40° C. n-Heptane (119 kg) was added at 35° C. to 40° C., at a reference rate of 40 to 60 kg/hour, and the resulting mixture was slowly cooled to 0° C. to 10° C., at a reference rate of 10° C. to 15° C./hour. After the mixture had been stirred at 0° C. to 10° C. for 2 hours, it was filtered, and the filter cake was rinsed with n-heptane (23.6 kg). The collected solid was dried at 35° C. to 40° C. until residual tert-butyl methyl ether was: 5000 ppm, residual n-heptane was: 5000 ppm, and Karl Fischer analysis revealed water content of 50.1%. The resulting material was cooled to 15° C. to 30° C., affording D6 as a white solid. Yield: 19.35 kg, 98.3% by assay; corrected weight: 19.02 kg, 63.97 mol, 83%. HPLC purity: 99% (HPLC conditions. Column: Waters XBridge BEH C18, 4.6×150 mm, 3.5 μm; Mobile phase A: 10 mM ammonium acetate in water; Mobile phase B: acetonitrile; Gradient: 30% to 95% B over 10 minutes; Flow rate: 1.0 mL/minute). Retention time for D6: 4.98 minutes. ¹H NMR (401 MHz, chloroform-d) δ 4.79-4.52 (m, 2H), 3.72-3.53 (m, 4H), 3.06 (s, 3H), 2.36-2.11 (m, 2H), 1.46 (s, 9H).

### Step 7. Synthesis of tert-butyl (3S,5S)-3-azido-5-fluoropiperidine-1-carboxylate (D7)

N,N-Dimethylformamide (174 kg) and D6 (18.45 kg; corrected for assay 18.14 kg, 61.01 mol) were charged into a reactor and stirred at 15° C. to 30° C. until a solution was obtained, whereupon sodium azide (6.05 kg, 93.1 mol) was added at 15° C. to 30° C. The reaction mixture was heated to 78° C. to 88° C., at a reference rate of 20° C. to 35° C./hour, and then allowed to react at 78° C. to 88° C. After 6 to 12 hours, the reaction mixture was sampled every 2 to 8 hours for HPLC analysis, until the area percent of D6 was less than 0.5% (HPLC conditions. Column: Waters XBridge BEH C18, 4.6×150 mm, 3.5 μm; Mobile phase A: water containing 0.1% ammonium acetate; Mobile phase B: acetonitrile; Gradient: 20% to 95% B over 10 minutes; Flow rate: 1.0 mL/minute). Typical retention time for D6: 6.4 minutes.

After the mixture had been cooled to 10° C. to 20° C., tert-butyl methyl ether (68.7 kg) and water (185 kg) were added, at a reference rate of 35 to 85 kg/hour, and stirring was continued for 10 to 20 minutes. The mixture was filtered, and the aqueous layer of the filtrate was extracted with tert-butyl methyl ether (2×69 kg, 93 L, 5 volumes). The combined organic layers were washed with water (2×56 kg, 56 L, 3 volumes) to afford D7 as a light yellow solution in tert-butyl methyl ether. Yield: 185.4 kg; solution in tert-butyl methyl ether, assumed to contain 14.90 kg, 61.01 mol of D7, 100%. HPLC purity: 80% (HPLC conditions. Column: Waters XBridge BEH C18, 4.6×150 mm, 3.5 μm; Mobile phase A: water containing 0.1% ammonium acetate; Mobile phase B: acetonitrile; Gradient: 20% to 95% B over 10 minutes; Flow rate: 1.0 mL/minute). Retention time for D7: 8.37 minutes.

### Step 8. Synthesis of tert-butyl (3S,5S)-3-amino-5-fluoropiperidine-1-carboxylate (P3)

Methanol (30.2 kg) and D7 (185.4 kg of the tert-butyl methyl ether solution from the previous step; assumed to contain 61.01 mmol D7) were charged into a reactor at 15° C. to 30° C. The mixture was purged with nitrogen via a subsurface pipe to 0.2 to 0.3 MPa, then vented to 0.02 to 0.05 MPa; this purge/vent operation was carried out 5 to 8 times, until the oxygen content was less than 1.0%. Palladium on charcoal (10%, 0.95 kg) was added at 15° C. to 30° C., whereupon the addition funnel was rinsed with water (0.15 kg). The resulting mixture was purged with nitrogen via a subsurface pipe to 0.2 to 0.3 MPa, then vented to 0.02 to 0.05 MPa at 15° C. to 30° C. This purge/vent procedure was repeated not less than 9 times. The same procedure was then carried out 5 to 8 times, except that hydrogen was used in place of nitrogen. The reaction mixture was then purged with hydrogen via a subsurface pipe to 0.1 to 0.2 MPa, and allowed to react at 20° C. to 30° C. The hydrogen was exchanged twice every 1 to 3 hours in the following manner: the mixture was purged with hydrogen via a subsurface pipe to 0.1 to 0.2 MPa, then vented to 0.02-0.05 MPa and finally purged with hydrogen to 0.1 to 0.2 MPa. After 6 to 12 hours at 20° C. to 30° C., the reaction mixture was sampled every 3 to 12 hours for HPLC analysis, until the area percent of D7 was ≤1.0% (HPLC conditions. Column: Waters XBridge BEH C18, 4.6×150 mm, 3.5 μm; Mobile phase A: water containing 0.1% ammonium acetate; Mobile phase B: acetonitrile; Gradient: 20% to 95% B over 10 minutes; Flow rate: 1.0 mL/minute). Typical retention time for D7: 8.6 minutes.

The reaction mixture was then purged with nitrogen to 0.2 to 0.3 MPa and vented to 0.02 to 0.05 MPa at 15° C. to 30° C. This purge/vent procedure was carried out not less than 9 times. Filtration at 20° C. to 30° C., followed by rinsing of the filter cake with tert-butyl methyl ether (30.0 kg, 40.5 L, 2 volumes relative to D6), provided a filtrate, which was concentrated at −0.08 MPa to a volume of 20 to 30 L while the temperature was maintained below 40° C. n-Heptane (25.0 kg) was added at 15° C. to 30° C., and the resulting mixture was concentrated in the same manner to a volume of 19 to 30 L. n-Heptane (25.0 kg) was again added, and concentration was carried out in the same manner to a volume of 35 to 40 L; this was heated to 40° C. to 50° C., stirred at that temperature for 1 to 2 hours, and filtered at 48° C. to 53° C. The collected solid was dried at 35° C. to 45° C. under a flow of nitrogen. After 6 to 12 hours, the material was sampled for analysis every 2 to 12 hours until residual tert-butyl methyl ether was ≤5000 ppm, residual n-heptane was ≤5000 ppm, and residual methanol was ≤3000 ppm. The solid was then cooled to 15° C. to 30° C., sieved until the appearance of the product was uniform, and dissolved in dichloromethane (187 kg) at 20° C. to 30° C. After this had been stirred for 1 to 2 hours, it was filtered, and the organic layer was concentrated at −0.08 MPa to a volume of 25 to 35 L while the temperature was maintained below 40° C. The resulting mixture was diluted with n-heptane (3 volumes) and concentrated to a volume of 18 to 22 L (approximately 3 volumes); this operation was repeated a total of three times, effecting exchange of n-heptane for dichloromethane. The resulting mixture was allowed to stir and crystallize at 20° C. to 30° C.; it was then filtered to isolate P3 as an off-white solid. Yield: 5.60 kg, 98% by assay; corrected weight: 5.48 kg, 25.1 mol, 41% over 2 steps. HPLC purity: 99% (HPLC conditions. Column: Waters XBridge BEH C18, 4.6×150 mm, 3.5 μm; Mobile phase A: water containing 0.1% ammonium acetate; Mobile phase B: acetonitrile; Gradient: 20% to 95% B over 10 minutes; Flow rate: 1.0 mL/minute). Retention time for P3: 4.93 minutes. ¹H NMR (401 MHz, chloroform-d), characteristic peaks: δ 4.78 (br d, J_{HF}=46.7 Hz, 1H), 4.27-3.91 (m, 2H), 3.21-3.11 (m, 1H), 3.02-2.84 (m, 1H), 2.62-2.35 (m, 1H), 2.29-2.17 (m, 1H), 1.44 (s, 9H).

Preparation P4

### 2-(5-((3-(Ethoxy-d₅)pyridin-2-yl)oxy)pyridin-3-yl)pyrimidine-5-carboxylic acid (P4)

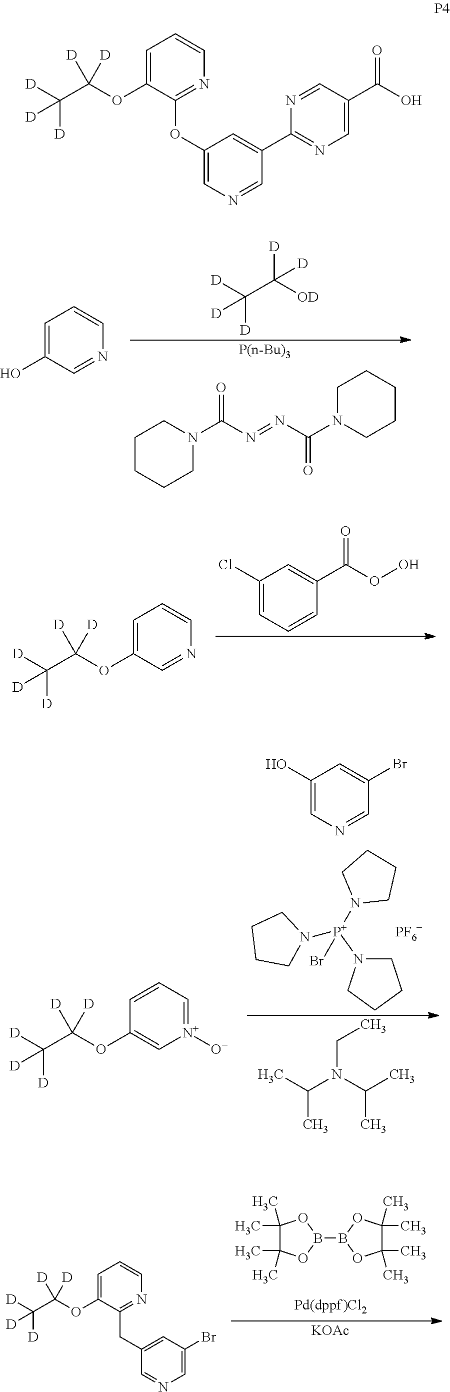

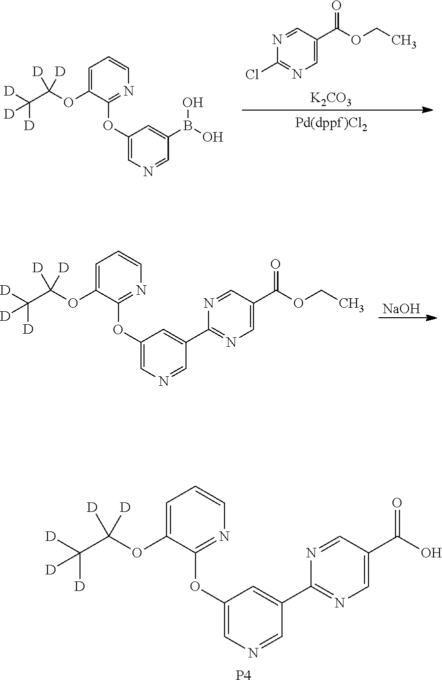

### Step 1. Synthesis of 3-(Ethoxy-d₅)pyridine

The reaction was carried out in two parallel batches; example batch preparation follows: Tri-n-butylphosphine (8.40 mL, 33.6 mmol, 2.0 equiv.) was added to a solution of 3-hydroxypyridine (1600 mg, 16.8 mmol, 1.0 equiv.) and ethanol-d₆ (1.18 mL, 20.2 mmol, 1.2 equiv.) in tetrahydrofuran (60 mL) at 25° C. 1,1′-(Azodicarbonyl) dipiperidine (8490 mg, 33.6 mmol, 2.0 equiv.) was then added and the yellow solution was stirred at 40° C. for 16 hours. The reaction mixture was filtered and to the filtrate was added water (50 mL) and the mixture was extracted with ethyl acetate (2×50 mL). The organic layer was washed with brine (50 mL), dried over sodium sulfate filtered and concentrated to afford the crude product. The crude product was purified by silica gel column chromatography (ISCO 80 g, 3% EtOAc in petroleum ether) to give the title compound as a yellow oil. The combined batches yielded 3.70 g (86%). ¹H NMR (400 MHz, CDCl₃) δ 8.29 (d, J=2.5 Hz, 1H), 8.19 (d, J=4.0 Hz, 1H), 7.12-7.24 (m, 2H).

### Step 2. Synthesis of 3-(Ethoxy-d₅)pyridine-1-oxide

m-Chloroperoxybenzoic acid (7620 mg, 37.5 mmol, 1.3 equiv.) was added to a solution of 3-(ethoxy-d₅)pyridine (3700 mg, 28.9 mmol, 1.0 equiv.) in dichloromethane (150 mL) at 0° C. The reaction mixture was stirred at 15° C. for 3 days. Aqueous sodium thiosulfate (100 mL) was added. The reaction mixture was stirred at 15° C. for 0.5 hours. The mixture was extracted with dichloromethane (100 mL). The organic layer dried over sodium sulfate filtered and concentrated to afford the crude product. The crude product was purified by silica gel column chromatography (dichloromethane-10:1 dichloromethane:methanol) to give the title compound (2500.0 mg, 60.1%) as a red solid. ¹H NMR (400 MHz, CDCl₃) δ ppm 7.93-8.00 (m, 1H), 7.86-7.92 (m, 1H), 7.15 (dd, J=8.6, 6.4 Hz, 1H), 6.87 (ddd, J=8.6, 2.2, 0.7 Hz, 1H).

### Step 3. Synthesis of 2-((5-Bromopyridin-3-yl)oxy)-3-(ethoxy-d₅)pyridine

Diisopropylethylamine (11.3 mL, 65.0 mmol, 3.75 equiv.) and bromotripyrrolidinophosphonium hexafluorophosphate (10.5 g, 22.5 mmol, (1.3 equiv.) were added to a stirred solution of 3-(ethoxy-d₅)pyridine-1-oxide (2500 mg, 17.3 mmol, 1.0 equiv.) and 3-bromo-5-hydroxypyridine (3020 mg, 17.3 mmol, 1.0 equiv.) in tetrahydrofuran (60 mL) at 0° C. The reaction mixture was stirred at 15° C. for 18 hours. The reaction mixture was concentrated to dryness and dissolved in dichloromethane (150 mL). The organic layer was washed with 1N sodium hydroxide (150 mL), water (100 mL), and brine (100 mL). The organic layer was dried over sodium sulfate, filtered and concentrated to give an oil. The crude oil was purified by silica gel column chromatography (petroleum ether-80:20 petroleum ether:ethyl acetate) to give product (3600.0 mg, 69.2%) as a white solid. ¹H NMR (400 MHz, CDCl₃) δ ppm 8.48 (d, J=1.8 Hz, 1H), 8.44 (d, J=2.3 Hz, 1H), 7.65-7.74 (m, 2H), 7.19-7.29 (m, 1H), 7.03 (dd, J=7.9, 4.9 Hz, 1H).

### Step 4. Synthesis of (5-((3-(Ethoxy-d₅)pyridin-2-yl)oxy)pyridin-3-yl)boronic acid

Bis(pinacolato)diboron (3800 mg, 15.0 mmol, 1.2 equiv.), potassium acetate (3670 mg, 37.4 mmol, 3.0 equiv.) and [1,1′-bis(diphenylphosphino)ferrocene]dichloropalladium(II) (Pd(dppf)Cl₂; 456 mg, 0.62 mmol, 0.05 equiv.) were added to a solution of 2-((5-bromopyridin-3-yl)oxy)-3-(ethoxy-d₅)pyridine (3740 mg, 12.5 mmol, 1.0 equiv.) in dioxane (120 mL). The resulting mixture was degassed and purged with nitrogen 3 times and then stirred at 100° C. under N₂ for 2 hrs. The resulting mixture was concentrated and the residue was diluted with ethyl acetate (200 mL) and washed with brine (100 mL). The organic phase was dried over sodium sulfate, filtered and concentrated to dryness to give the crude product (7000.0 mg) as brown oil, which was used for the next step directly. MS (ES+) 265.8 (M+H).

### Step 5. Synthesis of Ethyl 2-(5-((3-(ethoxy-d₅)pyridin-2-yl)oxy)pyridin-3-yl)pyrimidine-5-carboxylate

The reaction was carried out in two parallel batches; example batch preparation follows: Ethyl 2-chloropyrimidine-5-carboxylate (1000 mg, 5.4 mmol, 1.5 equiv.), potassium carbonate (980 mg, 7.1 mmol, 2.0 equiv.) and [1,1′-bis (diphenylphosphino)ferrocene]dichloropalladium(II), dichloromethane complex (130 mg, 0.18 mmol, 0.05 equiv.) were added to a solution of (5-((3-(ethoxy-d₅)pyridin-2-yl)oxy)pyridin-3-yl)boronic acid (1880 mg, 3.6 mmol, 1.0 equiv.) in dioxane (25 mL) and water (2.5 mL). The resulting mixture was flushed with nitrogen and then stirred at 90° C. for 2 hours. The reaction was filtered and the filtrate was concentrated. The residue was diluted with ethyl acetate (200 mL) and washed with brine (2×100 mL). The organic layer was dried over sodium sulfate, filtered and concentrated to give the crude product. The crude material was purified by silica gel column chromatography (petroleum ether: ethyl acetate; 70:30) to give product as a yellow solid. The combined batches yielded 2.3 g (50%). ¹H NMR (400 MHz, CDCl₃) δ ppm 9.57 (d, J=1.7 Hz, 1H), 9.34 (s, 2H), 8.68 (d, J=2.7 Hz, 1H), 8.61 (dd, J=2.6, 1.8 Hz, 1H), 7.73 (dd, J=4.8, 1.6 Hz, 1H), 7.28-7.28 (m, 1H), 7.04 (dd, J=8.1, 4.9 Hz, 1H), 7.00-7.08 (m, 1H), 4.48 (q, J=7.1 Hz, 2H), 1.46 (t, J=7.1 Hz, 4H). MS (ES+) 372.1 (M+H).

### Step 6: 2-(5-((3-(Ethoxy-d₅)pyridin-2-yl)oxy)pyridin-3-yl)pyrimidine-5-carboxylic acid (P4)

The reaction was carried out in two parallel batches; example batch preparation follows: Sodium hydroxide (1080 mg, 26.9 mmol, 5.0 equiv.) was added to a solution of ethyl 2-(5-((3-(ethoxy-d₅)pyridin-2-yl)oxy)pyridin-3-yl)pyrimidine-5-carboxylate (2000 mg, 5.4 mmol, 1.0 equiv.) in tetrahydrofuran (70 mL) and water (35 mL) at 15° C. The resulting solution was stirred at 15° C. for 1 hour. The mixture was concentrated to remove the tetrahydrofuran. The aqueous mixture was acidified to pH of 4 with 4M hydrochloric acid, diluted with water (50 mL) and stirred at 15° C. for 20 minutes. The solid was filtered, washed with water (3×10 mL), and dried to yield 2-(5-((3-(ethoxy-d₅)pyridin-2-yl)oxy)pyridin-3-yl)pyrimidine-5-carboxylic acid as a green solid. The combined batches yielded 1.28 g (60%). ¹H NMR (400 MHz, DMSO-d₆) δ ppm 9.41 (d, J=1.7 Hz, 1H), 9.33 (s, 2H) 8.67 (d, J=2.7 Hz, 1H), 8.33-8.41 (m, 1H) 7.70 (dd, J=4.9, 1.5 Hz, 1H), 7.58 (dd, J=7.8, 1.5 Hz, 1H), 7.19 (dd, J=7.8, 4.9 Hz, 1H). HRMS (TOF) 344.1402 (M+H).

### Example 1

### 2-{5-[(3-Ethoxy-5-fluoropyridin-2-yl)oxy]pyridin-3-yl}-N-[(3R,4S)-4-fluoropiperidin-3-yl]pyrimidine-5-carboxamide, bis(trifluoroacetate) salt (1)

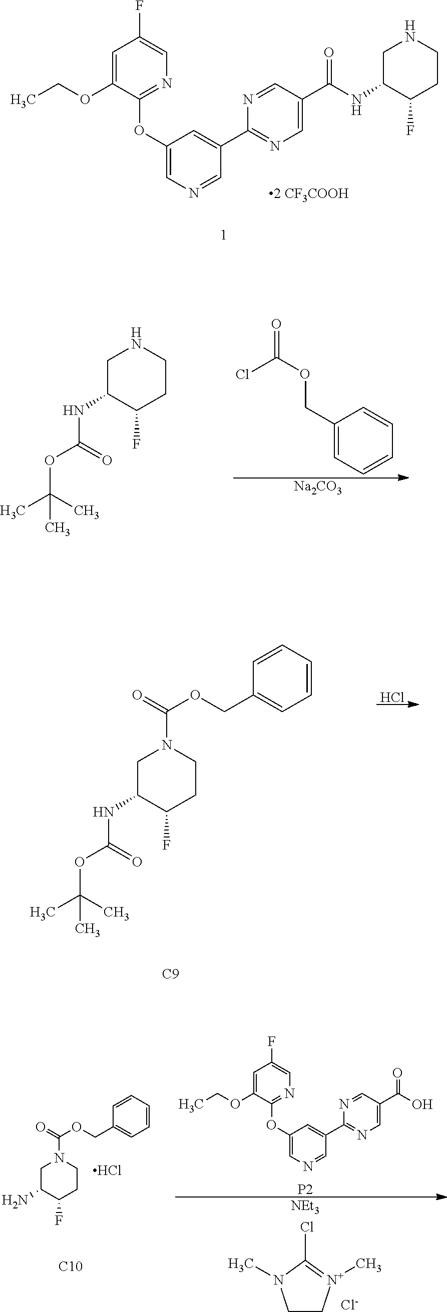

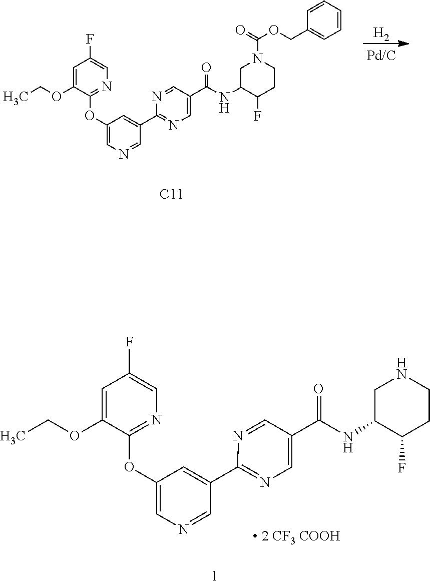 [SMILES: CCOc1cc(F)cnc1Oc1cncc(-c2ncc(C(=O)NC3CN(C(=O)OCc4ccccc4)CCC3F)cn2)c1 ; "H2" ; "C11" ; CCOc1cc(F)cnc1Oc1cncc(-c2ncc(C(=O)N[C@@H]3CNCC[C@@H]3F)cn2)c1 ; "· 2 CF3 COOH"]

### Step 1. Synthesis of benzyl (3R,4S)-3-[(tert-butoxycarbonyl)amino]-4-fluoropiperidine-1-carboxylate (C9)

Benzyl chloroformate (0.116 mL, 0.813 mmol) was added to a 0° C. mixture of tert-butyl [(3R,4S)-4-fluoropiperidin-3-yl]carbamate (150 mg, 0.69 mmol) and sodium carbonate (146 mg, 1.38 mmol) in tetrahydrofuran (8 mL), and the reaction mixture was stirred at 25° C. for three days. It was then treated with water (20 mL) and extracted with ethyl acetate (2×20 mL). The combined organic layers were washed with saturated aqueous sodium chloride solution (50 mL), dried over sodium sulfate, filtered, and concentrated in vacuo to afford C9 as a colorless oil (290 mg). This material contained impurities, by ¹H NMR analysis, but was used in the next step without additional purification. ¹H NMR (400 MHz, chloroform-d), characteristic peaks: δ 4.95-4.76 (m, 2H), 3.87-3.68 (m, 1H), 3.12-2.99 (m, 1H), 2.11-1.96 (m, 1H), 1.45 (s, 9H).

### Step 2. Synthesis of benzyl (3R,4S)-3-amino-4-fluoropiperidine-1-carboxylate, hydrochloride Salt (C10)

A mixture of C₉ (from the previous step; 290 mg, ≤0.69 mmol) and hydrogen chloride (4 M solution in 1,4-dioxane; 6.0 mL) was stirred at 15° C. for 1 hour, whereupon it was concentrated in vacuo, affording C10 as a white solid. Yield: 200 mg, 0.69 mmol, quantitative over 2 steps. ¹H NMR (400 MHz, deuterium oxide) δ 7.48-7.33 (m, 5H), 5.14 (s, 2H), 5.11 (br d, J_{HF}=48 Hz, 1H), 4.11-3.94 (m, 1H), 3.88-3.28 (m, 4H), 2.14-2.01 (m, 1H), 2.01-1.81 (m, 1H).

### Step 3. Synthesis of benzyl (3R,4S)-3-{[(2-{5-[(3-ethoxy-5-fluoropyridin-2-yl)oxy]pyridin-3-yl}pyrimidin-5-yl)carbonyl]amino}-4-fluoropiperidine-1-carboxylate (C11)

To a 25° C. solution of P2 (50.0 mg, 0.140 mmol) in N,N-dimethylacetamide (3.0 mL) were added C₁₀ (48.6 mg, 0.168 mmol), triethylamine (58.7 μL, 0.421 mmol), and 2-chloro-1,3-dimethyl-4,5-dihydro-1H-imidazol-3-ium chloride (71.2 mg, 0.421 mmol). After the reaction mixture had been stirred at 50° C. for 1 hour, it was diluted with water (20 mL) and extracted with ethyl acetate (20 mL). The organic layer was washed with saturated aqueous sodium chloride solution (20 mL), dried over sodium sulfate, filtered, and concentrated in vacuo. Chromatography on silica gel (Eluent: 9:1 ethyl acetate/petroleum ether) provided C₁₁ as a yellow solid. Yield: 80.0 mg, 0.135 mmol, 96%. LCMS m/z 591.2 [M+H]⁺.

### Step 4. Synthesis of 2-{5-[(3-ethoxy-5-fluoropyridin-2-yl)oxy]pyridin-3-yl}-N-[(3R,4S)-4-fluoropiperidin-3-yl]pyrimidine-5-carboxamide, bis(trifluoroacetate) salt (1)

10% Palladium on carbon (60.0 mg) was added to a solution of C11 (60.0 mg, 0.102 mmol) in tetrahydrofuran (10 mL), whereupon the mixture was degassed under vacuum and then purged with hydrogen; this evacuation-purge cycle was carried out a total of three times. The reaction mixture was then stirred under a balloon of hydrogen for 2 hours at 25° C., at which time it was combined with a similar reaction carried out using C11 (20.0 mg, 33.9 μmol) and filtered. The filtrate was concentrated in vacuo and purified using reversed-phase HPLC (Column: Agela Durashell C18, 5 μm; Mobile phase A: 0.1% trifluoroacetic acid in water; Mobile phase B: acetonitrile; Gradient: 14% to 44% B) to afford 2-{5-[(3-ethoxy-5-fluoropyridin-2-yl)oxy]pyridin-3-yl}-N-[(3R,4S)-4-fluoropiperidin-3-yl]pyrimidine-5-carboxamide, bis(trifluoroacetate) salt as a yellow gum. Combined yield: 28.1 mg, 41.1 μmol, 30%. LCMS m/z 457.4 [M+H]⁺. ¹H NMR (400 MHz, chloroform-d), characteristic peaks: δ 9.52 (br s, 1H), 9.16 (br s, 2H), 8.80 (s, 1H), 8.78-8.57 (m, 2H), 7.54 (s, 1H), 7.06 (dd, J=9.2, 2.3 Hz, 1H), 4.99 (br d, J_{HF}=50 Hz, 1H), 4.91-4.70 (m, 1H), 4.12 (q, J=6.9 Hz, 2H), 2.41-2.09 (m, 2H), 1.48 (t, J=6.9 Hz, 3H).

### Example 2

### 2-{5-[(3-Ethoxy-5-fluoropyridin-2-yl)oxy]pyridin-3-yl}-N-[(3S,5S)-5-fluoropiperidin-3-yl]pyrimidine-5-carboxamide (2)

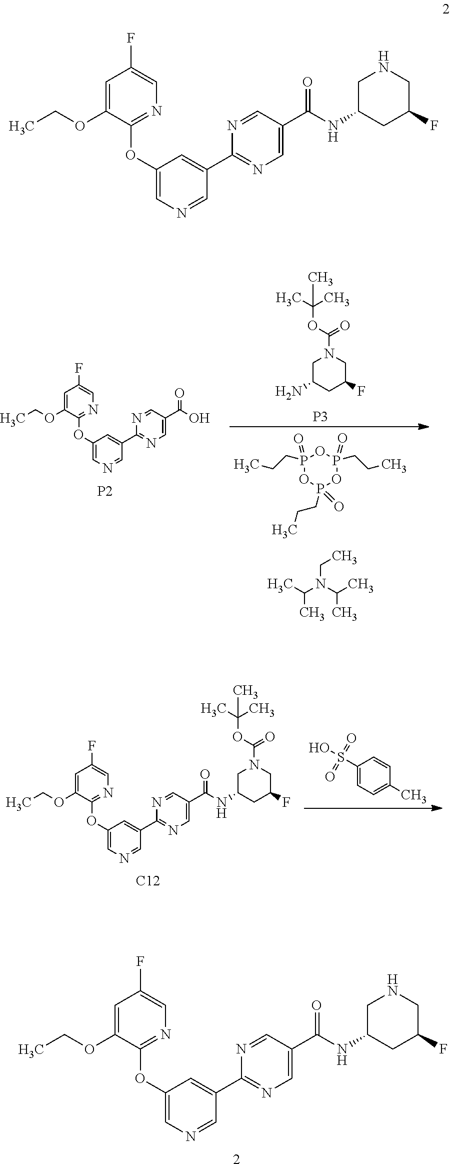

### Step 1. Synthesis of tert-butyl (3S,5S)-3-{[(2-{5-[(3-ethoxy-5-fluoropyridin-2-yl)oxy]pyridin-3-yl}pyrimidin-5-yl)carbonyl]amino}-5-fluoropiperidine-1-carboxylate (C12)

N,N-Diisopropylethylamine (2.79 mL, 16.0 mmol) and P3 (500 mg, 2.29 mmol) were added to a room temperature solution of P2 (816 mg, 2.29 mmol) in N,N-dimethylformamide (10 mL). After the resulting solution had been cooled to 0° C., 2,4,6-tripropyl-1,3,5,2,4,6-trioxatriphosphinane 2,4,6-trioxide (T3P; 50% solution in ethyl acetate; 1.6 mL, 2.7 mmol) was added, and the reaction mixture was allowed to stir at room temperature for 2 hours. LCMS analysis at this point indicated the presence of C12: LCMS m/z 557.4 [M+H]⁺. The reaction mixture was partitioned between water and ethyl acetate, and the aqueous layer was extracted twice with ethyl acetate. The combined organic layers were washed twice with water, once with saturated aqueous sodium bicarbonate solution, and once with saturated aqueous sodium chloride solution, then dried over sodium sulfate, filtered, and concentrated in vacuo to provide C12 as a yellow solid. Yield: 1.00 g, 1.80 mmol, 79%.

### Step 2. Synthesis of 2-{5-[(3-ethoxy-5-fluoropyridin-2-yl)oxy]pyridin-3-yl}-N-[(3S,5S)-5-fluoropiperidin-3-yl]pyrimidine-5-carboxamide (2)

p-Toluenesulfonic acid monohydrate (684 mg, 3.60 mmol) was added to a solution of C12 (1.00 g, 1.80 mmol) in ethyl acetate (10 mL), and the mixture was allowed to stir at room temperature until a solution was obtained. After the reaction mixture had been stirred at reflux for 2 hours, and then at room temperature for 2 hours, the solvent was decanted off of the resulting gum, and the gum was triturated four times with ethyl acetate, and twice with heptane. The obtained solid was partitioned between ethyl acetate and 1 M aqueous sodium hydroxide solution, and the aqueous layer was extracted four times with ethyl acetate; the combined organic layers were dried over magnesium sulfate, filtered, and concentrated in vacuo. The resulting material was dissolved in ethyl acetate (approximately 70 mL) at reflux, and treated with heptane (300 mL) until the mixture became slightly cloudy, whereupon the mixture was allowed to cool to room temperature and stir overnight. Filtration, followed by washing of the filter cake with 1:1 ethyl acetate/heptane, afforded 2-{5-[(3-ethoxy-5-fluoropyridin-2-yl)oxy]pyridin-3-yl}-N-[(3S,5S)-5-fluoropiperidin-3-yl]pyrimidine-5-carboxamide as a white solid. Yield: 580 mg, 1.27 mmol, 71%. LCMS m/z 457.2 [M+H]⁺. ¹H NMR (400 MHz, DMSO-d₆) δ 9.38 (d, J=1.7 Hz, 1H), 9.26 (s, 2H), 8.63 (d, J=2.7 Hz, 1H), 8.59 (br d, J=7.8 Hz, 1H), 8.35 (dd, J=2.7, 1.7 Hz, 1H), 7.71 (d, half of AB quartet, J=2.7 Hz, 1H), 7.68 (dd, component of ABX system, J=9.8, 2.7 Hz, 1H), 4.82 (br d, J_{HF}=48 Hz, 1H), 4.20 (q, J=7.0 Hz, 2H), 4.18-4.08 (m, 1H), 3.02-2.86 (m, 2H), 2.77-2.62 (m, 1H), 2.5-2.43 (m, 1H, assumed; partially obscured by solvent peak), 2.19-2.08 (m, 1H), 1.91-1.72 (m, 1H), 1.37 (t, J=7.0 Hz, 3H).

### Example 3

### 2-{5-[(3-Ethoxypyridin-2-yl)oxy]pyridin-3-yl}-N-[(3R,4S)-4-fluoropiperidin-3-yl]pyrimidine-5-carboxamide, bis(trifluoroacetate) salt (3)

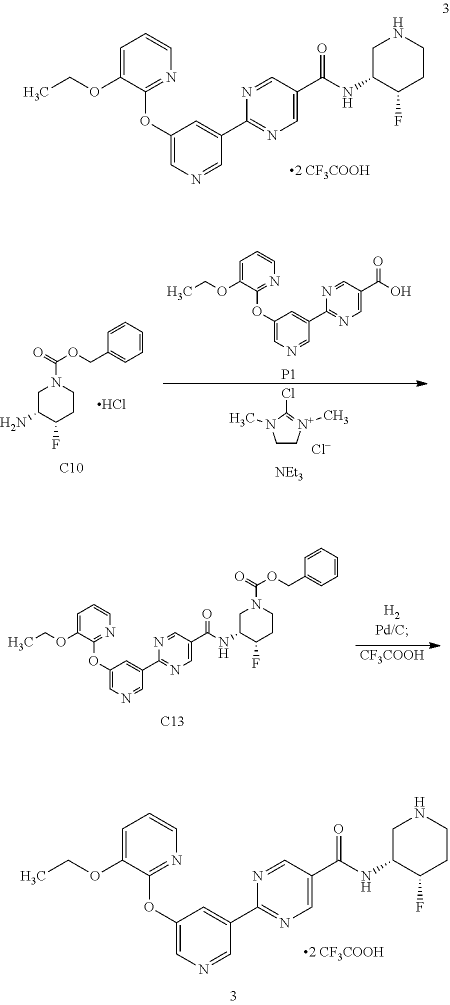

### Step 1. Synthesis of benzyl (3R,4S)-3-{[(2-{5-[(3-ethoxypyridin-2-yl)oxy]pyridin-3-yl}pyrimidin-5-yl)carbonyl]amino}-4-fluoropiperidine-1-carboxylate (C13)

To a 25° C. solution of P1 (50.0 mg, 0.148 mmol) in N,N-dimethylacetamide (3.0 mL) were added C10 (51.2 mg, 0.177 mmol), 2-chloro-1,3-dimethyl-4,5-dihydro-1H-imidazol-3-ium chloride (75.0 mg, 0.444 mmol), and triethylamine (61.8 μL, 0.443 mmol). The reaction mixture was stirred at 50° C. for 1 hour, whereupon water (20 mL) was added, and the resulting mixture was extracted with ethyl acetate (20 mL). The organic layer was washed with saturated aqueous sodium chloride solution (20 mL), dried over sodium sulfate, filtered, concentrated in vacuo, and subjected to silica gel chromatography (Eluent: 4:1 ethyl acetate/petroleum ether), affording C13 as a yellow solid. Yield: 80.0 mg, 0.140 mmol, 95%. LCMS m/z 573.2 [M+H]⁺.

### Step 2. Synthesis of 2-{5-[(3-ethoxypyridin-2-yl)oxy]pyridin-3-yl}-N-[(3R,4S)-4-fluoropiperidin-3-yl]pyrimidine-5-carboxamide, bis(trifluoroacetate) salt (3)

To a solution of C13 (60.0 mg, 0.105 mmol) in tetrahydrofuran (10 mL) was added 10% palladium on carbon (60.0 mg), whereupon the mixture was degassed under vacuum and then purged with hydrogen; this evacuation-purge cycle was carried out a total of three times. The reaction mixture was stirred under a balloon of hydrogen for 5 hours at 25° C. and then combined with a similar reaction carried out using C₁₃ (20.0 mg, 34.9 μmol). After this mixture had been filtered, the filtrate was concentrated in vacuo and purified using reversed-phase HPLC (Column: YMC-Actus Triart C18, 5 μm; Mobile phase A: water containing 0.05% ammonium hydroxide; Mobile phase B: acetonitrile; Gradient: 24% to 64% B). The free base 2-{5-[(3-ethoxypyridin-2-yl)oxy]pyridin-3-yl}-N-[(3R,4S)-4-fluoropiperidin-3-yl]pyrimidine-5-carboxamide was obtained as a white solid. Combined yield of free base 2-{5-[(3-ethoxypyridin-2-yl)oxy]pyridin-3-yl}-N-[(3R,4S)-4-fluoropiperidin-3-yl]pyrimidine-5-carboxamide: 12 mg, 27 μmol, 19%. LCMS m/z 439.1 [M+H]⁺. ¹H NMR (400 MHz, chloroform-d) δ 9.52 (d, J=1.8 Hz, 1H), 9.17 (s, 2H), 8.65 (d, J=2.7 Hz, 1H), 8.58-8.54 (m, 1H), 7.70 (dd, J=4.9, 1.5 Hz, 1H), 7.27-7.23 (m, 1H, assumed; partially obscured by solvent peak), 7.02 (dd, J=7.9, 4.9 Hz, 1H), 6.68 (br d, J=8.8 Hz, 1H), 4.93 (br d, J_{HF}=49 Hz, 1H), 4.48-4.31 (m, 1H), 4.18 (q, J=7.0 Hz, 2H), 3.10 (dd, J=12.1, 4.5 Hz, 1H), 3.00-2.80 (m, 3H), 2.15-2.01 (m, 1H), 1.97-1.77 (m, 1H), 1.50 (t, J=7.0 Hz, 3H).

The free base 2-{5-[(3-ethoxypyridin-2-yl)oxy]pyridin-3-yl}-N-[(3R,4S)-4-fluoropiperidin-3-yl]pyrimidine-5-carboxamide (12 mg, 27 μmol) was dissolved in an aqueous solution of trifluoroacetic acid (0.1% trifluoroacetic acid in water; 12 mL) and then lyophilized for 16 hours to provide 2-{5-[(3-ethoxypyridin-2-yl)oxy]pyridin-3-yl}-N-[(3R,4S)-4-fluoropiperidin-3-yl]pyrimidine-5-carboxamide, bis(trifluoroacetate) salt as a yellow gum. Combined yield of 2-{5-[(3-ethoxypyridin-2-yl)oxy]pyridin-3-yl}-N-[(3R,4S)-4-fluoropiperidin-3-yl]pyrimidine-5-carboxamide, bis(trifluoroacetate) salt: 12.9 mg, 19.4 μmol, 14%. LCMS m/z 439.4 [M+H]⁺. ¹H NMR (400 MHz, chloroform-d) δ 10.56-10.28 (br s, 1H), 9.80-9.55 (br s, 1H), 9.71 (s, 1H), 9.24 (s, 2H), 9.09 (br s, 1H), 8.86 (br d, J=7.9 Hz, 1H), 8.77 (d, J=2.5 Hz, 1H), 7.72 (dd, J=4.9, 1.5 Hz, 1H), 7.32 (dd, J=8.1, 1.5 Hz, 1H), 7.14 (dd, J=8.0, 4.9 Hz, 1H), 4.97 (br d, J_{HF}=49 Hz, 1H), 4.96-4.78 (m, 1H), 4.18 (q, J=7.0 Hz, 2H), 3.83-3.72 (m, 1H), 3.5-3.2 (m, 3H), 2.42-2.11 (m, 2H), 1.50 (t, J=7.0 Hz, 3H).

### Example 4

### 2-{5-[(3-Ethoxypyridin-2-yl)oxy]pyridin-3-yl}-N-[(3S,5S)-5-fluoropiperidin-3-yl]pyrimidine-5-carboxamide (4)

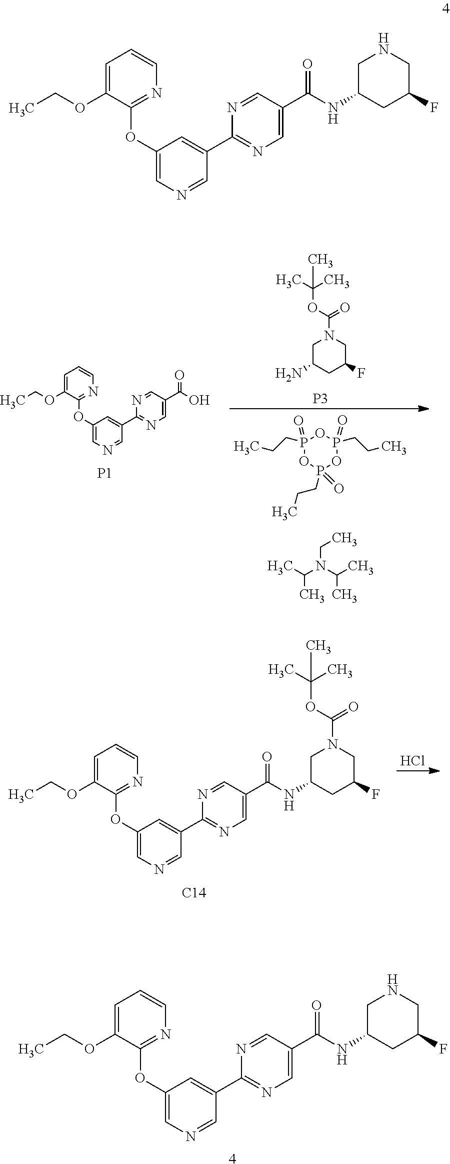

### Step 1. Synthesis of tert-butyl (38,5S)-3-{[(2-{5-[(3-ethoxypyridin-2-yl)oxy]pyridin-3-yl}pyrimidin-5-yl)carbonyl]amino}-5-fluoropiperidine-1-carboxylate (C14)

N,N-Diisopropylethylamine (53.1 mL, 305 mmol) and P3 (9.50 g, 43.5 mmol) were added to a solution of P1 (14.7 g, 43.4 mmol) in acetonitrile (210 mL). The mixture was cooled to 0° C., and then 2,4,6-tripropyl-1,3,5,2,4,6-trioxatriphosphinane 2,4,6-trioxide (T3P; 50% solution in ethyl acetate; 30.5 mL, 51.2 mmol) was added via syringe, over approximately 4 minutes. After the reaction mixture had been stirred at 0° C. for 45 minutes, the ice bath was removed and the reaction mixture was allowed to come to room temperature and stir for 17 hours. It was then concentrated in vacuo, the residue was partitioned between water and ethyl acetate, and the aqueous layer was extracted twice with ethyl acetate. The combined organic layers were washed sequentially with saturated aqueous sodium bicarbonate solution and saturated aqueous sodium chloride solution; a precipitate that appeared during the saturated aqueous sodium chloride wash was removed via filtration and discarded. The saturated aqueous sodium chloride layer was extracted once with ethyl acetate, and the combined organic layers were concentrated in vacuo. The residue was dissolved in a mixture of methylene chloride and methanol and pre-adsorbed onto silica gel. Silica gel chromatography (Gradient: 30% to 100% ethyl acetate in heptane) was carried out, and it was observed that the product was of limited solubility in the ethyl acetate/heptane eluent. This purification afforded C14 as an off-white solid. Yield: 19.1 g, 35.5 mmol, 82%. LCMS m/z 539.3 [M+H]⁺. ¹H NMR (400 MHz, chloroform-d) δ 9.48 (d, J=1.8 Hz, 1H), 9.12 (s, 2H), 8.63 (d, J=2.6 Hz, 1H), 8.55 (dd, J=2.7, 1.8 Hz, 1H), 7.70 (dd, J=4.9, 1.5 Hz, 1H), 7.26 (dd, J=7.8, 1.5 Hz, 1H), 7.03 (dd, J=7.9, 4.9 Hz, 1H), 6.97-6.37 (v br m, 1H), 4.78 (br d, J_{HF}=46.7 Hz, 1H), 4.46-4.33 (m, 1H), 4.18 (q, J=7.0 Hz, 2H), 4.08-3.05 (v br m, 4H), 2.41-2.11 (m, 1H), 2.02-1.79 (m, 1H), 1.49 (t, J=7.0 Hz, 3H), 1.49 (s, 9H).

### Step 2. Synthesis of 2-{5-[(3-ethoxypyridin-2-yl)oxy]pyridin-3-yl}-N-[(3S,5S)-5-fluoropiperidin-3-yl]pyrimidine-5-carboxamide (4)

A solution of hydrogen chloride in 1,4-dioxane (4 M; 520 mL, 2.1 mol) was added over 10 minutes to a room temperature solution of C14 (159 g, 295 mmol) in tetrahydrofuran (850 mL); the reaction temperature increased to 35° C. to 40° C., and this temperature was maintained using a heating mantle. After the addition had been completed, the reaction mixture was stirred for 3 hours at 35° C. to 40° C. LCMS analysis indicated that 20% of the starting material remained, so a solution of hydrogen chloride in 1,4-dioxane (4 M; 150 mL, 600 mmol) was again added to the reaction mixture, and stirring was continued at 35° C. to 40° C. for 30 minutes. At this point, 5% of the starting material remained via LCMS analysis, and the reaction mixture was treated with a solution of hydrogen chloride in 1,4-dioxane (4 M; 60 mL, 240 mmol). After an additional 45 minutes at 35° C. to 40° C., the reaction mixture was concentrated in vacuo, and the resulting solid was dissolved in water (1 L). This solution was treated with aqueous sodium hydroxide solution (1 M; 900 mL, 900 mmol) and then diluted with water (400 mL) to facilitate stirring; after 15 minutes at room temperature, the precipitate was collected via filtration and washed with water (4×250 mL). This solid was brought to a total volume of 800 mL by addition of water, and then slurried with methanol (800 mL) at room temperature for 2 hours, using an overhead stirrer. The slurry was filtered, and the filter cake was washed with a mixture of methanol and water (1:1, 1 L). This solid was combined with the product from several similar reactions carried out using C14 (5946 mmol); the combined batches were suspended in ethyl acetate (1.1 L) and stirred for 1 hour at room temperature using a mechanical stirrer. After the solid had been collected by filtration, it was washed with ethyl acetate to afford 2-{5-[(3-ethoxypyridin-2-yl)oxy]pyridin-3-yl}-N-[(3S,5S)-5-fluoropiperidin-3-yl]pyrimidine-5-carboxamide as an off-white solid. Yield: 330 g, 753 mmol, 61% over 2 steps. LCMS m/z 439.3 [M+H]⁺. ¹H NMR (400 MHz, DMSO-d₆) δ 9.38 (d, J=1.8 Hz, 1H), 9.26 (s, 2H), 8.64 (d, J=2.7 Hz, 1H), 8.60 (br d, J=7.9 Hz, 1H), 8.36 (dd, J=2.7, 1.8 Hz, 1H), 7.68 (dd, J=4.9, 1.5 Hz, 1H), 7.56 (dd, J=8.1, 1.5 Hz, 1H), 7.17 (dd, J=8.0, 4.8 Hz, 1H), 4.81 (br d, J_{HF}=48.3 Hz, 1H), 4.22-4.07 (m, 1H), 4.17 (q, J=7.0 Hz, 2H), 3.02-2.86 (m, 2H), 2.69 (br dd, J=35.1, 14.2 Hz, 1H), 2.5-2.38 (m, 2H, assumed; partially obscured by solvent peak), 2.19-2.07 (m, 1H), 1.91-1.72 (m, 1H), 1.37 (t, J=7.0 Hz, 3H).

Powder X-ray diffraction analysis was conducted on the solid of this example using a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 3.00 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.01 degrees and a step time of 1.0 second. The antiscatter screen was set to a fixed distance of 1.5 mm. Samples were rotated at 15/min during collection. Samples were prepared by placing them in a silicon low background sample holder and rotated during collection.

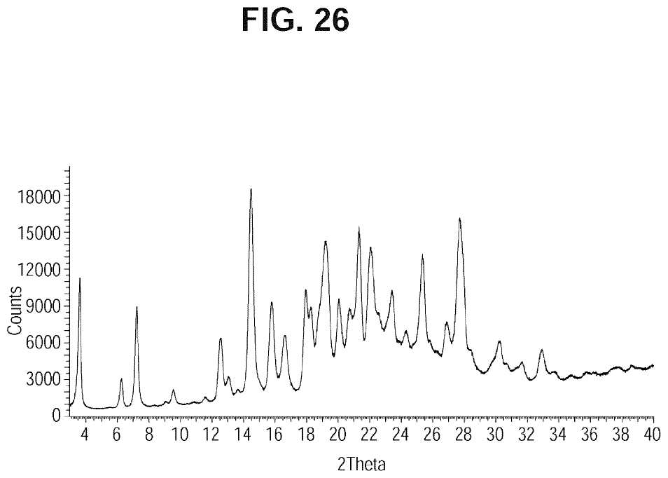

Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked, and peak positions were adjusted to the peak maximum. Peaks with relative intensity 3% were generally chosen. The peaks which were not resolved or were consistent with noise were not selected. A typical error associated with the peak position from PXRD stated in USP up to ±0.2° 2θ (USP-941). Some variation in relative peak heights is expected based on changes with crystal sizes and morphologies. Characteristic x-ray powder diffraction patterns is provided in FIG. 26. The PXRD data from this figure is further described below.

TABLE A
PXRD peaks for crystalline material of Example 4, Form 1

| Angle | Relative | Angle | Relative | Angle | Relative |
|---|---|---|---|---|---|
| 2Θ (°) | intensity (%) | 2Θ (°) | intensity (%) | 2Θ (°) | intensity (%) |
| 3.6 | 62 | 18.0 | 48 | 25.4 | 59 |
| 6.3 | 14 | 18.3 | 39 | 26.9 | 26 |
| 7.2 | 48 | 19.2 | 70 | 27.7 | 76 |
| 9.6 | 8 | 20.1 | 41 | 28.4 | 13 |
| 12.6 | 30 | 20.7 | 35 | 30.2 | 17 |
| 13.1 | 10 | 21.3 | 75 | 30.7 | 7 |
| 13.7 | 4 | 22.1 | 64 | 31.6 | 8 |
| 14.5 | 100 | 22.6 | 32 | 32.9 | 15 |
| 15.8 | 44 | 23.4 | 42 | 33.8 | 5 |
| 16.6 | 27 | 24.3 | 21 | | |

TABLE B
Key PXRD peaks to characterize crystalline material of Example 4,
Form 1
Example 4, Form 1

Angle 2Θ (°) ± 0.2°
7.2, 14.5, 15.8, 27.7

### Example 4, Hydrochloride Salt

### 2-{5-[(3-Ethoxypyridin-2-yl)oxy]pyridin-3-yl}-N-[(3S,5S)-5-fluoropiperidin-3-yl]pyrimidine-5-carboxamide, hydrochloride salt (4, hydrochloride salt

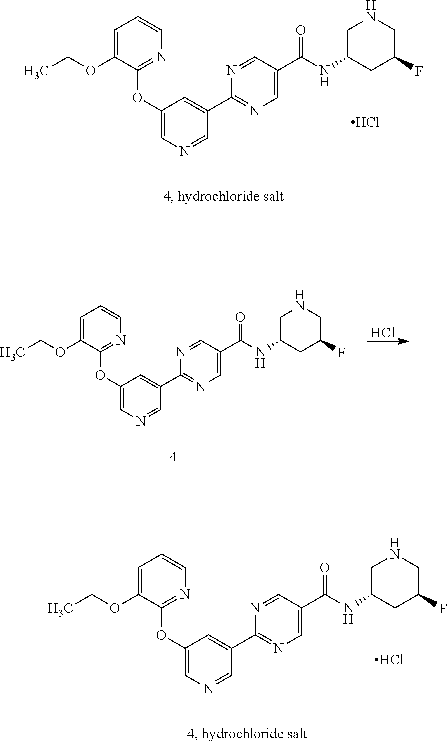 [SMILES: CCOc1cccnc1Oc1cncc(-c2ncc(C(=O)N[C@@H]3CNC[C@@H](F)C3)cn2)c1 ; Cl ; CCOc1cccnc1Oc1cncc(-c2ncc(C(=O)N[C@@H]3CNC[C@@H](F)C3)cn2)c1 ; Cl ; CCOc1cccnc1Oc1cncc(-c2ncc(C(=O)N[C@@H]3CNC[C@@H](F)C3)cn2)c1 ; Cl]

A suspension of 2-{5-[(3-ethoxypyridin-2-yl)oxy]pyridin-3-yl}-N-[(3S,5S)-5-fluoropiperidin-3-yl]pyrimidine-5-carboxamide (4.0 g, 9.1 mmol) in ethyl acetate (40 mL) was warmed to approximately 50° C., whereupon a solution of hydrogen chloride in 1,4-dioxane (4 M; 2.5 mL, 10 mmol) was added, and the reaction mixture was stirred at room temperature for 4 days. It was then filtered, and the filter cake was washed twice with warm ethyl acetate, affording 2-{5-[(3-ethoxypyridin-2-yl)oxy]pyridin-3-yl}-N-[(3S,5S)-5-fluoropiperidin-3-yl]pyrimidine-5-carboxamide, hydrochloride salt as a white solid. Yield: 4.1 g, 8.6 mmol, 94%. ¹H NMR (400 MHz, DMSO-d₆) δ 9.90 (br d, J=11 Hz, 1H), 9.39 (d, J=1.8 Hz, 1H), 9.35 (s, 2H), 9.26 (br d, J=7.7 Hz, 1H), 9.24-9.10 (m, 1H), 8.65 (d, J=2.7 Hz, 1H), 8.37 (dd, J=2.7, 1.8 Hz, 1H), 7.69 (dd, J=4.8, 1.5 Hz, 1H), 7.57 (dd, J=8.0, 1.5 Hz, 1H), 7.18 (dd, J=8.0, 4.9 Hz, 1H), 5.23 (br d, J_{HF}=45.3 Hz, 1H), 4.54-4.40 (m, 1H), 4.18 (q, J=7.0 Hz, 2H), 3.53-3.41 (m, 1H), 3.39-3.15 (m, 2H), 3.03-2.88 (m, 1H), 2.35-2.21 (m, 1H), 2.13-1.90 (m, 1H), 1.37 (t, J=7.0 Hz, 3H).

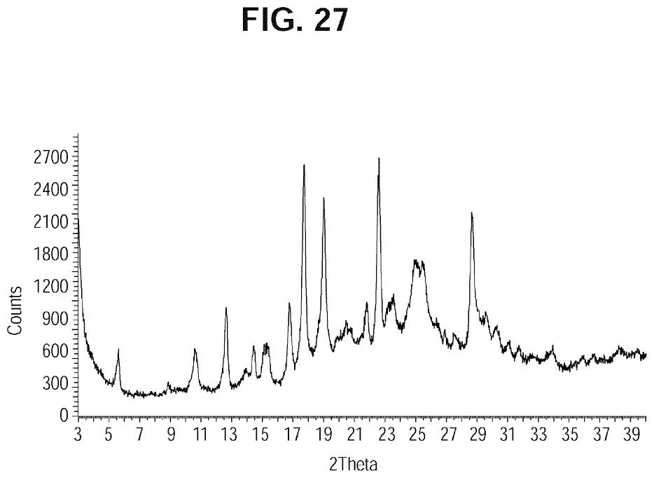

Powder X-ray diffraction analysis was conducted on the solid of this experiment using a Bruker AXS D4 Endeavor diffractometer equipped with a Cu radiation source. The divergence slit was set at 0.6 mm while the secondary optics used variable slits. Diffracted radiation was detected by a PSD-Lynx Eye detector. The X-ray tube voltage and amperage were set to 40 kV and 40 mA respectively. Data was collected in the Theta-2Theta goniometer at the Cu wavelength from 3.0 to 40.0 degrees 2-Theta using a step size of 0.020 degrees and a step time of 0.3 second. Samples were prepared by placing them in a silicon low background sample holder and rotated during collection. Data were collected using Bruker DIFFRAC Plus software and analysis was performed by EVA diffract plus software. Characteristic x-ray powder diffraction pattern is provided in FIG. 27.

### Example 4, p-toluenesulfonate Salt

### 2-{5-[(3-Ethoxypyridin-2-yl)oxy]pyridin-3-yl}-N-[(3S,5S)-5-fluoropiperidin-3-yl]pyrimidine-5-carboxamide, p-toluenesulfonate salt (4, p-toluenesulfonate Salt

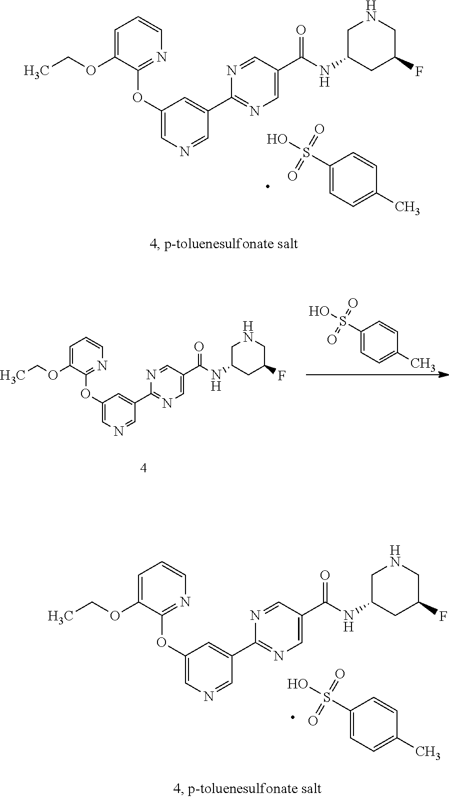

A suspension of 2-{5-[(3-ethoxypyridin-2-yl)oxy]pyridin-3-yl}-N-[(3S,5S)-5-fluoropiperidin-3-yl]pyrimidine-5-carboxamide (4.0 g, 9.1 mmol) in ethyl acetate (40 mL) was warmed to approximately 50° C., whereupon p-toluenesulfonic acid monohydrate (1.9 g, 10 mmol) was added, and the reaction mixture was stirred at room temperature for 3 days. The resulting chunky solid was broken up with a spatula, and the suspended solids were vigorously stirred vigorously for 1 day at room temperature. Filtration provided a filter cake, which was washed twice with warm ethyl acetate to provide 2-{5-[(3-ethoxypyridin-2-yl)oxy]pyridin-3-yl}-N-[(3S,5S)-5-fluoropiperidin-3-yl]pyrimidine-5-carboxamide, p-toluenesulfonate salt as a white solid. Yield: 5.3 g, 8.7 mmol, 96%. LCMS m/z 439.2 [M+H]⁺. ¹H NMR (400 MHz, DMSO-d₆) δ9.40 (d, J=1.8 Hz, 1H), 9.28 (s, 2H), 9.24-9.03 (br m, 2H), 8.98 (br d, J=7.6 Hz, 1H), 8.65 (d, J=2.7 Hz, 1H), 8.37 (dd, J=2.7, 1.8 Hz, 1H), 7.68 (dd, J=4.9, 1.5 Hz, 1H), 7.57 (dd, J=8.0, 1.5 Hz, 1H), 7.48 (d, J=8.0 Hz, 2H), 7.18 (dd, J=8.0, 4.8 Hz, 1H), 7.11 (d, J=7.9 Hz, 2H), 5.24 (br d, J_{HF}=45.1 Hz, 1H), 4.51-4.38 (m, 1H), 4.18 (q, J=7.0 Hz, 2H), 3.59-3.47 (m, 1H), 3.43-3.17 (m, 2H), 2.98-2.85 (m, 1H), 2.36-2.24 (m, 1H), 2.28 (s, 3H), 2.06-1.85 (m, 1H), 1.37 (t, J=7.0 Hz, 3H).

### Crystallization of Example 4, p-toluenesulfonate Salt

### 2-{5-[(3-Ethoxypyridin-2-yl)oxy]pyridin-3-yl}-N-[(3S,5S)-5-fluoropiperidin-3-yl]pyrimidine-5-carboxamide, p-toluenesulfonate salt (4, p-toluenesulfonate Salt

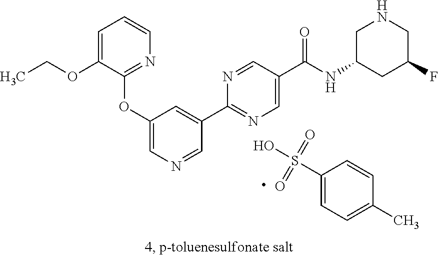

Treatment of Example 4, p-toluenesulfonate salt (19.1 g, 31.3 mmol) with a mixture of water and ethanol (9:1, 300 mL) was followed by minimal warming with a heat gun, until a solution was obtained. This was allowed to cool to room temperature overnight, and then stirred for an additional 24 hours, whereupon the solvent ratio was adjusted to approximately 4:1 water/ethanol by addition of ethanol (35 mL). The resulting mixture was heated to 85° C. to afford a solution, which was cooled to room temperature over 3 hours and then stirred at room temperature overnight. Collection of the precipitate via filtration afforded a solid, which was dried in a vacuum oven that was equipped with a nitrogen bleed, and had been pre-heated to 40° C. 2-{5-[(3-Ethoxypyridin-2-yl)oxy]pyridin-3-yl}-N-[(3S,5S)-5-fluoropiperidin-3-yl]pyrimidine-5-carboxamide, p-toluenesulfonate salt was obtained as a white powder. Yield: 11.8 g, 19.3 mmol, 62%. LCMS m/z 439.2 [M+H]⁺. ¹H NMR (400 MHz, DMSO-d₆) δ 9.39 (d, J=1.8 Hz, 1H), 9.27 (s, 2H), 9.13 (br s, 2H), 8.99 (br d, J=7.6 Hz, 1H), 8.65 (d, J=2.7 Hz, 1H), 8.37 (dd, J=2.7, 1.8 Hz, 1H), 7.68 (dd, J=4.8, 1.5 Hz, 1H), 7.57 (dd, J=8.1, 1.5 Hz, 1H), 7.49 (br d, J=8.0 Hz, 2H), 7.18 (dd, J=8.0, 4.8 Hz, 1H), 7.11 (br d, J=8.0 Hz, 2H), 5.24 (br d, J_{HF}=45.1 Hz, 1H), 4.52-4.38 (m, 1H), 4.18 (q, J=7.0 Hz, 2H), 3.59-3.47 (m, 1H), 3.44-3.18 (m, 2H), 2.92 (dd, J=11.9, 11.8 Hz, 1H), 2.37-2.22 (m, 1H), 2.27 (s, 3H), 2.08-1.84 (m, 1H), 1.37 (t, J=7.0 Hz, 3H).

Most of this material (11.6 g) was combined with another sample of 2-{5-[(3-ethoxypyridin-2-yl)oxy]pyridin-3-yl}-N-[(3S,5S)-5-fluoropiperidin-3-yl]pyrimidine-5-carboxamide, p-toluenesulfonate salt (7.4 g); the individual samples had exhibited the same diffraction pattern by powder X-ray diffraction analysis. Mixture of the two samples provided a fluffy white solid (19.0 g).

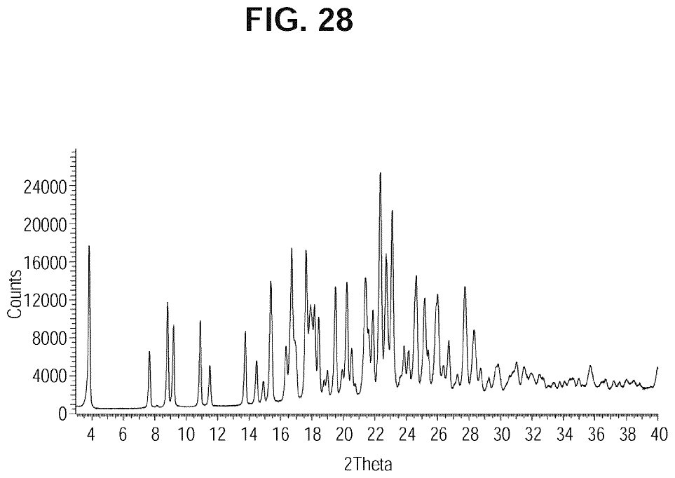

Powder X-ray diffraction analysis was conducted on the solid of this example using a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 3.00 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.01 degrees and a step time of 1.0 second. The antiscatter screen was set to a fixed distance of 1.5 mm. Samples were rotated at 15/min during collection. Samples were prepared by placing them in a silicon low background sample holder and rotated during collection. Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made, the output of automated assignments was visually checked, and peak positions were adjusted to the peak maximum. Peaks with relative intensity 3% were generally chosen. The peaks which were not resolved or were consistent with noise were not selected. A typical error associated with the peak position from PXRD stated in USP up to ±0.2° 2θ (USP-941). Some variation in relative peak heights is expected based on changes with crystal sizes and morphologies. Characteristic x-ray powder diffraction patterns is provided in FIG. 28. The PXRD data from this figure is further described below.

TABLE C
PXRD peaks for crystalline material of Example
4, p-toluenesulfonate salt, Form 1

| Angle | Relative | Angle | Relative | Angle | Relative |
|---|---|---|---|---|---|
| 2Θ (°) | intensity (%) | 2Θ (°) | intensity (%) | 2Θ (°) | intensity (%) |
| 3.8 | 74 | 19.5 | 50 | 26.7 | 23 |
| 7.7 | 26 | 19.9 | 12 | 27.3 | 7 |
| 8.8 | 48 | 20.2 | 51 | 27.7 | 47 |
| 9.2 | 38 | 20.5 | 21 | 28.3 | 27 |
| 10.9 | 39 | 20.8 | 5 | 28.7 | 10 |
| 11.5 | 19 | 21.4 | 53 | 29.3 | 6 |
| 13.8 | 33 | 21.6 | 29 | 29.8 | 12 |
| 14.5 | 19 | 21.9 | 38 | 31.0 | 12 |
| 14.9 | 10 | 22.4 | 100 | 31.5 | 10 |
| 15.4 | 55 | 22.7 | 63 | 32.0 | 7 |
| 16.4 | 25 | 23.1 | 83 | 32.5 | 6 |
| 16.7 | 69 | 23.6 | 7 | 32.7 | 5 |
| 16.9 | 27 | 23.9 | 20 | 35.0 | 5 |
| 17.6 | 67 | 24.2 | 18 | 35.7 | 10 |
| 17.9 | 42 | 24.6 | 52 | 37.2 | 4 |
| 18.2 | 42 | 25.2 | 41 | 37.6 | 3 |
| 18.4 | 36 | 25.4 | 18 | 38.0 | 5 |
| 18.8 | 8 | 26.0 | 44 | 25.9 | 38 |
| 19.0 | 12 | 26.4 | 11 | 36.7 | 4 |

TABLE D
Key PXRD peaks to characterize crystalline material of Example 4,
p-toluenesulfonate salt, Form 1
Example 4, p-toluenesulfonate salt, Form 1

Angle 2Θ (°) ± 0.2°
3.8, 7.7, 8.8, 22.4, 24.6

### Alternate Synthesis of Example 4, p-toluenesulfonate Salt

### 2-{5-[(3-Ethoxypyridin-2-yl)oxy]pyridin-3-yl}-N-[(3S,5S)-5-fluoropiperidin-3-yl]pyrimidine-5-carboxamide, p-toluenesulfonate salt (4, p-toluenesulfonate Salt

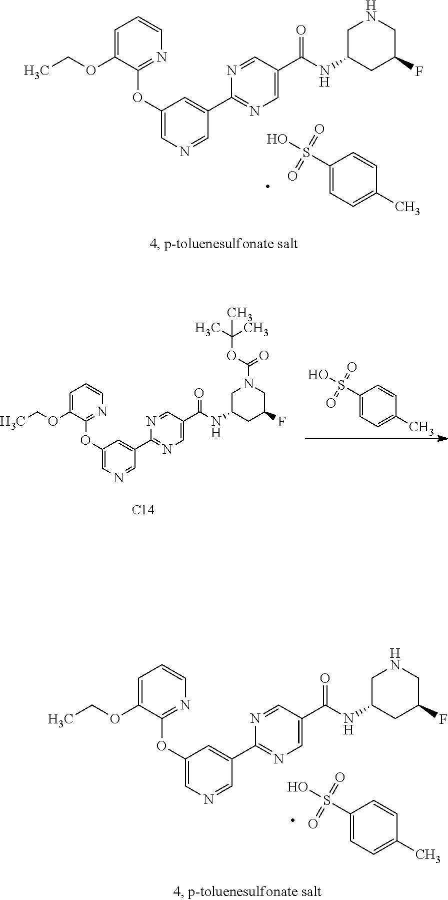

A solution of C14 (9.47 g, 17.6 mmol), p-toluenesulfonic acid monohydrate (98%, 3.58 g, 18.4 mmol), and water (4.74 mL, 263 mmol) in acetonitrile (90.0 mL) was heated to 90° C. over 10 minutes (internal reaction temperature 76° C.). After 12 hours, the reaction mixture was cooled to 25° C. over 10 minutes, and held at 25° C. overnight. It was then cooled to 10° C., and filtered. The filter cake was rinsed twice with 1 volume of a 95:5 acetonitrile/water mixture that had been cooled to 10° C., affording 2-{5-[(3-ethoxypyridin-2-yl)oxy]pyridin-3-yl}-N-[(3S,5S)-5-fluoropiperidin-3-yl]pyrimidine-5-carboxamide, p-toluenesulfonate salt as a yellow solid. Yield: 8.30 g, 13.6 mmol, 77%. ¹H NMR (400 MHz, DMSO-d₆) δ 9.40 (d, J=1.8 Hz, 1H), 9.27 (s, 2H), 9.17-9.07 (br s, 2H), 8.97 (br d, J=7.6 Hz, 1H), 8.65 (d, J=2.7 Hz, 1H), 8.37 (dd, J=2.7, 1.7 Hz, 1H), 7.68 (dd, J=4.9, 1.5 Hz, 1H), 7.57 (dd, J=8.0, 1.5 Hz, 1H), 7.47 (br d, J=8.0 Hz, 2H), 7.18 (dd, J=7.9, 4.9 Hz, 1H), 7.11 (br d, J=7.9 Hz, 2H), 5.24 (br d, J_{HF}=45.1 Hz, 1H), 4.52-4.38 (m, 1H), 4.18 (q, J=7.0 Hz, 2H), 3.59-3.47 (m, 1H), 3.44-3.19 (m, 2H), 2.90 (dd, J=11.8, 11.8 Hz, 1H), 2.36-2.25 (m, 1H), 2.28 (s, 3H), 2.06-1.84 (m, 1H), 1.37 (t, J=7.0 Hz, 3H).

### Example 5

### 2-{5-[(3-Ethoxypyridin-2-yl)oxy]pyridin-3-yl}-N-[(3R,4R)-4-fluoropiperidin-3-yl]pyrimidine-5-carboxamide (5)

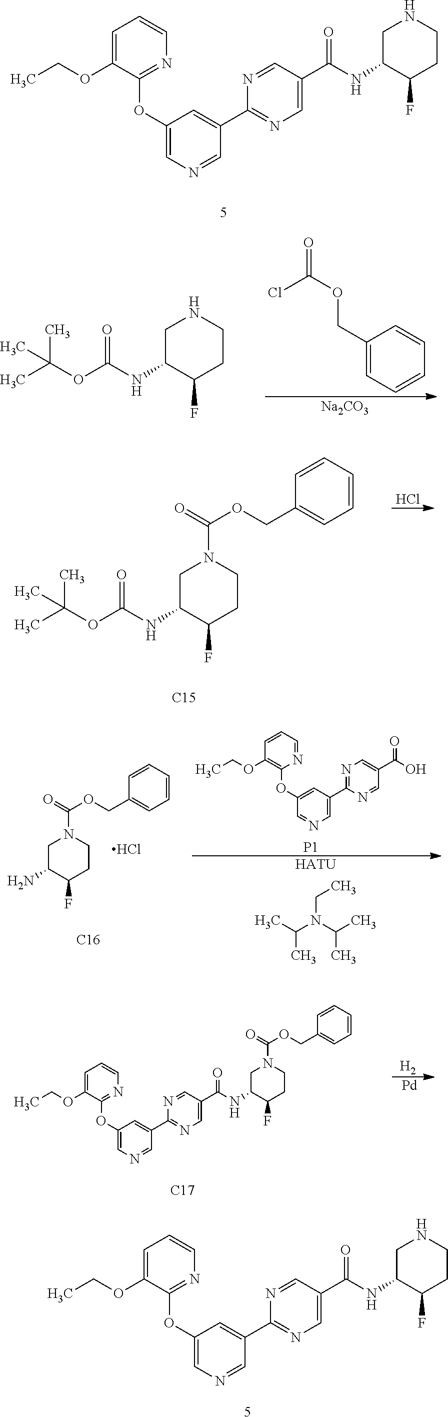

### Step 1. Synthesis of benzyl (3R,4R)-3-[(tert-butoxycarbonyl)amino]-4-fluoropiperidine-1-carboxylate (C15)

Benzyl chloroformate (258 mg, 1.51 mmol) was added to a 0° C. mixture of tert-butyl [(3R,4R)-4-fluoropiperidin-3-yl]carbamate (300 mg, 1.37 mmol) in tetrahydrofuran (15 mL) and aqueous sodium carbonate solution (1 M; 2.75 mL, 2.75 mmol). After the reaction mixture had been stirred at 15° C. for 16 hours, water (20 mL) was added, and the resulting mixture was extracted with ethyl acetate (2×30 mL). The combined organic layers were washed with saturated aqueous sodium chloride solution (50 mL), dried over sodium sulfate, filtered, and concentrated in vacuo to provide C15 as a white solid. Yield: 485 mg, 1.38 mmol, quantitative. ¹H NMR (400 MHz, chloroform-d) δ 7.43-7.28 (m, 5H), 5.14 (AB quartet, J_{AB}=12.3 Hz, Δ□_{AB}=14.2 Hz, 2H; downfield doublet is broadened), 4.83-4.53 (m, 2H), 3.87-3.33 (m, 4H), 2.06-1.75 (m, 2H), 1.44 (s, 9H).

### Step 2. Synthesis of benzyl (3R,4R)-3-amino-4-fluoropiperidine-1-carboxylate, hydrochloride salt (C16)

A solution of C15 (485 mg, 1.38 mmol) in methanol (6 mL) was treated with hydrogen chloride (solution in ethyl acetate; 12 mL). After the reaction mixture had been stirred at 20° C. for 1 hour, it was concentrated in vacuo, affording C16 as a white solid. Yield: 370 mg, 1.28 mmol, 93%. ¹H NMR (400 MHz, deuterium oxide) δ 7.50-7.39 (m, 5H), 5.17 (s, 2H), 4.93-4.71 (m, 1H, assumed; partially obscured by solvent peak), 4.42-4.27 (m, 1H), 4.24-3.98 (m, 1H), 3.51-3.39 (m, 1H), 3.21-2.99 (m, 2H), 2.29-2.16 (m, 1H), 1.86-1.71 (m, 1H).

### Step 3. Synthesis of benzyl (3R,4R)-3-{[(2-{5-[(3-ethoxypyridin-2-yl)oxy]pyridin-3-yl}pyrimidin-5-yl)carbonyl]amino}-4-fluoropiperidine-1-carboxylate (C17)

To a mixture of P1 (170 mg, 0.502 mmol), C16 (145 mg, 0.502 mmol), and N,N-diisopropylethylamine (0.263 mL, 1.51 mmol) in N,N-dimethylformamide (8 mL) was added O-(7-azabenzotriazol-1-yl)-N,N,N′,N′-tetramethyluronium hexafluorophosphate (HATU; 287 mg, 0.755 mmol). The reaction mixture was stirred at 18° C. for 2 hours, whereupon it was combined with two similar reactions carried out using C16 (42.7 mg, 0.148 mmol and 171 mg, 0.592 mmol), diluted with water (50 mL), and extracted with ethyl acetate (30 mL). The organic layer was washed with saturated aqueous sodium chloride solution (50 mL), dried over sodium sulfate, filtered, and concentrated under reduced pressure. Upon silica gel chromatography (Gradient: 0% to 100% ethyl acetate in petroleum ether), C17 was obtained as a yellow solid. Combined yield: 540 mg, 0.943 mmol, 76%. LCMS m/z 573.1 [M+H]⁺.

### Step 4. Synthesis of 2-{5-[(3-ethoxypyridin-2-yl)oxy]pyridin-3-yl}-N-[(3R,4R)-4-fluoropiperidin-3-yl]pyrimidine-5-carboxamide (5)

A mixture of C17 (300 mg, 0.524 mmol) and 10% palladium on carbon (300 mg) in ethanol (20 mL) was stirred under a balloon of hydrogen for 2 hours at 15° C. After the reaction mixture had been combined with two similar reactions carried out using C17 (200 mg, 0.349 mmol and 40 mg, 70 μmol), it was filtered through a pad of diatomaceous earth. The filtrate was concentrated, and the residue was purified using reversed-phase HPLC (Column: Agela Durashell C18, 5 μm; Mobile phase A: 0.05% ammonium hydroxide in water; Mobile phase B: acetonitrile; Gradient: 30% to 50% B), affording 2-{5-[(3-ethoxypyridin-2-yl)oxy]pyridin-3-yl}-N-[(3R,4R)-4-fluoropiperidin-3-yl]pyrimidine-5-carboxamide as a white solid. Combined yield: 174 mg, 0.397 mmol, 42%. LCMS m/z 439.2 [M+H]⁺. ¹H NMR (400 MHz, chloroform-d) δ 9.52 (d, J=1.8 Hz, 1H), 9.20 (s, 2H), 8.65 (d, J=2.8 Hz, 1H), 8.56 (dd, J=2.7, 1.8 Hz, 1H), 7.71 (dd, J=4.9, 1.5 Hz, 1H), 7.28-7.23 (m, 1H, assumed; partially obscured by solvent peak), 7.17 (br d, J=8 Hz, 1H), 7.02 (dd, J=7.9, 4.9 Hz, 1H), 4.86-4.66 (m, 1H), 4.37-4.26 (m, 1H), 4.18 (q, J=7.0 Hz, 2H), 3.36 (ddd, J=12.3, 3.4, 3.4 Hz, 1H), 3.09-2.99 (m, 1H), 2.86-2.75 (m, 2H), 2.11-1.81 (m, 2H), 1.50 (t, J=7.0 Hz, 3H).

### Example 6

### 2-{5-[(3-Ethoxy-5-fluoropyridin-2-yl)oxy]pyridin-3-yl}-N-[(3R,4R)-4-fluoropiperidin-3-yl]pyrimidine-5-carboxamide (6)

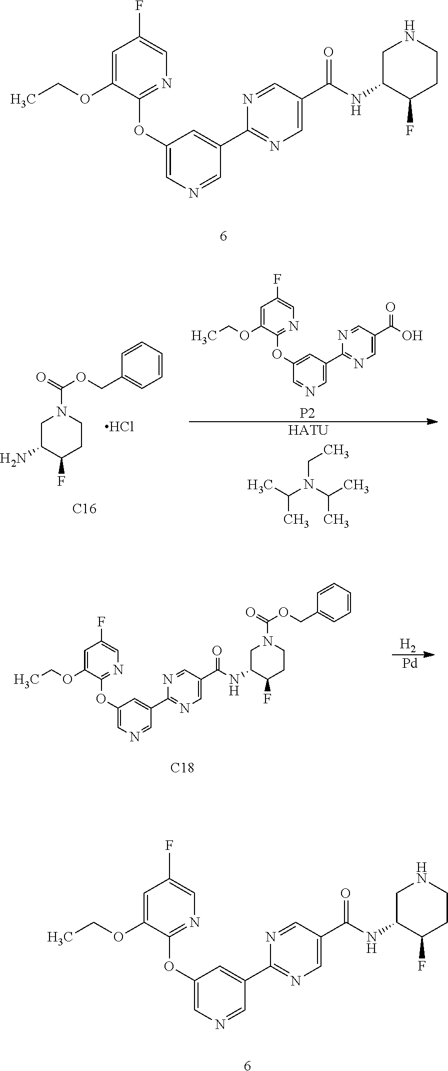

### Step 1. Synthesis of benzyl (3R,4R)-3-{[(2-{5-[(3-ethoxy-5-fluoropyridin-2-yl)oxy]pyridin-3-yl}pyrimidin-5-yl)carbonyl]amino}-4-fluoropiperidine-1-carboxylate (C18)

To a mixture of P2 (50 mg, 0.14 mmol), C16 (40.5 mg, 0.140 mmol), and N,N-diisopropylethylamine (73.3 μL, 0.421 mmol) in N,N-dimethylformamide (2 mL) was added O-(7-azabenzotriazol-1-yl)-N,N,N′,N′-tetramethyluronium hexafluorophosphate (HATU; 80 mg, 0.21 mmol). After the reaction mixture had been stirred at 18° C. for 2 hours, it was combined with a similar reaction carried out using C16 (24.3 mg, 84.2 μmol), then quenched with water (20 mL) and extracted with ethyl acetate (20 mL). The organic layer was washed with saturated aqueous sodium chloride solution (50 mL), dried over sodium sulfate, filtered, and concentrated in vacuo. Purification of the residue via preparative thin-layer chromatography (Eluent: ethyl acetate) afforded C18 as a yellow solid. Combined yield: 90 mg, 0.152 mmol, 68%. LCMS m/z 591.1 [M+H]⁺.

### Step 2. Synthesis of 2-{5-[(3-ethoxy-5-fluoropyridin-2-yl)oxy]pyridin-3-yl}-N-[(3R,4R)-4-fluoropiperidin-3-yl]pyrimidine-5-carboxamide (6)

A mixture of C18 (70 mg, 0.12 mmol) and 10% palladium on carbon (100 mg) in ethanol (20 mL) was stirred under a balloon of hydrogen for 2 hours at 15° C., whereupon it was combined with a similar reaction carried out using C18 (20 mg, 34 μmol) and filtered through a pad of diatomaceous earth. After the filtrate had been concentrated in vacuo, the residue was purified using reversed-phase HPLC (Column: Agela Durashell C18, 5 μm; Mobile phase A: 0.05% ammonium hydroxide in water; Mobile phase B: acetonitrile; Gradient: 30% to 50% B); this afforded 2-{5-[(3-ethoxy-5-fluoropyridin-2-yl)oxy]pyridin-3-yl}-N-[(3R,4R)-4-fluoropiperidin-3-yl]pyrimidine-5-carboxamide as a white solid. Yield: 14.2 mg, 31.1 μmol, 20%. LCMS m/z 457.1 [M+H]⁺. ¹H NMR (400 MHz, chloroform-d) δ 9.52 (d, J=1.8 Hz, 1H), 9.20 (s, 2H), 8.63 (d, J=2.7 Hz, 1H), 8.53 (dd, J=2.7, 1.8 Hz, 1H), 7.58 (d, J=2.6 Hz, 1H), 7.16-7.09 (br m, 1H), 7.06 (dd, J=9.2, 2.6 Hz, 1H), 4.86-4.68 (m, J_{HF}=47.2 Hz, 1H), 4.37-4.27 (m, 1H), 4.16 (q, J=7.0 Hz, 2H), 3.36 (ddd, J=12.2, 3.5, 3.4 Hz, 1H), 3.09-2.99 (m, 1H), 2.87-2.76 (m, 2H), 2.11-1.82 (m, 2H), 1.51 (t, J=7.0 Hz, 3H).

### Examples 7-25

TABLE 1
Method of synthesis and structure for Examples 7-25. The examples
below were made from analogous processes to the Example(s)
identified and from appropriate analogous starting materials.

| | Method of synthesis; | |
|---|---|---|
| Example | Non-commercial starting | |
| Number | materials | Structure |
| 7 | Example 5¹; P1 | 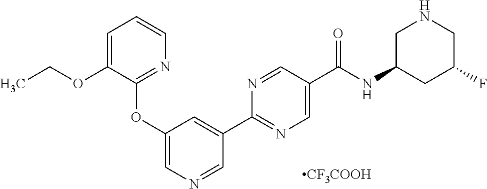 [SMILES: CCOc1cccnc1Oc1cncc(-c2ncc(C(=O)N[C@H]3CNC[C@H](F)C3)cn2)c1 ; "·CF3COOH"] |
| 8 | Example 1^{2,3,4}; P1 | 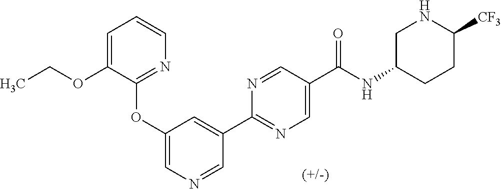 [SMILES: CCOc1cccnc1Oc1cncc(-c2ncc(C(=O)N[C@H]3CC[C@H](C(F)(F)F)NC3)cn2)c1 ; "(+/-)"] |
| | | 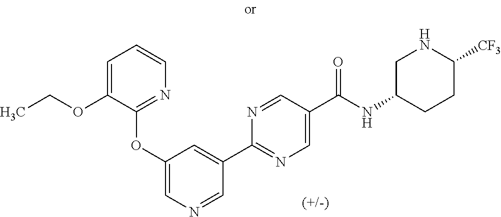 [SMILES: CCOc1cccnc1Oc1cncc(-c2ncc(C(=O)N[C@H]3CC[C@@H](C(F)(F)F)NC3)cn2)c1 ; "(+/-)"] |
| 9 | Example 1^{2,3,4}; P1 | 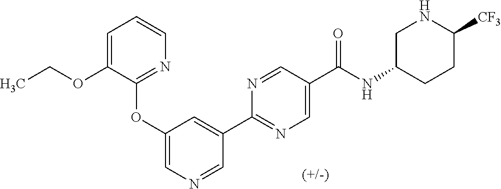 [SMILES: CCOc1cccnc1Oc1cncc(-c2ncc(C(=O)N[C@H]3CC[C@H](C(F)(F)F)NC3)cn2)c1 ; "(+/-)"] |
| | | 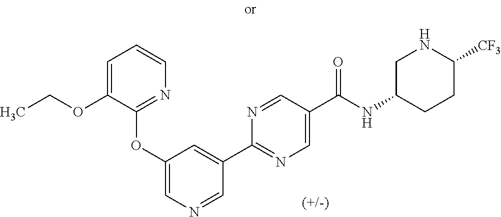 [SMILES: CCOc1cccnc1Oc1cncc(-c2ncc(C(=O)N[C@H]3CC[C@@H](C(F)(F)F)NC3)cn2)c1 ; "(+/-)"] |
| 10 | Example 9⁵ | 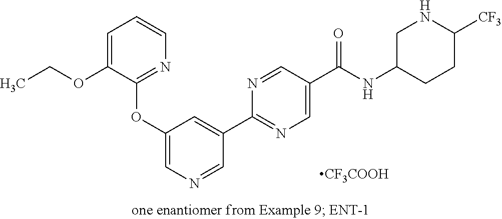 |
| 11 | Example 9⁵ | 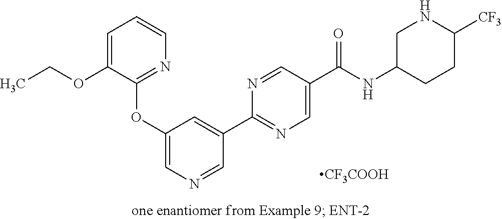 |
| 12 | Example 1; P1 | 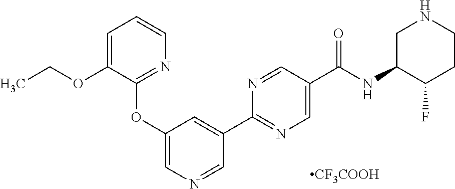 [SMILES: CCOc1cccnc1Oc1cncc(-c2ncc(C(=O)N[C@H]3CNCC[C@@H]3F)cn2)c1 ; "·CF3COOH"] |
| 13 | Example 5⁶; P2 | 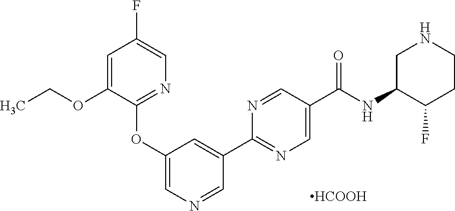 [SMILES: CCOc1cc(F)cnc1Oc1cncc(-c2ncc(C(=O)N[C@H]3CNCC[C@@H]3F)cn2)c1 ; "·HCOOH"] |
| 14 | Example 5¹; P1 | 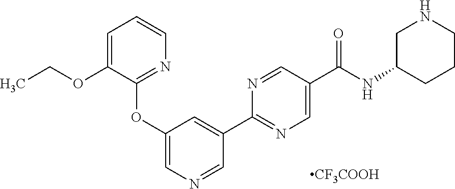 [SMILES: CCOc1cccnc1Oc1cncc(-c2ncc(C(=O)N[C@H]3CCCNC3)cn2)c1 ; "·CF3COOH"] |
| 15 | Example 14⁷; P1 | 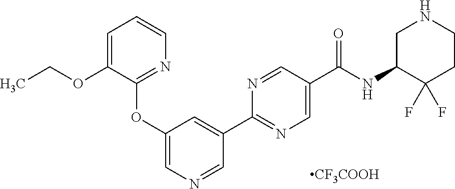 [SMILES: CCOc1cccnc1Oc1cncc(-c2ncc(C(=O)N[C@H]3CNCCC3(F)F)cn2)c1 ; "·CF3COOH"] |
| | | 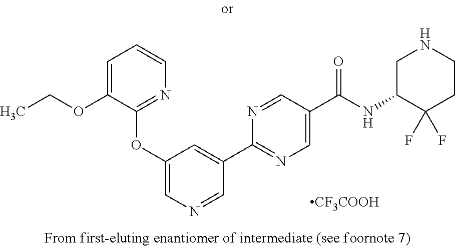 [SMILES: CCOc1cccnc1Oc1cncc(-c2ncc(C(=O)N[C@@H]3CNCCC3(F)F)cn2)c1 ; "·CF3COOH"] |
| 16 | Example 14⁷; P1 | 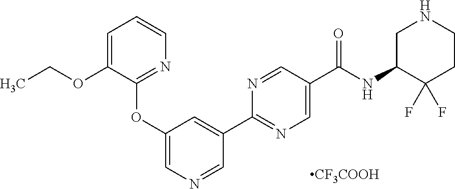 [SMILES: CCOc1cccnc1Oc1cncc(-c2ncc(C(=O)N[C@H]3CNCCC3(F)F)cn2)c1 ; "·CF3COOH"] |
| | | 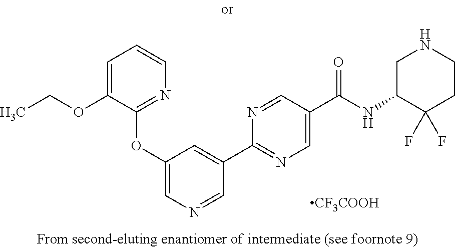 [SMILES: CCOc1cccnc1Oc1cncc(-c2ncc(C(=O)N[C@@H]3CNCCC3(F)F)cn2)c1 ; "·CF3COOH"] |
| 17 | Example 1^{1,3}; P1 | 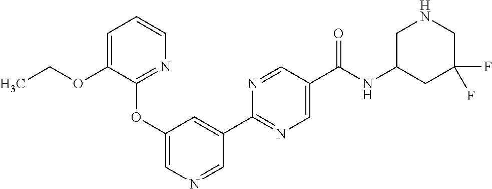 [SMILES: CCOc1cccnc1Oc1cncc(-c2ncc(C(=O)NC3CNCC(F)(F)C3)cn2)c1] |
| 18 | Example 17⁸ | 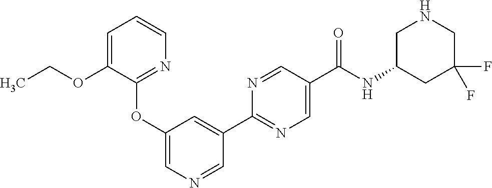 [SMILES: CCOc1cccnc1Oc1cncc(-c2ncc(C(=O)N[C@@H]3CNCC(F)(F)C3)cn2)c1] |
| | | 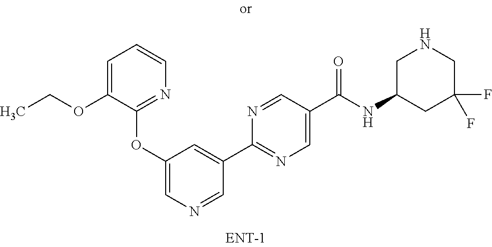 [SMILES: CCOc1cccnc1Oc1cncc(-c2ncc(C(=O)N[C@H]3CNCC(F)(F)C3)cn2)c1] |
| 19 | Example 17⁸ | 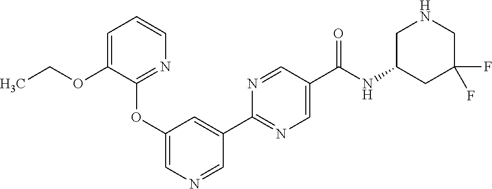 [SMILES: CCOc1cccnc1Oc1cncc(-c2ncc(C(=O)N[C@@H]3CNCC(F)(F)C3)cn2)c1] |
| | | 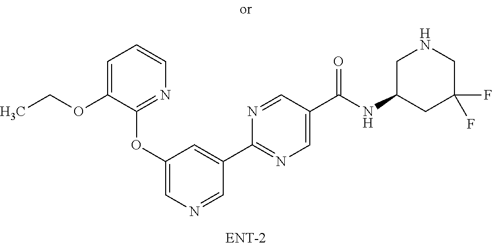 [SMILES: CCOc1cccnc1Oc1cncc(-c2ncc(C(=O)N[C@H]3CNCC(F)(F)C3)cn2)c1] |
| 20 | Example 14; P1 | 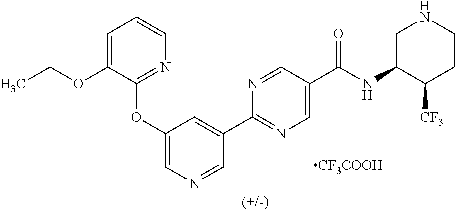 |
| 21 | Example 14; P1 | 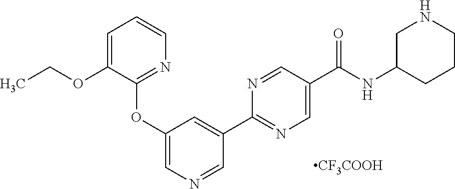 |
| 22 | Example 14; P1 | 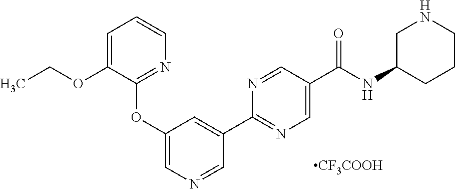 |
| 23 | Example 14; P1 | 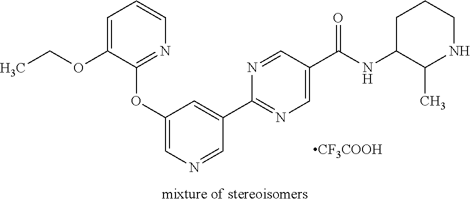 |
| 24 | Example 14; P1 | 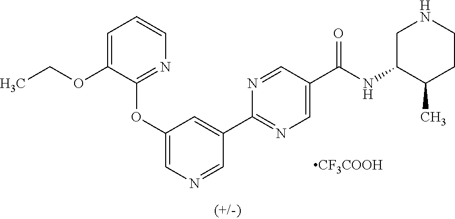 |
| 25 | Example 5⁹; P1 | 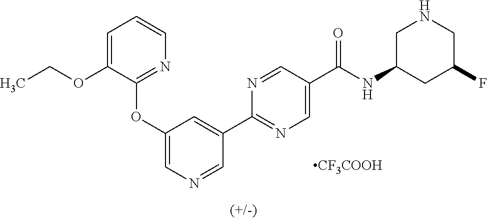 |

¹In this case, the nitrogen of the piperidine side chain reagent was protected with a tert-butoxycarbonyl group. After the amide coupling reaction, the product was deprotected using trifluoroacetic acid.
²Reaction of benzyl chloroformate with tert-butyl [6-(trifluoromethyl)piperidin-3-yl]carbamate in the presence of N,N-diisopropylethylamine provided benzyl 5-[(tert-butoxycarbonyl)amino]-2-(trifluoromethyl)piperidine-1-carboxylate, which was deprotected with trifluoroacetic acid to afford the requisite benzyl 5-amino-2-(trifluoromethyl)piperidine-1-carboxylate as a mixture of stereoisomers.
³In this case, the amide coupling was mediated by 2-chloro-1-methylpyridinium iodide (Mukaiyama reagent) and triethylamine.
⁴The racemic cis and trans products were separated using reversed-phase HPLC (Column: Waters XBridge C18 OBD, 5 μm; Mobile phase A: water containing 0.04% ammonium hydroxide and 10 mM ammonium bicarbonate; Mobile phase B: acetonitrile; Gradient: 23% to 53% B). The first-eluting isomer was designated as Example 8, and the second-eluting isomer as Example 9.
⁵Example 9 was separated into its component enantiomers using supercritical fluid chromatography {Column: Phenomenex Lux Amylose-1, 5 μm; Mobile phase: 7:3 carbon dioxide/[ethanol containing 0.2% (7M ammonia in methanol)]}. The first-eluting enantiomer was designated as Example 10, and the second-eluting enantiomer as Example 11. Retention time for Example 10: 6.48 minutes [Column: Phenomenex Lux Amylose-1, 4.6 × 250 mm, 5 um; Mobile phase A: carbon dioxide; Mobile phase B: ethanol containing 0.2% (7M ammonia in methanol); Gradient: 5% B for 1.0 minute, then 5% to 60% B over 8.0 minutes; Flow rate: 3.0 mL/minute; Back pressure: 120 bar]. Retention time for Example 11: 6.68 minutes (Analytical conditions identical to those used for Example 10). These two compounds are enantiomers of one another, but of undetermined relative and absolute stereochemistry.
⁶The piperidine side chain employed for Example 13 was tert-butyl (3S,4S)-3-amino-4-fluoropiperidine-1-carboxylate; after the amide coupling, deprotection was carried out using trifluoroacetic acid.
⁷Reaction of tert-butyl rac-(3R,4R)-3-amino-4-hydroxypiperidine-1-carboxylate with benzyl chloroformate and sodium carbonate, followed by oxidation of the product with 1,1,1-tris(acetyloxy)-1,1-dihydro-1,2-benziodoxol-3-(1H)-one (Dess-Martin periodinane), afforded tert-butyl 3-{[(benzyloxy)carbonyl]amino}-4-oxopiperidine-1-carboxylate. Difluorination of this material using (diethylamino)sulfur trifluoride provided tert-butyl 3-{[(benzyloxy)carbonyl]amino}-4,4-difluoropiperidine-1-carboxylate, which was separated into its component enantiomers via supercritical fluid chromatography [Column: Chiral Technologies Chiralpak AD, 10 μm; Mobile phase: 85:15 carbon dioxide/(ethanol containing 0.15% ammonium hydroxide)]. The first-eluting enantiomer had a retention time of 2.73 minutes (Column: Chiral Technologies Chiralcel OJ-H, 4.6 × 250 mm, 5 μm; Mobile phase A: carbon dioxide; Mobile phase B: ethanol containing 0.05% diethylamine; Gradient: 5% to 40% B over 5 minutes; Flow rate: 2.5 mL/minute). The second-eluting enantiomer exhibited a retention time of 3.08 minutes under the same conditions. The first-eluting enantiomer was deprotected by hydrogenation over palladium hydroxide, to afford one enantiomer of tert-butyl 3-amino-4,4-difluoropiperidine-1-carboxylate; this material exhibited a negative (−) rotation, and was used in the synthesis of Example 15. The second-eluting enantiomer was deprotected in the same manner to provide the other enantiomer of tert-butyl 3-amino-4,4-difluoropiperidine-1-carboxylate, which exhibited a positive (+) rotation, and was used in the synthesis of Example 16.
⁸Example 17 was separated into its component enantiomers using supercritical fluid chromatography [Column: Chiral Technologies Chiralpak IC, 3 μm; Mobile phase: 3.2 carbon dioxide/(ethanol containing 0.05% diethylamine); Flow rate 2.5 mL/minute]. The second-eluting enantiomer was designated as Example 19, and gave a retention time of 4.42 minutes (Analytical conditions identical to those used for Example @521).
⁹The piperidine side chain employed for Example 25 was tert-butyl rac-(3R,5S)-3-amino-5-fluoropiperidine-1-carboxylate; after the amide coupling, deprotection was carried out using trifluoroacetic acid.

TABLE 2
Compound name and physicochemical data for Examples 7-25.

| | | ¹H NMR (400 MHz, CDCl₃); Mass spectrum, |
|---|---|---|
| | | observed ion m/z [M + H]⁺ or HPLC retention time; |
| Example | | Mass spectrum m/z [M + H]⁺ (unless otherwise |
| Number | Compound Name | indicated) |
| 7 | 2-{5-[(3-ethoxypyridin-2-yl)oxy]pyridin-3- | 1.82 minutes¹; 439.1 |
| | yl}-N-[(3R,5R)-5-fluoropiperidin-3- | |
| | yl]pyrimidine-5-carboxamide, | |
| | trifluoroacetate salt | |
| 8 | First-eluting isomer (see footnote 4 in | First-eluting isomer; characteristic peaks: 9.62- |
| | Table 1); | 9.42 (m, 1H), 9.13 (s, 2H), 8.73-8.59 (m, 1H), |
| | rac-2-{5-[(3-ethoxypyridin-2-yl)oxy]pyridin- | 8.56 (s, 1H), 7.70 (dd, J = 5.0, 1.5 Hz, 1H), 7.29- |
| | 3-yl}-N-[(3R,6S)-6- | 7.22 (m, 1H, assumed; partially obscured by |
| | (trifluoromethyl)piperidin-3-yl]pyrimidine-5- | solvent peak), 7.03 (dd, J = 7.9, 4.9 Hz, 1H), 6.26 |
| | carboxamide | (br d, J = 7.7 Hz, 1H), 4.23-4.09 (m, 1H), 4.18 |
| | or | (q, J = 7.0 Hz, 2H), 3.51 (br d, J = 12 Hz, 1H), |
| | rac-2-{5-[(3-ethoxypyridin-2-yl)oxy]pyridin- | 3.24-3.12 (m, 1H), 2.63-2.54 (m, 1H), 2.29- |
| | 3-yl}-N-[(3R,6R)-6- | 2.18 (m, 1H), 2.08-1.96 (m, 1H), 1.78-1.66 |
| | (trifluoromethyl)piperidin-3-yl]pyrimidine-5- | (m, 1H), 1.50 (t, J = 7.0 Hz, 3H); 489.3 |
| | carboxamide | |
| 9 | Second-eluting isomer (see footnote 4 in | Second-eluting isomer; 9.60-9.48 (m, 1H), 9.19 |
| | Table 1); | (s, 2H), 8.71-8.61 (m, 1H), 8.57 (br s, 1H), 7.71 |
| | rac-2-{5-[(3-ethoxypyridin-2-yl)oxy]pyridin- | (dd, J = 4.8, 1.5 Hz, 1H), 7.3-7.23 (m, 1H, |
| | 3-yl}-N-[(3R,6S)-6- | assumed; partially obscured by solvent peak), |
| | (trifluoromethyl)piperidin-3-yl]pyrimidine-5- | 7.20 (br d, J = 7.8 Hz, 1H), 7.02 (dd, J = 7.9, 4.9 |
| | carboxamide | Hz, 1H), 4.40-4.32 (m, 1H), 4.18 (q, J = 7.0 Hz, |
| | or | 2H), 3.28-3.19 (m, 1H), 3.16 (br d, J = 12 Hz, |
| | rac-2-{5-[(3-ethoxypyridin-2-yl)oxy]pyridin- | 1H), 3.01 (br d, J = 12 Hz, 1H), 2.24-2.13 (m, |
| | 3-yl}-N-[(3R,6R)-6- | 1H), 1.86-1.65 (m, 3H, assumed; partially |
| | (trifluoromethyl)piperidin-3-yl]pyrimidine-5- | obscured by water peak), 1.50 (t, J = 7.0 Hz, 3H); |
| | carboxamide | 489.3 |
| 10 | First-eluting enantiomer from separation of | 1.91 minutes¹; 489.4 |
| | Example 9 (see footnote 5 in Table 1); | |
| | 2-{5-[(3-ethoxypyridin-2-yl)oxy]pyridin-3- | |
| | yl}-N-[6-(trifluoromethyl)piperidin-3- | |
| | yl]pyrimidine-5-carboxamide, | |
| | trifluoroacetate salt, ENT-1 | |
| 11 | Second-eluting enantiomer from | 1.91 minutes¹; 489.4 |
| | separation of Example 9 (see footnote 5 in | |
| | Table 1); | |
| | 2-{5-[(3-ethoxypyridin-2-yl)oxy]pyridin-3- | |
| | yl}-N-[6-(trifluoromethyl)piperidin-3- | |
| | yl]pyrimidine-5-carboxamide, | |
| | trifluoroacetate salt, ENT-2 | |
| 12 | 2-{5-[(3-ethoxypyridin-2-yl)oxy]pyridin-3- | 9.83 (br s, 2H), 9.63 (s, 1H), 9.35 (s, 2H), 9.12 |
| | yl}-N-[(3S,4S)-4-fluoropiperidin-3- | (br d, J = 7.9 Hz, 1H), 9.05 (dd, J = 2.5, 1.5 Hz, |
| | yl]pyrimidine-5-carboxamide, | 1H), 8.77 (d, J = 2.5 Hz, 1H), 7.70 (dd, J = 4.8, |
| | trifluoroacetate salt | 1.5 Hz, 1H), 7.31 (dd, J = 8.0, 1.5 Hz, 1H), 7.12 |
| | | (dd, J = 8.0, 4.8 Hz, 1H), 4.92 (br d, J_{HF} = 46.1 |
| | | Hz, 1H), 4.82-4.72 (m, 1H), 4.17 (q, J = 7.0 Hz, |
| | | 2H), 3.58 (br AB quartet, J_{AB} = 13 Hz, Δν_{AB} = 55 |
| | | Hz, 2H), 3.44-3.29 (m, 2H), 2.52-2.31 (m, |
| | | 1H), 2.26-2.11 (m, 1H), 1.49 (t, J = 7.0 Hz, 3H); |
| | | 439.4 |
| 13 | 2-{5-[(3-ethoxy-5-fluoropyridin-2- | Characteristic peaks: 9.48 (d, J = 1.7 Hz, 1H), |
| | yl)oxy]pyridin-3-yl}-N-[(3S,4S)-4- | 8.89 (s, 2H), 8.62 (d, J = 2.7 Hz, 1H), 8.52 (dd, J = |
| | fluoropiperidin-3-yl]pyrimidine-5- | 2.7, 1.7 Hz, 1H), 7.56 (d, J = 2.5 Hz, 1H), 7.05 |
| | carboxamide, formate salt | (dd, J = 9.1, 2.5 Hz, 1H), 4.77-4.44 (m, 1H), |
| | | 4.38-4.21 (m, 1H), 4.15 (q, J = 7.0 Hz, 2H), |
| | | 3.44-3.34 (m, 1H), 2.30-2.12 (m, 1H), 1.95- |
| | | 1.71 (m, 1H), 1.50 (t, J = 7.0 Hz, 3H); 457.1 |
| 14 | 2-{5-[(3-ethoxypyridin-2-yl)oxy]pyridin-3- | 2.29 minutes²; 421 |
| | yl}-N-[(3S)-piperidin-3-yl]pyrimidine-5- | |
| | carboxamide, trifluoroacetate salt | |
| 15 | From first-eluting enantiomer of | 2.50 minutes²; 457 |
| | intermediate (see footnote 7, Table 1); | |
| | N-[(3S)-4,4-difluoropiperidin-3-yl]-2-{5-[(3- | |
| | ethoxypyridin-2-yl)oxy]pyridin-3- | |
| | yl}pyrimidine-5-carboxamide, | |
| | trifluoroacetate salt | |
| | or | |
| | N-[(3R)-4,4-difluoropiperidin-3-yl]-2-{5-[(3- | |
| | ethoxypyridin-2-yl)oxy]pyridin-3- | |
| | yl}pyrimidine-5-carboxamide, | |
| | trifluoroacetate salt | |
| 16 | From second-eluting enantiomer of | 2.48 minutes²; 457 |
| | intermediate (see footnote 7, Table 1); | |
| | N-[(3S)-4,4-difluoropiperidin-3-yl]-2-{5-[(3- | |
| | ethoxypyridin-2-yl)oxy]pyridin-3- | |
| | yl}pyrimidine-5-carboxamide, | |
| | trifluoroacetate salt | |
| | or | |
| | N-[(3R)-4,4-difluoropiperidin-3-yl]-2-{5-[(3- | |
| | ethoxypyridin-2-yl)oxy]pyridin-3- | |
| | yl}pyrimidine-5-carboxamide, | |
| | trifluoroacetate salt | |
| 17 | N-(5,5-difluoropiperidin-3-yl)-2-{5-[(3- | 9.51 (d, J = 1.8 Hz, 1H), 9.16 (s, 2H), 8.65 (d, J = |
| | ethoxypyridin-2-yl)oxy]pyridin-3- | 2.8 Hz, 1H), 8.58-8.54 (m, 1H), 7.71 (br d, J = |
| | yl}pyrimidine-5-carboxamide | 4.8 Hz, 1H), 7.3-7.23 (m, 1H, assumed; |
| | | partially obscured by solvent peak), 7.02 (dd, J = |
| | | 7.9, 4.8 Hz, 1H), 6.96 (br d, J = 8.5 Hz, 1H), 4.58- |
| | | 4.48 (m, 1H), 4.18 (q, J = 7.0 Hz, 2H), 3.28- |
| | | 3.16 (m, 1H), 3.07-2.89 (m, 3H), 2.39-2.12 |
| | | (m, 2H), 1.50 (t, J = 7.0 Hz, 3H); 457.4 |
| 18 | First-eluting enantiomer (see footnote 8 in | 9.55-9.49 (m, 1H), 9.16 (s, 2H), 8.68-8.62 (m, |
| | Table 1); | 1H), 8.56 (br s, 1H), 7.71 (br d, J = 5.0 Hz, 1H), |
| | N-[(3S)-5,5-difluoropiperidin-3-yl]-2-{5-[(3- | 7.3-7.23 (m, 1H, assumed; partially obscured |
| | ethoxypyridin-2-yl)oxy]pyridin-3- | by solvent peak), 7.02 (dd, J = 7.9, 4.9 Hz, 1H), |
| | yl}pyrimidine-5-carboxamide | 6.95 (br d, J = 8 Hz, 1H), 4.58-4.49 (m, 1H), |
| | or | 4.18 (q, J = 7.0 Hz, 2H), 3.28-3.17 (m, 1H), |
| | N-[(3R)-5,5-difluoropiperidin-3-yl]-2-{5-[(3- | 3.08-2.89 (m, 3H), 2.39-2.11 (m, 2H), 1.50 (t, |
| | ethoxypyridin-2-yl)oxy]pyridin-3- | J = 7.0 Hz, 3H); 457.4 |
| | yl}pyrimidine-5-carboxamide | |
| 19 | Second-eluting enantiomer(see footnote 8 | 9.52 (d, J = 1.7 Hz, 1H), 9.16 (s, 2H), 8.65 (d, J = |
| | in Table 1); | 2.7 Hz, 1H), 8.56 (dd, J = 2, 2 Hz, 1H), 7.73- |
| | N-[(3S)-5,5-difluoropiperidin-3-yl]-2-{5-[(3- | 7.69 (m, 1H), 7.3-7.23 (m, 1H, assumed; |
| | ethoxypyridin-2-yl)oxy]pyridin-3- | partially obscured by solvent peak), 7.03 (dd, J = |
| | yl}pyrimidine-5-carboxamide | 7.9, 5.0 Hz, 1H), 6.92 (br d, J = 8 Hz, 1H), 4.57- |
| | or | 4.49 (m, 1H), 4.18 (q, J = 7.0 Hz, 2H), 3.28- |
| | N-[(3R)-5,5-difluoropiperidin-3-yl]-2-{5-[(3- | 3.17 (m, 1H), 3.06-2.89 (m, 3H), 2.39-2.12 |
| | ethoxypyridin-2-yl)oxy]pyridin-3- | (m, 2H), 1.50 (t, J = 7.0 Hz, 3H); 457.4 |
| | yl}pyrimidine-5-carboxamide | |
| 20 | rac-2-{5-[(3-ethoxypyridin-2-yl)oxy]pyridin- | 2.38 minutes²; 435 |
| | 3-yl}-N-[(3R,4R)-4-methylpiperidin-3- | |
| | yl]pyrimidine-5-carboxamide, | |
| | trifluoroacetate salt | |
| 21 | 2-{5-[(3-ethoxypyridin-2-yl)oxy]pyridin-3- | 2.37 minutes²; 421 |
| | yl}-N-(piperidin-3-yl)pyrimidine-5- | |
| | carboxamide, trifluoroacetate salt | |
| 22 | 2-{5-[(3-ethoxypyridin-2-yl)oxy]pyridin-3- | 2.64 minutes²; 421 |
| | yl}-N-[(3R)-piperidin-3-yl]pyrimidine-5- | |
| | carboxamide, trifluoroacetate salt | |
| 23 | 2-{5-[(3-ethoxypyridin-2-yl)oxy]pyridin-3- | 2.39 minutes²; 435 |
| | yl}-N-(2-methylpiperidin-3-yl)pyrimidine-5- | |
| | carboxamide, trifluoroacetate salt, mixture | |
| | of stereoisomers | |
| 24 | rac-2-{5-[(3-ethoxypyridin-2-yl)oxy]pyridin- | 2.42 minutes²; 435 |
| | 3-yl}-N-[(3R,4S)-4-methylpiperidin-3- | |
| | yl]pyrimidine-5-carboxamide, | |
| | trifluoroacetate salt | |
| 25 | rac-2-{5-[(3-ethoxypyridin-2-yl)oxy]pyridin- | ¹H NMR (400 MHz, DMSO-d₆) δ 9.40 (d, J = 1.8 |
| | 3-yl}-N-[(3R,5S)-5-fluoropiperidin-3- | Hz, 1H), 9.27 (s, 2H), 9.15-8.93 (br s, 2H), 8.79 |
| | yl]pyrimidine-5-carboxamide, | (br d, J = 6.9 Hz, 1H), 8.65 (d, J = 2.7 Hz, 1H), |
| | trifluoroacetate salt | 8.38 (dd, J = 2.7, 1.8 Hz, 1H), 7.68 (dd, J = 4.9, |
| | | 1.5 Hz, 1H), 7.58 (dd, J = 8.0, 1.5 Hz, 1H), 7.18 |
| | | (dd, J = 8.0, 4.8 Hz, 1H), 5.01 (br d, J_{HF} = 45.8 |
| | | Hz, 1H), 4.36-4.25 (m, 1H), 4.18 (q, J = 7.0 Hz, |
| | | 2H), 3.57-3.45 (m, 1H), 3.39 (br d, J = 12.4 Hz, |
| | | 1H), 3.31-3.19 (m, 1H), 3.06 (dd, J = 12.4, 8.1 |
| | | Hz, 1H), 2.43-2.29 (m, 1H), 2.08-1.95 (m, |
| | | 1H), 1.37 (t, J = 7.0 Hz, 3H); 439.2 |

¹Conditions for analytical HPLC. Column: Waters Atlantis dC18, 4.6 × 50 mm, 5 μm; Mobile phase A: 0.05% trifluoroacetic acid in water (v/v); Mobile phase B: 0.05% trifluoroacetic acid in acetonitrile (v/v); Gradient: 5.0% to 95% B, linear over 4.0 minutes; Flow rate: 2 mL/minute.
²Conditions for analytical HPLC. Column: Waters XBridge C18, 2.1 × 50 mm, 5 μm; Mobile phase A: 0.0375% trifluoroacetic acid in water; Mobile phase B: 0.01875% trifluoroacetic acid in acetonitrile; Gradient: 1% to 5% B over 0.6 minutes; 5% to 100% B over 3.4 minutes; Flow rate: 0.8 mL/minute.

### Examples D1-D3

Table 3 includes three prophetic examples incorporating a deuterated ethyl group. The preparation of these compounds would employ variations of the methods described above using ordinary skill in the art. Example D1 could be prepared from intermediates P3 and P4, in a manner analogous to that described for Example 4. Examples D2 and D3 could be prepared from a deuterated version of P2 via the methods employed for Examples 2 and 1, respectively.

TABLE 3
Compound name and structure for Examples D1-D3

| Example | | |
|---|---|---|
| Number | Compound Name | Structure |
| D1 | 2-{5-[((3-Ethoxy-d₅)pyridin-2- yl)oxy]pyridin-3-yl}-N-[(3S,5S)-5- fluoropiperidin-3-yl]pyrimidine-5- carboxamide | 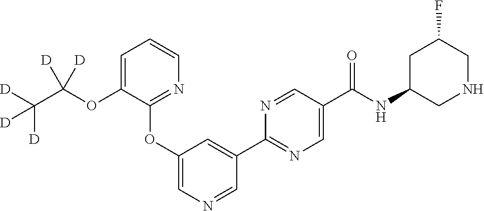 [SMILES: [2H]C([2H])([2H])C([2H])([2H])Oc1cccnc1Oc1cncc(-c2ncc(C(=O)N[C@@H]3CNC[C@@H](F)C3)cn2)c1] |
| D2 | 2-{5-[((3-Ethoxy-d₅)-5- fluoropyridin-2-yl)oxy]pyridin-3- yl}-N-[(3S,5S)-5-fluoropiperidin- 3-yl]pyrimidine-5-carboxamide | 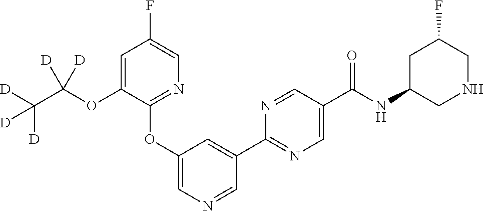 [SMILES: [2H]C([2H])([2H])C([2H])([2H])Oc1cc(F)cnc1Oc1cncc(-c2ncc(C(=O)N[C@@H]3CNC[C@@H](F)C3)cn2)c1] |
| D3 | 2-{5-[((3-Ethoxy-d₅)-5- fluoropyridin-2-yl)oxy]pyridin-3- yl}-N-[(3R,4S)-4-fluoropiperidin- 3-yl]pyrimidine-5-carboxamid | 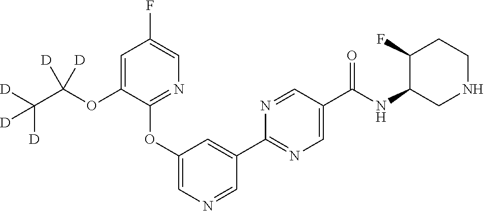 [SMILES: [2H]C([2H])([2H])C([2H])([2H])Oc1cc(F)cnc1Oc1cncc(-c2ncc(C(=O)N[C@@H]3CNCC[C@@H]3F)cn2)c1] |

### Example 26: Preparation of 4-(4-(1-isopropyl-7-oxo-1,4,6,7-tetrahydrospiro[indazole-5,4′-piperidine]-1′-carbonyl)-6-methoxypyridin-2-yl)benzoic acid, Compound A (ACCi Compound)

A preparation of 4-(4-(1-Isopropyl-7-oxo-1,4,6,7-tetrahydrospiro[indazole-5,4′-piperidine]-1′-carbonyl)-6-methoxypyridin-2-yl)benzoic acid is in Example 9 of U.S. Pat. No. 8,859,577, hereby incorporated herein by reference in its entireties for all purposes. Preparation of [(1R,5S,6R)-3-{2-[(2S)-2-methylazetidin-1-yl]-6-(trifluoromethyl) pyrimidin-4-yl}-3-azabicyclo[3.1.0.0]hex-6-yl]acetic acid (including a crystalline free acid form thereof) is described in Example 4 of U.S. Pat. No. 9,809,579.

In the preparation of Compound A, it is noted that some of the preparation methods described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991. Furthermore, this invention is not limited to specific synthetic methods provided herein that may vary.

### Intermediate A1: 1-Isopropyl-4,6-dihydrospiro[Indazole-5,4′-piperldin]-7(1H)-one, Hydrochloride Salt

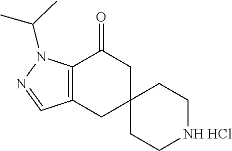

### Step 1. tert-Butyl 9-oxo-3-azaspiro[5.5]Undec-7-ene-3-carboxylate

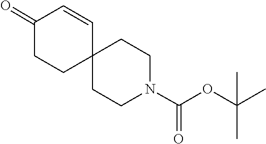 [SMILES: CC(C)(C)OC(=O)N1CCC2(C=CC(=O)CC2)CC1]

A dry reactor was charged with tert-butyl 4-formylpiperidine-1-carboxylate (108 Kg), cyclohexane (1080 L) and pyrrolidine (64.8 Kg) at 25-30° C. The mixture was stirred 5-10 min, and was then heated to reflux for 12-16 h, while collecting water using a Dean-Stark trap. The reaction mixture was then cooled to 50-60° C., at which temperature vacuum was applied to distill excess pyrrolidine and cyclohexane. The reaction mixture was then cooled to 25-30° C., and cyclohexane (648 L) was charged, followed by methyl vinyl ketone (49.63 Kg). The mixture was stirred for 12-16 h, then filtered and the filtrate was charged into a clean and dry reactor. The solution was cooled to 10-15° C., then a solution of acetic acid (54.75 Kg) in water (54 L) was slowly added, maintaining the temperature below 15° C. At the end of the addition, the mixture was warmed up to 25-30° C. and stirred for 12-16 h. The layers were separated and the aqueous was extracted with ethyl acetate (324 L). Combined organic layers were washed with a solution of sodium bicarbonate (32.34 Kg) in water (324 L), then dried over sodium sulfate. The solids were washed with ethyl acetate (54 L), and combined filtrates were concentrated under reduced pressure at below 40° C. n-Heptane (216 L) was charged into the reactor and distillation was pursued under reduced pressure and at below 40° C. until dryness. The mixture was cooled to 25-30° C. and n-heptane (216 L) was charged in the reactor. The mixture was stirred for 1-2 h after formation of solids. The solids were then filtered, washed with n-heptane (54 L) and dried at 40-50° C. for 10-12 h to generate the desired material (90.1 Kg, 67% yield).

### Step 2. (E)-tert-Butyl 10-((dimethylamino)methylene)-9-oxo-3-azaspiro[5.5]undec-7-ene-3-carboxylate

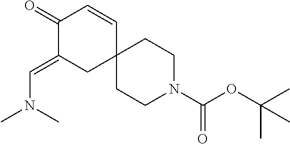 [SMILES: CN(C)/C=C1\CC2(C=CC1=O)CCN(C(=O)OC(C)(C)C)CC2]

A clean and dry reactor was charged with tert-butyl 9-oxo-3-azaspiro[5.5]undec-7-ene-3-carboxylate (50 Kg), N,N-dimethylformamide (500 L) and N,N-dimethylformamide dimethyl acetal (135 Kg) at 25-30° C. under nitrogen atmosphere. The reaction mixture was stirred 5-10 min then heated to 120-130° C. for 20 h. the mixture was then cooled to 50-60° C., and the solvent was distilled under high vacuum at below 60° C. Mix-xylenes (200 L) was charged at below 45° C. and the solvent was distilled under high vacuum at below 60° C. This operation was repeated with another lot of mix-xylenes (200 L). Toluene (200 L) was then charged into the reactor and the solvent was distilled under high vacuum at below 60° C. This operation was repeated with a second lot of toluene (200 L). Methyl tert-butyl ether (100 L) was then charged at below 30° C. and the solvent was distill under high vacuum at below 40° C. The mixture was cooled down to 15-20° C. and methyl tert-butyl ether (100 L) was charged at below 20° C. The mixture was stirred for 20-30 min and the solids were filtered, washed with methyl tert-butyl ether (50 L) and dried without vacuum at 50-55° C. for 10 h to provide the desired compound (52.1 Kg, 87% yield). ¹H NMR (400 MHz, CDCl₃) δ ppm 7.48 (s, 1H), 6.57 (d, J=9.97 Hz, 1H), 5.99 (d, J=10.16 Hz, 1H), 3.32-3.51 (m, 4H), 3.06 (s, 6H), 2.72 (s, 2H), 1.57-1.66 (m, 2H), 1.41-1.53 (m, 11H).

### Step 3. tert-Butyl 1-isopropyl-1,4-dihydrospiro[Indazole-5,4′-piperidine]-1′-carboxylate

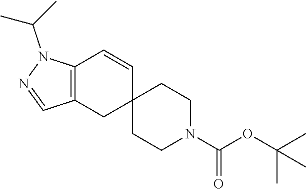 [SMILES: CC(C)n1ncc2c1C=CC1(CCN(C(=O)OC(C)(C)C)CC1)C2]

A clean and dry reactor was charged with (E)-tert-butyl 10-((dimethylamino)methylene)-9-oxo-3-azaspiro[5.5]undec-7-ene-3-carboxylate (80 Kg), toluene (704 L) and trimethylamine (16 L) at 25-30° C. The reaction mixture was warmed up to 70-80° C., and a solution of isopropyl hydrazine hydrochloride salt in methanol (1.25 equiv., 141 Kg total) was added over 4-5 h. The reaction mixture was then stirred for 8-10 h at 70-80° C., prior cooling to 15-25° C. A solution of citric acid (48 Kg) in water (480 L) was then slowly added, maintaining internal temperature below 25° C. Ethyl acetate (208 L) was added and the mixture was stirred for 10 min. Layers were separated and the organic layer was successively washed with a solution of citric acid (48 Kg) in water (480 L), then with only water (320 L). Combined aqueous layers were extracted with ethyl acetate (320 L). Combined organic layers were then dried over sodium sulfate (8 Kg) and the solvents were evaporated to dryness under reduce pressure and at below 40° C. Dichloromethane (240 L) was charged into the reactor and the mixture was stirred at 25-30° C. until clear. Activated carbon (1.84 Kg), magnesium silicate (1.84 Kg) and silica gel (32 Kg, 100-200 mesh) were successively charged at 25-30° C. and the heterogeneous mixture was stirred for 1 h. The slurry was then filter on a Hyflow bed, prepared by mixing Hyflow supercell (8 Kg) and dichloromethane (40 L). The cake was washed with dichloromethane (three times 120 L). The combined filtrates were charged back in the reactor and the solvent was evaporated under reduced pressure at below 40° C. n-Heptane (160 L) was then charged and distilled under reduced pressure at below 40° C. n-Heptane (200 L) was charged in the reactor and the mixture was cooled down to 0-5° C. After stirring for 12-15 h, the solids were filtered at 0° C., washed with chilled (0-5° C.) n-heptane (160 L) and dried under vacuum at 40-50° C. to provide the title compound (82.4 Kg, 75%). ¹H NMR (400 MHz, CDCl₃) δ ppm 7.25 (s, 1H), 6.42 (dd, J=10.05, 0.49 Hz, 1H) 5.84 (d, J=9.95 Hz, 1H), 4.42-4.52 (m, 1H), 3.36-3.53 (m, 4H), 2.62 (s, 2H) 1.56-1.68 (m, 2H) 1.45-1.55 (m, 17H).

### Step 4. 1-Isopropyl-4,6-dihydrospiro[Indazole-5,4′-piperldin]-7(1H)-One, Hydrochloride Salt

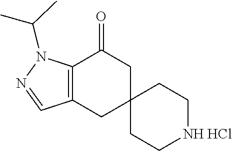

A clean and dry reactor was charged with tert-butyl 1-isopropyl-1,4-dihydrospiro[indazole-5,4′-piperidine]-1′-carboxylate (60 Kg) and methanol (600 L) at 25-30° C. N-Bromosuccinimide (32.4 Kg) was added in 5 portions over 30-40 min at 25-30° C. and stirring was continued for 30-60 min. A solution of sodium thiosulfate pentahydrate (5.4 Kg) in water (102 L) was slowly added, maintaining internal temperature below 30° C. The mixture was stirred for 20-30 min then the solvent was evaporated under reduced pressure at below 45° C. The residue was cooled down to 25-30° C. and 2-methyltetrahydrofuan (420 L) was charged in the reactor, along with water (90 L). The mixture was stirred for 15-20 min, then the layers were separated, the aqueous layer was further extracted with 2-methyltetrahydrofuran (120 L). Combined organic extracts were treated for 15-20 min at 25-30° C. with a solution of sodium hydroxide (4.8 Kg) in water (120 L). Layers were separated and the organic layer was washed with water (120 L), followed by a solution of sodium chloride (12 Kg) in water (120 L) and then dried over sodium sulfate (6 Kg). After filtration, the cake was washed with 2-methyltetrahydrofuran (30 L) and combined filtrate were charged back into the reactor. The solvent was completely distilled at below 45° C. under reduced pressure and the residue was solubilized in tetrahydrofuran (201 L). In another clean and dry reactor was charged potassium tert-butoxide (60.6 Kg) and tetrahydrofuran (360 L) at 25-30° C. To that mixture was slowly added the solution of the residue in tetrahydrofuran maintaining a temperature below 30° C. The reaction mixture was then warmed up to 60-65° C. and kept at this temperature for 1-2 h. Upon completion, the mixture was cooled to 0-10° C., and slowly quenched with a solution of hydrochloric acid (1 N, 196 L), maintaining internal temperature below 10° C. The reaction mixture was allowed to warm up to 25-30° C., and ethyl acetate (798 L) was charged. After stirring for 15-20 min, the layers were separated, and the aqueous layer was further extracted with ethyl acetate (160 L). Combined organic layers were washed with water (160 L), dried over sodium sulfate (8 Kg), filtered, and the cake was washed with ethyl acetate (300 L). The solvents were entirely distilled under reduced pressure at below 45° C., and ethyl acetate (540 L) was charged into the reactor at 25-30° C., followed by methanol (156 L). The mixture was cooled to 0-5° C., at which point acetyl chloride (79.8 Kg) was slowly added, maintaining the temperature in the specified range. The mixture was then allowed to warm up to 20-25° C. and was kept at this temperature for 4-5 h with stirring. The resulting slurry was filtered and the solids were washed with ethyl acetate (120 L), then dried at 40-45° C. for 8-10 h to furnish the desired crude product (33.5 Kg, 65%).

A final purification step was performed by solubilizing this crude solid (56.8 Kg) in methanol (454.4 L) in a clean a dried reactor at 25-30° C. The solution was stirred for 30-45 min, then passed through a 0.2 micron cartridge filter into a clean and dry reactor at 25-30° C. Methanol was distilled under reduced pressure at below 50° C. until ˜1 vol solvent remains. The reaction mixture was cooled to 25-30° C. and fresh acetonitrile (113.6 L) was charged through a 0.2 micron cartridge filter. The solvents were distilled under reduced pressure at below 50° C. until ˜1vol solvent remains. The reaction mixture was cooled to 25-30° C. and fresh acetonitrile (190 L) was charged into the reactor through a 0.2 micron cartridge filter. The mixture was warmed up to 65-70° C. and stirred for 45 min, then cooled down to 25-30° C. and stirred for 1 h. the resulting slurry was filtered, and the cake was washed with chilled (15° C.) acetonitrile (56.8 L). The solids were dried under reduced pressure at 40-50° C. for 8 h to afford Intermediate A1 (36.4 Kg, 64%). ¹H NMR (400 MHz, CD₃OD) b ppm 7.43 (s, 1H), 5.32-5.42 (m, 1H), 3.15-3.25 (m, 4H), 2.89 (s, 2H), 2.64 (s, 2H), 1.69-1.90 (m, 4H), 1.37-1.45 (m, 6H); ESI [M+H]⁺=248.

Intermediate A2: 2-(4-(tert-Butoxycarbonyl)phenyl)-6-methoxyisonicotinic acid.

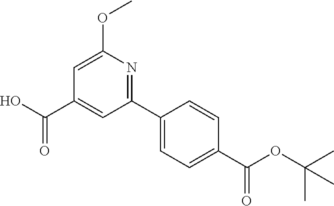 [SMILES: COc1cc(C(=O)O)cc(-c2ccc(C(=O)OC(C)(C)C)cc2)n1]

A clean and dried reactor was charged with 2,6-dichloroisonicotinic acid (30 Kg) and methanol (120 L) at 20-25° C. The slurry was stirred for 5 min then heated up to 65° C. (reflux). A solution of sodium methoxide in methanol (30%, 87.2 Kg) was then slowly charged over at least 4 h via addition funnel. The funnel was rinsed with methanol (15 L), and stirring was pursued at 65° C. for at least 15 h. the mixture was then cooled down to 45° C. and distilled under reduced pressure until a residual volume of −90 L. A solution of potassium bicarbonate (28.2 Kg) and potassium carbonate (21.6 Kg) in water (180 L) was then charged into the reactor at 40-45° C. The reactor containing the aqueous solution was rinsed with water (21 L) and the wash was charged into the reaction mixture. The mixture was distilled under reduced pressure at below 80° C. until a residual volume of −240 L, then cooled down to 20-25° C.

Another clean and dry reactor was charged with tert-butyl 4-(4,4,5,5-tetramethyl-1,3,2-dioxoborolan-2-yl) benzoate (52.3 Kg) and dioxane (340 Kg), and stirred at 2-25° C. until complete dissolution. The content of the former reactor was then heated at 40° C. to ensure complete solubility and transferred into this new reactor. The reaction mixture was cooled down to 20-25° C., and a deoxygenation step was performed via vacuum/nitrogen cycles. The mixture was further cooled down to 0-10° C. and palladium acetate (0.65 Kg) was charged into the reactor followed by triphenylphosphine (2.46 Kg) under nitrogen flow. The mixture was warmed up to 20-25° C. and another deoxygenation step was performed via vacuum/nitrogen cycles. The mixture was then heated to 80° C. and maintained at this temperature for at least 18 h. the mixture was cooled down to 20-25° C., then methyl tert-butyl ether (133.2 Kg) and water (30 L) were successively charged into the reactor. The layers were separated, and the aqueous was diluted with water (110 L), then extracted with methyl tert-butyl ether (110 L). Combined organic extracts were washed with a solution of citric acid (52 Kg) in water (84 L), and the layers were separated. The aqueous layer was further extracted with methyl tert-butyl ether (88.8 Kg) and organic layers were combined, then washed three times with a third of a solution of sodium chloride (43 Kg) in water (80 L). After final layer separation, the organic layer was filtered through pall filter containing a charcoal cartridge, and the cake was washed with methyl tert-butyl ether (11.2 Kg). The filtrate was distilled under reduced pressure at below 50° C. down to ˜90 L, and was then successively co-distilled with heptane (120 L), at below 50° C. and down to ˜120 L. the mixture was then cooled down to 20-25° C. over 1 h, then stirred at this temperature for another 1 h. The slurry was filtered and the cake was washed three times with heptane (3×18 L), then three times with acetonitrile (3×18 L). The resulting wet solid was dried under vacuum and nitrogen flow at below 45° C. for at least 15 h to afford Intermediate A2 (44.6 Kg, 87% yield). ¹H NMR (400 MHz, CDCl₃) δ ppm 8.13 (s, 2H), 8.09 (s, 2H), 7.97 (d, J=1.17 Hz, 1H), 7.34 (d, J=0.98 Hz, 1H), 4.08 (s, 3H), 1.61 (s, 9H); ESI [M+H]*=330.

Intermediate A3: tert-Butyl 4-(4-(1-isopropyl-7-oxo-1,4,6,7-tetrahydrospiro[indazole-5,4′-piperidine]-1′-carbonyl)-6-methoxypyridin-2-yl)benzoate.

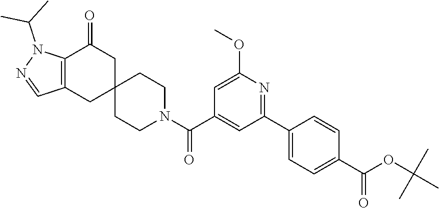 [SMILES: COc1cc(C(=O)N2CCC3(CC2)CC(=O)c2c(cnn2C(C)C)C3)cc(-c2ccc(C(=O)OC(C)(C)C)cc2)n1]

A round bottomed flask was charged with 2-(4-(tert-butoxycarbonyl)phenyl)-6-methoxyisonicotinic acid (Intermediate A2, 15.2 g, 46.2 mmol) and ethyl acetate (140 mL). 1,1′-Carbonyldiimidazole (8.98 g, 55.4 mmol) was added in one portion and stirred for 1 h at rt. 1-isopropyl-4,6-dihydrospiro[indazole-5,4′-piperldin]-7(1H)-one hydrochloride (Intermediate A1, 14.8 g, 52.2 mmol) was added followed by N,N-diisopropylethylamine (9.1 mL, 52.2 mL) and the reaction stirred for 18 h at rt. Aqueous 2 M HCl (40 mL) was added, followed by 1 M potassium hydrogensulfate (40 mL) and 50 mL of heptane. The obtained mixture was stirred for 1 h at rt. The mixture was transferred to separation funnel. The organic phase was separated, washed successively with water (20 mL), saturated sodium bicarbonate (30 mL), water (20 mL), brine (20 mL), dried over 20 g of magnesium sulfate and 10 g of silica gel, filtered, and concentrated in vacuo. Solid began to form towards the end of concentration. The residue was stirred in 40 mL of ethyl acetate at 80° C. and heptane (120 mL) was added slowly dropwise. The mixture was stirred at 80° C. for 1 h, then slowly cooled to room temperature with stirring over 1 h and stirred for 18 h at rt. The solid was collected via filtration, washed with water and ethyl acetate-heptane (1:3), and dried under vacuum at 50° C. for 18 h to obtain Intermediate A3 (19.64 g, 76% yield).

### Alternative Preparation of Intermediate A3

A clean and dry reactor was charged with acetonitrile (219 Kg) and 2-(4-(tert-butoxycarbonyl)phenyl)-6-methoxyisonicotinic acid (Intermediate A2, 34.8 Kg) at 20-25° C. The mixture was stirred for 5 min, then 1,1-carbodiimidazole (18.9 Kg) was charged in three successive portions. The slurry was further stirred at 20-25° C. for at least 1 h, then 1-isopropyl-4,6-dihydrospiro[indazole-5,4′-piperldin]-7(1H)-one hydrochloride salt (Intermediate A1, 33.0 Kg) was charged into the reactor, followed by N,N-diisopropylethylamine (20.5 Kg) via pump. The reagent pump as well as the walls of the reactor were washed with acetonitrile (13.7 Kg), and stirring was pursued at 20-25° C. for at least 2 h. Upon completion, the mixture was seeded with tert-butyl 4-(4-(1-isopropyl-7-oxo-1,4,6,7-tetrahydrospiro[indazole-5,4′-piperidine]-1′-carbonyl)-6-methoxypyridin-2-yl)benzoate (Intermediate A3, 209 g) and stirred for at least 30 min. After confirmation of crystallization start, a solution of citric acid monohydrate (58.5 Kg) in water (257 L) was charged over 1 h. The resulting slurry was further stirred at 20-25° C. for at least 2 h, then filtered and the cake was washed with a mixture of acetonitrile (68.4 Kg) and water (87 L). This wash was used to rinse the reactor as well. The solids were dried under reduced pressure at below 55° C., affording Intermediate A3 (43.44 Kg, 73% yield).

### Compound A (as the free acid): 4-(4-(1-isopropyl-7-oxo-1,4,6,7-tetrahydrospiro[indazole-5,4′-piperidine]-1′-carbonyl)-6-methoxypyridin-2-yl)benzoic acid

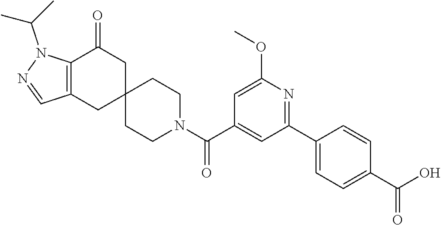 [SMILES: COc1cc(C(=O)N2CCC3(CC2)CC(=O)c2c(cnn2C(C)C)C3)cc(-c2ccc(C(=O)O)cc2)n1]

A round bottomed flask was charged with tert-butyl 4-(4-(1-isopropyl-7-oxo-1,4,6,7-tetrahydrospiro[indazole-5,4′-piperidine]-1′-carbonyl)-6-methoxypyridin-2-yl)benzoate (3.7 g, 6.6 mmol) and toluene (25 mL). 85% Phosphoric acid (3.0 mL) was added dropwise with stirring and the reaction was heated to 60° C. for 4 hours. A colorless thick gum formed. The reaction was cooled to rt and water was added. White solids were observed. The toluene organic layer was discarded, reserving the aqueous layer and solids. Ethyl acetate was added (60 mL) and 4N NaOH solution was added to adjust pH to ˜7. The layers were separated and the aqueous was extracted with ethyl acetate (50 mL). The combined ethyl acetate organic layers were dried over sodium sulfate, filtered, and concentrated in vacuo to provide white solids. These were dissolved in ethyl acetate (80 mL) at 50° C. and heptane (90 mL) was added slowly. The heat was removed and the mixture was cooled to rt and stirred for 16 h. The resultant solids were collected via filtration, rinsed with the mother liquor, and dried to provide the title compound (Compound A free form, 2.15 g, 65% yield) as a white solid.

### Alternative Preparation of Compound a (as the Free Acid)

A clean a dry reactor was charged with acetonitrile (130.4 Kg) and tert-butyl 4-(4-(1-isopropyl-7-oxo-1,4,6,7-tetrahydrospiro[indazole-5,4′-piperidine]-1′-carbonyl)-6-methoxypyridin-2-yl)benzoate (Intermediate A3, 20.72 Kg) at 20-25° C. The mixture was stirred for 5 min, then p-toluenesulfonic acid (8.5 Kg) was charged under a gentle nitrogen sweep. The reaction mixture was warmed up to 70° C. and maintained at this temperature for at least 6.5 h. Upon completion, the mixture was cooled down to 40° C., seeded with Compound A (104 g) and water (83 L) was slowly charged over at least 1 h. the mixture was further stirred at 40° C. for a minimum of 4 h, then cooled down to 20-25° C. over 2 h. Further stirring for at least 2 h was followed by filtration, and the cake was rinsed with a solution of acetonitrile (33 Kg) and water (41 L). This wash was used to rinse the reactor as well. The resulting solids were dried under reduced pressure at below 55° C. to afford Compound A (16.5 Kg, 89% yield).

### Preparation of Form 1 of Compound a—Anhydrous Mono-Tris of Compound A

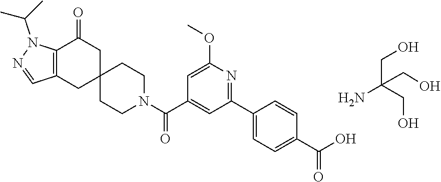 [SMILES: COc1cc(C(=O)N2CCC3(CC2)CC(=O)c2c(cnn2C(C)C)C3)cc(-c2ccc(C(=O)O)cc2)n1 ; NC(CO)(CO)CO]

A vial was charged with 4-(4-(1-isopropyl-7-oxo-1,4,6,7-tetrahydrospiro[indazole-5,4′-piperidine]-1′-carbonyl)-6-methoxypyridin-2-yl)benzoic acid (151 mg, 0.300 mmol) and 3 mL of ethanol. The mixture was heated to 80° C. for 5 minutes to dissolve the solid and then cooled to rt. Tris(hydroxymethyl)aminomethane (39 mg, 0.32 mmol) was added, and the mixture was stirred overnight at rt. Heptane (2.25 mL) was added dropwise to produce a slurry that was heated to 50° C. to produce a clear solution. The mixture was cooled to rt overnight with stirring. White solids were observed, and the mixture was stirred for an additional 3 days. The material was filtered and dried in a vacuum oven at 50° C. overnight to produce Form 1 (151 mg, 0.242 mmol, 81% yield).

### Alternative Preparation of Form 1 of Compound A: Anhydrous Mono-Tris of Compound A

To a clean and dry reactor was charged ethanol (83 L), followed by the addition of Compound A (9.43 Kg) and tris (2.55 kg) while the mixture was maintained at a temperature of 20-25° C. The tank walls were rinsed with ethanol (2 L), and the resulting mixture was heated at 65-70° C., maintained at this temperature for at least 30 min until all solids dissolved, then cooled down to 45-50° C. A warm filtration through a 10 μm in-line polypropylene filter was performed, and the reactor as well as the filter were washed with ethanol (9 L). n-Heptane (24 L) was charged into the warm solution through the same in-line filter, and the mixture was seeded with 4-(4-(1-isopropyl-7-oxo-1,4,6,7-tetrahydrospiro[indazole-5,4′-piperidine]-1′-carbonyl)-6-methoxypyridin-2-yl)benzoic acid anhydrous tris salt (100 g) in ethanol (0.5 L) at 45-50° C. The temperature was held for at least 2 h before cooling down to 20-25° C. over at least 2 h. Stirring was pursued for at least 5 days. The slurry was then filtered, and the cake was washed with a mixture of ethanol (13 L) and n-heptane (6 L). The solids were dried under reduced pressure at below 45° C. for at least 12 h, affording example 26 (11.7 Kg, 77%).

### Preparation of Form 2 of Compound A—Trihydrate of the mono-tris salt of Compound A

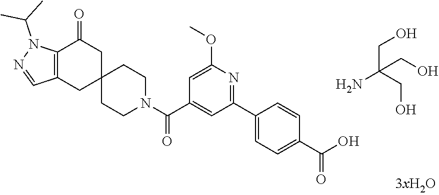 [SMILES: COc1cc(C(=O)N2CCC3(CC2)CC(=O)c2c(cnn2C(C)C)C3)cc(-c2ccc(C(=O)O)cc2)n1 ; NC(CO)(CO)CO]

Form 2 of Compound A was obtained from conversion from Form 1 of Compound A. Into a 50 mL EasyMax reactor was added Form 1 (1.7214 g, 2.760 mmol), Isopropanol (16.50 mL, 215.8 mmol), and Water (688 μL, 38.190 mmol). The mixture was stirred (300 rpm) for about 72 hr with a reactor jacket temperature of 25° C. The reaction mixture was then warmed to 40° C. over 15 min and held at 40° C. for about 24 hours, cooling once to 20° C. to remove a sample for testing. A mixture of forms was seen by PXRD; therefore, additional water Water (688 μL, 38.190 mmol) was added. The stir rate was increased to 400 rpm and the slurry was allowed to stir for 6 hours and was then cooled to 15° C. The solids were isolated on a 60 mL/40 M filter and washed with 96/4 isopropanol/water. The resulting material was consistent with Form 2 of Compound A by PXRD.

### Alternative Preparation of Form 2 of Compound a—Trihydrate of the Mono-Tris Salt of Compound A

A clean and dry reactor was charged with isopropanol (60.4 Kg), and Compound A (16.68 Kg) and tris (4.42 kg) were added while the mixture was maintained at a temperature of 20-25° C. The mixture was stirred for 5 min, then water (6.7 Kg) was charged and the slurry was warmed up to 55° C. The now clear solution was filtered into a pre-warmed clean and dry reactor (50-55° C.) through an in-line 10 μm polypropylene filter. The solution was then seeded with the mono-tris salt of Compound A as a trihydrate (167 g). After verification that the seed persisted, the mixture was cooled down to 15° C. over at least 2 h, then maintained at 15° C. for a minimum of 16 h. The slurry was filtered and the cake washed with chilled isopropanol (13.1 Kg). The solids were then dried under reduced pressure at below 25° C. to afford only Form 2 of Compound A (22.1 Kg, 98% yield).

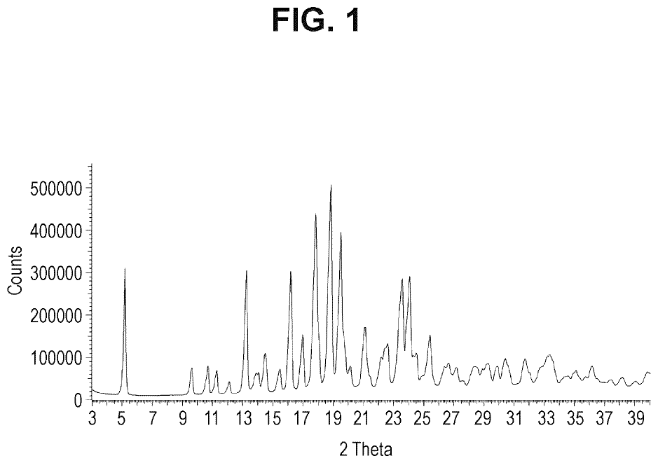

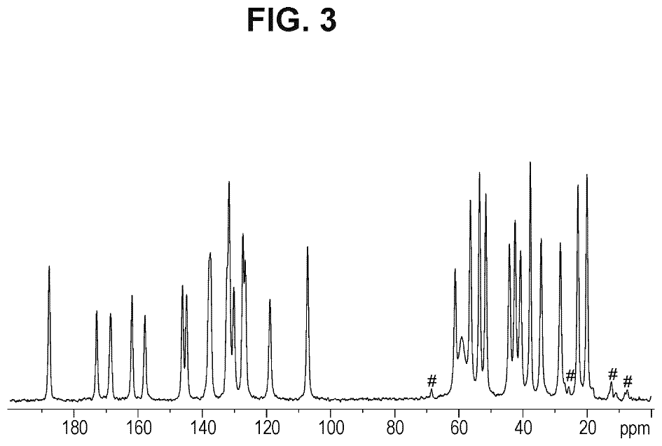

Form 1 of Compound A is anhydrous and is thermodynamically stable below a water activity of about 0.2 (20% RH) at ambient temperature. Form 1 of Compound A has a PXRD pattern substantially the same as that shown in FIG. 3 of Compound A. Characteristic PXRD peaks of Form 1 of Compound A, expressed as 2⊖+0.20° 2⊖ are at 9.6, 10.7, and 11.3. Peak locations and intensities for the PXRD pattern in FIG. 1 are provided in Table 4.

TABLE 4
PXRD Peaks and Relative Intensities of Form 1 of Compound A

| Degrees | | Degrees | | Degrees | |
|---|---|---|---|---|---|
| 2Θ ± | Relative | 2Θ ± | Relative | 2Θ ± | Relative |
| 0.2° 2Θ | Intensity (%) | 0.2° 2Θ | Intensity (%) | 0.2° 2Θ | Intensity (%) |
| 5.2 | 62 | 17.8 | 86 | 25.4 | 25 |
| 9.6 | 13 | 18.9 | 100 | 26.4 | 9 |
| 10.7 | 14 | 19.5 | 77 | 26.6 | 11 |
| 11.3 | 11 | 20.1 | 11 | 27.2 | 9 |
| 12.1 | 6 | 21.1 | 29 | 28.3 | 9 |
| 13.3 | 60 | 22.2 | 15 | 29.3 | 10 |
| 13.9 | 9 | 22.4 | 19 | 29.9 | 9 |
| 14.0 | 10 | 22.6 | 21 | 30.4 | 13 |
| 15.5 | 11 | 23.6 | 53 | 31.7 | 13 |
| 16.2 | 58 | 24.1 | 54 | 33.4 | 15 |
| 17.0 | 27 | 24.5 | 16 | | |

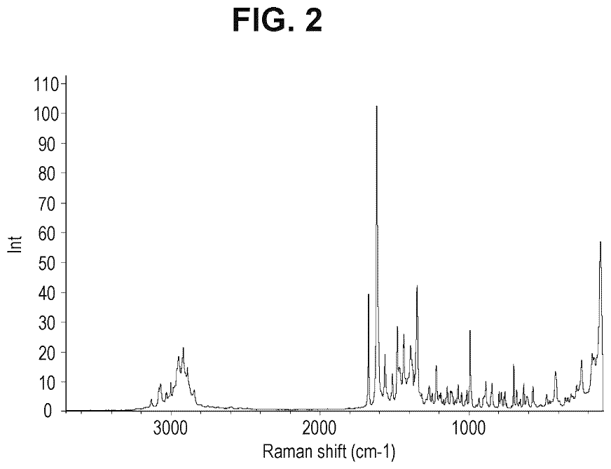

Form 1 of Compound A has a Raman spectrum substantially the same as that shown in FIG. 2. Form 1 of Compound A has characteristic Raman peak shifts, expressed as cm⁻¹, at 568, 698, 989, 1218, 1511, 1561, and 1615, ±2 cm⁻¹. Peak positions (±2 cm⁻¹) and normalized intensity (W=weak, M=medium, S=strong) of Form 1 of Compound A in FIG. 2 are listed in Table 5.

TABLE 5
Raman Peaks and Normalized Intensity of Form 1 of Compound A

| Raman | | Raman | | Raman | |
|---|---|---|---|---|---|
| Peak | | Peak | | Peak | |
| Position | Normalized | Position | Normalized | Position | Normalized |
| (cm⁻¹) | Intensity | (cm⁻¹) | Intensity | (cm⁻¹) | Intensity |
| 115 | M | 794 | W | 1363 | W |
| 156 | W | 842 | W | 1388 | W |
| 170 | W | 885 | W | 1435 | W |
| 241 | W | 929 | W | 1466 | W |
| 274 | W | 989 | W | 1478 | W |
| 311 | W | 1011 | W | 1511 | W |
| 334 | W | 1047 | W | 1561 | W |
| 350 | W | 1071 | W | 1615 | S |
| 417 | W | 1090 | W | 1671 | M |
| 456 | W | 1119 | W | 2840 | W |
| 476 | W | 1143 | W | 2885 | W |
| 568 | W | 1169 | W | 2914 | W |
| 608 | W | 1187 | W | 2945 | W |
| 628 | W | 1196 | W | 2998 | W |
| 653 | W | 1218 | W | 3027 | W |
| 678 | W | 1244 | W | 3066 | W |
| 698 | W | 1265 | W | 3129 | W |
| 755 | W | 1315 | W | | |
| 779 | W | 1345 | M | | |

Form 1 of Compound A has a ¹³C ssNMR spectrum substantially the same as that shown in FIG. 3. Form 1 of Compound A has characteristic ¹³C ssNMR chemical shifts, expressed as ppm, at 22.9, 146.2, 157.9, 161.9, and 172.9, +0.2 ppm. ¹³C chemical shifts (±0.2 ppm) of Form 1 of Compound A as shown in FIG. 3 are listed in Table 6.

TABLE 6
¹³C chemical shifts and Intensity of Form 1 of Compound A

| ¹³C chemical | Inten- | ¹³C chemical | Inten- | ¹³C chemical | Inten- |
|---|---|---|---|---|---|
| shifts (ppm) | sity | shifts (ppm) | sity | shifts (ppm) | sity |
| 20.1 | 95 | 56.4 | 84 | 137.5 | 62 |
| 22.9 | 90 | 59.1 | 27 | 137.9 | 59 |
| 28.4 | 66 | 61.2 | 55 | 144.9 | 44 |
| 34.3 | 68 | 107.1 | 65 | 146.2 | 48 |
| 37.7 | 100 | 118.9 | 42 | 157.9 | 36 |
| 40.8 | 63 | 126.6 | 59 | 161.9 | 44 |
| 42.5 | 76 | 127.3 | 70 | 168.6 | 36 |
| 44.3 | 66 | 130.2 | 47 | 172.9 | 38 |
| 51.6 | 87 | 131.7 | 92 | 187.7 | 56 |
| 53.6 | 96 | 132.3 | 56 | | |

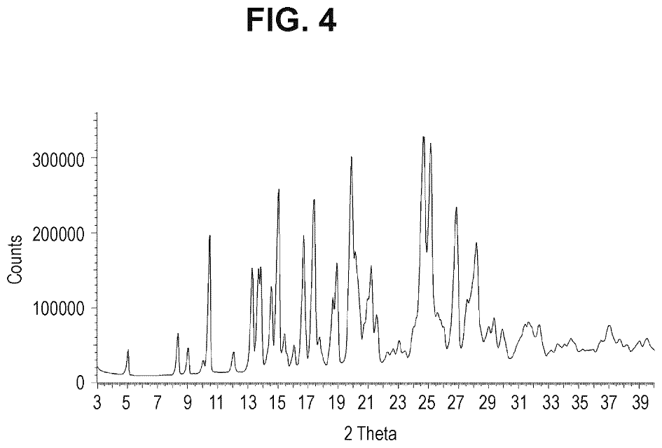

Form 2 of Compound A is a trihydrate and is thermodynamically stable above a water activity of about 0.2 at ambient temperature and 20% RH. Form 2 of Compound A has a PXRD pattern substantially the same as that shown in FIG. 4. Characteristic PXRD peaks of Form 2 of Compound A, expressed as 2⊖+0.2° 2⊖ are at 8.4, 9.0, 10.5, 15.0, and 24.7. Peak locations and intensities for the PXRD pattern in FIG. 4 are provided in Table 7.

TABLE 7
PXRD Peaks and Relative Intensities of Form 2 of Compound A

| Degrees | | Degrees | | Degrees | |
|---|---|---|---|---|---|
| 2Θ ± | Relative | 2Θ ± | Relative | 2Θ ± | Relative |
| 0.2° 2Θ | Intensity (%) | 0.2° 2Θ | Intensity (%) | 0.2° 2Θ | Intensity (%) |
| 5.0 | 11 | 16.1 | 10 | 26.9 | 71 |
| 8.4 | 18 | 16.7 | 59 | 28.2 | 52 |
| 9.0 | 12 | 17.4 | 74 | 29.0 | 15 |
| 10.0 | 6 | 17.8 | 13 | 29.4 | 18 |
| 10.5 | 62 | 18.6 | 30 | 29.9 | 13 |
| 12.1 | 9 | 18.9 | 45 | 31.4 | 15 |
| 13.3 | 46 | 19.9 | 93 | 31.7 | 16 |
| 13.7 | 45 | 20.1 | 50 | 32.4 | 14 |
| 13.9 | 46 | 21.2 | 46 | 33.6 | 5 |
| 14.6 | 37 | 21.5 | 21 | 34.5 | 7 |
| 15.0 | 80 | 24.7 | 100 | 37.0 | 12 |
| 15.4 | 15 | 25.2 | 97 | | |

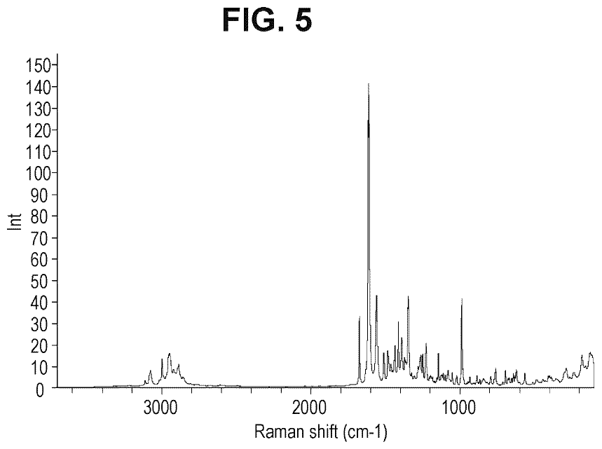

Form 2 of Compound A has a Raman spectrum substantially the same as that shown in FIG. 5. Form 2 of Compound A has characteristic Raman peak shift, expressed as cm⁻¹, at 562, 692, 984, 1225, 1507, 1557, and 1610±2 cm⁻¹. Peak positions (±2 cm⁻¹) and normalized intensity (W=weak, M=medium, S=strong) of Form 2 of Compound A in FIG. 5 are listed in Table 8.

TABLE 8
Raman Peaks and Normalized Intensity of Form 2 of Compound A

| Raman | | Raman | | Raman | |
|---|---|---|---|---|---|
| Peak | | Peak | | Peak | |
| Position | Normalized | Position | Normalized | Position | Normalized |
| (cm⁻¹) | Intensity | (cm⁻¹) | Intensity | (cm⁻¹) | Intensity |
| 123 | W | 864 | W | 1344 | W |
| 179 | W | 884 | W | 1369 | W |
| 232 | W | 931 | W | 1387 | W |
| 284 | W | 984 | W | 1410 | W |
| 405 | W | 1019 | W | 1433 | W |
| 441 | W | 1048 | W | 1460 | W |
| 481 | W | 1077 | W | 1480 | W |
| 562 | W | 1097 | W | 1507 | W |
| 620 | W | 1109 | W | 1557 | M |
| 628 | W | 1118 | W | 1610 | S |
| 639 | W | 1140 | W | 1670 | W |
| 650 | W | 1194 | W | 2884 | W |
| 667 | W | 1225 | W | 2916 | W |
| 692 | W | 1246 | W | 2946 | W |
| 710 | W | 1261 | W | 2995 | W |
| 758 | W | 1277 | W | 3073 | W |
| 790 | W | 1305 | W | 3108 | W |
| 839 | W | 1321 | W | | |

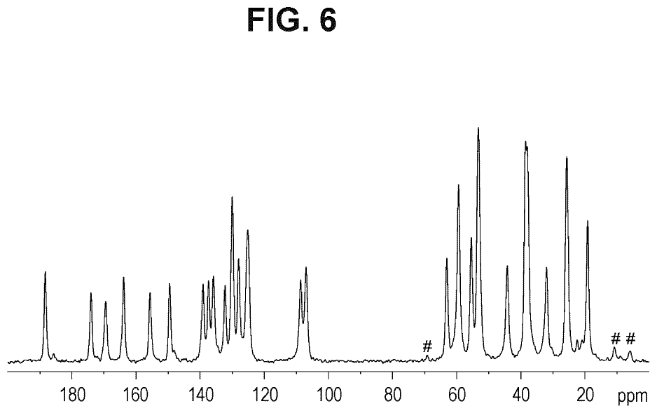

Form 2 of Compound A has a ¹³C ssNMR spectrum substantially the same as that shown in FIG. 6. Form 2 of Compound A has characteristic ¹³C ssNMR chemical shifts, expressed as ppm, at 19.2, 149.5, 155.6, 163.8, and 188.3, ±0.2 ppm. ¹³C chemical shifts (±0.2 ppm) of Form 2 of Compound A as shown in FIG. 6 are listed in Table 9.

TABLE 9
¹³C chemical shifts and Intensity of Form 2 of Compound A

| | | | | ¹³C | |
|---|---|---|---|---|---|
| ¹³C chemical | Inten- | ¹³C chemical | Inten- | chemical | Inten- |
| shifts (ppm) | sity | shifts (ppm) | sity | shifts (ppm) | sity |
| 19.2 | 60 | 63.1 | 44 | 139.1 | 33 |
| 25.7 | 87 | 107.0 | 40 | 149.5 | 33 |
| 32.0 | 40 | 108.7 | 35 | 155.6 | 30 |
| 38.0 | 92 | 125.1 | 56 | 163.8 | 36 |
| 38.5 | 94 | 128.0 | 44 | 169.5 | 26 |
| 44.2 | 41 | 130.0 | 70 | 174.0 | 29 |
| 53.2 | 100 | 132.3 | 33 | 188.3 | 39 |
| 55.5 | 53 | 135.9 | 37 | | |
| 59.4 | 76 | 137.4 | 35 | | |

Based on the disclosure provided herein, one of ordinary skill in the art would appreciate that each Form 1 and Form 2 of Compound A can be uniquely identified by several different spectral peaks or patterns in varying combinations. Described below are exemplary combinations of characteristic peak values that can be used to separately identify Form 1 and Form 2 of Compound A but in no way should these exemplary combinations be viewed as limiting other peak value combinations disclosed herein.

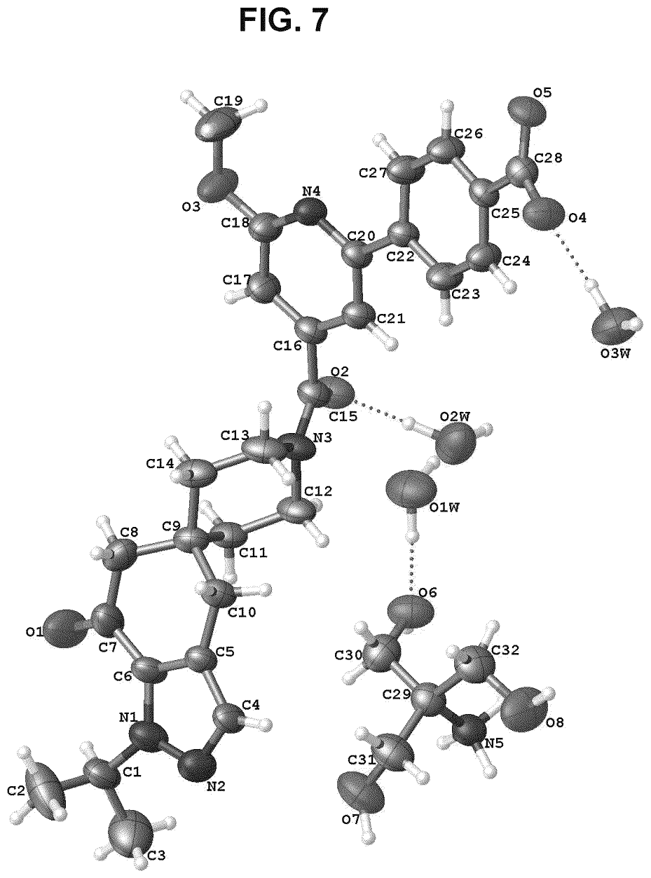

To confirm the presence of three water molecules in Form 2 of Compound A, data was collected using a Bruker D8 Venture diffractometer at room temperature. See FIG. 7. The structure was solved by intrinsic phasing using SHELX software suite in the Monoclinic class space group P21/c (Version 5.1, Bruker AXS, 1997). The structure was subsequently refined by the full-matrix least squares method. All non-hydrogen atoms were found and refined using anisotropic displacement parameters.

The hydrogen atoms located on nitrogen and oxygen were found from the Fourier difference map and refined with distances restrained. The remaining hydrogen atoms were placed in calculated positions and were allowed to ride on their carrier atoms.

The final R-index was 7.2%. A final difference Fourier revealed no missing or misplaced electron density.

Table 10 provides data collected with regard to Form 2 of Compound A:

TABLE 10

| | | |
|---|---|---|
| Empirical formula | C₂₈H₃₀N₄O₅. C₄H₁₁NO₃. 3H₂O | |
| Formula weight | 677.74 | |
| Temperature | RT | |
| Wavelength | 1.54178 Å | |
| Crystal system | Monoclinic | |
| Space group | P2₁/c | |
| Unit cell dimensions | a = 17.6927(9) Å | α = 90°. |
| | b = 13.2753(7) Å | β = 92.451(3)°. |
| | c = 14.6480(8) Å | α = 90°. |
| Volume | 3437.3(3) Å³ | |
| Z | 4 | |
| Density (calculated) | 1.310 Mg/m³ | |
| Goodness-of-fit on F² | 1.053 | |
| Final R indices [I > | R1 = 0.0723, wR2 = 0.1835 | |
| 2sigma(I)] | | |
| R indices (all data) | R1 = 0.1244, wR2 = 0.2110 | |

A crystalline 2-amino-2-(hydroxymethyl)propane-1,3-diol salt of is 4-(4-(1-isopropyl-7-oxo-1,4,6,7-tetrahydrospiro[indazole-5,4′-piperidine]-1′-carbonyl)-6-methoxypyridin-2-yl)benzoic acid. This crystalline salt is generally referred to as the tris salt of Compound A.

The crystalline tris salt of Compound A, wherein the ratio of 4-(4-(1-isopropyl-7-oxo-1,4,6,7-tetrahydrospiro[indazole-5,4′-piperidine]-1′-carbonyl)-6-methoxypyridin-2-yl)benzoic acid and the salt is 1:1.

The crystalline tris salt of Compound A, wherein the crystalline salt is an anhydrous crystalline salt.

The anhydrous crystalline tris salt of Compound A, wherein said anhydrous crystalline salt has a PXRD pattern comprising peaks at diffraction angles of 9.6, 10.7, and 11.3 2⊖, 0.2° 2⊖.

The anhydrous crystalline tris salt of Compound A, wherein said anhydrous crystalline salt has a Raman spectrum comprising peak shifts at 1511, 1561, and 1615 cm⁻¹, ±2 cm⁻¹.

The anhydrous crystalline tris salt of Compound A, wherein said anhydrous crystalline salt has a ¹³C ssNMR spectrum comprising chemical shifts at 22.9, 146.2, and 161.9 ppm, ±0.2 ppm.

The anhydrous crystalline tris salt of Compound A, wherein said anhydrous crystalline salt has an analytical parameter selected from the group consisting of a Raman spectrum comprising peak shifts at 1511 and 1615 cm⁻¹, ±2 cm⁻¹, and a ¹³C ssNMR spectrum comprising at least one chemical shift at 22.9, 146.2, or 161.9 ppm, ±0.2 ppm.

The anhydrous crystalline tris salt of Compound A, where said anhydrous crystalline salt is substantially pure.

The crystalline tris salt of Compound A, wherein the crystalline salt is a trihydrate crystalline salt.

The trihydrate crystalline tris salt of Compound A, wherein said trihydrate crystalline salt has a PXRD pattern comprising peaks at diffraction angles of 8.4, 9.0, and 10.5 2⊖, +0.2° 2⊖.

The trihydrate crystalline tris salt of Compound A, wherein said trihydrate crystalline salt has a Raman spectrum comprising peak shifts at 1507, 1557, and 1610 cm⁻¹, ±2 cm⁻¹.

The trihydrate crystalline tris salt of Compound A, wherein said trihydrate crystalline salt has a ¹³C ssNMR spectrum comprising chemical shifts at 19.2, 149.5, and 163.8 ppm, 10.2 ppm.

The trihydrate crystalline tris salt of Compound A, wherein said trihydrate crystalline salt has an analytical parameter selected from the group consisting of
a PXRD pattern comprising peaks at diffraction angles of 8.4 and 9.0 2⊖, ±0.2° 2⊖,
a Raman spectrum comprising peak shifts at 1557 and 1610 cm⁻¹, ±2 cm⁻¹, and
a ¹³C ssNMR spectrum comprising at least one chemical shift at 19.2, 149.5, or 163.8 ppm, ±0.2 ppm.

The trihydrate crystalline tris salt of Compound A, wherein said trihydrate crystalline salt has an analytical parameter selected from the group consisting of a PXRD pattern comprising peaks at diffraction angles of 8.4 and 9.0 2⊖, ±0.2° 2⊖, and a Raman spectrum comprising at least one peak shift at 1507, 1557, or 1610 cm⁻¹, ±2 cm⁻¹.

The trihydrate crystalline tris salt of Compound A, wherein said trihydrate crystalline salt has an analytical parameter selected from the group consisting of a PXRD pattern comprising peaks at diffraction angles of 8.4 and 9.0 2⊖, +0.2° 2⊖, and a ¹³C ssNMR spectrum comprising at least one chemical shift at 19.2, 149.5, or 163.8 ppm, ±0.2 ppm.

### Example 27 (DGAT2i Compound/Compound D): (S)-2-(5-((3-ethoxypyridin-2-yl)oxy)pyridin-3-yl)-N-(tetrahydrofuran-3-yl)pyrimidine-5-carboxamide

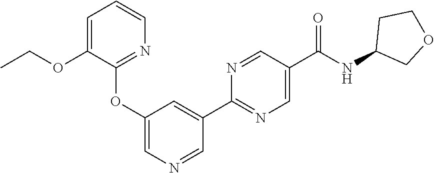 [SMILES: CCOc1cccnc1Oc1cncc(-c2ncc(C(=O)N[C@H]3CCOC3)cn2)c1]

The compound of Example 27 (Compound D) including crystal forms thereof, and its methods of preparation were disclosed in Example 1 of U.S. Pat. No. 10,071,992 which issued on Sep. 11, 2018, and claims the benefit of U.S. Provisional Patent Application No. 62/377,137, filed on Aug. 19, 2016, all of which are hereby incorporated herein by reference in their entireties for all purposes.

### Pharmacological Data

The following protocols may of course be varied by those skilled in the art.

### Generation of Human DGAT2 (hDGAT2) Construct

A construct for hDGAT2 was generated with an N-terminal FLAG tag (an octapeptide with the amino acid sequence of AspTyrLysAspAspAspAspLys). For the FLAG-tagged hDGAT2 construct, the cDNA for hDGAT2 was custom-synthesized at Genscript and cloned into the pFastBac1 vector (Invitrogen) by using BamHI/Xhol restriction enzymes to generate an N-terminally FLAG-tagged pFastBac1-FLAG-hDGAT2 construct (amino acids 1-388). The construct was confirmed by sequencing in both directions.

### DGAT2 Expression and Preparation of the DGAT2 Membrane Fraction

Recombinant baculovirus for the FLAG-tagged hDGAT2 was generated in SF9 insect cells using Bac-to-Bac baculovirus expression system (Invitrogen) according to the manufacturer's protocol. For the expression of hDGAT2, SF9 cells (20 L) grown in Sf90011 media were infected with hDGAT2 baculovirus at a multiplicity of infection of 1 in a Wave Bioreactor System 20/50P wave bag (GE Healthcare). After 40 hours of infection, the cells were then harvested by centrifugation at 5,000×g. The cell pellets were washed by resuspending in phosphate buffered saline (PBS) and collected by centrifugation at 5,000×g. The cell paste was flash frozen in liquid N₂ and stored at −80° C. until needed. All operations below were at 4° C. unless otherwise noted. The cells were resuspended in lysis buffer (50 mM Tris-HCl, pH 8.0, 250 mM sucrose) including 1 mM ethylenediaminetetraacetic acid (EDTA) and the complete protease inhibitor cocktail (Roche Diagnostics) at a ratio of 3 mL buffer per 1 g cell paste. The cells were lysed by dounce homogenizer. The cell debris was removed by centrifugation at 1,000×g for 20 min, and the supernatant was centrifuged at 100,000×g for 1 hour. The resulting pellet was rinsed three times by filling ultracentrifuge tubes to the top with ice cold PBS before decanting. The washed pellet was resuspended with gentle stirring for 1 hour in lysis buffer containing 8 mM 3-[(3-cholamidopropyl)dimethylammonio]-1-propanesulfonate (CHAPS) at a ratio of 1 mL buffer per 1 g of original cell paste and centrifuged again at 100,000×g for 1 hour. The resulting supernatant was aliquotted, flash frozen in liquid N₂, and stored at −80° C. until use.

### In Vitro DGAT2 Assay and Determination of IC₅₀ Values for DGAT2 Inhibitors

For determination of IC₅₀ values, the reactions were carried out in 384-well white polypropylene plates (Nunc) in a total volume of 20 μL. To 1 μL of compounds dissolved in 100% DMSO and spotted at the bottom of each well, 5 μL of 0.04% bovine serum albumin (BSA) (fatty acid free, Sigma Aldrich) was added and the mixture was incubated at room temperature for 15 minutes. hDGAT2 membrane fractions were diluted in 100 mM Hepes-NaOH, pH 7.4, 20 mM MgCl₂ containing 200 nM methyl arachidonyl fluorophosphonate (Cayman Chemical, dried from ethyl acetate stock solution under argon gas and dissolved in DMSO as 5 mM stock). 10 μL of this enzyme working solution was added to the plates and incubation continued for 2 hours at room temperature. DGAT2 reactions were initiated by the addition of 4 μL of substrates containing 30 μM [1-¹⁴C]decanoyl-CoA (custom-synthesized by Perkin Elmer, 50 mCi/mmol) and 125 μM 1,2-didecanoyl-sn-glycerol (Avanti Polar Lipids) dissolved in 12.5% acetone. The reaction mixtures were incubated at room temperature for 40 min and the reactions were stopped by addition of 5 μL of 1% H₃PO₄. After the addition of 45 μL MicroScint-E (Perkin-Elmer), plates were sealed with Top Seal-A covers (Perkin-Elmer) and phase partitioning of substrates and products was achieved using a HT-91100 microplate orbital shaker (Big Bear Automation, Santa Clara, CA). Plates were centrifuged at 2,000×g for 1 minute in an Allegra 6R Centrifuge (Beckman Coulter) and then were sealed again with fresh covers before reading in a 1450 Microbeta Wallac Trilux Scintillation Counter (Perkin Elmer). DGAT2 activity was measured by quantifying the generated product [¹⁴C]tridecanoylglycerol in the upper organic phase.

Background activity obtained using 50 μM of ((R)-1-(2-((S)-1-(4-chloro-1H-pyrazol-1-yl)ethyl)-3H-imidazo[4,5-b]pyridin-5-yl)piperidin-3-yl)(pyrrolidin-1-yl)methanone (WO 2013150416, Example 196-A) for complete inhibition of DGAT2 was subtracted from all reactions. Inhibitors were tested at eleven different concentrations to generate IC₅₀ values for each compound. The eleven inhibitor concentrations employed typically included 50, 15.8, 5, 1.58, 0.50, 0.16, 0.05, 0.016, 0.005, 0.0016, and 0.0005 μM. The data were plotted as percentage of inhibition versus inhibitor concentration and fit to the equation, y=100/[1+(x/IC₅₀)^{z}], where IC₅₀ is the inhibitor concentration at 50% inhibition and z is the Hill slope (the slope of the curve at its inflection point).

Table 11 below provides the IC₅₀ values of the Examples for inhibition of DGAT2 in accordance with the above-described assay. Results are reported as geometric mean IC₅₀ values, with the number of replicates (n) shown.

TABLE 11
IC₅₀ values of Examples for inhibition of DGAT2

| | DGAT2 | | | DGAT2 | |
|---|---|---|---|---|---|
| Example | IC₅₀ [nM] | n | Example | IC₅₀ [nM] | n |
| 1 | 5.8 | 8 | 2 | 8.6 | 13 |
| 3 | 40 | 15 | 4 | 10 | 22 |
| 5 | 110 | 11 | 6 | 80 | 7 |
| 7 | 42 | 5 | 8 | 650 | 3 |
| 9 | 19 | 6 | 10 | 39 | 3 |
| 11 | 4.4 | 3 | 12 | 590 | 9 |
| 13 | 57 | 3 | 14 | 3100 | 3 |
| 15 | 3300 | 3 | 16 | 480 | 3 |
| 17 | 230 | 3 | 18 | 140 | 3 |
| 19 | 240 | 3 | 20 | 2100 | 3 |
| 21 | 5000 | 3 | 22 | 21000 | 1 |
| 23 | 14000 | 1 | 24 | 16000 | 1 |
| 25 | 200 | 5 | | | |

### Determination of IC₅₀ Values for DGAT2 Inhibitors in Human Hepatocytes

For evaluation of the effects of DGAT2 inhibitors in a cell-based setting, cryopreserved human hepatocytes (Lot DOO, Celsis, Baltimore, MD) were thawed and plated onto type I collagen-coated plates according to the manufacturer's instructions. After 18 hours overnight recovery period, the cells were overlayed with media containing 250 μg/mL Geltrex Basement Membrane Matrix (Thermo Fisher). The following day, media was aspirated and replaced with serum-free Williams Media E (Thermo Fisher) containing 400 μM sodium dodecanoate (Sigma-Aldrich, St. Louis, MO) and 2 mM GlutaMAX (Thermo Fisher). Forty-five minutes later, a selective DGAT1 inhibitor (Example 2, WO2009016462, prepared as a 100× stock in 25% DMSO, 75% PBS) was added to all wells at a final concentration (3 μM) that completely suppressed endogenous DGAT1 activity. DGAT2 inhibitors were then added to the desired final concentration. After a 15 minute preincubation, 0.2 μCi [¹⁴C(U)]-glycerol (Perkin Elmer) was added to each well followed by a 3 hour incubation. At this point the media was removed, and the cells lysed via orbital shaking in isopropyl alcohol: tetrahydrofuran (9:1) for 15 minutes prior to centrifugation at 3000 rpm for 10 minutes. Radiolabeled lipids were resolved using a solvent system by thin layer chromatography with the solvent consisting of hexanes: diethyl ether: glacial acetic acid (75:23:2, v/v/v). After separation, radiolabeled lipids were visualized using the Typhoon 9500 phosphorimaging system (GE). The half maximal inhibitory concentrations (IC₅₀ values) were determined by nonlinear regression analysis of the % inhibition dose response curve using GraphPad Prism (GraphPad Software, Inc., La Jolla, CA).

Table 12 below provides the IC₅₀ values of selected Examples for inhibition of DGAT2 in human hepatocytes in accordance with the above-described assay. Results are reported as geometric mean IC₅₀ values, with the number of replicates (n) shown.

TABLE 12
IC₅₀ values of Examples for inhibition
of DGAT2 in human hepatocytes

| | DGAT2 | | | DGAT2 | |
|---|---|---|---|---|---|
| Example | IC₅₀ [nM] | n | Example | IC₅₀ [nM] | n |
| 2 | 4.1 | 4 | 3 | 23 | 3 |
| 4 | 11 | 13 | 5 | 26 | 6 |
| 6 | 2.4 | 2 | 7 | 100 | 3 |
| 13 | 3.8 | 2 | 18 | 20 | 2 |
| 25 | >130 | 3 | | | |

### In Vivo Effects of DGAT2 Inhibitors on Plasma and Hepatic Triglyceride Levels

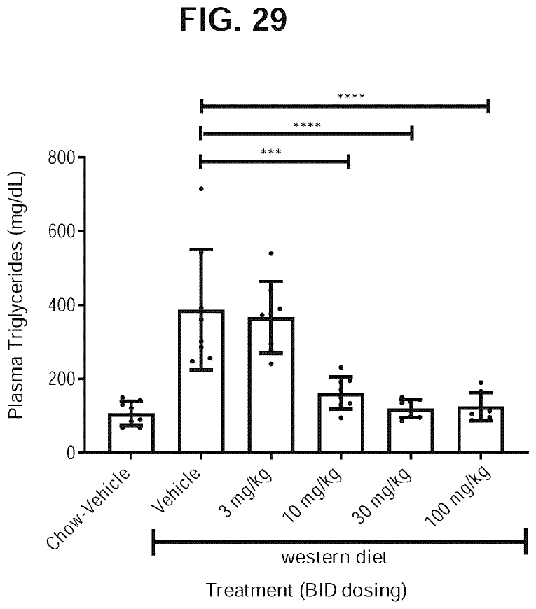

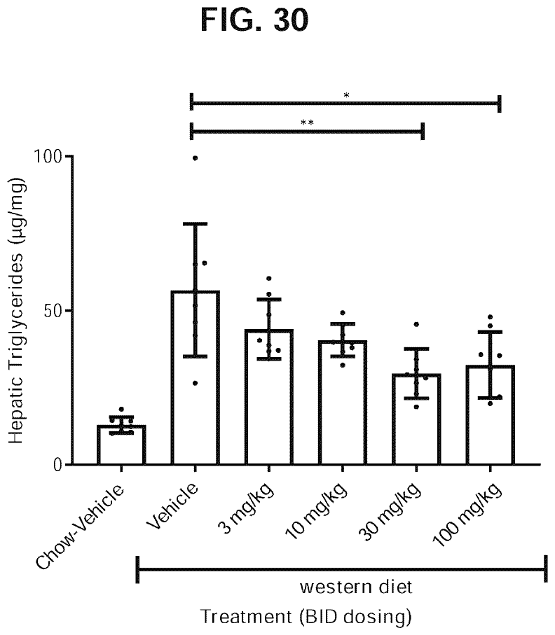

The rat western diet model was utilized to assess the effects of DGAT2 inhibitor treatment on plasma triglyceride production and hepatic triglyceride content in vivo. Male Sprague-Dawley rats were housed under standard laboratory conditions on a 12-hour light, 12-hour dark cycle (lights on at 06:00). Two weeks prior to study start animals were placed on a high-fat, high sucrose, high-cholesterol diet (D12079b, provided by Research Diets, New Brunswick, NJ). This diet provides ˜43% of kilocalories from carbohydrate and ˜41% of kilocalories from fat. Example 4 was administered orally as a solution (10 mL/kg dosing volume) in 0.5% methylcellulose in deionized water, pH 7.0-7.5 (methylcellulose was obtained from Sigma-Aldrich, St. Louis, MO). Vehicle-treated animals received an aqueous solution of 0.5% methylcellulose in deionized water alone, pH 7.0-7.5. Each treatment was administered orally twice daily for 7 days at 08:00 and 16:00 at 3, 10, 30 and 100 mg/kg, for a total daily dose of 6, 20, 60 and 200 mg/kg/day. On day 8, animals were dosed with vehicle or Example 4 at 10:00 and sacrificed 2 hours post-dose. Rats were sacrificed by carbon dioxide asphyxiation and blood collected via lateral tail vein. Plasma TG levels were determined using a Roche Hitachi Chemistry analyzer according to the manufacturer's instructions (Roche Diagnostics Corporation, Indianapolis, IN) and data was analyzed using GraphPad Prism (GraphPad Software, Inc., La Jolla, CA). The liver was collected at sacrifice for determination of hepatic triglyceride and the tissue was immediately frozen in liquid nitrogen, and held at −80° C. until analysis. For assessment of hepatic triglyceride levels, a section of liver wrapped in aluminum foil was pulverized with a hammer, on an aluminum heat block in a liquid nitrogen bath. Pulverization of the liver tissue produced a homogeneous powder. Homogenization buffer, Tris pH 7.4, 98.9 milliliters 0.9% NaCl and 100 microliters of Triton X 100, was mixed on a stir plate for 10 minutes prior to using. Sample weights of approximately one-hundred milligrams of homogenous liver tissue were weighed and placed in Lysing Matrix D tube (MP Biomedicals, Cat #6913-100) with 1 mL of homogenization buffer. All samples were then placed in the FastPrep FP120 (MP Biomedicals, Cat #6001-120) for 2 minutes or until tissue was homogenized. All samples were then spun for 30 seconds at 10,000 g, to clear foam from homogenization. 50 Microliters of sample was transferred to a sterile mixing plate with 450 microliters of Dulbecco's phosphate-buffered saline (DPBS) to create a 1:10 dilution. Upon re-suspension of the new sample, all samples were transferred to sampling tubes for the Siemens Advia XPT Clinical Analyzer. The triglyceride assay was performed through absorbance and reported as milligrams per deciliter. Triglycerides were then normalized per gram of tissue in Microsoft excel. As summarized in FIGS. 29 and 30, there was a dose dependent reduction in plasma (up to ˜70%) and liver (up to 48%) triglycerides in rats administered Example 4. In the instance of the circulating triglyceride response, the resulting levels observed with Example 4 approached those of the chow-fed vehicle animals.

FIG. 29 plots the multiple dose effects of Example 4 on Plasma Triglyceride in Western Diet Fed Sprague-Dawley Rats where plasma triglyceride levels were determined from blood drawn from the lateral tail vein 2-hours following the final dose of Example 4. Data are presented as mean±standard deviation where individual points represent a unique animals. After testing for normality the data was log transformed and an ANOVA was performed taking account for unequal variance with a Dunnett post hoc test then applied to adjust for multiple comparisons against the western diet vehicle treated group. **=p<0.01 versus western diet vehicle-treated animals; *=p<0.05 versus western diet vehicle-treated animals.

FIG. 30 plots the multiple dose effects of Example 4 on Hepatic Triglyceride in Western Diet Fed Sprague-Dawley Rats where hepatic triglyceride levels were determined from blood drawn from the lateral tail vein 2-hours following the final dose of Example 4. Data are presented as mean±standard deviation where individual points represent a unique animal. After testing for normality the data was log transformed and an ANOVA was performed taking account for unequal variance with a Dunnett post hoc test then applied to adjust for multiple comparisons against the western diet vehicle treated group. ****=p<0.0001 versus western diet vehicle-treated animals and ***=p<0.001 versus western diet vehicle-treated animals.

### Determination of pKa

The exemplified compounds were designed to be basic inhibitors of DGAT2. The pKa of selected examples was determined by Analiza, Inc. (Cleveland, OH), according to the capilliary electrophoresis method described in Shalaeva, M., et al. 2008, J. Pharm. Sci., 97, 2581-2606. Table 6 below shows the most basic pKa determined for the examples and are presented as the mean along with the number of replicates (n). Basic compounds are associated with higher volume of distribution in vivo (Obach, R. S., et al. 2009, Drug Metab. Dispos., 36, 1385-1405; Smith, D. A., et al. 2015, J. Med. Chem., 58. 5691-5698).

TABLE 13
Most basic pKa for selected examples

| Example | Basic pKa | n | Example | Basic pKa | n |
|---|---|---|---|---|---|
| 1 | 7.7 | 1 | 2 | 7.3 | 3 |
| 3 | 8.1 | 3 | 4 | 7.5 | 3 |
| 5 | 7.3 | 2 | 6 | 7.4 | 2 |
| 7 | 7.5 | 1 | 8 | 5.3 | 1 |
| 9 | 4.0 | 1 | 10 | 4.7 | 1 |
| 11 | 4.5 | 1 | 12 | 7.5 | 1 |
| 13 | 6.7 | 2 | 14 | 9.5 | 1 |
| 16 | 6.4 | 1 | 17 | 5.4 | 2 |
| 18 | 5.2 | 1 | 19 | 5.2 | 1 |
| 21 | 9.1 | 1 | 25 | 6.8 | 1 |

### Determination of Intrinsic Clearance in Human Hepatocytes (Relay Method)

For intrinsic clearance (CLᵢₙₜ) measurement, hepatocyte relay method was used (Di, L., et al. 2012, Drug Metab. Dispos., 40, 1860-1865 and Di, L., et al. 2013, Drug Metab. Dispos., 41, 2018-2023). Cryopreserved human hepatocytes (Lot DCM from BioreclamationIVT) were used. Upon thawing, the hepatocytes were resuspended in Williams' medium E (custom formula number 91-5233EA; Gibco, Grand Island, NY) supplemented with Hepes and Na₂CO₃. The cells were counted using the trypan blue exclusion method, and the 24-well hepatocyte plates containing 0.5 million cells/mL were spiked with the test compound at a final concentration of 1 μM (dimethyl sulfoxide, final concentration 0.025%; methanol, final concentration 0.1125%), in a final incubation volume of 0.50 mL. The plates were incubated at 37° C. with 95% air/5% CO₂, 75% relative humidity for 4 h at 150 rpm in a humidified incubator. At time 0 and 4 h, 25 μL of hepatocyte suspension was removed from the incubation and added to 50 μL of ice-cold acetonitrile (containing metoprolol, indomethacin, and terfenadine as internal standards) to quench the reaction. The samples were centrifuged (Eppendorf, Hauppauge, NY) at 3000 rpm (1439×g) for 10 min at room temperature, and 50 μL of supernatant was transferred to a clean plate, dried completely, and reconstituted before liquid chromatography/tandem mass spectrometry (LC-MS/MS) analysis. The remaining hepatocyte suspensions in the incubation plate were centrifuged (3000 rpm, 1439×g, 10 min, room temperature). The supernatant of 300 μL was transferred to a clean 24-well plate and stored at −80° C. until the next relay experiment. For the second relay experiment, the supernatant plates were warmed first to room temperature for 30 minutes, then to 37° C. for 30 min, and hepatocytes were added to the samples to give a final cell density of 0.5 million cells/mL. The plates were incubated at 37° C. for 4 h, sampled, and processed as described above. Five relays were performed to give a total incubation time of 20 h, with sampling points at (0, 4, 8, 12, 16 and 20 h). The concentrations of test compound determined at each time point by the LC-MS/MS analysis were used to calculate the intrinsic clearance.

Table 14 below shows the intrinsic clearance for selected Examples as determined by the method described above. The data are presented as the mean+/− standard deviation, with the number of replicates (n) shown.

TABLE 14
CL_{int, u} in the relay hepatocyte assay

| Example | CLᵢₙₜ (μL/min/million cells) | |
|---|---|---|
| number | (+/− Std Dev) | n |
| 4 | 0.98 +/− 0.22 | 2 |
| 5 | 1.9 +/− 0.47 | 2 |

### High Throughput Determination of Intrinsic Clearance in Human Hepatocytes

The high throughput human hepatocyte stability assay was performed in a 384-well format (Di, L., et al. 2012, Eur. J. Med. Chem., 57, 441-448). Pooled cryopreserved human hepatocytes of 10 donors were purchased from BioreclamationlVT (Baltimore, MD, Lot DCM). The cryopreserved human hepatocytes were thawed, and re-suspended in Williams E medium (WEM GIBCO, custom formula #A28859EA) supplemented with HEPES and Na₂CO₃. The cells were counted using the Trypan Blue exclusion method. The Multidrop® liquid dispenser (Multidrop DW, Thermo Scientific, Waltham, MA) was used to add the hepatocyte suspensions to the 384-well plates. The cell plates were covered and transferred to a Sciclone® ALH 3000 workstation (Caliper Life Sciences, Hopkinton, MA), equipped with two 6-position Mecour heat exchangers. Test compounds were diluted on the Sciclone® with buffer and added to the hepatocytes. The final incubation contained 0.5 million cells/mL and 1 μM test compound in 15 μL total volume with 0.01% DMSO. The incubation was carried out at 37° C. At various time points (0, 3, 10, 30, 60, 120, 240 min), the reactions were quenched with cold acetonitrile containing internal standard (Example 39A, WO1999/57125). The samples were centrifuged (Eppendorf, Hauppauge, NY) at 3000 rpm for 5 min at 4° C. The supernatants were transferred using the BioMek® FX liquid handler (Beckman Coulter, Inc. Danvers MA) to new plates with water addition, which were sealed prior to LC-MS/MS analysis. Table 15 below shows the intrinsic clearance for selected examples as determined in the high throughput human hepatocyte assay described above. The data are presented as the mean+/− standard deviation, with the number of replicates (n) shown.

TABLE 15
CLᵢₙₜ in the high throughput human hepatocyte assay

| Exam- | CLᵢₙₜ (μL/min/10⁶ | | Exam- | CLᵢₙₜ (μL/min/10⁶ | |
|---|---|---|---|---|---|
| ple | cells) (+/− Std Dev) | n | ple | cells) (+/− Std Dev) | n |
| 1 | <2.7 +/− 0.72 | 10 | 2 | 3.6 +/− 1.2 | 22 |
| 3 | <1.9 +/− 0.35 | 20 | 4 | <1.8 +/− 0.041 | 14 |
| 5 | <1.9 +/− 0.39 | 10 | 6 | 4.7 +/− 1.0 | 12 |
| 7 | <1.8 +/− 0.30 | 10 | 8 | 12 +/− 1.6 | 2 |
| 9 | 54 +/− 8.4 | 2 | 10 | 27 +/− 3.6 | 2 |
| 11 | 38 +/− 2.0 | 2 | 12 | <2.6 +/− 0.56 | 10 |
| 13 | 12 +/− 0.23 | 2 | 14 | <1.8 +/− 0.11 | 10 |
| 15 | 5.3 +/− 0.68 | 2 | 16 | 4.7 +/− 1.4 | 10 |
| 17 | 8.3 +/− 0.71 | 2 | 18 | 9.3 +/− 1.2 | 2 |
| 19 | 14 +/− 1.4 | 2 | 25 | 6.5 +/− 2.1 | 10 |

### Determination of Intrinsic Clearance in Human Liver Microsomes

The high throughput human microsomal stability assay was performed in a 384-well format (Di, L., et al. 2012, Eur. J. Med. Chem., 57, 441-448). All liquid handling and incubation were conducted with a Biomek FX (Beckman Coulter, Inc., Indianapolis, IN), equipped with one 3-position Mecour heated deck positions. Pooled human liver microsomes of 50 donors (Lot: HLM-103) were purchased from BD Biosciences (Bedford, MA). Each incubation contained test compound (1 μM), human liver microsomes (0.25 μM CYP protein equivalent to 0.806 mg/mL protein concentration), NADPH 20.9 mM, MgCl₂ (3.3 mM) and potassium phosphate buffer (100 mM at pH 7.4). The final reaction volume was 45 μL containing 0.1% DMSO. The incubations were conducted at 37° C. At various time points (e.g. 1, 4, 7, 12, 20, 25, 45 and 60 min), cold acetonitrile with mass spectrometry (MS) internal standard (Example 39A, WO1999/57125) was added to quench the reaction. The plates were centrifuged at 3000 rpm for 1 min at 4° C. (Sorvall RC 3C Plus, Thermo Scientific, Waltham, MA). The plates were sealed and subsequently analyzed using LC-MS/MS. Control plates were prepared in the same manner without adding the NADPH cofactor to monitor any non-CYP/FMO catalyzed decline. Table 16 below shows the intrinsic clearance for selected examples as determined in the human liver microsome assay described above. The data are presented as the mean+/− standard deviation, with the number of replicates (n) shown.

TABLE 16
CLᵢₙₜ in the human liver microsome assay

| Exam- | CLᵢₙₜ (μL/min/mg) | | Exam- | CLᵢₙₜ (μL/min/mg) | |
|---|---|---|---|---|---|
| ple | (+/− STD Dev) | n | ple | (+/− STD Dev) | n |
| 1 | <7 | 1 | 2 | <7 | 1 |
| 3 | <7 | 3 | 4 | <7 | 1 |
| 5 | <7 | 3 | 6 | <7.8 +/− 1.5 | 3 |
| 7 | <7 | 1 | 8 | <14 +/− 10 | 2 |
| 9 | >290 | 2 | 10 | 270 +/− 12 | 2 |
| 11 | >290 | 2 | 12 | <7 | 1 |
| 13 | 35 +/− 3.4 | 3 | 14 | <8 | 1 |
| 15 | 21 | 1 | 16 | 17 | 1 |
| 17 | 23 | 1 | 18 | 19 +/− 1.5 | 2 |
| 19 | 34 +/− 1.9 | 2 | 20 | 8.9 | 1 |
| 21 | <8 | 1 | 22 | <8 | 1 |
| 23 | <8 | 1 | 24 | <8 | 1 |
| 25 | 11 +/− 2.3 | 3 | | | |

### Determination of Thermodynamic Solubility in Various Media

The solubility of active pharmaceutical ingredients is an important characteristic in determining biological performance and ease of formulation during drug development, with high solubility being preferred (Klein, S. 2010, The AAPS Journal, 12, 397-406; Di, L., et al 2012, Drug Disc. Today, 17. 486-495). Thermodynamic solubility was measured in various biorelevant media, as shown in Table 17. A test sample of crystalline solid (˜7 mg) was combined in a vial with 1 mL of the relevant buffer solution and the mixture vortexed to combine. If the solid dissolved completely, additional solid was added with vortexing until a saturated solution was obtained. The saturated solution/solid mixture was capped and subjected to temperature cycling as follows: 1 min at 25° C.; 8 h at 40° C.; 5 h at 15° C. and 12 h at 25° C. The mixture was filtered in a centrifugal filtration device (0.22 μm PVDF filter, MilliporeSigma, Milwaukee, WI) at 13,000 rpm and the concentration of the test compound in the filtrate was determined by HPLC/UPLC with reference to a three point standard curve. Phosphate buffered saline (PBS, pH 6.8, 50 mM phosphate buffer, 250 mM NaCl). Simulated gastric fluid (SGN pH 1.2, USP recipe). Fasted state simulated intestinal fluid (FaSSIF, 3 mM sodium taurocholate, 0.75 mM phospholipid from soybean lecithin, 50 mM phosphate buffer, ionic strength adjusted to 250 mM with NaCl, pH 6.8) and fed state simulated intestinal fluid (FeSSIF, 15 mM sodium taurocholate, 3.75 mM phospholipid from soybean lecithin, 144 mM acetic acid, 50 mM phosphate buffer, ionic strength adjusted to 250 mM with NaCl, pH 6.8).

TABLE 17
Thermodynamic Solubility in Biorelevant Media

| Example | | Solubility |
|---|---|---|
| number | Buffer | (mg/mL) |
| 4 | PBS, pH 6.8 | 0.58 |
| 4 | SGN pH 1.2 | >27 |
| 4 | FaSSIF pH 6.8 | 0.7 |
| 4 | FaSSIF pH 6.8 | 1.21 |

### Pre-Clinical Data

A randomized, vehicle-controlled, 8-parallel arm study was conducted in male Sprague-Dawley rats (Charles River (Boston, MA)) to obtain circulating and hepatic TG levels. Standard laboratory conditions were used to house 96 rats (˜200 g); they were double housed and kept under 12:12-hour reverse light-dark schedule (lights off at 8:00 AM). Rats were randomized into chow or Western diet groups and dose groups upon arrival and were given a 14-day lead-in period on either standard rat chow or Western diet prior to the start of the study. Standard Laboratory Rodent chow diet, 5053, was from LabDiet (PMI, St Louis, Missouri). Western Diet, D12079Bi, was from Research Diets (New Brunswick, NJ).

For these studies, vehicle was prepared to make a 0.5% (wt/volume) of methyl cellulose (MC, Sigma Aldrich, 274429) in deionized water. The stock solutions with either Compound A (prepared from the tris salt) or Compound D were prepared with the respective compound to provide a concentration such that 10 ml of the solution would deliver the desired mg/kg dosage amount, where the average weight of the rats used was about 200 g.

Starting on Day 1 of the study, rats were dosed orally (10 mL/kg) with either vehicle control (0.5% MC (wt/volume %) in dionized water), low or high doses of Compound A (1 mg/kg or 10 mg/kg QD, respectively), low or high doses of Compound D (5 mg/kg or 30 mg/kg BID respectively), or co-administration of low doses (Compound A at 1 mg/kg QD and Compound D at 5 mg/kg BID), or co-administration of high doses (Compound A at 10 mg/kg QD and Compound D at 30 mg/kg BID)

Fed plasma analytes: Blood for determining fed plasma TG concentrations was collected 2 hours post dose (2 hours into the dark cycle) via lateral tail vein, transferred to BD Microtainer tubes coated with dipotassium ethylenediaminetetraacetic acid (K2EDTA) (PN365974), and centrifuged at 4° C. The resulting plasma samples were then analyzed on a Siemens Chemistry XPT clinical analyzer (Malven, PA) using Siemens triglycerides_2 assay reagents (ref 10335892).

Fasted plasma analytes: Blood for determining fasted plasma TG was collected after a 4 hour fast, 2 hours post-dose (2 hours into the dark cycle) via lateral tail vein, transferred to BD Microtainer tubes coated with K2EDTA (PN365974), and centrifuged at 4° C. The resulting plasma samples were then analyzed on a Siemens Chemistry XPT clinical analyzer (Malven, PA) using Siemens triglycerides 2 assay reagents (ref 10335892).

On the last day of the study (Day 28), rats were sacrificed for tissue collection after a 4 hour fast, 2 hours post-dose: two hours post-dose, blood for determining plasma analytes was collected via lateral tail vein and then the animals were sacrificed by CO₂ asphyxiation. Blood was transferred to BD Microtainer tubes coated with K2EDTA (PN365974), centrifuged at 4° C. and the plasma transferred to a 96-well microtitre plate and stored at −20° C. Livers were rapidly removed, freeze-clamped in a Wollenberg clamp pre-cooled in liquid N₂ individually wrapped in aluminum foil and subsequently stored at −80° C.

Tissue pulverization: Frozen livers were rapidly pulverized on an aluminum block cooled in liquid N₂, ensuring the tissue remained frozen throughout the pulverization. The pulverized tissues were transferred and stored in 7 mL polypropylene conical tubes at −80° C. until analysis.

Extraction for hepatic triglyceride: Approximately 50 to 100 mg of pulverized tissue was added to a 2 mL lysing matrix D tube (MP Bio) containing 800 μL ice cold 1:1 CHCl₃:MEOH. Samples were immediately extracted at 4° C. using Qiagen Tissue Lyser II (Qiagen Cat No. 85300) for 4 minutes at 30 Hz. The homogenate was then transferred to 13×100 mm glass tubes and placed on ice. The lysis tubes were then rinsed with 800 μL of 1:1 CHCl₃:MeOH, vortexed for 30 seconds and added to the 13×100 mm glass tubes. While on ice, 2.4 ml of 100% CHCl₃ was added to all glass vials to bring the ratio of CHCl₃:MeOH to 4:2. Samples were then placed in the −20° C. freezer overnight. On the following day, 1.75 mL 1 M KCl H₂O was added to bring the ratio to 4:2:1.75 ratio CHCl₃:MeOH:H₂O. Samples were then vortexed for 30 seconds and centrifuged at 1500 rpm×15 min., at 4° C. After centrifugation, the organic phase was transferred to a fresh 13×100 mm extraction tube, dried down at 37° C. under N₂ and re-suspended in 750 μL CHCl₃. Aminopropyl solid phase extraction (SPE) cartridges (Waters Cat No. 054560, 6 mL, 500 mg) were wetted and washed with 5 mL hexane. After the wash, 200 μL of sample extract in CHCl₃ was applied to the cartridge and removed by vacuum without drying the column. The neutral lipids were then eluted with 5 ml 2:1 CHCl₃: isopropanol/50 μM butylated hydroxytoluene. Samples were then dried down at 37° C. under N₂ and re-suspended with 1.75 ml of 98:2 Isooctane: Isopropanol. Samples were filtered through 0.2 μM syringe filter, before injection onto an HPLC Cyanopropy column (3.5 μM particle size-4.6×150 mm column Agilent Zorbax Eclipse XBD-CN). Running method was a 4 μL injection with a 27 minutes run time using solvent A (1000:1:2, isooctane:isopropanol:acetic acid) and solvent B (50:50 isopropanol:methyl tert-butyl ether). From minute 0-3, solvent composition was held at 100% solvent A. From minute 3-8, solvent composition was changed from 100% solvent A to 95% solvent A and 5% solvent B. From minute 8-18, solvent composition was changed to a 50:50 ratio. From minute 18-19, solvent composition was changed back to 100% solvent A and held at that composition from minutes 19-27.

Nuclear and Membrane fractions were prepared by ultracentrifugation using standard methods from a portion of the pulverized liver samples, that were pooled per treatment group. Samples from the nuclear extract and the membrane fractions were analyzed by Western blotting for SREBP1. Western blots for Calnexin was used as a marker for the membrane fraction, actin as a marker for total sample loading, and Histone 2B as a marker for the nuclear fraction. Nuclear SREBP1 levels were quantified using relative units and normalized to Histone 2B to control for sample loss during the nuclear fractionation and gel loading.

Another portion of the pulverized liver was processed and analyzed for lipogenic gene expression. Rat taqman probes against ACC1, FASN, SCD1, PCSK9 and SREBP-1c were all assessed using Actb as housekeeping gene on qPCR.

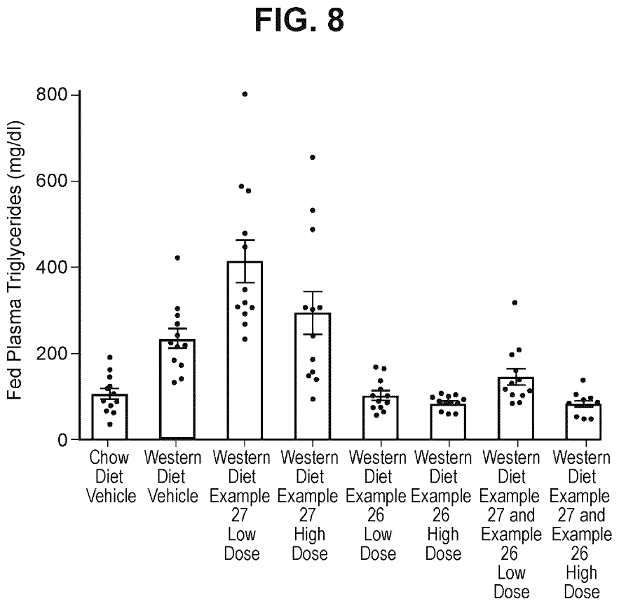

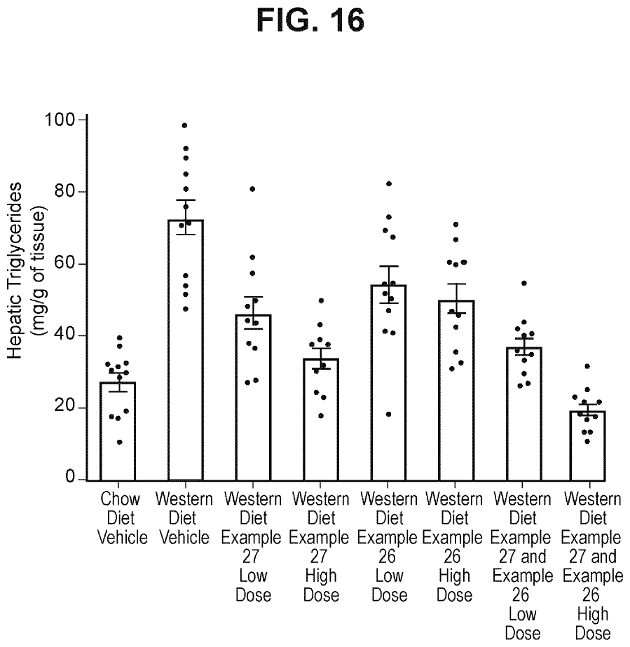

Administration of (S)-2-(5-((3-ethoxypyridin-2-yl)oxy)pyridin-3-yl)-N-(tetrahydrofuran-3-yl)pyrimidine-5-carboxamide (Compound D), or pharmaceutically acceptable salt thereof, in combination with 4-(4-(1-isopropyl-7-oxo-1,4,6,7-tetrahydrospiro[indazole-5,4′-piperidine]-1′-carbonyl)-6-methoxypyridin-2-yl)benzoic acid (Compound A), or pharmaceutically acceptable salt thereof, has resulted in significantly decreased plasma (FIG. 8, 9) and liver TG (FIG. 16) levels compared to the plasma and liver TG levels when administering with Compound A as monotherapy.

Western diet feeding resulted in a 2.2 fold increase in fed-state plasma TG, relative to chow fed rats (FIG. 8). Oral administration of either the low dose (1 mg/kg QD) or the high dose (10 mg/kg QD) of only Compound A (monotherapy) resulted in a 1.7 fold and 1.3 fold increase, respectively, in plasma TG in the fed state, relative to vehicle-administered Western diet fed rats. Conversely, oral administration of either the low dose (5 mg/kg BID) or the high dose (30 mg/kg BID) of only Compound D (monotherapy) reduced plasma TG in the fed state by 55% and 63%, respectively, relative to vehicle-administered Western diet fed rats. Co-administration of Compound A and Compound D resulted in complete blockade of the Compound A mediated increases plasma TG in the fed state. Oral co-administration of Compound A and Compound D at either both at the low dose or both at the high dose reduced fed-state plasma TG levels by 37% and 64%, relative to vehicle-administered Western diet fed rats.

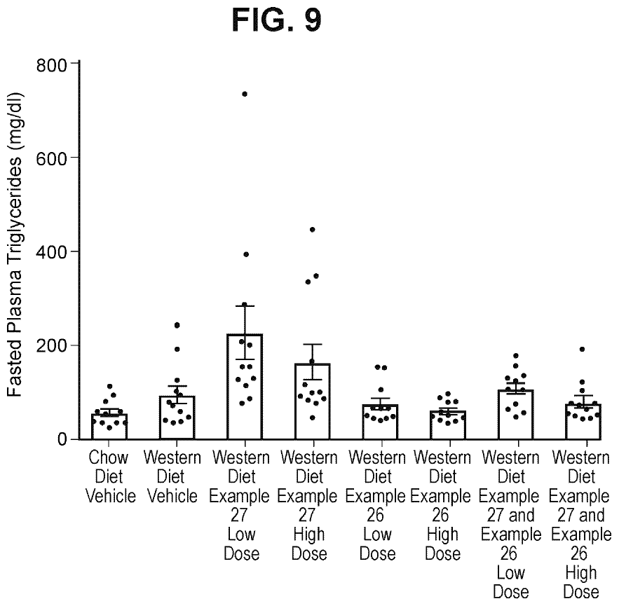

Western diet feeding resulted in a 1.6 fold increase in fasted plasma TG, relative to chow fed rats (FIG. 9). Oral administration of the low and high doses of Compound A as a monotherapy resulted in a 2.4 fold and 1.7 fold increase, respectively, in fasted plasma TG, relative to vehicle administered Western diet fed rats. Conversely, oral administration of the low and high doses of Compound D as a monotherapy reduced fasted plasma TG by 20% and 35%, respectively, relative to vehicle administered Western diet fed rats. Oral co-administration of Compound A and Compound D at both the low dose of each or the high dose of each fully mitigated the Compound A mediated increase in fasted plasma TG observed when administering only Compound A. Fasted plasma TG levels for both the low dose group (109 mg/dl) and high dose group (81 mg/dl) of the co-administered Compound A and Compound D were similar to vehicle administered Western diet fed rats (96 mg/dl).

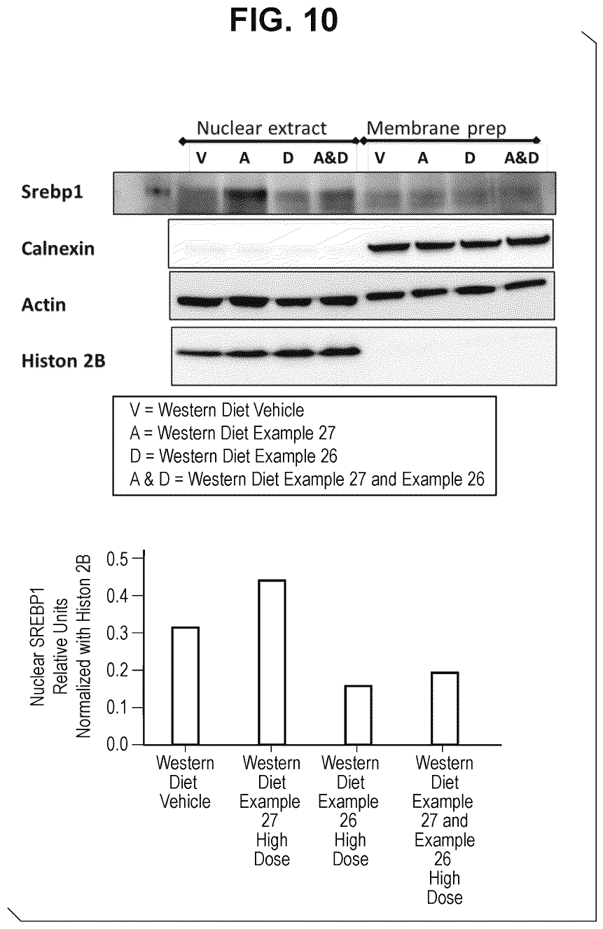

Nuclear SREBP-1 localization was compared in samples from Western diet fed rats administered vehicle, high dose Compound A monotherapy, high dose Compound D monotherapy, or co-administered high dose Compound A and high dose Compound D (FIG. 10). Relative to vehicle treated Western diet fed rats, administration of Compound A produced increased nuclear localization of SREBP-1 indicative of increased SREBP-1 activation. Conversely, administration of Compound D reduced SREBP-1 nuclear localization and SREBP-1 activation. Co-administration of Compound A and Compound D blocked the Compound A mediated increase in nuclear SREBP-1 localization producing a 50% decrease compared with monotherapy of only Compound A.

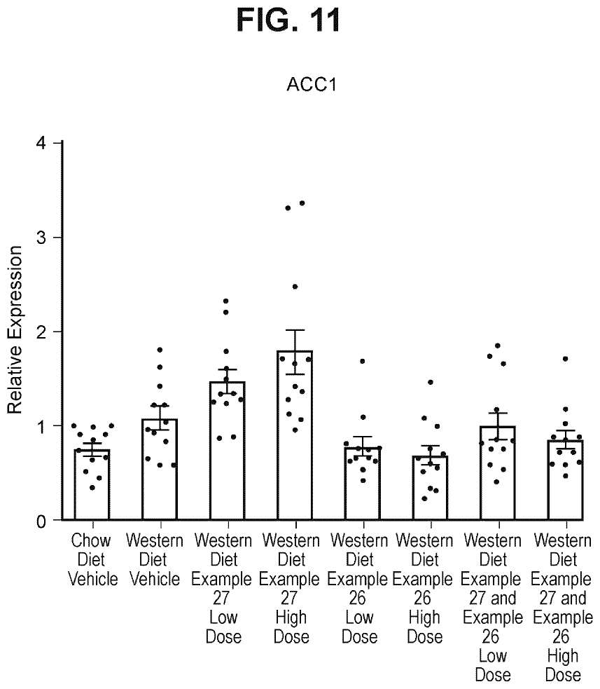

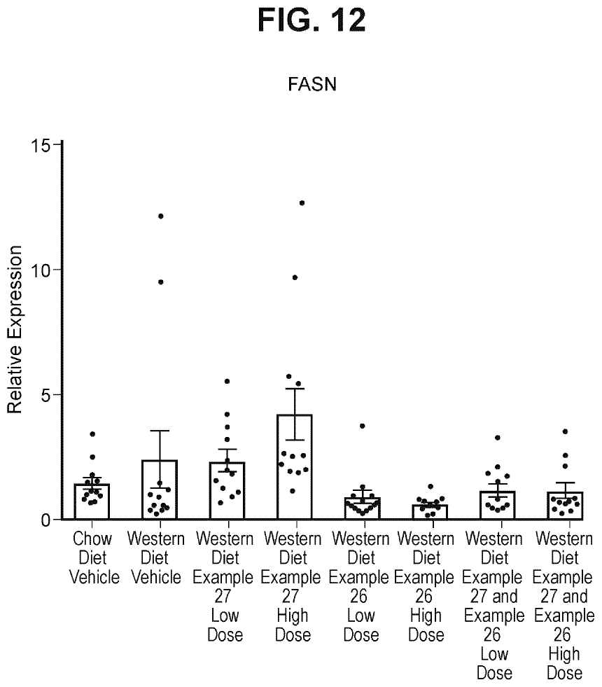

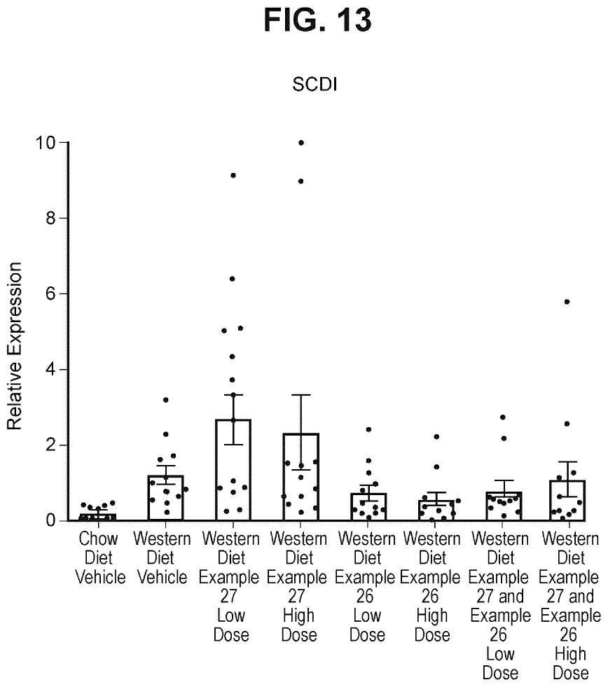

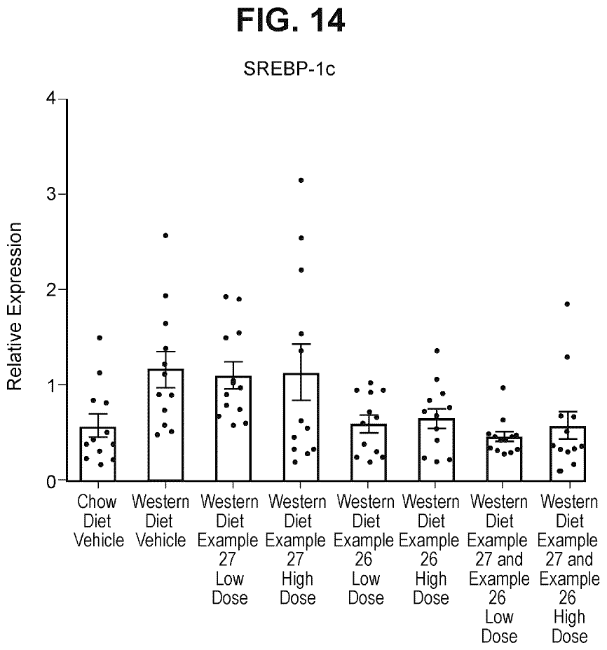

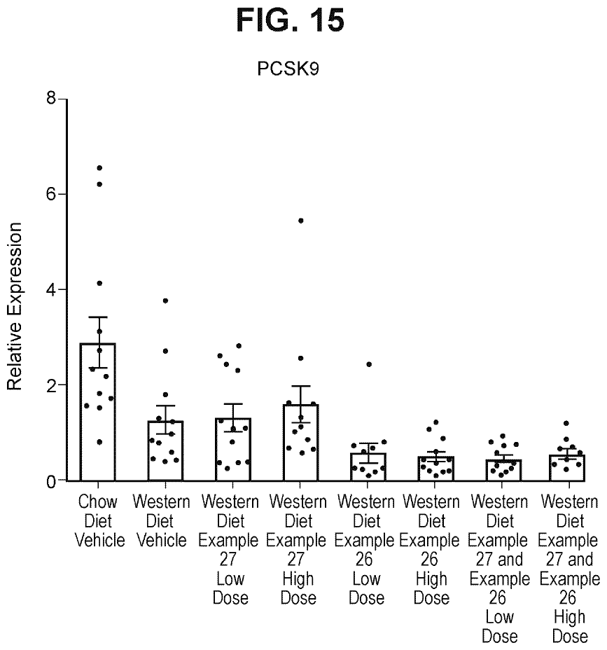

Relative to chow fed vehicle treated rats, animals fed a Western diet and treated with vehicle trended to show increased expression of the lipogenic genes: ACC1 (FIG. 11), FASN (FIG. 12), SCD1 (FIG. 13) and SREBP1 (FIG. 14), but not PCSK9 (FIG. 15), which was lower in the Western diet fed rats. Administration of Compound A trended to further increase relative to Western Diet fed and vehicle treated animals, the expression of ACC1, FASN (Compound A high dose only), SCD, but not PCSK9 and SREBP1. Conversely, administration of Compound D decreased expression of all of the lipogenic genes. Co-administration of Compound A and Compound D resulted in expression levels being comparable or lower than those observed in vehicle treated Western diet fed rats.

Relative to chow fed rats, vehicle administered Western diet fed rats showed about a 2.7-fold increase in hepatic triglyceride accumulation (FIG. 16). Oral administration of the low dose and high dose of Compound A produced a 36% and 53% reduction, respectively, in steatosis, relative to vehicle administered Western diet fed rats. Similarly, oral administration of the low dose and high dose of Compound D reduced steatosis by 25% and 30%, respectively, relative to vehicle administered Western diet fed rats. Oral co-administration of either the low or high dose of both Compound A and Compound D reduced steatosis by 50% and 73%, respectively, relative to vehicle administered Western diet fed rats. Oral administration of either the low dose or high dose combination of Compound A and Compound D produced greater reductions in steatosis than that observed with administration of either Compound A or Compound D as monotherapy at the same dose levels.

A randomized, vehicle-controlled, 5-parallel arm study was conducted in male Wistar-Han rats (Charles River (Boston, MA)) fed a choline-deficient and high fat diet (CDAHFD) (Research diets; A16092003) to identify differences in improvements in markers of hepatic inflammation and fibrosis when administering either Compound A or Compound D alone as monotherapy or in combination. Standard laboratory conditions were used to house 60 rats (˜200 g); they were double housed and kept under 12:12-hour reverse light-dark schedule (lights off at 8:00 AM). Rats were fed choline deficient and high fat diet (CDAHFD) beginning 6-weeks prior to initiation of the study. Rats, randomized into 4 dosing groups (n=12/group), received twice daily administration of vehicle, Compound A (5 mg/kg) monotherapy, Compound D (30 mg/kg) monotherapy, or co-administration Compound A (5 mg/kg) and Compound D (30 mg/kg) for a period of 6-weeks. Animals (n=12) remaining on normal chow throughout the study and administered twice-daily vehicle were used as a control group. Blood samples were collected prior to starting compound administration and 3- and 6-week post compound administration for the assessment of circulating markers. Shearwave elastography (Aixplorer Ultimate imager, Supersoinc imagine) measurements were made at Week −3, Week 0 (prior to 1^{st} dose), Week 3 and Week 6 to assess inflammation and fibrosis progression over time. Histology was assessed following 6-weeks of drug administration which corresponded to 12 weeks on the CDAHFD. Results are provided as an average of animals per each dosing group.

Following the 12-weeks on CDAHFD, the animals were sacrificed by CO₂ asphyxiation. The right lateral, medial and left lateral lobes of the liver were harvested. Sections were taken from the left lateral, right medial and right lateral lobes and fixed in formalin and processed to paraffin blocks per animal. One section of left lateral lobe per animal was cryopreserved in optimal cutting temperature (OCT) compound. The remainder of the liver from each animal was frozen and rapidly pulverized on an aluminum block cooled in liquid N₂, ensuring the tissue remained frozen throughout the pulverization. The pulverized tissue was transferred and stored at −80° C. until analysis. A portion of the pulverized liver sample from each animal was processed and analyzed for gene expression markers of fibrogenesis. Rat taqman probes against αSMA and COL1A1 were all assessed using Actb as housekeeping gene on qPCR.

The following endpoints were evaluated by qualitative histologic evaluation by a board certified veterinary pathologist and quantitative histomorphometry: hepatic stellate cell activation and differentiation into myofibroblasts by αSMA immunohistochemistry (IHC); Collagen as a correlate of fibrosis by Picrosirius Red stain. Images were analyzed using Visiopharm software. Visiopharm applications with threshold parameters were applied uniformly to identify tissue sections and to quantify the targets on each IHC (DAB (3,3′-diaminobenzidine) positive) or histochemically stained slides as percent area: stain area of interest/Total tissue ROI−whitespace)×100%. Non-parametric statistics were used to analyze data from this study. Group values were reported as mean+/− the standard error of the mean.

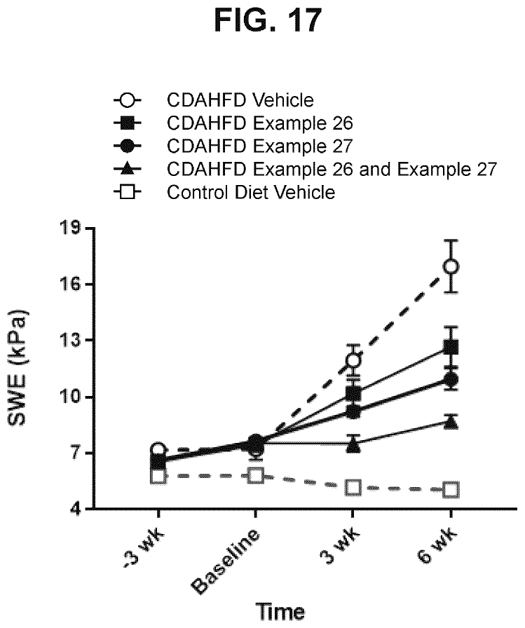

Relative to control animals fed a chow diet and administered vehicle, animals that received CDAHFD and administered vehicle showed a marked increase in liver stiffness (assessed using shearwave elastography (SWE), measured in kilopascals (kPa)) over the duration of the study, indicative of progressive hepatic inflammation and fibrosis (FIG. 17). Administration of Compound A or Compound D as monotherapy each reduced liver stiffness suggestive of reduced hepatic inflammation and/or fibrosis. Co-administration of Compound A and Compound D produced greater reductions in liver stiffness than either agent as monotherapy (FIG. 17).

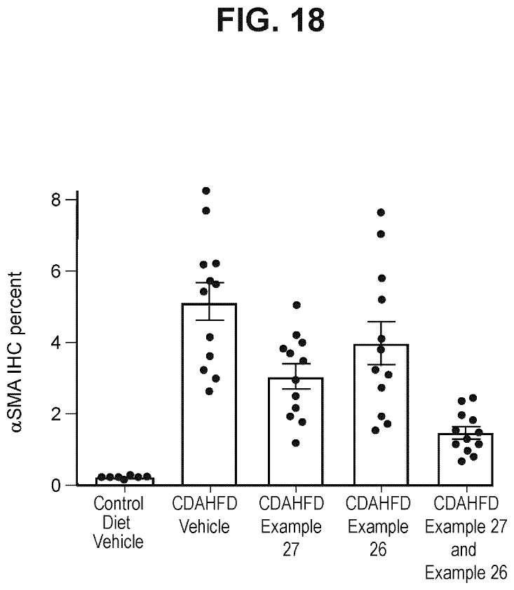

Relative to control animals fed a chow diet and administered vehicle, animals that received CDAHFD and administered vehicle showed a marked increase in liver alpha smooth muscle actin (αSMA) staining, indicative of myofibroblast activation and fibrogenesis (FIG. 18). Administration of Compound A or Compound D as monotherapy each reduced αSMA staining by 41% and 23%, respectively, suggestive of reduced hepatic myofibroblast activation and fibrogenesis. Co-administration of Compound A and Compound D produced greater reductions in αSMA staining than either agent as monotherapy, reducing staining by 72% (FIG. 18).

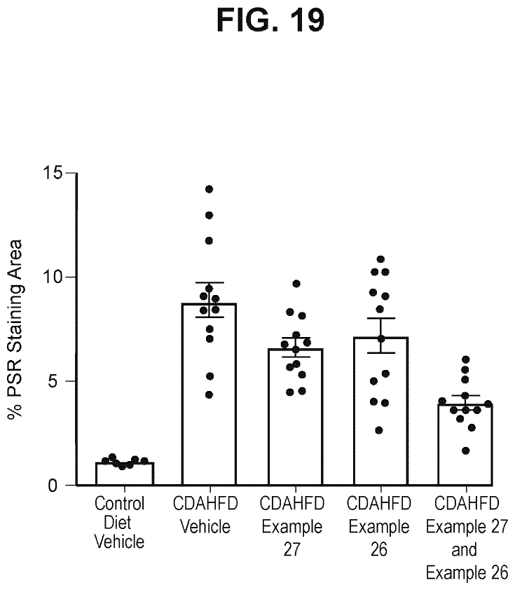

Relative to control, animals fed a chow diet and administered vehicle, animals that received CDAHFD and administered vehicle showed a marked increase in Picosrius red (PSR) staining, indicative of collagen deposition and fibrosis (FIG. 19). Administration of Compound A or Compound D as monotherapy each reduced PSR staining by 26% and 20%, respectively, suggestive of reduced collagen deposition and fibrosis. Co-administration of Compound A and Compound D produced greater reductions in PSR staining than either agent as monotherapy, reducing staining by 56% (FIG. 19).

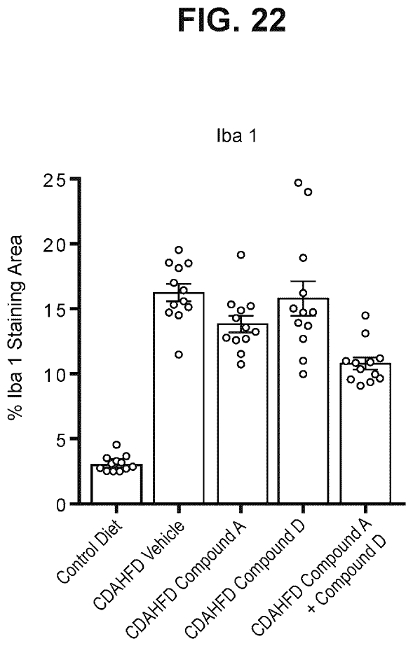

Relative to control, animals fed a chow diet and administered vehicle, animals that received CDAHFD and administered vehicle showed a marked increase in Ionized calcium binding adaptor molecule 1 (Iba1) staining, indicative of hepatic macrophage activation (FIG. 22). Administration of Compound A as monotherapy reduced Iba1 staining by 15%, suggestive of reduced hepatic inflammatory tone. While administration of D as monotherapy did not alter Iba1 staining, co-administration of A and D produced greater reductions in Iba1 staining than Compound A administered as monotherapy, decreasing staining by 33% (FIG. 22)

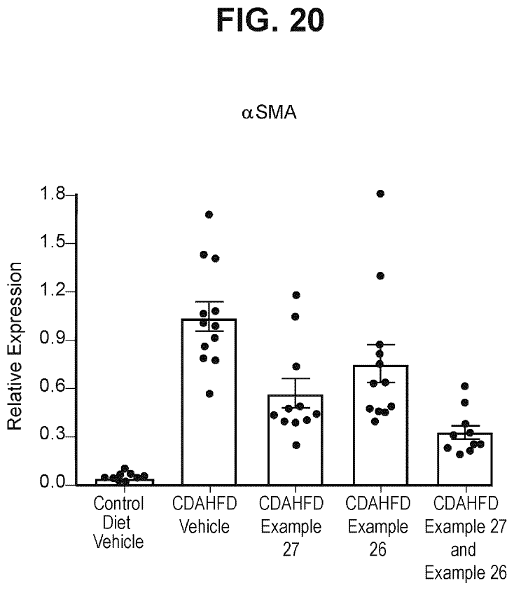

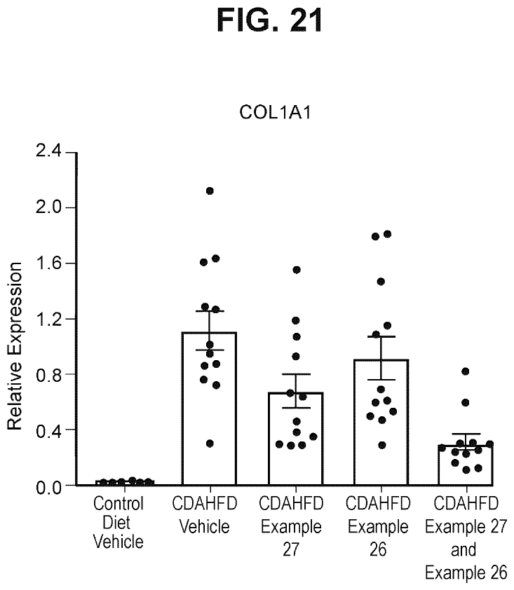

Relative to control animals fed a chow diet and administered vehicle, animals that received CDAHFD and administered vehicle showed a marked increase in liver alpha smooth muscle actin (αSMA) (FIG. 20) and collagen A1A (COL1A1) (FIG. 21) gene expression, indicative of myofibroblast activation and fibrogenesis. Administration of Compound A or Compound D as monotherapy each reduced hepatic αSMA and COL1A1 gene expression, suggestive of reduced hepatic myofibroblast activation and fibrogenesis. Co-administration of Compound A and Compound D produced greater reductions in hepatic αSMA (FIG. 20) and COL1A1 (FIG. 21) gene expression than either agent as monotherapy.

Potency data for Compound D (Example 27 and Compound of Example 4 is shown in Table 18 below:

| | | Primary human hepatocyte |
|---|---|---|
| Compound | Biochemical IC50 (nM), (n) | IC50 (nM), (n) |
| Compound D | 17 (n = 6) | 2.8 (n = 10) |
| Example 4 | 10 (n = 22) | 11 (n = 13) |

### Phase II Pharmacodynamic, Safety and Tolerability Study

A Phase 2A, randomized, double blind, placebo controlled, parallel group study was conducted to assess the pharmacodynamics, safety and tolerability of the compound of Example 26 (Compound A) and the compound of Example 27 (Compound D) co-administered for six weeks in adults with non-alcoholic fatty liver disease (NAFLD). The co-administration of the compounds included oral administration of Compound D as a 300 mg dose (3 tablets of 100 mg each) every 12 hours and oral administration of Compound A was administered as a 15 mg dose (3 tablets of 5 mg each) every 12 hours.

It was hypothesized that inhibition of acetyl-CoA (coenzyme A) carboxylase 1 and 2 by

Compound A and inhibition of diacylglycerol acyltransferase 2 by Compound D would modulate lipid metabolism in distinct and complementary ways, suggesting that co-administration of the two compounds may lead to an improved benefit-risk profile compared with either agent administered alone. The results for the primary PD endpoint of whole liver fat (WLF), key secondary endpoints regarding safety (platelets) and tolerability, and key tertiary/exploratory PD and biomarker endpoints including lipids (e.g. triglycerides, TG) and liver function tests (LFTs) are summarized in Table 19 below:

TABLE 19
Summary of Treatment Effects of Whole Liver Fat, Triglycerides and
Platelets (Change from Baseline to Day 42, Placeb-adjusted).

| | | Placebo-adjusted % Change from Baseline (N = 12) on Day 42 | | |
|---|---|---|---|---|
| | | Liver Fat PDFF | Triglycerides | Platelets |
| Treatment | N | LSMean (90% CI) | LSMean (90% CI) | LSMean (90% CI) |
| Cmpd A 15 mg Q12H | 22 | −44.52 (−54.97, −31.65) | 47.30 (21.77, 78.19) | −7.12 (−14.51, 0.91) |
| Cmpd D 300 mg Q12H | 24 | −35.40 (−47.40, −20.68) | −8.63 (−24.37, 10.38) | 7.50 (−0.99, 16.71) |
| Cmpd A 15 mg Q12H + | 26 | −44.64 (−54.80, −32.19) | 6.00 (−12.21, 27.99) | 1.23 (−6.75, 9.91) |
| Cmpd D 300 mg Q12H | | | | |

CI = Confidence Interval

Key findings for whole liver fat (WLF) and triglycerides on day 42 show: i) all treatment arms met the prespecified decision criteria for WLF (i.e. ≥95% confidence that the treatment arm is better than placebo; and observed placebo-adjusted reductions greater than the target value of 30%); ii) monotherapy with Compound A induced a marked triglyceride elevation, and monotherapy with Compound D led to a modest decrease in triglycerides; and iii) co-administration of Compound A and Compound D led to triglyceride values minimally changed compared with placebo, resulting in robust mitigation of the triglyceride elevation seen with the Compound A monotherapy.

Key safety and tolerability findings show: i) all treatment arms were generally safe and well tolerated over the course of the study; and ii) no clinically meaningful change in platelets was observed in any treatment arm. Compared with placebo, a decrease in platelets was observed with Compound A monotherapy arm. This was not apparent with Compound D. Surprising, the decrease in platelets was ameliorated in the Compound A/Compound D co-administration arms.

In summary, co-administration of Compound A/Compound D was generally safe and well tolerated, provided greater WLF reduction than administration of Compound D alone and substantially mitigated induced triglyceride elevation associated with monotherapy with Compound A.

### Baseline Characteristics

Table 20 below provides information on the key demographic and baseline characteristics of the study population.

TABLE 20

| | | | | Compound A |
|---|---|---|---|---|
| | | Compound A | Compound | (15 mg Q12H) + |
| | Placebo | (15 mg | D (300 mg | Compound |
| | Q12H | Q12H) | Q12H) | D (300 mg |
| N | 14 | 29 | 29 | 28 |
| Age | 56.9 (13.3) | 55.3 (10.6) | 53.5 (11.0) | 53.2 (9.6) |
| Gender: Male | 7 (50.0) | 15 (51.7) | 13 (46.4) | 18 (64.3) |
| Race: White | 13 | 25 | 26 | 25 |
| Other | 1 | 4 | 2 | 3 |
| Weight(kg) | 95.94 | 99.68 | 100.48 | 104.69 |
| BMI (kg/m²) | 34.69 | 35.01 | 36.16 | 36.96 |
| Hemoglobin A1c (%) | 5.7 (0.4) | 6.1 (0.5) | 5.8 (0.5) | 5.8 (0.6) |
| Platelets (10³/mm³) | 271.4 (40.2) | 269.8 (57.2) | 263.4 (55.7) | 250.5 (49.4) |
| [130-394] | | | | |
| Fasting Glucose (mg/dL) | 99.1 (10.3) | 115.1 (29.3) | 109.4 (22.0) | 105.9 (22.3) |
| Whole liver fat (%) | 15.6 (7.6) | 16.0 (6.6) | 17.7 (8.2) | 16.1 (6.9) |

Liver Function Tests

| | | | | |
|---|---|---|---|---|
| ALT (U/L) | 35.0 (23.0) | 31.9 (15.7) | 36.0 (18.5) | 32.8 (16.1) |
| AST(U/L) | 25.9 (9.5) | 24.0 (8.1) | 26.0 (8.5) | 23.5 (7.4) |
| Alkaline Phosphatase | 91.9 (37.4) | 85.8 (32.3) | 82.4 (15.8) | 84.0 (18.3) |
| (U/L) | | | | |
| GGT (U/L) | 38.0 (25.0) | 32.9 (17.0) | 37.2 (17.7) | 34.6 (19.3) |

Lipids & Apolipoproteins

| | | | | |
|---|---|---|---|---|
| Triglycerides (mg/dL) | 164.2 (82.1) | 214.2 (134.9) | 173.3 (90.5) | 175.3 (66.8) |
| Total Cholesterol | 193.4 (57.5) | 182.7 (41.4) | 193.9 (29.1) | 194.5 (36.6) |
| HDL-Cholesterol(mg/dL) | 46.3 (8.4) | 43.4 (9.7) | 47.6 (11.1) | 43.1 (8.7) |
| Direct LDL-Cholesterol | 129.8 (50.6) | 112.8 (39.8) | 126.9 (24.6) | 129.3 (31.7) |
| (mg/dL) | | | | |
| Apolipoproteins C3 | 13.6 (4.5) | 15.2 (5.9) | 14.2 (5.6) | 13.9 (7.0) |

Mean (SD) is presented forcontinuousvariables; count (%) is presented forgender

### Primary Whole Liver Fat by MRI-PDFF

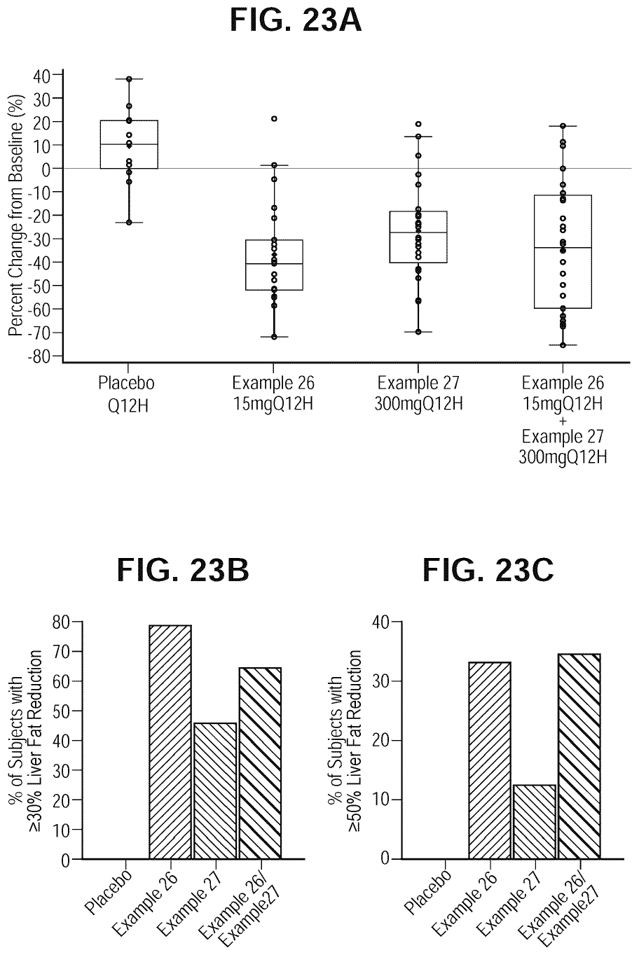

Log-transformed relative change from baseline to Day 42 in WLF by MRI-PDFF was analyzed using the analysis of covariance (ANCOVA). The model included log-transformed baseline WLF by MRI-PDFF as a covariate. Model-derived estimates were back-transformed from the log scale and converted to percent change. FIG. 23a shows a Box-and-Whisker plot of the WLF data by treatment arm and Table 21 provides the results from the ANCOVA model.

TABLE 21

| | | | | | % Change of Combination | |
|---|---|---|---|---|---|---|
| | | % Change from | | | vs Each Monotherapy | |
| | | Baseline | % Change from Placebo | | LS Mean | |
| | | LS Mean | LS Mean | 2-sided | (50% CI)* | 2-sided |
| Treatment | N | (90% CI) | (90% CI) | P-value | (90% CI) | P-value |
| Placebo | 12 | 8.14 | | | | |
| | | (−8.56, 27.89) | | | | |
| Cmpd A 15 mg | 22 | −40.01 | −44.52 | <0.0001 | −0.21 | 0.9836 |
| | | (−47.00, −32.09) | (−54.97, −31.65) | | (−6.82, 6.87) | |
| | | | | | (−15.66, 18.08) | |
| Cmpd D 300 mg | 24 | −30.14 | −35.40 | 0.0007 | −14.30 | 0.1233 |
| | | (−37.97, −21.33) | (−47.40, −20.68) | | (−19.86, −8.35) | |
| | | | | | (−27.32, 1.06) | |
| Cmpd A 15 mg + | 26 | −40.13 | −44.64 | <0.0001 | | |
| Cmpd D 300 mg | | (−46.58, −32.90) | (−54.80, −32.19) | | | |

All treatment arms lead to a reduction in WLF from baseline on Day 42, as compared to placebo which showed a numerical increase. Each treatment arm met the prespecified decision criteria for WLF versus placebo (i.e. ≥95% confidence that the treatment arm is better than placebo; and observed placebo-adjusted reductions greater than the target value of 30%). Furthermore, comparison of the magnitude of reduction in WLF across arms showed evidence that the Compound A and the Compound D co-administration leads to a numerically greater reduction than monotherapy with Compound D and comparable reduction to monotherapy with Compound A [−0.21% (50% C₁-6.82, 6.87)] (see Table 21).

FIGS. 23b and 23c demonstrate the proportion of participants that meet liver fat reduction thresholds of ≥30% or ≥50% relative reduction in liver fat.

### Serum Triglycerides

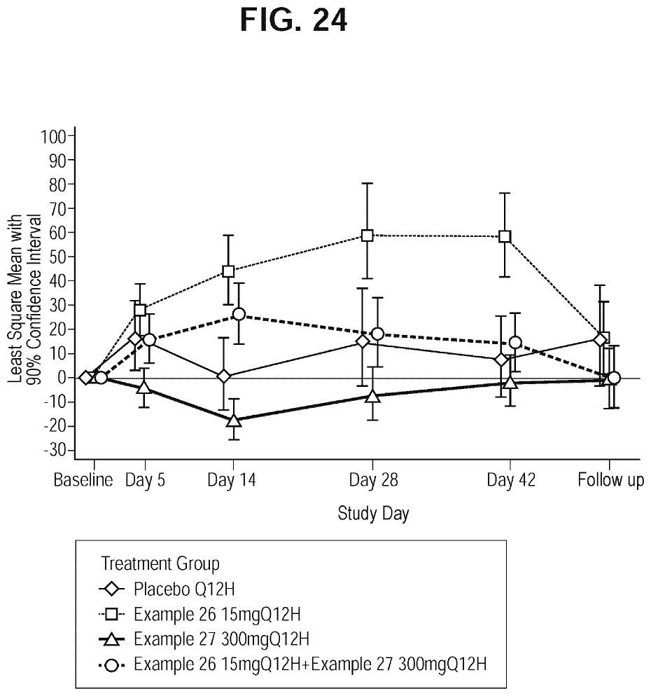

Log-transformed relative change from baseline in serum triglycerides were analyzed using a mixed model for repeated measurements (MMRM). The model included log-transformed baseline TG and baseline whole liver fat as covariates. Model-derived estimates were back-transformed from the log scale and converted to percent change. Table 22a provides a statistical analysis of the percent change from baseline in serum triglycerides—FAS. FIG. 24 is a plot of least square means and 90% Cls for percent change from baseline in serum triglycerides—FAS.

TABLE 22a

| | | | % Change from | | | % Change of Combination | |
|---|---|---|---|---|---|---|---|
| | | | Baseline | % Change from Placebo | | vs Each Monotherapy | |
| Study | | | LS Mean | LS Mean | 2-sided | LS Mean | 2-sided |
| Day | Treatment | N | (90% CI) | (90% CI) | P-value | (90% CI) | P-value |
| 5 | Placebo | 13 | 16.72 | | | | |
| | | | (3.18, 32.03) | | | | |
| | Cmpd A 15 mg | 29 | 27.89 | 9.57 | 0.3117 | −9.53 | 0.1706 |
| | | | (17.67, 38.99) | (−5.63, 27.22) | | (−13.87, −4.98) | |
| | Cmpd D 300 mg | 26 | −4.59 | −13.26 | 0.0286 | 21.26 | 0.0107 |
| | | | (−12.52, 4.05) | (−29.69, −4.97) | | (15.34, 27.50) | |
| | Cmpd A 15 mg + | 26 | 15.70 | −0.87 | 0.9231 | | |
| | Cmpd D 300 mg | | (6.05, 26.22) | (−14.76, 15.27) | | | |
| 14 | Placebo | 12 | 0.42 | | | | |
| | | | (−13.37, 16.40) | | | | |
| | Cmpd A 15 mg | 27 | 43.67 | 43.06 | 0.0012 | −12.58 | 0.1176 |
| | | | (30.12, 58,62) | (19.72, 70.96) | | (−17.47, −7.39) | |
| | Cmpd D 300 mg | 27 | −17.53 | −17.87 | 0.0689 | 52.29 | <0.0001 |
| | | | (−25.31,−8.93) | (−31.25, −1.90) | | (43.76, 61.32) | |
| | Cmpd A 15 mg + | 26 | 25.60 | 25.07 | 0.0405 | | |
| | Cmpd D 300 mg | | (13.54, 38.93) | (4.59, 49.56) | | | |
| 28 | Placeb | 12 | 14.74 | | | | |
| | | | (−3.60, 36.57) | | | | |
| | Cmpd A 15 mg | 24 | 59.36 | 38.89 | 0.0121 | −25.99 | 0.0043 |
| | | | (41.06, 80.03) | (12.25, 71.85) | | (−30.96, −20.67) | |
| | Cmpd D 300 mg | 26 | −7.35 | −19.25 | 0.0951 | 27.29 | 0.0193 |
| | | | (−17.72, 4.33) | (−34.59, −0.31) | | (18.86, 36.31) | |
| | Cmpd A 15 mg + | 26 | 17.93 | 2.79 | 0.8290 | | |
| | Cmpd D 300 mg | | (4.69, 32.86) | (−16.77, 26.93) | | | |
| 42 | Placebo | 12 | 7.38 | | | | |
| | | | (−8.06, 25.41) | | | | |
| | Cmpd A 15 mg | 22 | 58.17 | 47.30 | 0.0011 | −28.03 | 0.0006 |
| | | | (41.76, 76.48) | (21.77, 78.19) | | (−32.39, −23.40) | |
| | Cmpd D 300 mg | 24 | −1.89 | −8.63 | 0.4290 | 16.02 | 0.1081 |
| | | | (−11.94, 9.32) | (−24.37, 10.38) | | (9.05, 23.43) | |
| | Cmpd A 15 mg + | 26 | 13.83 | 6.00 | 0.6080 | | |
| | Cmpd D 300 mg | | (2.27, 26.69) | (−12.21, 27.99) | | | |

Table 22a shows the results from the MMRM model at each time-point. The data shows that an ACCi-induced triglyceride elevation was observed with monotherapy administration with Compound A. However, consistent with the study hypothesis, co-administration of Compound A/Compound D led to mitigation of the triglyceride elevation. The placebo-adjusted increase in triglycerides at Day 42 was 47.30% (21.77%, 78.19%) on Compound A treatment arm but only 6.00% (−12.21%, 27.99%) on the Compound A/Compound D combination arm. This equates to a statistically significant reduction of 28.03% (23.40%, 32.39%) in triglyceride levels on the combination arm relative to Compound A monotherapy arm. Triglyceride elevation at Day 5 and Day 14 in the combination arm was of lower magnitude than that seen in Compound A monotherapy arm, with values at Day 28 and Day 42 similar to placebo (see FIG. 24).

In addition, Table 22b provides a summary of triglyceride abnormalities in NAFLD patients. Data is presented as the total number (%) of subjects exceeding triglyceride threshold values. The data set shows mitigation of Compound A-mediated triglyceride abnormalities at >400 mg/dl; >600 mg/dl; and >800 mg/dl. Specifically, the data shows complete blockade of the Compound A-mediated triglyceride abnormalities (>600 mg/dl; and >800 mg/dl) by the co-administration of Compound A and Compound D.

TABLE 22b

| | | ACCi | DGAT2i | ACCi:DGAT2i |
|---|---|---|---|---|
| | Placebo | (15 mg | (300 mg | (15 mg:300 mg |
| | (BID) | BID) | BID) | BID) |
| Number of subjects | 14 | 29 | 27 | 26 |
| evaluable | | | | |
| >400 mg/dl | 2 (14.3) | 11 (37.9) | 2 (7.4) | 3 (11.5) |
| >600 mg/dl | 1 (7.1) | 4 (13.8) | 1 (3.7) | 0 |
| >800 mg/dl | 0 | 3 (10.3) | 0 | 0 |

### Other Lipids and Apolipoproteins

A summary of the statistical analysis of percent change from baseline in other fasting serum lipid parameters on day 42—FAS is provided in Table 23.

TABLE 23

| | | | % Change | | | % Change of Combination | |
|---|---|---|---|---|---|---|---|
| | | | from Baseline | % Change from Placebo | | vs Each Monotherapy | |
| | | | LS Mean | LS Mean | 2-sided | LS Mean | 2-sided |
| Day 42 | Treatment | N | (90% CI) | (90% CI) | P-value | (90% CI) | P-value |
| Total | Placebo | 12 | 1.98 | | | | |
| Cholesterol | | | (−4.27, 8.63) | | | | |
| | Cmpd A 15 mg | 22 | −2.72 | −4.61 | 0.3153 | −5.22 | 0.1583 |
| | | | (−7.02, 1.77) | (−11.73, 3.09) | | (−10.97, 0.91) | |
| | Cmpd D 300 mg | 24 | −5.87 | −7.70 | 0.0878 | −2.05 | 0.5795 |
| | | | (−9.93, −1.63) | (−14.55, −0.30) | | (−7.92, 4.20) | |
| | Cmpd A 15 mg + | 26 | −7.80 | −9.58 | 0.0317 | | |
| | Cmpd D 300 mg | | (−11.71, −3.71) | (−16.26, −2.38) | | | |
| HDL- | Placebo | 12 | −3.19 | | | | |
| Cholesterol | | | (−9.87, 3.98) | | | | |
| | Cmpd A 15 mg | 22 | −15.56 | −12.77 | 0.0110 | −3.14 | 0.4517 |
| | | | (−19.70, −11.20) | (−20.08, −4.80) | | (−9.70, 3.90) | |
| | Cmpd D 300 mg | 24 | −13.43 | −10.58 | 0.0354 | −5.52 | 0.1890 |
| | | | (−17.62, −9.03) | (−18.02, −2.45) | | (−11.91, 1.33) | |
| | Cmpd A 15 mg + | 26 | −18.21 | −15.51 | 0.0018 | | |
| | Cmpd D 300 mg | | (−22.13, −14.09) | (−22.53, −7.85) | | | |
| LDL- | Placebo | 12 | 2.04 | | | | |
| Cholesterol | | | (−9.52, 15.09) | | | | |
| | Cmpd A 15 mg | 22 | −19.74 | −21.35 | 0.0081 | 12.81 | 0.0953 |
| | | | (−26.24, −12.67) | (−32.09, −8.92) | | (0.17, 27.07) | |
| | Cmpd D 300 mg | 24 | −5.22 | −7.12 | 0.4030 | −4.46 | 0.5198 |
| | | | (−12.81, 3.02) | (−19.77, 7.52) | | (−15.08, 7.47) | |
| | Cmpd A 15 mg + | 26 | −9.45 | −11.27 | 0.1775 | | |
| | Cmpd D 300 mg | | (−16.68, −1.59) | (−23.34, 2.72) | | | |
| Non-HDL | Placebo | 12 | 2.17 | | | | |
| Cholesterol | | | (−5.74, 10.74) | | | | |
| | Cmpd A 15 mg | 22 | 1.86 | −0.30 | 0.9600 | −5.94 | 0.2046 |
| | | | (−3.82, 7.87) | (−9.68, 10.06) | | (−13.15, 1.86) | |
| | Cmpd D 300 mg | 24 | −3.51 | −5.56 | 0.3355 | −0.70 | 0.8819 |
| | | | (−8.78, 2.05) | (−14.40, 4.19) | | (−8.22, 7.43) | |
| | Cmpd A 15 mg + | 26 | −4.19 | −6.22 | 0.2775 | | |
| | Cmpd D 300 mg | | (−9.34, 1.25) | (−14.96, 3.41) | | | |

The impact of the three treatment arms (Compound A monotherapy, Compound D monotherapy, and Compound A/Compound D co-administration) on key lipid parameters (Total Cholesterol, HDL-Cholesterol, LDL-Cholesterol, Non-HDL-Cholesterol) is shown above in Table 13 for Day 42, comparing each arm to placebo and Compound A/Compound D co-administration arm to each monotherapy arm. For total cholesterol, all treatment arms trended lower than placebo with a statistically significant decrease seen in the Compound A/Compound D co-administration arm [−9.58% (−16.26, −2.38)]. No statistically significant difference was observed when comparing each monotherapy arm with the combination arm. HDL-cholesterol decreased with similar magnitude in all treatment arms, as compared to placebo. Co-administration of Compound A/Compound D did not significantly decrease HDL-Cholesterol lower than each monotherapy. LDL-cholesterol was statistically significantly decreased in the Compound A monotherapy arm, with numerical lowering trends in the Compound D monotherapy and Compound A/Compound D co-administration arms that were not statistically significant. Non-HDL cholesterol was not significantly different from placebo in any treatment arm.

The impact of the three treatment arms (Compound A, Compound D, and Compound A/Compound D co-administration) on ApoA1, ApoB and ApoC3 was measured. The results for the statistical analysis of percent change from baseline in apolipoproteins C3 (ApoC3) on Day 42—FAS is shown in Table 24, comparing each arm to placebo and the Compound A/Compound D co-administration arm to each monotherapy arm. The LS Mean (and 90% Cls) percent change from placebo on Day 42 are given.

TABLE 24

| | | | % Change from | | | % Change of Combination | |
|---|---|---|---|---|---|---|---|
| | | | Baseline | % Change from Placebo | | vs Each Monotherapy | |
| | | | LS Mean | LS Mean | 2-sided | LS Mean | 2-sided |
| Day 42 | Treatment | N | (90% CI) | (90% CI) | P-value | (90% CI) | P-value |
| ApoC3 | Placebo | 12 | 1.82 | | | | |
| | | | (−9.64, 14.72) | | | | |
| | Cmpd A 15 mg | 21 | 45.31 | 42.71 | 0.0002 | −24.66 | 0.0002 |
| | | | (32.75, 59.05) | (22.86, 65.77) | | (−33.31, −14.88) | |
| | Cmpd D 300 mg | 23 | −6.52 | −8.19 | 0.3365 | 17.11 | 0.0302 |
| | | | (−14.25, 1.90) | (−20.75, 6.36) | | (3.96, 31.93) | |
| | Cmpd A 15 mg + | 26 | 9.48 | 7.52 | 0.4064 | | |
| | Cmpd D 300 mg | | (0.91, 18.77) | (−6.96, 24.26) | | | |

ApoC3 increased in a statistically significant manner in the Compound A monotherapy arm [42.71% (22.86, 65.77)]. Surprisingly, ApoC3 did not increase with Compound D monotherapy arm [−8.19% (−20.75, 6.36)], where it remained similar to placebo. Also surpassingly, in the Compound A/Compound D co-administration arm, ApoC3 levels were also similar to placebo [7.52% (−6.96, 24.26)]; thus, Compound A-induced elevation in ApoC3 was mitigated by co-administration with Compound D.

### Other Endpoints of Interest—Liver Function Tests

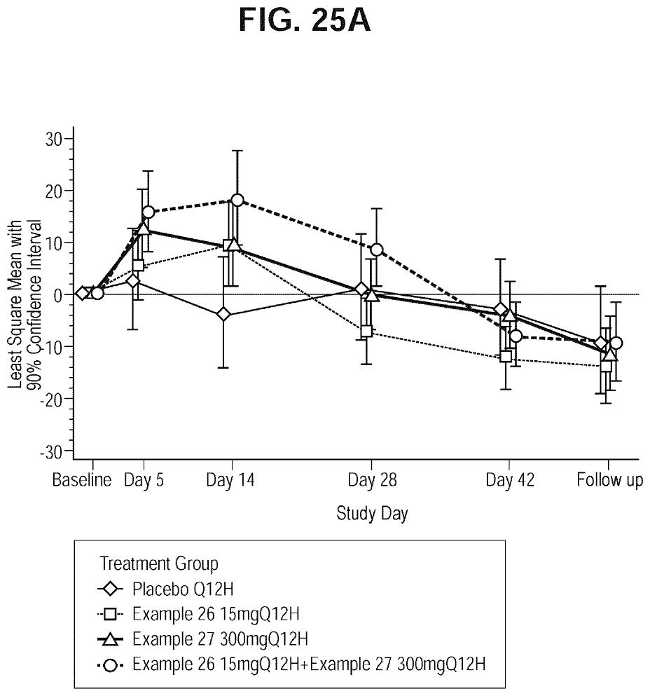

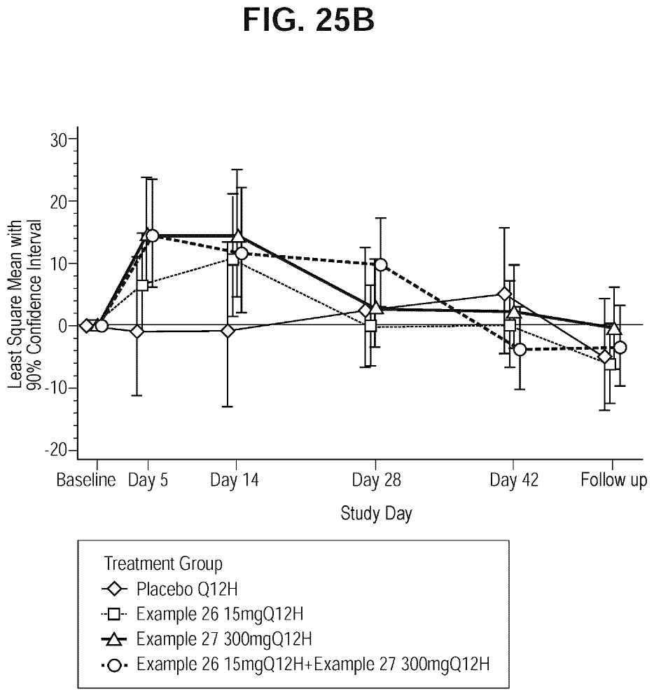

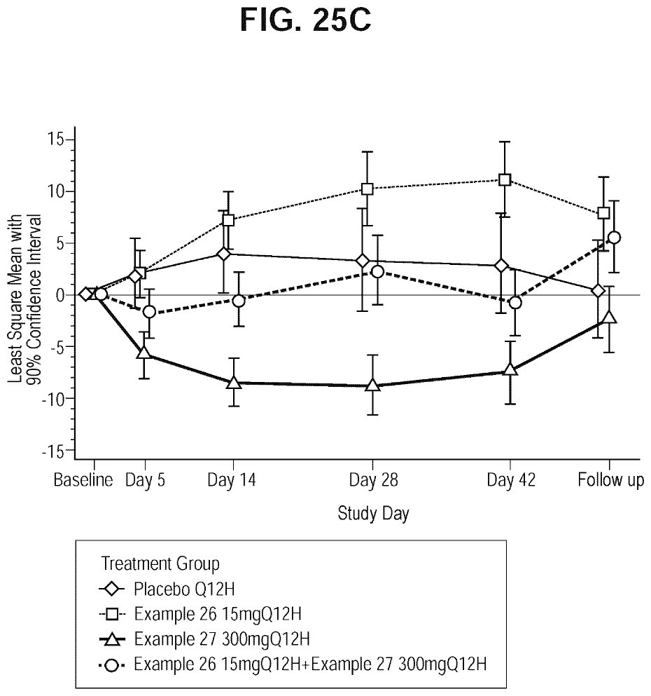

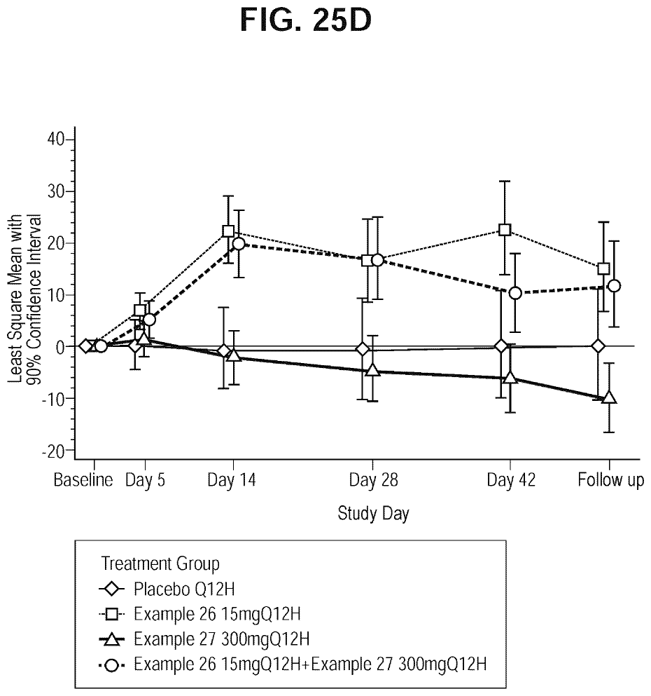

A summary of other endpoints of interest is provided in Table 25. The LS Mean (and 90% CIs) percent change from placebo on Day 42 are given. Table 25 provides a statistical analysis of percent change from baseline in liver function tests on Day 42-FAS. FIGS. 25a-25d provide plots of least square means and 90% CIs for percent change from baseline in liver function tests—FAS. FIG. 25a provides the plot for alanine aminotransferase (ALT); FIG. 25b provides the plot for aspartate aminotransferase (AST); FIG. 25c provides the plot for alkaline phosphotate; and FIG. 25d provides the plot for gamma glutamyl transferase (GGT).

TABLE 25

| | | | % Change from | | | % Change of Combination | |
|---|---|---|---|---|---|---|---|
| | | | Baseline | % Change from Placebo | | vs Each Monotherapy | |
| | | | LS Mean | LS Mean | 2-sided | LS Mean | 2-sided |
| Day 42 | Treatment | N | (90% CI) | (90% CI) | P-value | (90% CI) | P-value |
| ALT | Placebo | 12 | −3.01 | | | | |
| | | | (−11.76, 6.61) | | | | |
| | Cmpd A 15 mg | 22 | −12.41 | −9.70 | 0.1508 | 5.15 | 0.3842 |
| | | | (−18.24, −6.18) | (−19.67, 1.51) | | (−4.42, 15.67) | |
| | Cmpd D 300 mg | 24 | −4.15 | −1.18 | 0.8647 | −3.92 | 0.4803 |
| | | | (−10.30, 2.42) | (−11.95, 10.91) | | (−12.51, 5.53) | |
| | Cmpd A 15 mg + | 24 | −7.91 | −5.05 | 0.4575 | | |
| | Cmpd D 300 mg | | (−13.80, −1.61) | (−15.40, 6.57) | | | |
| AST | Placebo | 12 | 5.13 | | | | |
| | | | (−4.44, 15.65) | | | | |
| | Cmpd A 15 mg | 22 | −0.03 | −4.91 | 0.4798 | −3.84 | 0.5062 |
| | | | (−6.73, 7.15) | (−15.50, 7.01) | | (−12.77, 6.01) | |
| | Cmpd D 300 mg | 22 | 2.21 | −2.77 | 0.6923 | −5.95 | 0.3011 |
| | | | (−4.67, 9.59) | (−13.59, 9.40) | | (−14,73, 3.74) | |
| | Cmpd A 15 mg + | 23 | −3.87 | −8.56 | 0.2100 | | |
| | Cmpd D 300 mg | | (−10.25, 2.96) | (−18.72, 2.88) | | | |
| Alkaline | Placebo | 12 | 2.93 | | | | |
| Phosphatase | | | (−1.78, 7.87) | | | | |
| | Cmpd A 15 mg | 22 | 11.20 | 8.03 | 0.0276 | −10.74 | 0.0001 |
| | | | (7.59, 14.92) | (2.01, 14.40) | | (−14.75, −6.53) | |
| | Cmpd D 300 mg | 24 | −7.51 | −10.15 | 0.0025 | 7.32 | 0.0118 |
| | | | (−10.46, −4.46) | (−15.12, −4.88) | | (2.53, 12.33) | |
| | Cmpd A 15 mg + | 26 | −0.74 | −3.57 | 0.2903 | | |
| | Cmpd D 300 mg | | (−3.88, 2.50) | (−8.90, 2.07) | | | |
| GGT | Placebo | 12 | 0.01 | | | | |
| | | | (−9.77, 10.85) | | | | |
| | Cmpd A 15 mg | 22 | 22.63 | 22.62 | 0.0085 | −10.16 | 0.0808 |
| | | | (14.06, 31.84) | (8.12, 39.07) | | (−18.77, −0.63) | |
| | Cmpd D 300 mg | 24 | −6.31 | −6.32 | 0.3867 | 17.60 | 0.0082 |
| | | | (−12.70, 0.54) | (−17.31, 6.13) | | (6.45, 29.91) | |
| | Cmpd A 15 mg + | 26 | 10.17 | 10.17 | 0.1995 | | |
| | Cmpd D 300 mg | | (2.71, 18.18) | (−2.73, 24.78) | | | |

In all treatment arms, a transient increase in ALT and AST was observed, spanning the first 4 weeks, as compared to placebo. By day 42 both were below baseline values. For alkaline phosphatase, a modest increase is noted in the placebo arm with a clear and statistically significant elevation in the Compound A monotherapy arm. Surprisingly, a clear and statistically significant decrease in alkaline phosphatase was noted in the Compound D treatment arm. In the Compound A/Compound D co-administration arm alkaline phosphatase values fluctuated around the baseline value, with no statistically significant change throughout the study. GGT showed no significant change from baseline in the placebo or the Compound D monotherpay arms, while, surprisingly, there was an increase observed in the Compound A monotherapy arms and Compound A/Compound D co-administration arms. The increase in GGT observed in the co-administration arm was significantly lower than the Compound A monotherapy arm on Day 42 only.

### Safety and Tolerability

Table 26 shows the number of adverse events (all causalities) for subjects in the safety analysis set. Specifically, Table 26 shows treatment emergent adverse events (AEs) (All causalities)—safety analysis set.

TABLE 26

| | | Cmpd A | Cmpd | Cmpd A15 mg + |
|---|---|---|---|---|
| | Placebo | 15 mg | D300 mg | Cmpd D 300 mg |
| Number (%)ofSubjects | n (%) | n (%) | n (%) | n (%) |
| Subjectsevaluable foradverse events | 14 | 29 | 28 | 28 |
| Numberof adverse events | 7 | 17 | 22 | 15 |
| Subjects withadverse events | 3 (21.4) | 10 (34.5) | 10 (35.7) | 10 (35.7) |
| Subjects with seriousadverse events | 0 | 0 | 0 | 1 (3.6) |
| Subjects with severe adverse events | 0 | 1 (3.4) | 0 | 0 |
| Subjects discontinuedfromstudy dueto | 0 | 1 (3.4) | 1 (3.6) | 0 |
| adverse events (a) | | | | |
| Subjectsdiscontinued study drug due to | 0 | 0 | 1 (3.6) | 0 |
| AE and continue Study (b) | | | | |
| Subjects with dose reduced or temporary | 0 | 1 (3.4) | 0 | 0 |
| discontinuationdue to adverseevents | | | | |

(a) Subjects who have an AE record that indicates that the AE caused the subject to be discontinued from the study
(b) Subjects who have an AE record that indicates that action taken with study treatment was drug withdrawn but AE did not cause the subject to be discontinued from study

Incidence of AEs was similar between the Compound A/Compound D co-administration arm and both monotherapy treatment arms. One SAE of ‘Mandibular Abcess’ was reported in the Compound A/Compound D co-administration arm (deemed not treatment related). Two subjects were discontinued from study drug due to AEs. One subject in the Compound A monotherapy arm was discontinued due to severe AE of TG elevation (deemed treatment related); the subject remained asymptomatic. Another subject in the Compound D monotherapy arm was discontinued from study drug due to mild AE of creatine kinase and AST elevations (deemed not treatment related). With the exception of the TG, CK and AST elevations noted above, no major laboratory abnormalities were noted. Overall, all treatments were safe and well tolerated.

### Other Safety Endpoints of Interest

Table 27 provides a statistical analysis of percent change from baseline in platelets on Day 42—FAS.

TABLE 27

| | | | % Change from | | | % Change of Combination | |
|---|---|---|---|---|---|---|---|
| | | | Baseline | % Change from Placebo | | vs Each Monotherapy | |
| | | | LS Mean | LS Mean | 2-sided | LS Mean | 2-sided |
| Day 42 | Treatment | N | (90% CI) | (90% CI) | P-value | (90% CI) | P-value |
| Platelets | Placebo | 12 | −2.40 | | | | |
| | | | (−8.76, 4.40) | | | | |
| | Cmpd A 15 mg | 22 | −9.35 | −7.12 | 0.1423 | 9.00 | 0.0371 |
| | | | (−13.66, −4.82) | (−14.51, 0.91) | | (1.87, 16.62) | |
| | Cmpd D 300 mg | 24 | 4.92 | 7.50 | 0.1473 | −5.83 | 0.1351 |
| | | | (0.10, 9.97) | (−0.99, 16.71) | | (−11.86, 0.62) | |
| | Cmpd A 15 mg + | 25 | −1.2 | 1.23 | 0.8044 | | |
| | Cmpd D 300 mg | | (−5.70, 3.52) | (−6.75, 9.91) | | | |

A numerical trend for decrease over time in platelets was observed in the Compound A monotherapy treatment arm as compared to placebo. No decrease in platelets was noted in the Compound D monotherapy arm. It was surprisingly discovered that no decrease in platelets was also observed with Compound A/Compound D co-administration arms, relative to placebo.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-5. (canceled)

6. A pharmaceutical composition comprising:
2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

7. A pharmaceutical composition comprising:
a therapeutically effective amount from about 10 mg to about 1000 mg of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof, and a therapeutically effective amount of from about 5 mg to about 60 mg of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

8. The pharmaceutical composition according to claim 6, wherein the composition is administered once a day.

9. The pharmaceutical composition according to claim 6, wherein the composition is administered twice a day.

10. The pharmaceutical composition according to claim 6, wherein the 2-{5-[(3-ethoxypyridin-2-yl)oxy]pyridin-3-yl}-N-[(3S,5S)-5-fluoropiperidin-3-yl]pyrimidine-5-carboxamide is a crystal having the structure: 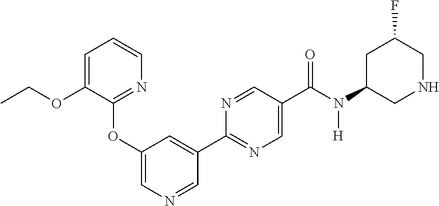 [SMILES: CCOc1cccnc1Oc1cncc(-c2ncc(C(=O)N[C@@H]3CNC[C@@H](F)C3)cn2)c1]
or a pharmaceutically acceptable salt thereof.

11. The pharmaceutical composition of claim 10, wherein the crystal comprises a p-toluenesulfonate salt of the compound.

12. The pharmaceutical composition of claim 10, having a powder x-ray diffraction pattern comprising 2-theta values of (CuKα radiation, wavelength of 1.54056 Å) 7.2±0.2, 14.5±0.2, 15.8±0.2, and 27.7±0.2.

13. The pharmaceutical composition of claim 11, having a powder x-ray diffraction pattern comprising 2-theta values of (CuKα radiation, wavelength of 1.54056 Å) 3.8±0.2, 7.7±0.2, 8.8±0.2, 22.4±0.2, and 24.6±0.2.

14. The pharmaceutical composition according to claim 6, wherein the 4-(4-(1-Isopropyl-7-oxo-1,4,6,7-tetrahydrospiro[indazole-5,4′-piperidine]-1′-carbonyl)-6-methoxypyridin-2-yl)benzoic acid is a crystalline solid of structure: 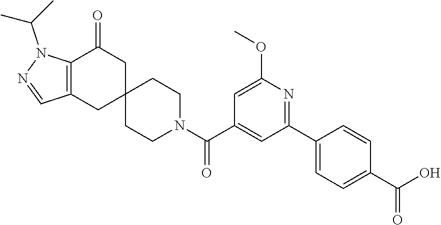 [SMILES: COc1cc(C(=O)N2CCC3(CC2)CC(=O)c2c(cnn2C(C)C)C3)cc(-c2ccc(C(=O)O)cc2)n1]
or a pharmaceutically acceptable salt thereof.

15. The pharmaceutical composition of claim 14, wherein the crystalline solid is 2-amino-2-(hydroxymethyl) propane-1,3-diol salt of 4-(4-(1-isopropyl-7-oxo-1,4,6,7-tetrahydrospiro[indazole-5,4′-piperidine]-1′-carbonyl)-6-methoxypyridin-2-yl)benzoic acid.

16. A method of treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma, the method comprising administering to a human in need of such treatment a therapeutically effective amount of the composition of claim 6.

17. A method for the reduction of at least one point in severity of nonalcoholic fatty liver disease (NAFLD) Activity Score (NAS) from baseline comprising the step of measuring the baseline NAS in a human, administering to said human a therapeutically effective amount of the composition of claim 6- and measuring the NAS of said human.

18. A method for the reduction of at least two points in severity of nonalcoholic fatty liver disease (NAFLD) Activity Score (NAS) from baseline comprising the step of measuring the baseline NAS in a human, administering to said human a therapeutically effective amount of the composition of claim 6 and measuring the NAS of said human.

19.-25. (canceled)

26. The method according to claim 16, wherein the composition is administered once a day.

27. The method according to claim 16, wherein the composition is administered twice a day.

28. The method according to claim 16, wherein the 2-{5-[(3-ethoxypyridin-2-yl)oxy]pyridin-3-yl}-N-[(3S,5S)-5-fluoropiperidin-3-yl]pyrimidine-5-carboxamide is a crystal having the structure: 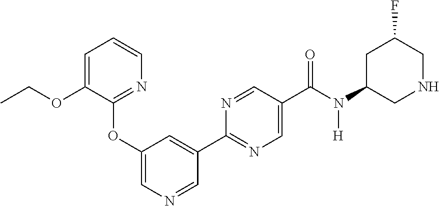 [SMILES: CCOc1cccnc1Oc1cncc(-c2ncc(C(=O)N[C@@H]3CNC[C@@H](F)C3)cn2)c1]
or a pharmaceutically acceptable salt thereof.

29. The method of claim 28, wherein the crystal comprises a p-toluenesulfonate salt of the compound.

30. The method of claim 28, wherein the crystal has a powder x-ray diffraction pattern comprising 2-theta values of (CuKα radiation, wavelength of 1.54056 Å) 7.2±0.2, 14.5±0.2, 15.8±0.2, and 27.7±0.2.

31. The method of claim 29, wherein the crystal has a powder x-ray diffraction pattern comprising 2-theta values of (CuKα radiation, wavelength of 1.54056 Å) 3.8±0.2, 7.7±0.2, 8.8±0.2, 22.4±0.2, and 24.6±0.2.

32. The method according to claim 16, wherein the 4-(4-(1-Isopropyl-7-oxo-1,4,6,7-tetrahydrospiro[indazole-5,4′-piperidine]-1′-carbonyl)-6-methoxypyridin-2-yl)benzoic acid is a crystalline solid of structure: 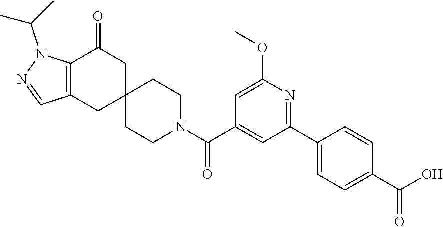 [SMILES: COc1cc(C(=O)N2CCC3(CC2)CC(=O)c2c(cnn2C(C)C)C3)cc(-c2ccc(C(=O)O)cc2)n1]
or a pharmaceutically acceptable salt thereof.

33. The method of claim 32, wherein the crystalline solid is 2-amino-2-(hydroxymethyl) propane-1,3-diol salt of 4-(4-(1-isopropyl-7-oxo-1,4,6,7-tetrahydrospiro[indazole-5,4′-piperidine]-1′-carbonyl)-6-methoxypyridin-2-yl)benzoic acid.

34. A method for treating fatty liver, nonalcoholic fatty liver disease, nonalcoholic steatohepatitis, nonalcoholic steatohepatitis with liver fibrosis, nonalcoholic steatohepatitis with cirrhosis or nonalcoholic steatohepatitis with cirrhosis and hepatocellular carcinoma, the method comprising administering to a human in need of such treatment a first composition comprising a therapeutically effective amount of 2-{5-[(3-ethoxypyridin-2-yl)oxy]pyridin-3-yl}-N-[(3S,5S)-5-fluoropiperidin-3-yl]pyrimidine-5-carboxamide, or pharmaceutically acceptable salt thereof in combination with a second composition comprising a therapeutically effective amount of 4-(4-(1-Isopropyl-7-oxo-1,4,6,7-tetrahydrospiro[indazole-5,4′-piperidine]-1′-carbonyl)-6-methoxypyridin-2-yl)benzoic acid or a pharmaceutically acceptable salt thereof.

35. The method of claim 34, wherein the first and second compositions are administered simultaneously.

36. The method of claim 34, wherein the first and second compositions are administered sequentially.

37. (canceled)
